(19) United States
(12) Patent Application Publication
(10) Pub. No.: US 2025/0205207 A1
(43) Pub. Date: Jun. 26, 2025

(54) MATRIX TABLETS OF DEXPRAMIPEXOLE AND METHODS OF MANUFACTURING AND USE THEREOF

(71) Applicant: Areteia Therapeutics, Inc., Chapel Hill, NC (US)

(72) Inventors: Samir Thakorbhai DESAI, Suwanee, GA (US); Gregory C. WILLIAMS, Mendham, NJ (US)

(73) Assignee: Areteia Therapeutics, Inc., Chapel Hill, NC (US)

(21) Appl. No.: 18/990,723

(22) Filed: Dec. 20, 2024

Related U.S. Application Data
(60) Provisional application No. 63/706,522, filed on Oct. 11, 2024.
(60) Provisional application No. 63/612,916, filed on Dec. 20, 2023.

(51) Int. Cl. A61K 31/428; A61K 9/20; A61K 9/28

(52) U.S. Cl. CPC A61K 31/428; A61K 9/2054; A61K 9/284

(57) ABSTRACT

The present disclosure relates to sustained release pharmaceutical compositions of dexpramipexole, or a pharmaceutically acceptable salt thereof. In some aspects, the present disclosure relates to sustained release pharmaceutical compositions in the form of orally deliverable tablets. In some aspects, the sustained release pharmaceutical compositions of the present disclosure comprise one or more anionic polymers at specific amounts. In some aspects, the sustained release pharmaceutical compositions of the present disclosure comprise one or more neutral polymers at specific amounts. The present disclosure further relates to methods of manufacturing the sustained release pharmaceutical compositions (or intermediate products thereof) and methods of using the sustained release pharmaceutical compositions to treat and prevent certain diseases, such as eosinophilic disorders, in a human subject.

### RELATED APPLICATIONS

This application claims priority to U.S. Provisional Application No. 63/706,522 filed Oct. 11, 2024, which is incorporated hereby by reference in its entirety. This application claims priority to U.S. Provisional Application No. 63/612,916 filed Dec. 20, 2023, which is incorporated hereby by reference it is entirety.

### FIELD OF THE DISCLOSURE

The present disclosure relates to sustained release pharmaceutical compositions of dexpramipexole, or a pharmaceutically acceptable salt thereof. In certain particular aspects, the present disclosure relates to sustained release pharmaceutical compositions in the form of orally deliverable tablets. The present disclosure further relates to methods of manufacturing the sustained release pharmaceutical compositions (or intermediate products thereof), as well as uses of the sustained release pharmaceutical compositions to treat or prevent certain diseases or conditions in human subjects, in particular such diseases or conditions that are related to elevated eosinophilic levels.

### BACKGROUND OF THE DISCLOSURE

Dexpramipexole ((6R)-2-amino-4,5,6,7-tetrahydro-6-(propylamino)benzothiazole; (formula I)) is the enantiomer of pramipexole ((6S)-2-amino-4,5,6,7-tetrahydro-6-(propylamino)benzothiazole; (formula II)).

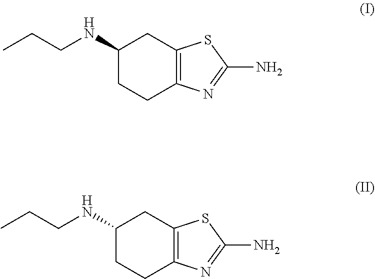

Pramipexole is a dopamine D2 receptor agonist used in the treatment of Parkinson's disease. Pramipexole is sold in the form of orally deliverable tablets under the brand Mirapex®, among others. Mirapex® tablets are available as immediate release (IR) and extended release (ER) formulations, and contain pramipexole as the dihydrochloride monohydrate salt. Mirapex® ER tablets for oral administration are available at a dose of 0.375 mg, 0.75 mg, 1.5 mg, 2.25 mg, 3 mg, 3.75 mg, or 4.5 mg of pramipexole dihydrochloride monohydrate per tablet given once daily (QD; quaque die). Inactive ingredients are, according to the manufacturer, hypromellose (hydroxypropyl methylcellulose), cornstarch, carbomer homopolymer, colloidal silicon dioxide, and magnesium stearate. The term “extended release” or “ER” is used herein synonymously with “sustained release.”

In contrast, the affinity of dexpramipexole for dopamine receptors was found to be greatly reduced when compared to pramipexole, which renders dexpramipexole unsuitable as a dopamine agonist. Instead, dexpramipexole was shown to selectively and significantly lower eosinophil counts in human blood and tissues. The drug is currently under clinical development for eosinophil-associated diseases, including eosinophilic asthma and chronic obstructive pulmonary disease (COPD).

In literature reporting testing of dexpramipexole compositions, the dexpramipexole compositions were immediate release (IR) tablets that are given twice a day (BID; bis in die). Doses evaluated include 37.5 mg, 75 mg and 150 mg of dexpramipexole dihydrochloride equivalent per tablet (corresponding to a daily dose of 75 mg, 150 mg, and 300 mg, respectively, which is up to 800-fold higher than the lowest daily dose of 0.375 mg pramipexole salt provided by Mirapex® ER tablets).

Because of the relatively high amount of dexpramipexole that is typically used per day (e.g., about 75 mg, about 150 mg, or about 300 mg dexpramipexole dihydrochloride equivalent per day), it could not have been reasonably expected that formulating the drug as a composition suitable for once-daily oral administration to a human would be possible. A pharmaceutical composition that is intended for oral use, such as a tablet, must be swallowable by a human. This means, a certain maximum composition weight (typically about 1500 mg) should not be exceeded. If, however, the amount of the active ingredient is relatively high (such as in the event of dexpramipexole, or a pharmaceutically acceptable salt thereof, at doses typically used), the amount of additional ingredients that can be added to the pharmaceutical composition for formulation is limited and formulation possibilities are thus restricted. This can be problematic because, in general, a higher amount of active ingredient also requires a higher amount of additional ingredients such as release-controlling polymers, diluents, glidants, lubricants, and/or binders to allow a pharmaceutical composition (such as a tablet) to be manufactured and sufficiently slow down the release of the active ingredient for a once daily formulation. See, for example, Encyclopedia of Pharmaceutical Technology, Volume 1, Third Edition, edited by James Swarbrick, Chapter “Drug Delivery: Controlled Release” by Chien and Lin. In contrast, as outlined above, for pramipexole, significantly lower amounts of drug are used per day (the highest dose in Mirapex® ER once daily formulations being 4.5 mg pramipexole dihydrochloride monohydrate).

Further, in the context of matrix tablets for which the drug release mechanism is based on a combination of diffusion and erosion, the higher the solubility of a drug, the greater are the challenges for developing a matrix tablet that is suitable for once daily oral administration to a human. Diffusion is related to solubility of the drug contained in the matrix tablet. The higher the solubility, the greater the driving force for diffusion, and hence the harder it is to slow down the drug release. In the case of dexpramipexole, the salt form that is commonly used, dexpramipexole dihydrochloride monohydrate, is freely soluble across the physiological pH range. Moreover, additional formulation challenges generally encountered with a sustained release matrix tablet are the achievement of a steady release across the physiological pH range, as well as a substantially complete release within a desired time period. Surprisingly, the present disclosure provides dexpramipexole, or a pharmaceutically acceptable salt thereof, in the form of an orally deliverable pharmaceutical composition (such as in the form of an orally deliverable tablet) that is suitable for once daily administration to a human, which would not have been expected in view of constraints on composition size coupled with the high amount of dexpramipexole, or a pharmaceutically acceptable salt thereof, needed for once daily administration, and in view of the high solubility of dexpramipexole and pharmaceutically acceptable salts thereof. Moreover, as shown herein, the orally deliverable matrix tablets of the present disclosure provide for a steady release across the physiological pH range, as well as high terminal release rates.

### BRIEF SUMMARY OF THE DISCLOSURE

Although, as outlined above, it could not have been reasonably expected that formulating dexpramipexole, or a pharmaceutically acceptable salt thereof, as a sustained release composition suitable for once daily oral administration to a human would be possible, the present disclosure inter alia provides such a sustained release pharmaceutical composition. The sustained release pharmaceutical compositions of the present disclosure allow simplification of a patient's administration scheme by reducing the number of recommended daily intakes compared to immediate release dexpramipexole formulations known in the art, which improves patient's compliance and attenuates potential adverse events that are related, e.g., to high plasma concentration peaks.

In some aspects, the present disclosure provides a pharmaceutical composition in the form of an orally deliverable tablet comprising dexpramipexole, or a pharmaceutically acceptable salt thereof, wherein the pharmaceutical composition provides for a sustained release of dexpramipexole, or a pharmaceutically acceptable salt thereof, that makes the tablet suitable for once daily oral administration to a human.

Further, in some aspects, the present disclosure provides sustained release pharmaceutical compositions in alternative dosage forms than tablets (such as in the form of an orally deliverable capsule) comprising dexpramipexole, or a pharmaceutically acceptable salt thereof, wherein the pharmaceutical composition provides for a sustained release of dexpramipexole, or a pharmaceutically acceptable salt thereof, that makes the tablet suitable for once daily oral administration to a human.

In some aspects, the present disclosure provides a sustained release pharmaceutical composition in the form of an orally deliverable tablet comprising a tablet core and dexpramipexole, or a pharmaceutically acceptable salt thereof, in an amount of about 50 mg to about 400 mg of dexpramipexole dihydrochloride equivalent, wherein the tablet core comprises a homogeneous mixture of the amount of dexpramipexole, or a pharmaceutically acceptable salt thereof, and one or more anionic polymers, wherein the one or more anionic polymers constitute about 30% to about 60% by weight of the tablet core, and wherein the weight of the tablet is about 1500 mg or less.

In some aspects, the present disclosure provides a sustained release pharmaceutical composition in the form of an orally deliverable tablet comprising a tablet core and dexpramipexole, or a pharmaceutically acceptable salt thereof, in an amount of about 50 mg to about 500 mg of dexpramipexole dihydrochloride equivalent, wherein the tablet core comprises a homogeneous mixture of the amount of dexpramipexole, or a pharmaceutically acceptable salt thereof, and one or more polymers, wherein the one or more anionic polymers constitute about 5% to about 60% by weight of the tablet core, and wherein the weight of the tablet is about 1500 mg or less.

In some aspects, the present disclosure provides a sustained release pharmaceutical composition in the form of an orally deliverable tablet comprising a tablet core and dexpramipexole, or a pharmaceutically acceptable salt thereof, in an amount of about 50 mg to about 400 mg of dexpramipexole dihydrochloride equivalent, wherein the tablet core comprises a homogeneous mixture of the amount of dexpramipexole, or a pharmaceutically acceptable salt thereof, and one or more neutral polymers, wherein the one or more neutral polymers constitute about 5% to about 60% by weight of the tablet core, and wherein the weight of the tablet is about 1500 mg or less.

As shown in the Example section (see in particular Examples 3 and 9), the matrix tablets comprising dexpramipexole, or a pharmaceutically acceptable salt thereof, according to the present disclosure provide for in vitro dissolution profiles of the drug that render the matrix tablets suitable for once daily oral administration to a human. Moreover, in addition to such beneficial in vitro dissolution profiles, the matrix tablets according to the present disclosure are of sufficient mechanical strength for efficient tablet manufacturing (in particular on a routine, large-scale basis).

Also matrix tablets that do not comprise the amounts or types of polymers as specified herein resulted in an in vitro drug dissolution profile unsuitable for a once daily sustained release formulation (the dexpramipexole pharmaceutically acceptable salt in the manufactured tablets dissolved too fast), even when prepared with different manufacturing methods such as direct compression and melt granulation (see in particular Example 2, in which the tablets were not formulated according to the present disclosure).

An in general beneficial in vitro dissolution profile was observed for matrix tablets that do not comprise the amounts or the types of one or more polymers as specified herein, but instead comprise corresponding amounts of a lipophilic material (such as about 50% Compritol 888 ATO by weight of the tablet core) when manufactured by melt granulation. However, although the in vitro dissolution profile was in principle suitable for a once daily sustained release formulation, the corresponding matrix tablets were mechanically weak rendering them unsuitable for manufacturing of such tablets (in particular on a routine, large-scale basis; see in particular Example 2, in which the tablets were not formulated according to the present disclosure).

In strong contrast to the formulations that did not result in a suitable in vitro dissolution profile for a once daily sustained release formulation and/or that resulted in mechanically weak tablets, it was shown that inclusion of one or more anionic polymers at the amounts specified herein in the tablet core (i.e., at about 30% to about 60% by weight of the tablet core) resulted in matrix tablets showing desirable properties (an in vitro dissolution suitable for once daily administration to a human and sufficient mechanic strength). In particular, such beneficial properties were observed over a wide % w/w-range of anionic polymers (such as from about 30% to about 50% by weight of the tablet core). Further, the desirable properties were observed with a single anionic polymer and also with a combination of different anionic polymers (such as tablets comprising a combination of sodium carboxymethylcellulose and a crosslinked acrylic acid polymer) in the tablet core at the amounts specified herein (such as from about 30% to about 50% by weight of the tablet core). It was shown that in vitro dissolution and mechanical properties were also generally independent of specific ratios of anionic polymers towards each other, if more than one anionic polymer was present in the tablet core (such as a weight ratio of an acrylic acid polymer to sodium carboxymethylcellulose of about 3:2 to about 2:8). See in particular Example 3. Tablets formulated according to the present disclosure were also stable for several weeks under controlled room temperature (CRT) conditions (see in particular Example 6). Additionally, the desirable properties were observed with a single hydrophilic neutral polymer in the tablet core at the amounts specified herein (such as from about 5% to about 25% by weight of the tablet core). Advantageously, tablets with a single neutral polymer demonstrated suitable in vitro dissolution profile for a once daily sustained release formulation and resulted in mechanically strong tablets, making them an ideal clinical candidate. In sum, it was discovered that these desirable properties could be replicated for tablets with two anionic polymers, a single anionic polymer, or a single neutral polymer. See in particular Example 9.

In some embodiments, the amount of dexpramipexole, or a pharmaceutically acceptable salt thereof, is about 75 mg to about 300 mg of dexpramipexole dihydrochloride equivalent. In some embodiments, the amount of dexpramipexole, or a pharmaceutically acceptable salt thereof, is about 50 mg to about 100 mg of dexpramipexole dihydrochloride equivalent. In some embodiments, the amount of dexpramipexole, or a pharmaceutically acceptable salt thereof, is about 75 mg of dexpramipexole dihydrochloride equivalent. In some embodiments, the amount of dexpramipexole, or a pharmaceutically acceptable salt thereof, is about 100 mg to about 350 mg of dexpramipexole dihydrochloride equivalent. In some embodiments, the amount of dexpramipexole, or a pharmaceutically acceptable salt thereof, is about 275 mg to about 325 mg of dexpramipexole dihydrochloride equivalent. In some embodiments, the amount of dexpramipexole, or a pharmaceutically acceptable salt thereof, is about 300 mg of dexpramipexole dihydrochloride equivalent. In some embodiments, the amount of dexpramipexole, or a pharmaceutically acceptable salt thereof, is about 125 mg to about 175 mg of dexpramipexole dihydrochloride equivalent. In some embodiments, the amount of dexpramipexole, or a pharmaceutically acceptable salt thereof, is about 150 mg of dexpramipexole dihydrochloride equivalent.

In some embodiments, the amount of dexpramipexole, or a pharmaceutically acceptable salt thereof, is about 150 mg to about 400 mg of dexpramipexole dihydrochloride equivalent. In some embodiments, the amount of dexpramipexole, or a pharmaceutically acceptable salt thereof, is about 200 mg to about 350 mg of dexpramipexole dihydrochloride equivalent. In some embodiments, the amount of dexpramipexole, or a pharmaceutically acceptable salt thereof, is about 300 mg of dexpramipexole dihydrochloride equivalent. In some embodiments, the amount of dexpramipexole, or a pharmaceutically acceptable salt thereof, is about 300 mg to about 400 mg of dexpramipexole dihydrochloride equivalent. In some embodiments, the amount of dexpramipexole, or a pharmaceutically acceptable salt thereof, is about 350 mg to about 385 mg of dexpramipexole dihydrochloride equivalent. In some embodiments, the amount of dexpramipexole, or a pharmaceutically acceptable salt thereof, is 376 mg of dexpramipexole dihydrochloride equivalent.

In some embodiments, the homogeneous mixture comprises one or more polymers. In some embodiments, the one or more polymers are anionic polymers. In some embodiments, the one or more polymers are neutral polymers. In some embodiments, the one or more anionic polymers constitute about 30% to about 60% by weight of the tablet core. In some embodiments, the one or more neutral polymers constitute about 5% to about 25% by weight of the tablet core. In some embodiments, the homogeneous mixture comprises no more than one anionic polymer. In some embodiments, the one anionic polymer constitutes about 20% to about 40% by weight of the tablet core. In some embodiments, the homogeneous mixture comprises no more than one neutral polymer. In some embodiments, the one neutral polymer constitutes about 5% to about 25% by weight of the tablet core.

In some embodiments, the weight ratio of dexpramipexole dihydrochloride equivalent to the one or more anionic polymers is about 0.5:1 to about 1:1. In some embodiments, the weight ratio of dexpramipexole dihydrochloride equivalent to the one or more anionic polymers is about 0.7:1 to about 0.9:1. In some embodiments, the weight ratio of dexpramipexole dihydrochloride equivalent to the one or more anionic polymers is about 0.7:1. In some embodiments, the weight ratio of dexpramipexole dihydrochloride equivalent to the one or more anionic polymers is about 0.9:1.

In some embodiments, the weight ratio of dexpramipexole dihydrochloride equivalent to the one or more polymers is about 0.5:1 to about 3.5:1. In some embodiments, the weight ratio of dexpramipexole dihydrochloride equivalent to the one or more polymers is about 0.7:1 to about 3.0:1. In some embodiments, the weight ratio of dexpramipexole dihydrochloride equivalent to the one or more polymers is about 0.7:1. In some embodiments, the weight ratio of dexpramipexole dihydrochloride equivalent to the one or more polymers is about 0.9:1.

In some embodiments, the weight ratio of dexpramipexole dihydrochloride equivalent to the one or more neutral polymers is about 0.5:1 to about 3.5:1. In some embodiments, the weight ratio of dexpramipexole dihydrochloride equivalent to the one anionic polymer is about 0.5:1 to about 3.5:1. In some embodiments, the weight ratio of dexpramipexole dihydrochloride equivalent to the one neutral polymer is about 0.5:1 to about 3.5:1.

In some embodiments, the pharmaceutically acceptable salt of dexpramipexole is a methane sulfonic acid salt, sulfuric acid salt, tartaric acid salt, p-toluene sulfonic acid salt, phosphoric acid salt, maleic acid salt, fumaric acid salt, malic acid salt, citric acid salt, succinic acid salt, or any combination thereof. In some embodiments, the pharmaceutically acceptable salt of dexpramipexole is dexpramipexole dihydrochloride or a hydrate thereof. In some embodiments, the pharmaceutically acceptable salt of dexpramipexole is dexpramipexole dihydrochloride monohydrate.

In some embodiments, the one or more anionic polymers are selected from carboxymethylcellulose, an acrylic acid polymer, a methyl methacrylic acid polymer, a methyl methacrylic acid copolymer, a methacrylic acid polymer, a methacrylic acid copolymer, alignate, carrageenan, xanthan gum, or arabic gum. In some embodiments, the one or more anionic polymers are selected from carboxymethylcellulose and an acrylic acid polymer. In some embodiments, carboxymethylcellulose is sodium carboxymethylcellulose. In some embodiments, the acrylic acid polymer is a crosslinked acrylic acid polymer.

In some embodiments, the one or more anionic polymers constitute about 35% to about 55% by weight of the tablet core. In some embodiments, the one or more anionic polymers constitute about 38% to about 42% by weight of the tablet core. In some embodiments, the one or more anionic polymers constitute about 40% by weight of the tablet core. In some embodiments, the one or more anionic polymers constitute about 45% to about 49% by weight of the tablet core. In some embodiments, the one or more anionic polymers constitute about 47% by weight of the tablet core.

In some embodiments, the one or more anionic polymers constitute about 25% to about 55% by weight of the tablet core. In some embodiments, the one or more anionic polymers constitute about 28% to about 48% by weight of the tablet core. In some embodiments, the one or more anionic polymers constitute about 28% by weight of the tablet core. In some embodiments, the one or more anionic polymers constitute about 40% to about 48% by weight of the tablet core. In some embodiments, the one or more anionic polymers constitute about 42% by weight of the tablet core. In some embodiments, the one or more anionic polymers constitute about 40% by weight of the tablet core.

In some embodiments, the one anionic polymer constitutes about 20% to about 40% by weight of the tablet core. In some embodiments, the one anionic polymer constitutes about 25% to about 35% by weight of the tablet core. In some embodiments, the one anionic polymer constitutes about 28% by weight of the tablet core.

In some embodiments, the one or more neutral polymers constitute about 5% to about 25% by weight of the tablet core. In some embodiments, the one or more neutral polymers constitute about 10% to about 20% by weight of the tablet core. In some embodiments, the one or more neutral polymers constitute about 15% by weight of the tablet core.

In some embodiments, the one or more neutral polymers constitute between about 5% and about 60% by weight of the tablet core. In some embodiments, the one or more neutral polymers constitute between about 10% and about 60% by weight of the tablet core. In some embodiments, the one or more neutral polymers constitute about 15% to about 60% by weight of the tablet core. In some embodiments, the one or more neutral polymers constitute between about 20% and about 60% by weight of the tablet core. In some embodiments, the one or more neutral polymers constitute about 25% to about 60% by weight of the tablet core. In some embodiments, the one or more neutral polymers constitute between about 30% and about 60% by weight of the tablet core. In some embodiments, the one or more neutral polymers constitute about 35% to about 60% by weight of the tablet core. In some embodiments, the one or more neutral polymers constitute between about 40% and about 60% by weight of the tablet core. In some embodiments, the one or more neutral polymers constitute about 45% to about 60% by weight of the tablet core. In some embodiments, the one or more neutral polymers constitute between about 50% and about 60% by weight of the tablet core. In some embodiments, the one or more neutral polymers constitute about 55% to about 60% by weight of the tablet core.

In some embodiments, the one or more neutral polymers constitute about 5% to about 55% by weight of the tablet core. In some embodiments, the one or more neutral polymers constitute about 5% to about 50% by weight of the tablet core. In some embodiments, the one or more neutral polymers constitute about 5% to about 45% by weight of the tablet core. In some embodiments, the one or more neutral polymers constitute about 5% to about 40% by weight of the tablet core. In some embodiments, the one or more neutral polymers constitute about 5% to about 35% by weight of the tablet core. In some embodiments, the one or more neutral polymers constitute about 5% to about 30% by weight of the tablet core.

In some embodiments, the one or more neutral polymers constitute about 20% by weight of the tablet core. In some embodiments, the one or more neutral polymers constitute about 25% by weight of the tablet core. In some embodiments, the one or more neutral polymers constitute about 30% by weight of the tablet core. In some embodiments, the one or more neutral polymers constitute about 35% by weight of the tablet core. In some embodiments, the one or more neutral polymers constitute about 40% by weight of the tablet core. In some embodiments, the one or more neutral polymers constitute about 45% by weight of the tablet core. In some embodiments, the one or more neutral polymers constitute about 50% by weight of the tablet core. In some embodiments, the one or more neutral polymers constitute about 55% by weight of the tablet core. In some embodiments, the one or more neutral polymers constitute about 60% by weight of the tablet core.

In some embodiments, the one neutral polymer constitutes about 5% to about 25% by weight of the tablet core. In some embodiments, the one neutral polymer constitutes about 10% to about 20% by weight of the tablet core. In some embodiments, the one neutral polymer constitutes about 15% by weight of the tablet core.

In some embodiments, the homogeneous mixture comprises the one or more anionic polymers at about 250 mg to about 550 mg. In some embodiments, the homogeneous mixture comprises the one or more anionic polymers at about 320 mg to about 360 mg. In some embodiments, the homogeneous mixture comprises the one or more anionic polymers at about 340 mg. In some embodiments, the homogeneous mixture comprises the one or more anionic polymers at about 400 mg to about 440 mg. In some embodiments, the homogeneous mixture comprises the one or more anionic polymers at about 425 mg.

In some embodiments, the homogeneous mixture comprises the one or more anionic polymers at about 150 mg to about 550 mg. In some embodiments, the homogeneous mixture comprises the one or more anionic polymers at about 200 mg to about 500 mg. In some embodiments, the homogeneous mixture comprises the one or more anionic polymers at about 210 mg. In some embodiments, the homogeneous mixture comprises the one or more anionic polymers at about 300 mg to about 480 mg. In some embodiments, the homogeneous mixture comprises the one or more anionic polymers at about 475 mg. In some embodiments, the homogeneous mixture comprises the one or more anionic polymers at about 425 mg. In some embodiments, the homogeneous mixture comprises the one or more anionic polymers at about 340 mg.

In some embodiments, the homogeneous mixture comprises no more than one anionic polymer. In some embodiments, the anionic polymer is carboxymethylcellulose. In some embodiments, the carboxymethylcellulose is sodium carboxymethylcellulose. In some embodiments, the amount of carboxymethylcellulose is about 23% to about 38% of sodium carboxymethylcellulose equivalent by weight of the tablet core. In some embodiments, the amount of carboxymethylcellulose is about 25% to about 30% of sodium carboxymethylcellulose equivalent by weight of the tablet core. In some embodiments, the amount of carboxymethylcellulose is about 28% of sodium carboxymethylcellulose equivalent by weight of the tablet core.

In some embodiments, the amount of carboxymethylcellulose is about 100 mg to about 300 mg of sodium carboxymethylcellulose equivalent. In some embodiments, the amount of carboxymethylcellulose is about 200 mg to about 250 mg of sodium carboxymethylcellulose equivalent. In some embodiments, the amount of carboxymethylcellulose is about 210 mg of sodium carboxymethylcellulose equivalent.

In some embodiments, the homogeneous mixture comprises a first anionic polymer and a second anionic polymer and wherein the first anionic polymer is carboxymethylcellulose and the second anionic polymer is an acrylic acid polymer. In some embodiments, the acrylic acid polymer is a crosslinked acrylic acid polymer. In some embodiments, carboxymethylcellulose is sodium carboxymethylcellulose.

In some embodiments, the amount of carboxymethylcellulose is about 23% to about 38% of sodium carboxymethylcellulose equivalent by weight of the tablet core and the amount of the acrylic acid polymer is about 7% to about 22% by weight of the tablet core. In some embodiments, the amount of carboxymethylcellulose is about 27% to about 34% of sodium carboxymethylcellulose equivalent by weight of the tablet core and the amount of the acrylic acid polymer is about 11% to about 15% by weight of the tablet core. In some embodiments, the amount of carboxymethylcellulose is about 27% to about 29% of sodium carboxymethylcellulose equivalent by weight of the tablet core and the amount of the acrylic acid polymer is about 11% to about 13% by weight of the tablet core. In some embodiments, the amount of carboxymethylcellulose is about 28% of sodium carboxymethylcellulose equivalent by weight of the tablet core and the amount of the acrylic acid polymer is about 12% by weight of the tablet core. In some embodiments, the amount of carboxymethylcellulose is about 30% of sodium carboxymethylcellulose equivalent by weight of the tablet core and the amount of the acrylic acid polymer is about 13% by weight of the tablet core. In some embodiments, the amount of carboxymethylcellulose is about 32% to about 34% of sodium carboxymethylcellulose equivalent by weight of the tablet core and the amount of the acrylic acid polymer is about 13% to about 15% by weight of the tablet core. In some embodiments, the amount of carboxymethylcellulose is about 33% of sodium carboxymethylcellulose equivalent by weight of the tablet core and the amount of the acrylic acid polymer is about 14% by weight of the tablet core.

In some embodiments, the amount of carboxymethylcellulose is about 190 mg to about 350 mg of sodium carboxymethylcellulose equivalent and the amount of the acrylic acid polymer is about 60 mg to about 200 mg. In some embodiments, the amount of carboxymethylcellulose is about 205 mg to about 335 mg of sodium carboxymethylcellulose equivalent and the amount of the acrylic acid polymer is about 100 mg to about 145 mg. In some embodiments, the amount of carboxymethylcellulose is about 333 mg of sodium carboxymethylcellulose equivalent and the amount of the acrylic acid polymer is about 143 mg. In some embodiments, the amount of carboxymethylcellulose is about 200 mg to about 250 mg of sodium carboxymethylcellulose equivalent and the amount of the acrylic acid polymer is about 75 mg to about 125 mg. In some embodiments, the amount of carboxymethylcellulose is about 235 mg to about 245 mg of sodium carboxymethylcellulose equivalent and the amount of the acrylic acid polymer is about 95 mg to about 105 mg. In some embodiments, the amount of carboxymethylcellulose is about 238 mg of sodium carboxymethylcellulose equivalent and the amount of the acrylic acid polymer is about 102 mg. In some embodiments, the amount of carboxymethylcellulose is about 275 mg to about 325 mg of sodium carboxymethylcellulose equivalent and the amount of the acrylic acid polymer is about 100 mg to about 150 mg. In some embodiments, the amount of carboxymethylcellulose is about 298 mg of sodium carboxymethylcellulose equivalent and the amount of the acrylic acid polymer is about 128 mg. In some embodiments, the amount of carboxymethylcellulose is about 295 mg to about 305 mg of sodium carboxymethylcellulose equivalent and the amount of the acrylic acid polymer is about 125 mg to about 135 mg. In some embodiments, the amount of carboxymethylcellulose is about 298 mg of sodium carboxymethylcellulose equivalent and the amount of the acrylic acid polymer is about 128 mg.

In some embodiments, the weight ratio of the acrylic acid polymer to sodium carboxymethylcellulose equivalent is about 3:2 to about 2:8. In some embodiments, the weight ratio of the acrylic acid polymer to sodium carboxymethylcellulose equivalent is about 2:3 to about 2:8. In some embodiments, the weight ratio of the acrylic acid polymer to sodium carboxymethylcellulose equivalent is about 3:7.

In some embodiments, the one or more polymers are neutral polymers. In some embodiments, the one or more neutral polymers is hydroxypropyl methylcellulose or silicified hydroxypropyl methylcellulose. In some embodiments, the one or more neutral polymers is hydroxypropyl methylcellulose. In some embodiments, the one or more neutral polymers is silicified hydroxypropyl methylcellulose. In some embodiments, the one or more neutral polymers constitute about 5% to about 25% by weight of the tablet core. In some embodiments, the neutral polymer constitute about 15% by weight of the tablet core.

In some embodiments, the homogeneous mixture comprises dexpramipexole, or a pharmaceutically acceptable salt thereof, and two anionic polymers. In some embodiments, the homogeneous mixture comprises dexpramipexole, or a pharmaceutically acceptable salt thereof, and no more than one anionic polymer. In some embodiments, the homogeneous mixture comprises dexpramipexole, or a pharmaceutically acceptable salt thereof, and two neutral polymers. In some embodiments, the homogeneous mixture comprises dexpramipexole, or a pharmaceutically acceptable salt thereof, and no more than one neutral polymer. In some embodiments, the homogeneous mixture comprises dexpramipexole, or a pharmaceutically acceptable salt thereof, and does not comprise an anionic polymer. In some embodiments, the homogeneous mixture comprises dexpramipexole, or a pharmaceutically acceptable salt thereof, and does not comprise a neutral polymer.

In some embodiments, the homogeneous mixture consists essentially of dexpramipexole, or a pharmaceutically acceptable salt thereof, and two anionic polymers. In some embodiments, the homogeneous mixture consists essentially of dexpramipexole, or a pharmaceutically acceptable salt thereof, and no more than one anionic polymer. In some embodiments, the homogeneous mixture consists essentially of dexpramipexole, or a pharmaceutically acceptable salt thereof, and two neutral polymers.

In some embodiments, the homogeneous mixture further comprises a microcrystalline cellulose, magnesium stearate, colloidal silicon dioxide, or any combination thereof. In some embodiments, the homogeneous mixture further comprises a microcrystalline cellulose, magnesium stearate and colloidal silicon dioxide. In some embodiments, the microcrystalline cellulose is silicified microcrystalline cellulose.

In some embodiments, the homogeneous mixture consists essentially of.
(a) about 315 mg to about 325 mg dexpramipexole dihydrochloride monohydrate;
(b) about 235 mg to about 245 mg sodium carboxymethylcellulose;
(c) about 95 mg to about 105 mg crosslinked acrylic acid polymer;
(d) about 175 mg to about 185 mg silicified microcrystalline cellulose;
(e) about 5 mg to about 7 mg magnesium stearate; and
(f) about 3 mg to about 5 mg colloidal silicon dioxide.

In some embodiments, the homogeneous mixture consists essentially of:
(a) about 319 mg dexpramipexole dihydrochloride monohydrate;
(b) about 238 mg sodium carboxymethylcellulose;
(c) about 102 mg crosslinked acrylic acid polymer;
(d) about 180 mg silicified microcrystalline cellulose;
(e) about 6 mg magnesium stearate; and
(f) about 4 mg colloidal silicon dioxide.

In some embodiments, the homogeneous mixture consists essentially of:
(a) about 315 mg to about 325 mg dexpramipexole dihydrochloride monohydrate;
(b) about 295 mg to about 305 mg sodium carboxymethylcellulose;
(c) about 125 mg to about 135 mg crosslinked acrylic acid polymer;
(d) about 140 mg to about 150 mg silicified microcrystalline cellulose;
(e) about 6 mg to about 8 mg magnesium stearate; and
(f) about 4 mg to about 6 mg colloidal silicon dioxide.

In some embodiments, the homogeneous mixture consists essentially of:
(a) about 319 mg dexpramipexole dihydrochloride monohydrate;
(b) about 298 mg sodium carboxymethylcellulose;
(c) about 128 mg crosslinked acrylic acid polymer;
(d) about 145 mg silicified microcrystalline cellulose;
(e) about 7 mg magnesium stearate; and
(f) about 5 mg colloidal silicon dioxide.

In some embodiments, the homogeneous mixture consists essentially of:
(a) about 390 mg to about 410 mg dexpramipexole dihydrochloride monohydrate;
(b) about 325 mg to about 341 mg sodium carboxymethylcellulose;
(c) about 135 mg to about 145 mg crosslinked acrylic acid polymer;
(d) about 230 mg to about 240 mg microcrystalline cellulose or silicified microcrystalline cellulose;
(e) about 5 mg to about 10 mg magnesium stearate; and
(f) about 4 mg to about 7 mg colloidal silicon dioxide.

In some embodiments, the homogeneous mixture consists essentially of:
(a) about 400 mg dexpramipexole dihydrochloride monohydrate;
(b) about 333 mg sodium carboxymethylcellulose;
(c) about 143 mg crosslinked acrylic acid polymer;
(d) about 236 mg microcrystalline cellulose or silicified microcrystalline cellulose;
(e) about 8 mg magnesium stearate; and
(f) about 6 mg colloidal silicon dioxide.

In some embodiments, the homogeneous mixture consists essentially of:
(a) about 315 mg to about 325 mg dexpramipexole dihydrochloride monohydrate;
(b) about 110 mg to about 120 mg hydroxypropyl methylcellulose or silicified hydroxypropyl methylcellulose;
(c) about 310 mg to about 320 mg microcrystalline cellulose or silicified microcrystalline cellulose;
(d) about 3 mg to about 5 mg magnesium stearate; and
(e) about 3 mg to about 5 mg colloidal silicon dioxide.

In some embodiments, the homogeneous mixture consists essentially of:
(a) about 319 mg dexpramipexole dihydrochloride monohydrate;
(b) about 113 mg hydroxypropyl methylcellulose or silicified hydroxypropyl methylcellulose;
(c) about 313 mg microcrystalline cellulose or silicified microcrystalline cellulose;
(d) about 4 mg magnesium stearate; and
(e) about 4 mg colloidal silicon dioxide.

In some embodiments, the homogeneous mixture consists essentially of:
(a) about 315 mg to about 325 mg dexpramipexole dihydrochloride monohydrate;
(b) about 110 mg to about 120 mg hydroxypropyl methylcellulose or silicified hydroxypropyl methylcellulose;
(c) about 305 mg to about 315 mg silicified microcrystalline cellulose;
(d) about 5 mg to about 7 mg magnesium stearate; and
(e) about 3 mg to about 5 mg colloidal silicon dioxide.

In some embodiments, the homogeneous mixture consists essentially of:
(a) about 319 mg dexpramipexole dihydrochloride monohydrate;
(b) about 113 mg hydroxypropyl methylcellulose or silicified hydroxypropyl methylcellulose;
(c) about 310 mg silicified microcrystalline cellulose;
(d) about 6 mg magnesium stearate; and
(e) about 4 mg colloidal silicon dioxide.

In some embodiments, the homogeneous mixture consists essentially of:
(a) about 315 mg to about 325 mg dexpramipexole dihydrochloride monohydrate;
(b) about 110 mg to about 120 mg hydroxypropyl methylcellulose or silicified hydroxypropyl methylcellulose;
(c) about 305 mg to about 315 mg microcrystalline cellulose;
(d) about 5 mg to about 7 mg magnesium stearate; and
(e) about 3 mg to about 5 mg colloidal silicon dioxide.

In some embodiments, the homogeneous mixture consists essentially of:
(a) about 319 mg dexpramipexole dihydrochloride monohydrate;
(b) about 113 mg hydroxypropyl methylcellulose or silicified hydroxypropyl methylcellulose;
(c) about 310 mg microcrystalline cellulose;
(d) about 6 mg magnesium stearate; and
(e) about 4 mg colloidal silicon dioxide.

In some embodiments, the homogeneous mixture consists essentially of:
(a) about 315 mg to about 325 mg dexpramipexole dihydrochloride monohydrate;
(b) about 200 mg to about 220 mg sodium carboxymethylcellulose;
(c) about 210 mg to about 220 mg microcrystalline cellulose or silicified microcrystalline cellulose;
(d) about 3 mg to about 5 mg magnesium stearate; and
(e) about 3 mg to about 5 mg colloidal silicon dioxide.

In some embodiments, the homogeneous mixture consists essentially of:
(a) about 319 mg dexpramipexole dihydrochloride monohydrate;
(b) about 210 mg sodium carboxymethylcellulose;
(c) about 214 mg microcrystalline cellulose or silicified microcrystalline cellulose;
(d) about 4 mg magnesium stearate; and
(e) about 4 mg colloidal silicon dioxide.

In some embodiments, the composition further comprises a film coating surrounding the tablet core. In some embodiments, the film coating comprises polyvinyl alcohol. In some embodiments, the film coating further comprises titanium dioxide, polyethylene glycol and talc.

In some embodiments, the weight of the tablet is about 1300 mg or less. In some embodiments, the weight of the tablet is about 1159 mg. In some embodiments, the weight of the tablet is about 1000 mg or less. In some embodiments, the weight of the tablet is about 927 mg. In some embodiments, the weight of the tablet is about 850 mg to about 950 mg. In some embodiments, the weight of the tablet is about 850 mg to about 900 mg. In some embodiments, the weight of the tablet is about 900 mg to about 950 mg. In some embodiments, the weight of the tablet is about 750 mg to about 900 mg. In some embodiments, the weight of the tablet is about 876 mg. In some embodiments, the weight of the tablet is about 773 mg.

In some embodiments, the tablet is a capsule shaped tablet. In some embodiments, the tablet is an oval shaped tablet. In some embodiments, the length of the short axis of the tablet is about 7.5 mm to about 9.5 mm, the length of the long axis of the tablet is about 18 mm to about 20 mm, and the thickness of the tablet is about 5 mm to about 7 mm.

In some embodiments, about 40% to about 80% of dexpramipexole, or the pharmaceutically acceptable salt thereof, is released at about 12 hours of incubation of the tablet in about 900 mL of 50 mM monobasic potassium phosphate buffer, pH 6.8, at a temperature of 37±0.5° C. as measured using an USP type I apparatus operated at a spindle rotation speed of about 100 rpm.

In some embodiments, dexpramipexole, or the pharmaceutically acceptable salt thereof, has a chiral purity for dexpramipexole, or the pharmaceutically salt thereof, of 99.96% or more. In some embodiments, the composition comprises 0.02% or less pramipexole, or a pharmaceutically acceptable salt thereof, by weight of the tablet. In some embodiments, the composition comprises 0.015% or less pramipexole, or a pharmaceutically acceptable salt thereof, by weight of the tablet.

It was further shown that the tablets according to the present disclosure (comprising one or more anionic polymers at the amounts specified herein) can be prepared by different production processes (including direct compression; wet granulation, in particular high shear wet granulation; and roller compaction) and at different scales without affecting the general advantageous properties (such as the beneficial in vitro dissolution profiles) of the tablets (see in particular Example 4).

However, although the in vitro dissolution profiles were similar, independent of how the matrix tablets with the one or more anionic polymers were manufactured, a manufacturing method comprising preparing densified granules (such as by dry or wet granulation) was shown to be a particular beneficial manufacturing method for the matrix tablets of the present disclosure (see in particular Examples 4 and 5).

Thus, the present disclosure is further directed to methods of manufacturing the pharmaceutical compositions comprising dexpramipexole, or a pharmaceutically acceptable salt thereof, of the present disclosure. In some aspects, the present disclosure provides a method of manufacturing a sustained release pharmaceutical composition in the form of an orally deliverable tablet comprising a tablet core with dexpramipexole, or a pharmaceutically acceptable salt thereof, the method comprising:
preparing a pre-blend by mixing dexpramipexole, or a pharmaceutically acceptable salt thereof, and one or more anionic polymers;
preparing densified granules from the pre-blend;
preparing a blend by mixing the densified granules with a glidant and a lubricant; and
compressing the blend into a tablet core;
wherein dexpramipexole, or the pharmaceutically acceptable salt thereof, constitutes about 30% to about 40% of dexpramipexole dihydrochloride monohydrate equivalent by weight of the blend and the one or more anionic polymers constitute about 30% to about 55% by weight of the blend.

In some aspects, the present disclosure provides a method of manufacturing a sustained release pharmaceutical composition in the form of an orally deliverable tablet comprising a tablet core with dexpramipexole, or a pharmaceutically acceptable salt thereof, the method comprising:
preparing a pre-blend by mixing dexpramipexole, or a pharmaceutically acceptable salt thereof, and one or more polymers;
preparing densified granules from the pre-blend;
preparing a final blend by mixing the densified granules with a glidant and a lubricant; and
compressing the blend into a tablet core;
wherein dexpramipexole, or the pharmaceutically acceptable salt thereof, constitutes about 30% to about 50% of dexpramipexole dihydrochloride monohydrate equivalent by weight of the densified granules and the one or more polymers constitute about 5% to about 60% by weight of the densified granules.

In some aspects, the present disclosure provides a method of manufacturing a sustained release pharmaceutical composition in the form of an orally deliverable tablet comprising a tablet core with dexpramipexole, or a pharmaceutically acceptable salt thereof, the method comprising:
preparing a pre-blend by mixing dexpramipexole, or a pharmaceutically acceptable salt thereof, and one or more polymers;
preparing a final blend by mixing the pre-blend with a glidant and a lubricant; and
compressing the blend into a tablet core;
wherein dexpramipexole, or the pharmaceutically acceptable salt thereof, constitutes about 30% to about 50% of dexpramipexole dihydrochloride monohydrate equivalent by weight of the densified granules and the one or more polymers constitute about 5% to about 60% by weight of the densified granules.

In some embodiments, the pre-blend further comprises additional excipients. In some embodiments, the pre-blend further comprises a microcrystalline cellulose, magnesium stearate, or a combination thereof. In some embodiments, the pre-blend further comprises a microcrystalline cellulose and magnesium stearate. In some embodiments, the microcrystalline cellulose is silicified microcrystalline cellulose.

In some embodiments, the final blend further comprises additional excipients. In some embodiments, the final further comprises a microcrystalline cellulose, magnesium stearate, colloidal silicon dioxide, or any combination thereof. In some embodiments, the final further comprises a microcrystalline cellulose, magnesium stearate and colloidal silicon dioxide. In some embodiments, the microcrystalline cellulose is silicified microcrystalline cellulose.

In some embodiments, the one or more polymers are anionic polymers. In some embodiments, the one or more anionic polymers constitute about 20% to about 55% by weight of the blend. In some embodiments, the one or more neutral polymers constitute about 5% to about 25% by weight of the blend.

In some embodiments, the pre-blend comprises dexpramipexole, or a pharmaceutically acceptable salt thereof, and no more than one anionic polymer. In some embodiments, the anionic polymer constitutes about 20% to about 55% by weight of the blend.

In some embodiments, the pre-blend comprises dexpramipexole, or a pharmaceutically acceptable salt thereof, and no more than one neutral polymer. In some embodiments, the anionic polymer constitutes about 5% to about 25% by weight of the blend.

In some embodiments, the pre-blend comprises dexpramipexole, or a pharmaceutically acceptable salt thereof, and does not comprise an anionic polymer. In some embodiments, the pre-blend comprises dexpramipexole, or a pharmaceutically acceptable salt thereof, and does not comprise a neutral polymer.

In some embodiments, the method further comprises coating the tablet core with a film coating. In some embodiments, the film coating comprises polyvinyl alcohol. In some embodiments, the film coating further comprises titanium dioxide, polyethylene glycol and talc.

In some embodiments, the glidant is colloidal silicon dioxide and the lubricant is magnesium stearate.

In some embodiments, the one or more anionic polymers constitute about 35% to about 50% by weight of the blend. In some embodiments, the one or more anionic polymers constitute about 38% to about 42% by weight of the blend. In some embodiments, the one or more anionic polymers constitute about 40% by weight of the blend. In some embodiments, the one or more anionic polymers constitute about 45% to about 49% by weight of the blend. In some embodiments, the one or more anionic polymers constitute about 47% by weight of the blend.

In some embodiments, the one or more anionic polymers constitute about 25% to about 50% by weight of the blend. In some embodiments, the one or more anionic polymers constitute about 28% to about 48% by weight of the blend. In some embodiments, the one or more anionic polymers constitute about 28% by weight of the blend. In some embodiments, the one or more anionic polymers constitute about 40% to about 48% by weight of the blend. In some embodiments, the one or more anionic polymers constitute about 48% by weight of the blend. In some embodiments, the one or more anionic polymers constitute about 43% by weight of the blend. In some embodiments, the one or more anionic polymers constitute about 40% by weight of the blend.

In some embodiments, the one or more anionic polymers are selected from carboxymethylcellulose, an acrylic acid polymer, a methacrylic acid polymer, a methacrylic acid copolymer, alignate, carrageenan, xanthan gum, or arabic gum. In some embodiments, the one or more anionic polymers are selected from carboxymethylcellulose and an acrylic acid polymer. In some embodiments, carboxymethylcellulose is sodium carboxymethylcellulose. In some embodiments, the acrylic acid polymer is a crosslinked acrylic acid polymer. In some embodiments, the pre-blend comprises a first anionic polymer and a second anionic polymer and wherein the first anionic polymer is carboxymethylcellulose and the second anionic polymer is an acrylic acid polymer. In some embodiments, the acrylic acid polymer is a crosslinked acrylic acid polymer. In some embodiments, carboxymethylcellulose is sodium carboxymethylcellulose.

In some embodiments, carboxymethylcellulose constitutes about 27% to about 34% of sodium carboxymethylcellulose equivalent by weight of the blend and the acrylic acid polymer constitutes about 11% to about 15% by weight of the blend. In some embodiments, carboxymethylcellulose constitutes about 30% of sodium carboxymethylcellulose equivalent by weight of the blend and the acrylic acid polymer constitutes about 13% by weight of the blend. In some embodiments, carboxymethylcellulose constitutes about 27% to about 29% of sodium carboxymethylcellulose equivalent by weight of the blend and the acrylic acid polymer constitutes about 11% to about 13% by weight of the blend. In some embodiments, carboxymethylcellulose constitutes about 28% of sodium carboxymethylcellulose equivalent by weight of the blend and the acrylic acid polymer constitutes about 12% by weight of the blend. In some embodiments, carboxymethylcellulose constitutes about 32% to about 34% of sodium carboxymethylcellulose equivalent by weight of the blend and the acrylic acid polymer constitutes about 13% to about 15% by weight of the blend. In some embodiments, carboxymethylcellulose constitutes about 33% of sodium carboxymethylcellulose equivalent by weight of the blend and the acrylic acid polymer constitutes about 14% by weight of the blend.

In some embodiments, the pre-blend comprises no more than one anionic polymer and wherein the anionic polymer is carboxymethylcellulose. In some embodiments, the carboxymethylcellulose is sodium carboxymethylcellulose. In some embodiments, the carboxymethylcellulose constitutes about 27% to about 29% of sodium carboxymethylcellulose equivalent by weight of the densified granules. In some embodiments, the carboxymethylcellulose constitutes about 28% of sodium carboxymethylcellulose equivalent by weight of the densified granules.

In some embodiments, the pre-blend comprises one or more neutral polymers. In some embodiments, the one or more neutrals polymer are hydroxypropyl methylcellulose or silicified hydroxypropyl methylcellulose. In some embodiments, the one or more neutral polymers constitute about 5% to about 25% by weight of the blend. In some embodiments, the one or more neutral polymers constitute about 15% by weight of the blend. In some embodiments, the pre-blend comprises no more than one neutral polymer. In some embodiments, the neutral polymer is hydroxypropyl methylcellulose or silicified hydroxypropyl methylcellulose. In some embodiments, the neutral polymer constitutes about 5% to about 25% by weight of the blend. In some embodiments, the neutral polymer constitutes about 15% by weight of the blend.

In some embodiments, the densified granules constitute about 99% by weight of the blend. In some embodiments, the glidant constitutes about 0.5% by weight of the blend and the lubricant constitutes about 0.5% by weight of the blend.

In some embodiments, the pharmaceutically acceptable salt of dexpramipexole is a methane sulfonic acid salt, sulfuric acid salt, tartaric acid salt, p-toluene sulfonic acid salt, phosphoric acid salt, maleic acid salt, fumaric acid salt, malic acid salt, citric acid salt, succinic acid salt, or any combination thereof. In some embodiments, the pharmaceutically acceptable salt of dexpramipexole is dexpramipexole dihydrochloride or a hydrate thereof. In some embodiments, the pharmaceutically acceptable salt of dexpramipexole is dexpramipexole dihydrochloride monohydrate.

In some embodiments, the blend consists essentially of:
(a) about 35% to about 40% dexpramipexole dihydrochloride monohydrate by weight of the blend;
(b) about 25% to about 30% sodium carboxymethylcellulose by weight of the blend;
(c) about 10% to about 15% crosslinked acrylic acid polymer by weight of the blend;
(d) about 18% to about 23% silicified microcrystalline cellulose by weight of the blend;
(e) about 0.5% to about 1.0% magnesium stearate by weight of the blend; and
(f) about 0.25% to about 0.75% colloidal silicon dioxide by weight of the blend.

In some embodiments, the blend consists essentially of:
(a) about 38% dexpramipexole dihydrochloride monohydrate by weight of the blend;
(b) about 28% sodium carboxymethylcellulose by weight of the blend;
(c) about 12% crosslinked acrylic acid polymer by weight of the blend;
(d) about 21% silicified microcrystalline cellulose by weight of the blend;
(e) about 0.75% magnesium stearate by weight of the blend; and
(f) about 0.5% colloidal silicon dioxide by weight of the blend.

In some embodiments, the blend consists essentially of:
(a) about 33% to about 38% dexpramipexole dihydrochloride monohydrate by weight of the blend;
(b) about 30% to about 35% sodium carboxymethylcellulose by weight of the blend;
(c) about 12% to about 17% crosslinked acrylic acid polymer by weight of the blend;
(d) about 13% to about 18% silicified microcrystalline cellulose by weight of the blend;
(e) about 0.5% to about 1.0% magnesium stearate by weight of the blend; and
(f) about 0.25% to about 0.75% colloidal silicon dioxide by weight of the blend.

In some embodiments, the blend consists essentially of:
(a) about 35% dexpramipexole dihydrochloride monohydrate by weight of the blend;
(b) about 33% sodium carboxymethylcellulose by weight of the blend;
(c) about 14% crosslinked acrylic acid polymer by weight of the blend;
(d) about 16% silicified microcrystalline cellulose by weight of the blend;
(e) about 0.75% magnesium stearate by weight of the blend; and
(f) about 0.5% colloidal silicon dioxide by weight of the blend.

In some embodiments, the blend consists essentially of:
(a) about 33% to about 39% dexpramipexole dihydrochloride monohydrate by weight of the blend;
(b) about 27% to about 33% sodium carboxymethylcellulose by weight of the blend;
(c) about 10% to about 16% crosslinked acrylic acid polymer by weight of the blend;
(d) about 18% to about 24% microcrystalline cellulose or silicified microcrystalline cellulose by weight of the blend;
(e) about 0.5% to about 1.0% magnesium stearate by weight of the blend; and
(f) about 0.25% to about 0.75% colloidal silicon dioxide by weight of the blend.

In some embodiments, the blend consists essentially of:
(a) about 36% dexpramipexole dihydrochloride monohydrate by weight of the blend;
(b) about 30% sodium carboxymethylcellulose by weight of the blend;
(c) about 13% crosslinked acrylic acid polymer by weight of the blend;
(d) about 21% microcrystalline cellulose or silicified microcrystalline cellulose by weight of the blend;
(e) about 0.75% magnesium stearate by weight of the blend; and
(f) about 0.5% colloidal silicon dioxide by weight of the blend.

In some embodiments, the blend consists essentially of:
(a) about 40% to about 46% dexpramipexole dihydrochloride monohydrate by weight of the blend;
(b) about 10% to about 20% hydroxypropyl methylcellulose or silicified hydroxypropyl methylcellulose by weight of the blend;
(c) about 38% to about 46% microcrystalline cellulose or silicified microcrystalline cellulose by weight of the blend;
(d) about 0.25% to about 0.75% magnesium stearate by weight of the blend; and
(e) about 0.25% to about 0.75% colloidal silicon dioxide by weight of the blend.

In some embodiments, the blend consists essentially of:
(a) about 43% dexpramipexole dihydrochloride monohydrate by weight of the blend;
(b) about 15% hydroxypropyl methylcellulose or silicified hydroxypropyl methylcellulose by weight of the blend;
(c) about 42% microcrystalline cellulose or silicified microcrystalline cellulose by weight of the blend;
(d) about 0.5% magnesium stearate by weight of the blend; and
(e) about 0.5% colloidal silicon dioxide by weight of the blend.

In some embodiments, the blend consists essentially of:
(a) about 40% to about 46% dexpramipexole dihydrochloride monohydrate by weight of the blend;
(b) about 10% to about 20% hydroxypropyl methylcellulose or silicified hydroxypropyl methylcellulose by weight of the blend;
(c) about 37% to about 45% microcrystalline cellulose or silicified microcrystalline cellulose by weight of the blend;
(d) about 0.5% to about 1.0% magnesium stearate by weight of the blend; and
(e) about 0.25% to about 0.75% colloidal silicon dioxide by weight of the blend.

In some embodiments, the blend consists essentially of:
(a) about 43% dexpramipexole dihydrochloride monohydrate by weight of the blend;
(b) about 15% hydroxypropyl methylcellulose or silicified hydroxypropyl methylcellulose by weight of the blend;
(c) about 41% microcrystalline cellulose or silicified microcrystalline cellulose by weight of the blend;
(d) about 0.75% magnesium stearate by weight of the blend; and
(e) about 0.5% colloidal silicon dioxide by weight of the blend.

In some embodiments, the blend consists essentially of:
(a) about 40% to about 46% dexpramipexole dihydrochloride monohydrate by weight of the blend;
(b) about 10% to about 20% hydroxypropyl methylcellulose or silicified hydroxypropyl methylcellulose by weight of the blend;
(c) about 37% to about 45% microcrystalline cellulose by weight of the blend;
(d) about 0.5% to about 1.0% magnesium stearate by weight of the blend; and
(e) about 0.25% to about 0.75% colloidal silicon dioxide by weight of the blend.

In some embodiments, the blend consists essentially of:
(a) about 43% dexpramipexole dihydrochloride monohydrate by weight of the blend;
(b) about 15% hydroxypropyl methylcellulose or silicified hydroxypropyl methylcellulose by weight of the blend;
(c) about 41% microcrystalline cellulose by weight of the blend;
(d) about 0.75% magnesium stearate by weight of the blend; and
(e) about 0.5% colloidal silicon dioxide by weight of the blend.

In some embodiments, the blend consists essentially of:
(a) about 40% to about 46% dexpramipexole dihydrochloride monohydrate by weight of the blend;
(b) about 24% to about 32% sodium carboxymethylcellulose by weight of the blend;
(c) about 25% to about 34% silicified microcrystalline cellulose by weight of the blend;
(d) about 0.25% to about 0.75% magnesium stearate by weight of the blend; and
(e) about 0.25% to about 0.75% colloidal silicon dioxide by weight of the blend.

In some embodiments, the blend consists essentially of:
(a) about 43% dexpramipexole dihydrochloride monohydrate by weight of the blend;
(b) about 28% sodium carboxymethylcellulose by weight of the blend;
(c) about 29% silicified microcrystalline cellulose by weight of the blend;
(d) about 0.5% magnesium stearate by weight of the blend; and
(e) about 0.5% colloidal silicon dioxide by weight of the blend.

In some embodiments, preparing the blend comprises:
mixing the densified granules with the glidant thereby providing a mixture of the densified granules and the glidant; and
mixing the mixture of the densified granules and the glidant with the lubricant thereby providing the blend.

In some embodiments, preparing the pre-blend comprises:
mixing dexpramipexole, or a pharmaceutically acceptable salt thereof, the one or more anionic polymers and a microcrystalline cellulose thereby providing a mixture of dexpramipexole, or a pharmaceutically acceptable salt thereof, the one or more anionic polymers and the microcrystalline cellulose; and
mixing the mixture of dexpramipexole, or a pharmaceutically acceptable salt thereof, the one or more anionic polymers and the microcrystalline cellulose with magnesium stearate thereby providing the pre-blend.

In some embodiments, preparing the pre-blend comprises:
mixing dexpramipexole, or a pharmaceutically acceptable salt thereof, the one or more polymers and a microcrystalline cellulose thereby providing a mixture of dexpramipexole, or a pharmaceutically acceptable salt thereof, the one or more anionic polymers and the microcrystalline cellulose; and
mixing the mixture of dexpramipexole, or a pharmaceutically acceptable salt thereof, the one or more polymers and the microcrystalline cellulose with magnesium stearate thereby providing the pre-blend.

In some embodiments, the microcrystalline cellulose is silicified microcrystalline cellulose.

In some embodiments, the pre-blend further comprises additional excipients. In some embodiments, the pre-blend further comprises a microcrystalline cellulose, magnesium stearate, or a combination thereof. In some embodiments, the pre-blend further comprises a microcrystalline cellulose and magnesium stearate. In some embodiments, the microcrystalline cellulose is silicified microcrystalline cellulose.

In some embodiments, the pre-blend comprises an antioxidant. In some embodiments, the antioxidant is capable of acting as a nitrite scavenger. Non-limiting examples of nitrite scavengers include, but are not limited to, ascorbic acid, L-cysteine, caffeic acid, cysteine HCL, methionine, tartaric acid, gallic acid, uric acid, and sodium sulphite. In some embodiments, the pre-blend comprises one nitrite scavenger. In another embodiment, the pre-blend comprises multiple nitrite scavengers. In some embodiments, the nitrite scavengers constitute about 0.1% to about 1.5% by weight of the tablet core. In some embodiments, the nitrite scavenger constitutes about 0.1% to about 1.0% by weight of the tablet core. In some embodiments, the nitrite scavenger constitutes about 0.1% to about 0.5% by weight of the tablet core. In some embodiments, the nitrite scavenger constitutes about 0.5% to about 1.5% by weight of the tablet core. In some embodiments, the nitrite scavenger constitutes about 1% to about 1.5% by weight of the tablet core. In some embodiments, the nitrite scavenger constitutes about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, or about 1.5% by weight of the tablet core.

In some embodiments, the densified granules are prepared by dry granulation. In some embodiments, the densified granules are prepared by roller compaction.

The sustained release pharmaceutical compositions comprising dexpramipexole, or a pharmaceutically acceptable salt thereof, of the present disclosure are generally not limited to the form of orally deliverable tablets, but can be provided in alternative dosage forms to tablets, such as capsules.

The present disclosure thus further provides also methods of manufacturing intermediate products (such as densified granules and blends) of sustained release pharmaceutical compositions comprising dexpramipexole, or a pharmaceutically acceptable salt thereof (which pharmaceutical compositions might, for example, be in the form of orally deliverable tablets or alternative dosage forms than tablets, such as capsules).

Thus, in some aspects, the present disclosure is also directed to a method of manufacturing densified granules for preparing a sustained release pharmaceutical composition comprising dexpramipexole, or a pharmaceutically acceptable salt thereof, the method comprising:
preparing a pre-blend by mixing dexpramipexole, or a pharmaceutically acceptable salt thereof, and one or more anionic polymers; and
preparing densified granules from the pre-blend;
wherein dexpramipexole, or the pharmaceutically acceptable salt thereof, constitutes about 30% to about 40% of dexpramipexole dihydrochloride monohydrate equivalent by weight of the densified granules and the one or more anionic polymers constitute about 30% to about 55% by weight of the densified granules.

In some aspects, the present disclosure is also directed to a method of manufacturing densified granules for preparing a sustained release pharmaceutical composition comprising dexpramipexole, or a pharmaceutically acceptable salt thereof, the method comprising:
preparing a pre-blend by mixing dexpramipexole, or a pharmaceutically acceptable salt thereof, and one or more polymers; and preparing densified granules from the pre-blend;
wherein dexpramipexole, or the pharmaceutically acceptable salt thereof, constitutes about 30% to about 50% of dexpramipexole dihydrochloride monohydrate equivalent by weight of the densified granules and the one or more polymers constitute about 5% to about 60% by weight of the densified granules.

In some aspects, the present disclosure is also directed to a method of manufacturing densified granules for preparing a sustained release pharmaceutical composition comprising dexpramipexole, or a pharmaceutically acceptable salt thereof, the method comprising:
preparing a pre-blend by mixing dexpramipexole, or a pharmaceutically acceptable salt thereof, and one or more polymers;
wherein dexpramipexole, or the pharmaceutically acceptable salt thereof, constitutes about 30% to about 50% of dexpramipexole dihydrochloride monohydrate equivalent by weight of the densified granules and the one or more polymers constitute about 5% to about 60% by weight of the densified granules.

In some embodiments, the pre-blend further comprises additional excipients. In some embodiments, the pre-blend further comprises a microcrystalline cellulose, magnesium stearate, or a combination thereof. In some embodiments, the pre-blend further comprises a microcrystalline cellulose and magnesium stearate. In some embodiments, the microcrystalline cellulose is silicified microcrystalline cellulose.

In some embodiments, dexpramipexole, or the pharmaceutically acceptable salt thereof, constitutes about 35% to about 44% of dexpramipexole dihydrochloride monohydrate equivalent by weight of the densified granules. In some embodiments, dexpramipexole, or the pharmaceutically acceptable salt thereof, constitutes about 35% to about 40% of dexpramipexole dihydrochloride monohydrate equivalent by weight of the densified granules.

In some embodiments, the one or more polymers are anionic polymers. In some embodiments, the one or more anionic polymers constitute about 20% to about 55% by weight of the densified granules.

In some embodiments, the one or more anionic polymers constitute about 25% to about 50% by weight of the densified granules. In some embodiments, the one or more anionic polymers constitute about 28% to about 48% by weight of the densified granules. In some embodiments, the one or more anionic polymers constitute about 28% by weight of the densified granules.

In some embodiments, the one or more anionic polymers constitute about 35% to about 50% by weight of the densified granules. In some embodiments, the one or more anionic polymers constitute about 40% to about 48% by weight of the densified granules. In some embodiments, the one or more anionic polymers constitute about 38% to about 42% by weight of the densified granules. In some embodiments, the one or more anionic polymers constitute about 46% to about 50% by weight of the densified granules. In some embodiments, the one or more anionic polymers constitute about 48% by weight of the densified granules. In some embodiments, the one or more anionic polymers constitute about 43% by weight of the densified granules. In some embodiments, the one or more anionic polymers constitute about 40% by weight of the densified granules.

In some embodiments, the one or more anionic polymers are selected from carboxymethylcellulose, an acrylic acid polymer, a methacrylic acid polymer, a methacrylic acid copolymer, alignate, carrageenan, xanthan gum, or arabic gum. In some embodiments, the one or more anionic polymers are selected from carboxymethylcellulose and an acrylic acid polymer. In some embodiments, carboxymethylcellulose is sodium carboxymethylcellulose. In some embodiments, the acrylic acid polymer is a crosslinked acrylic acid polymer.

In some embodiments, the pre-blend comprises a first anionic polymer and a second anionic polymer and wherein the first anionic polymer is carboxymethylcellulose and the second anionic polymer is an acrylic acid polymer. In some embodiments, the acrylic acid polymer is a crosslinked acrylic acid polymer. In some embodiments, carboxymethylcellulose is sodium carboxymethylcellulose. In some embodiments, carboxymethylcellulose constitutes about 27% to about 34% of sodium carboxymethylcellulose equivalent by weight of the densified granules and the acrylic acid polymer constitutes about 11% to about 15% by weight of the densified granules. In some embodiments, carboxymethylcellulose constitutes about 30% of sodium carboxymethylcellulose equivalent by weight of the densified granules and the acrylic acid polymer constitutes about 13% by weight of the densified granules. In some embodiments, carboxymethylcellulose constitutes about 27% to about 29% of sodium carboxymethylcellulose equivalent by weight of the densified granules and the acrylic acid polymer constitutes about 11% to about 13% by weight of the densified granules. In some embodiments, carboxymethylcellulose constitutes about 28% of sodium carboxymethylcellulose equivalent by weight of the densified granules and the acrylic acid polymer constitutes about 12% by weight of the densified granules. In some embodiments, carboxymethylcellulose constitutes about 32% to about 34% of sodium carboxymethylcellulose equivalent by weight of the densified granules and the acrylic acid polymer constitutes about 13% to about 15% by weight of the densified granules. In some embodiments, carboxymethylcellulose constitutes about 33% of sodium carboxymethylcellulose equivalent by weight of the densified granules and the acrylic acid polymer constitutes about 14% by weight of the densified granules.

In some embodiments, the pharmaceutically acceptable salt of dexpramipexole is a methane sulfonic acid salt, sulfuric acid salt, tartaric acid salt, p-toluene sulfonic acid salt, phosphoric acid salt, maleic acid salt, fumaric acid salt, malic acid salt, citric acid salt, succinic acid salt, or any combination thereof. In some embodiments, the pharmaceutically acceptable salt of dexpramipexole is dexpramipexole dihydrochloride or a hydrate thereof. In some embodiments, the pharmaceutically acceptable salt of dexpramipexole is dexpramipexole dihydrochloride monohydrate.

In some embodiments, the pre-blend comprises one anionic polymer and wherein the anionic polymer is carboxymethylcellulose. In some embodiments, the carboxymethylcellulose is sodium carboxymethylcellulose. In some embodiments, the carboxymethylcellulose constitutes about 27% to about 29% of sodium carboxymethylcellulose equivalent by weight of the densified granules. In some embodiments, the carboxymethylcellulose constitutes about 28% of sodium carboxymethylcellulose equivalent by weight of the densified granules.

In some embodiments, the one or more polymers are neutral polymers. In some embodiments, the one or more neutrals polymers is hydroxypropyl methylcellulose or silicified hydroxypropyl methylcellulose. In some embodiments, the one or more neutral polymers is hydroxypropyl methylcellulose. In some embodiments, the one or more neutral polymers is silicified hydroxypropyl methylcellulose. In some embodiments, the one or more neutral polymers constitute about 5% to about 25% by weight of the densified granules. In some embodiments, the one or more neutral polymers constitute about 15% by weight of the densified granules.

In some embodiments, the pre-blend comprises dexpramipexole, or a pharmaceutically acceptable salt thereof, and does not comprise an anionic polymer. In some embodiments, the pre-blend comprises dexpramipexole, or a pharmaceutically acceptable salt thereof, and does not comprise a neutral polymer.

In some embodiments, preparing the pre-blend comprises:
mixing dexpramipexole, or a pharmaceutically acceptable salt thereof, the one or more anionic polymers and a microcrystalline cellulose thereby providing a mixture of dexpramipexole, or a pharmaceutically acceptable salt thereof, the one or more anionic polymers and the microcrystalline cellulose; and
mixing the mixture of dexpramipexole, or a pharmaceutically acceptable salt thereof, the one or more anionic polymers and the microcrystalline cellulose with magnesium stearate thereby providing the pre-blend.

In some embodiments, preparing the pre-blend comprises:
mixing dexpramipexole, or a pharmaceutically acceptable salt thereof, the one or more polymers and a microcrystalline cellulose thereby providing a mixture of dexpramipexole, or a pharmaceutically acceptable salt thereof, the one or more polymers and the microcrystalline cellulose; and
mixing the mixture of dexpramipexole, or a pharmaceutically acceptable salt thereof, the one or more polymers and the microcrystalline cellulose with magnesium stearate thereby providing the pre-blend.

In some embodiments, the microcrystalline cellulose is silicified microcrystalline cellulose.

In some embodiments, the pre-blend further comprises additional excipients. In some embodiments, the pre-blend further comprises a microcrystalline cellulose, magnesium stearate, or a combination thereof. In some embodiments, the pre-blend further comprises a microcrystalline cellulose and magnesium stearate. In some embodiments, the microcrystalline cellulose is silicified microcrystalline cellulose.

In some embodiments, the densified granules are prepared by dry granulation. In some embodiments, the densified granules are prepared by roller compaction.

In some aspects, the present disclosure is further also directed to a method of manufacturing a blend for preparing a sustained release pharmaceutical composition comprising dexpramipexole, or a pharmaceutically acceptable salt thereof, the method comprising:
preparing a pre-blend by mixing dexpramipexole, or a pharmaceutically acceptable salt thereof, and one or more anionic polymers;
preparing densified granules from the pre-blend; and
preparing a blend by mixing the densified granules with a glidant and a lubricant;
wherein dexpramipexole, or the pharmaceutically acceptable salt thereof, constitutes about 30% to about 40% of dexpramipexole dihydrochloride monohydrate equivalent by weight of the blend and the one or more anionic polymers constitute about 30% to about 55% by weight of the blend.

In some aspects, the present disclosure is further also directed to a method of manufacturing a blend for preparing a sustained release pharmaceutical composition comprising dexpramipexole, or a pharmaceutically acceptable salt thereof, the method comprising:
preparing a pre-blend by mixing dexpramipexole, or a pharmaceutically acceptable salt thereof, and one or more polymers;
preparing densified granules from the pre-blend; and
preparing a final blend by mixing the densified granules with a glidant and a lubricant;
wherein dexpramipexole, or the pharmaceutically acceptable salt thereof, constitutes about 30% to about 50% of dexpramipexole dihydrochloride monohydrate equivalent by weight of the blend and the one or more polymers constitute about 5% to about 60% by weight of the blend.

In some aspects, the present disclosure is further also directed to a method of manufacturing a blend for preparing a sustained release pharmaceutical composition comprising dexpramipexole, or a pharmaceutically acceptable salt thereof, the method comprising:
preparing a pre-blend by mixing dexpramipexole, or a pharmaceutically acceptable salt thereof, and one or more polymers; and
preparing a final blend by mixing the pre-blend with a glidant and a lubricant;
wherein dexpramipexole, or the pharmaceutically acceptable salt thereof, constitutes about 30% to about 50% of dexpramipexole dihydrochloride monohydrate equivalent by weight of the blend and the one or more polymers constitute about 5% to about 60% by weight of the blend.

In some embodiments, the pre-blend further comprises additional excipients. In some embodiments, the final blend further comprises additional excipients.

In some embodiments, the glidant is colloidal silicon dioxide and the lubricant is magnesium stearate.

In some embodiments, the one or more polymers are anionic polymers. In some embodiments, the one or more anionic polymers constitute about 20% to about 55% by weight of the densified granules.

In some embodiments, the one or more polymers are neutral polymers. In some embodiments, the one or more polymers are hydroxypropyl methylcellulose or silicified hydroxypropyl methylcellulose. In some embodiments, the one or more neutral polymers is hydroxypropyl methylcellulose. In some embodiments, the one or more neutral polymers is silicified hydroxypropyl methylcellulose. In some embodiments, the one or more neutral polymers constitute about 5% to about 25% by weight of the blend.

In some embodiments, the one or more anionic polymers constitute about 35% to about 50% by weight of the blend. In some embodiments, the one or more anionic polymers constitute about 38% to about 42% by weight of the blend. In some embodiments, the one or more anionic polymers constitute about 40% by weight of the blend. In some embodiments, the one or more anionic polymers constitute about 45% to about 49% by weight of the blend. In some embodiments, the one or more anionic polymers constitute about 47% by weight of the blend. In some embodiments, the one or more anionic polymers constitute about 43% by weight of the blend. In some embodiments, the one or more anionic polymers constitute about 40% by weight of the blend. In some embodiments, the one or more anionic polymers constitute about 28% by weight of the blend.

In some embodiments, the one or more anionic polymers are selected from carboxymethylcellulose, an acrylic acid polymer, a methacrylic acid polymer, a methacrylic acid copolymer, alignate, carrageenan, xanthan gum, or arabic gum. In some embodiments, the one or more anionic polymers are selected from carboxymethylcellulose and an acrylic acid polymer. In some embodiments, carboxymethylcellulose is sodium carboxymethylcellulose. In some embodiments, the acrylic acid polymer is a crosslinked acrylic acid polymer. In some embodiments, the pre-blend comprises a first anionic polymer and a second anionic polymer and wherein the first anionic polymer is carboxymethylcellulose and the second anionic polymer is an acrylic acid polymer. In some embodiments, the acrylic acid polymer is a crosslinked acrylic acid polymer. In some embodiments, carboxymethylcellulose is sodium carboxymethylcellulose.

In some embodiments, carboxymethylcellulose constitutes about 27% to about 34% of sodium carboxymethylcellulose equivalent by weight of the blend and the acrylic acid polymer constitutes about 11% to about 15% by weight of the blend. In some embodiments, carboxymethylcellulose constitutes about 30% of sodium carboxymethylcellulose equivalent by weight of the blend and the acrylic acid polymer constitutes about 13% by weight of the blend. In some embodiments, carboxymethylcellulose constitutes about 27% to about 29% of sodium carboxymethylcellulose equivalent by weight of the blend and the acrylic acid polymer constitutes about 11% to about 13% by weight of the blend. In some embodiments, carboxymethylcellulose constitutes about 28% of sodium carboxymethylcellulose equivalent by weight of the blend and the acrylic acid polymer constitutes about 12% by weight of the blend. In some embodiments, carboxymethylcellulose constitutes about 32% to about 34% of sodium carboxymethylcellulose equivalent by weight of the blend and the acrylic acid polymer constitutes about 13% to about 15% by weight of the blend. In some embodiments, carboxymethylcellulose constitutes about 33% of sodium carboxymethylcellulose equivalent by weight of the blend and the acrylic acid polymer constitutes about 14% by weight of the blend.

In some embodiments, the densified granules constitute about 99% by weight of the blend. In some embodiments, the glidant constitutes about 0.5% by weight of the blend and the lubricant constitutes about 0.5% by weight of the blend.

In some embodiments, the pharmaceutically acceptable salt of dexpramipexole is a methane sulfonic acid salt, sulfuric acid salt, tartaric acid salt, p-toluene sulfonic acid salt, phosphoric acid salt, maleic acid salt, fumaric acid salt, malic acid salt, citric acid salt, succinic acid salt, or any combination thereof. In some embodiments, the pharmaceutically acceptable salt of dexpramipexole is dexpramipexole dihydrochloride or a hydrate thereof. In some embodiments, the pharmaceutically acceptable salt of dexpramipexole is dexpramipexole dihydrochloride monohydrate.

In some embodiments, the blend consists essentially of:
(a) about 35% to about 40% dexpramipexole dihydrochloride monohydrate by weight of the blend;
(b) about 25% to about 30% sodium carboxymethylcellulose by weight of the blend;
(c) about 10% to about 15% crosslinked acrylic acid polymer by weight of the blend;
(d) about 18% to about 23% silicified microcrystalline cellulose by weight of the blend;
(e) about 0.5% to about 1.0% magnesium stearate by weight of the blend; and
(f) about 0.25% to about 0.75% colloidal silicon dioxide by weight of the blend.

In some embodiments, the blend consists essentially of:
(a) about 38% dexpramipexole dihydrochloride monohydrate by weight of the blend;
(b) about 28% sodium carboxymethylcellulose by weight of the blend;
(c) about 12% crosslinked acrylic acid polymer by weight of the blend;
(d) about 21% silicified microcrystalline cellulose by weight of the blend;
(e) about 0.75% magnesium stearate by weight of the blend; and
(f) about 0.5% colloidal silicon dioxide by weight of the blend.

In some embodiments, the blend consists essentially of:
(a) about 33% to about 38% dexpramipexole dihydrochloride monohydrate by weight of the blend;
(b) about 30% to about 35% sodium carboxymethylcellulose by weight of the blend;
(c) about 12% to about 17% crosslinked acrylic acid polymer by weight of the blend;
(d) about 13% to about 18% silicified microcrystalline cellulose by weight of the blend;
(e) about 0.5% to about 1.0% magnesium stearate by weight of the blend; and
(f) about 0.25% to about 0.75% colloidal silicon dioxide by weight of the blend.

In some embodiments, the blend consists essentially of:
(a) about 35% dexpramipexole dihydrochloride monohydrate by weight of the blend;
(b) about 33% sodium carboxymethylcellulose by weight of the blend;
(c) about 14% crosslinked acrylic acid polymer by weight of the blend;
(d) about 16% silicified microcrystalline cellulose by weight of the blend;
(e) about 0.75% magnesium stearate by weight of the blend; and
(f) about 0.5% colloidal silicon dioxide by weight of the blend.

In some embodiments, the blend consists essentially of:
(a) about 33% to about 39% dexpramipexole dihydrochloride monohydrate by weight of the blend;
(b) about 27% to about 33% sodium carboxymethylcellulose by weight of the blend;
(c) about 10% to about 16% crosslinked acrylic acid polymer by weight of the blend;
(d) about 18% to about 24% microcrystalline cellulose or silicified microcrystalline cellulose by weight of the blend;
(e) about 0.5% to about 1.0% magnesium stearate by weight of the blend; and
(f) about 0.25% to about 0.75% colloidal silicon dioxide by weight of the blend.

In some embodiments, the blend consists essentially of:
(a) about 36% dexpramipexole dihydrochloride monohydrate by weight of the blend;
(b) about 30% sodium carboxymethylcellulose by weight of the blend;
(c) about 13% crosslinked acrylic acid polymer by weight of the blend;
(d) about 21% microcrystalline cellulose or silicified microcrystalline cellulose by weight of the blend;
(e) about 0.75% magnesium stearate by weight of the blend; and
(f) about 0.5% colloidal silicon dioxide by weight of the blend.

In some embodiments, the blend consists essentially of:
(a) about 40% to about 46% dexpramipexole dihydrochloride monohydrate by weight of the blend;
(b) about 10% to about 20% hydroxypropyl methylcellulose or silicified hydroxypropyl methylcellulose by weight of the blend;
(c) about 38% to about 46% microcrystalline cellulose or silicified microcrystalline cellulose by weight of the blend;
(d) about 0.25% to about 0.75% magnesium stearate by weight of the blend; and
(e) about 0.25% to about 0.75% colloidal silicon dioxide by weight of the blend.

In some embodiments, the blend consists essentially of:
(a) about 43% dexpramipexole dihydrochloride monohydrate by weight of the blend;
(b) about 15% hydroxypropyl methylcellulose or silicified hydroxypropyl methylcellulose by weight of the blend;
(c) about 42% microcrystalline cellulose or silicified microcrystalline cellulose by weight of the blend;
(d) about 0.5% magnesium stearate by weight of the blend; and
(e) about 0.5% colloidal silicon dioxide by weight of the blend.

In some embodiments, the blend consists essentially of:
(a) about 40% to about 46% dexpramipexole dihydrochloride monohydrate by weight of the blend;
(b) about 10% to about 20% hydroxypropyl methylcellulose or silicified hydroxypropyl methylcellulose by weight of the blend;
(c) about 37% to about 45% microcrystalline cellulose or silicified microcrystalline cellulose by weight of the blend;
(d) about 0.5% to about 1.0% magnesium stearate by weight of the blend; and
(e) about 0.25% to about 0.75% colloidal silicon dioxide by weight of the blend.

In some embodiments, the blend consists essentially of:
(a) about 43% dexpramipexole dihydrochloride monohydrate by weight of the blend;
(b) about 15% hydroxypropyl methylcellulose or silicified hydroxypropyl methylcellulose by weight of the blend;
(c) about 41% microcrystalline cellulose or silicified microcrystalline cellulose by weight of the blend;
(d) about 0.75% magnesium stearate by weight of the blend; and
(e) about 0.5% colloidal silicon dioxide by weight of the blend.

In some embodiments, the blend consists essentially of:
(a) about 40% to about 46% dexpramipexole dihydrochloride monohydrate by weight of the blend;
(b) about 10% to about 20% hydroxypropyl methylcellulose or silicified hydroxypropyl methylcellulose by weight of the blend;
(c) about 37% to about 45% microcrystalline cellulose by weight of the blend;
(d) about 0.5% to about 1.0% magnesium stearate by weight of the blend; and
(e) about 0.25% to about 0.75% colloidal silicon dioxide by weight of the blend.

In some embodiments, the blend consists essentially of:
(a) about 43% dexpramipexole dihydrochloride monohydrate by weight of the blend;
(b) about 15% hydroxypropyl methylcellulose or silicified hydroxypropyl methylcellulose by weight of the blend;
(c) about 41% microcrystalline cellulose by weight of the blend;
(d) about 0.75% magnesium stearate by weight of the blend; and
(e) about 0.5% colloidal silicon dioxide by weight of the blend.

In some embodiments, the blend consists essentially of:
(a) about 40% to about 46% dexpramipexole dihydrochloride monohydrate by weight of the blend;
(b) about 24% to about 32% sodium carboxymethylcellulose by weight of the blend;
(c) about 25% to about 34% microcrystalline cellulose or silicified microcrystalline cellulose by weight of the blend;
(d) about 0.25% to about 0.75% magnesium stearate by weight of the blend; and
(e) about 0.25% to about 0.75% colloidal silicon dioxide by weight of the blend.

In some embodiments, the blend consists essentially of:
(a) about 43% dexpramipexole dihydrochloride monohydrate by weight of the blend;
(b) about 28% sodium carboxymethylcellulose by weight of the blend;
(c) about 29% microcrystalline cellulose or silicified microcrystalline cellulose by weight of the blend;
(d) about 0.5% magnesium stearate by weight of the blend; and
(e) about 0.5% colloidal silicon dioxide by weight of the blend.

In some embodiments, preparing the blend comprises:
mixing the densified granules with the glidant thereby providing a mixture of the densified granules and the glidant; and
mixing the mixture of the densified granules and the glidant with the lubricant thereby providing the blend.

In some embodiments, preparing the pre-blend comprises:
mixing dexpramipexole, or a pharmaceutically acceptable salt thereof, the one or more anionic polymers and a microcrystalline cellulose thereby providing a mixture of dexpramipexole, or a pharmaceutically acceptable salt thereof, the one or more anionic polymers and the microcrystalline cellulose; and
mixing the mixture of dexpramipexole, or a pharmaceutically acceptable salt thereof, the one or more anionic polymers and the microcrystalline cellulose with magnesium stearate thereby providing the pre-blend.

In some embodiments, preparing the pre-blend comprises:
mixing dexpramipexole, or a pharmaceutically acceptable salt thereof, the one or more polymers and a microcrystalline cellulose thereby providing a mixture of dexpramipexole, or a pharmaceutically acceptable salt thereof, the one or more anionic polymers and the microcrystalline cellulose; and
mixing the mixture of dexpramipexole, or a pharmaceutically acceptable salt thereof, the one or more polymers and the microcrystalline cellulose with magnesium stearate thereby providing the pre-blend.

In some embodiments, the microcrystalline cellulose is silicified microcrystalline cellulose.

In some embodiments, the pre-blend further comprises a microcrystalline cellulose, magnesium stearate, or a combination thereof. In some embodiments, the pre-blend further comprises a microcrystalline cellulose and magnesium stearate. In some embodiments, the microcrystalline cellulose is silicified microcrystalline cellulose.

In some embodiments, the densified granules are prepared by dry granulation. In some embodiments, the densified granules are prepared by roller compaction.

In the context of the methods of manufacturing provided herein, preparing the densified granules by roller compaction was shown to be particular robust as it minimized the variability amongst different manufacturing batches and showed minimal formulation sensitivity (see in particular Examples 4 and 5).

As outlined above and demonstrated in the Examples with regards to orally deliverable tablets, an amount of dexpramipexole, or a pharmaceutically acceptable salt thereof, typically used (such as about 300 mg or about 376 mg dexpramipexole dihydrochloride equivalent) can be delivered in a sustained release pharmaceutical composition suitable for once daily oral administration to a human when mixed with one or more anionic polymers at the amounts specified herein.

Thus, in some aspects, the present disclosure is also directed to a mixture comprising dexpramipexole, or a pharmaceutically acceptable salt thereof, constituting about 30% to about 40% of dexpramipexole hydrochloride monohydrate equivalent by weight of the mixture and about 30% to about 55% of one or more anionic polymers by weight of the mixture. In some aspects, the present disclosure is also directed to a mixture comprising dexpramipexole, or a pharmaceutically acceptable salt thereof, constituting about 30% to about 50% of dexpramipexole hydrochloride monohydrate equivalent by weight of the mixture.

In some embodiments, the one or more polymers are anionic polymers.

In some embodiments, the one or more anionic polymers constitute about 35% to about 50% by weight of the mixture. In some embodiments, the one or more anionic polymers constitute about 38% to about 42% by weight of the mixture. In some embodiments, the one or more anionic polymers constitute about 40% by weight of the mixture. In some embodiments, the one or more anionic polymers constitute about 45% to about 49% by weight of the mixture. In some embodiments, the one or more anionic polymers constitute about 47% by weight of the mixture.

In some embodiments, the one or more anionic polymers constitute about 20% to about 55% by weight of the mixture. In some embodiments, the one or more anionic polymers constitute about 28% to about 48% by weight of the mixture. In some embodiments, the one or more anionic polymers constitute about 28% by weight of the mixture. In some embodiments, the one or more anionic polymers constitute about 40% to about 48% by weight of the mixture. In some embodiments, the one or more anionic polymers constitute about 40% by weight of the mixture. In some embodiments, the one or more anionic polymers constitute about 43% by weight of the mixture. In some embodiments, the one or more anionic polymers constitute about 48% by weight of the mixture.

In some embodiments, the pharmaceutically acceptable salt of dexpramipexole is a methane sulfonic acid salt, sulfuric acid salt, tartaric acid salt, p-toluene sulfonic acid salt, phosphoric acid salt, maleic acid salt, fumaric acid salt, malic acid salt, citric acid salt, succinic acid salt, or any combination thereof. In some embodiments, the pharmaceutically acceptable salt of dexpramipexole is dexpramipexole dihydrochloride or a hydrate thereof. In some embodiments, the pharmaceutically acceptable salt of dexpramipexole is dexpramipexole dihydrochloride monohydrate.

In some embodiments, the one or more anionic polymers are selected from carboxymethylcellulose, an acrylic acid polymer, a methacrylic acid polymer, a methacrylic acid copolymer, alignate, carrageenan, xanthan gum, or arabic gum. In some embodiments, the one or more anionic polymers are selected from carboxymethylcellulose and an acrylic acid polymer. In some embodiments, carboxymethylcellulose is sodium carboxymethylcellulose. In some embodiments, the acrylic acid polymer is a crosslinked acrylic acid polymer.

In some embodiments, the mixture comprises a first anionic polymer and a second anionic polymer, wherein the first anionic polymer is carboxymethylcellulose and the second anionic polymer is an acrylic acid polymer. In some embodiments, the acrylic acid polymer is a crosslinked acrylic acid polymer. In some embodiments, carboxymethylcellulose is sodium carboxymethylcellulose.

In some embodiments, carboxymethylcellulose constitutes about 27% to about 34% of sodium carboxymethylcellulose equivalent by weight of the mixture and the acrylic acid polymer constitutes about 11% to about 15% by weight of the mixture. In some embodiments, carboxymethylcellulose constitutes about 30% of sodium carboxymethylcellulose equivalent by weight of the mixture and the acrylic acid polymer constitutes about 13% by weight of the mixture. In some embodiments, carboxymethylcellulose constitutes about 27% to about 29% of sodium carboxymethylcellulose equivalent by weight of the mixture and the acrylic acid polymer constitutes about 11% to about 13% by weight of the mixture. In some embodiments, carboxymethylcellulose constitutes about 28% of sodium carboxymethylcellulose equivalent by weight of the mixture and the acrylic acid polymer constitutes about 12% by weight of the mixture. In some embodiments, carboxymethylcellulose constitutes about 32% to about 34% of sodium carboxymethylcellulose equivalent by weight of the mixture and the acrylic acid polymer constitutes about 13% to about 15% by weight of the mixture. In some embodiments, carboxymethylcellulose constitutes about 33% of sodium carboxymethylcellulose equivalent by weight of the mixture and the acrylic acid polymer constitutes about 14% by weight of the mixture.

In some embodiments, the mixture comprises one anionic polymer and wherein the anionic polymer is carboxymethylcellulose. In some embodiments, the carboxymethylcellulose is sodium carboxymethylcellulose. In some embodiments, the carboxymethylcellulose constitutes about 27% to about 29% of sodium carboxymethylcellulose equivalent by weight of the densified granules. In some embodiments, the carboxymethylcellulose constitutes about 28% of sodium carboxymethylcellulose equivalent by weight of the densified granules.

In some embodiments, the one or more polymers are neutral polymers. In some embodiments, the one or more neutral polymers are hydroxypropyl methylcellulose or silicified hydroxypropyl methylcellulose. In some embodiments, the one or more neutral polymers is hydroxypropyl methylcellulose. In some embodiments, the one or more neutral polymers is silicified hydroxypropyl methylcellulose. In some embodiments, the one or more neutral polymers constitute about 5% to about 25% by weight of the densified granules. In some embodiments, the one or more neutral polymers constitute about 15% by weight of the densified granules.

In some embodiments, the mixture comprises dexpramipexole, or a pharmaceutically acceptable salt thereof, and does not comprise an anionic polymer. In some embodiments, the mixture comprises dexpramipexole, or a pharmaceutically acceptable salt thereof, and does not comprise a neutral polymer.

In some embodiments, the mixture consists essentially of dexpramipexole, or a pharmaceutically acceptable salt thereof, and two anionic polymers.

In some embodiments, the mixture consists essentially of dexpramipexole, or a pharmaceutically acceptable salt thereof, and one anionic polymer.

In some embodiments, the mixture consists essentially of dexpramipexole, or a pharmaceutically acceptable salt thereof, and one neutral polymer.

In some embodiments, the mixture further comprises additional excipients. In some embodiments, the mixture further comprises a microcrystalline cellulose, magnesium stearate, colloidal silicon dioxide, or any combination thereof.

In some embodiments, dexpramipexole, or the pharmaceutically acceptable salt thereof, has a chiral purity for dexpramipexole, or the pharmaceutically salt thereof, of 99.96% or more. In some embodiments, the mixture comprises 0.02% or less pramipexole, or a pharmaceutically acceptable salt thereof, by weight of the mixture. In some embodiments, the mixture comprises 0.015% or less pramipexole, or a pharmaceutically acceptable salt thereof, by weight of the mixture.

The mixture of the present disclosure can be formulated into sustained release pharmaceutical compositions that is suitable for once daily oral administration to a human. Such sustained release pharmaceutical compositions comprising the mixture of the present disclosure can be, for example, in the form of orally deliverable capsules or orally deliverable tablets.

Thus, in some aspects, the present disclosure is further directed to a sustained release pharmaceutical composition comprising the mixture as described herein.

In some embodiments, the pharmaceutical composition comprises dexpramipexole, or a pharmaceutically acceptable salt thereof, in an amount of about 50 mg to about 500 mg of dexpramipexole dihydrochloride equivalent. In some embodiments, the pharmaceutical composition comprises dexpramipexole, or a pharmaceutically acceptable salt thereof, in an amount of about 50 mg to about 400 mg of dexpramipexole dihydrochloride equivalent. In some embodiments, the amount of dexpramipexole, or a pharmaceutically acceptable salt thereof, is about 75 mg to about 300 mg of dexpramipexole dihydrochloride equivalent. In some embodiments, the amount of dexpramipexole, or a pharmaceutically acceptable salt thereof, is about 75 mg of dexpramipexole dihydrochloride equivalent. In some embodiments, the amount of dexpramipexole, or a pharmaceutically acceptable salt thereof, is about 150 mg of dexpramipexole dihydrochloride equivalent. In some embodiments, the amount of dexpramipexole, or a pharmaceutically acceptable salt thereof, is about 300 mg of dexpramipexole dihydrochloride equivalent. In some embodiments, the amount of dexpramipexole, or a pharmaceutically acceptable salt thereof, is about 376 mg of dexpramipexole dihydrochloride equivalent.

In some embodiments, the weight of the composition is about 1500 mg or less. In some embodiments, the weight of the composition is about 1300 mg or less. In some embodiments, the weight of the composition is about 1000 mg or less.

In some embodiments, the composition is an orally deliverable capsule. In some embodiments, the capsule comprises the mixture enclosed in one or more beads.

In some embodiments, the composition is an orally deliverable tablet.

Further, the present disclosure provides methods of treating or preventing certain diseases in a human subject in need thereof, the methods comprising orally administering to the human subject the pharmaceutical compositions as described herein. As dexpramipexole lowers eosinophil counts in vivo, the pharmaceutical compositions as described herein can generally be applied for treating or preventing eosinophilic disorders in humans. Non-limiting examples of diseases that can be treated or prevented with the pharmaceutical compositions as described herein are asthma and chronic obstructive pulmonary disease (COPD).

Examples 5.4 and 7 describe formulation and preparation of different exemplary matrix tablets according to the present disclosure comprising anionic polymers at amounts specified herein (one providing for a comparably faster and the other providing for a comparably slower release of drug) for evaluation in clinical trials. Example 9 also describes formulation and preparation of different exemplary matrix tablets according to the present disclosure comprising one or more anionic polymers or a neutral polymer at amounts specified herein for evaluation in clinical trials. The exemplary tablet for evaluation in clinical trials providing for faster release comprises a comparably lower amount of anionic polymers, whereas the exemplary tablet for evaluation in clinical trials providing for slower release comprises a comparably higher amount of anionic polymers. A Phase I clinical trial for evaluation of the exemplary matrix tablets is conducted (see Example 8).

In some aspects, the present disclosure is further directed to a method of treating or preventing asthma in a human subject in need thereof, the method comprising orally administering to the human subject the composition as described herein. In some embodiments, the asthma is eosinophilic asthma.

In some aspects, the present disclosure is further directed to a method of treating or preventing chronic obstructive pulmonary disease in a human subject in need thereof, the method comprising orally administering to the human subject the composition as described herein.

In some aspects, the present disclosure is further directed to a method of treating or preventing an eosinophilic disorder in a human subject in need thereof, the method comprising orally administering to the human subject the composition as described herein. In some embodiments, the eosinophilic disorder is selected from the group consisting of hypereosinophilic syndrome, chronic rhinosinusitis with nasal polyps, nasal polyposis, atopic dermatitis, eosinophilic granulomatosis with polyangiitis, eosinophilic gastroenteritis, eosinophilic esophagitis, and any combination thereof.

In some embodiments, the composition is administered once daily.

In some aspects, the present disclosure is also directed to the composition as described herein for use as a medicament. In some aspects, the present disclosure is also directed to the composition as described for use in a method of treating or preventing as described herein.

In some aspects, the present disclosure is also directed to the use of the composition as described herein in the manufacture of a medicament for a method of treating or preventing as described herein.

### BRIEF DESCRIPTION OF THE DRAWINGS

FIG. 1 In vitro dissolution profiles of sustained release matrix tablets comprising dexpramipexole dihydrochloride monohydrate and hydrophilic neutral polymers in 50 mM monobasic potassium phosphate buffer, pH 6.8 (P: Paddles at 50 rpm; B: Baskets at 100 rpm). HPMC=Hydroxypropyl methylcellulose, HPC=Hydroxypropyl cellulose.

FIG. 2 Impact of manufacturing process (direct compression, DC, vs. twin-screw wet-granulation, TSWG) on the in vitro dissolution profiles of sustained release matrix tablets comprising dexpramipexole dihydrochloride monohydrate and hydrophilic neutral polymers in 50 mM monobasic potassium phosphate buffer, pH 6.8 (P: Paddles at 50 rpm).

FIG. 3 In vitro dissolution profiles of sustained release matrix tablets comprising dexpramipexole dihydrochloride monohydrate and lipophilic materials manufactured using direct compression (DC) in 50 mM monobasic potassium phosphate buffer, pH 6.8 (P: Paddles at 50 rpm). HPC=Hydroxypropyl cellulose.

FIG. 4 Comparison of the effects of direct compression (DC) and melt granulation (Melt) manufacturing processes on the in vitro dissolution profiles of sustained release matrix tablets comprising dexpramipexole dihydrochloride monohydrate and lipophilic materials in 50 mM monobasic potassium phosphate buffer, pH 6.8 (P: Paddles at 50 rpm). HPC=Hydroxypropyl cellulose.

FIG. 5 Comparison of effects of the lipophilic materials glycerol dibehenate (Compritol 888 ATO) and carnuba wax on the in vitro dissolution profiles of sustained release matrix tablets comprising dexpramipexole dihydrochloride monohydrate manufactured using melt granulation (Melt) in 50 mM monobasic potassium phosphate buffer, pH 6.8 (P: Paddles at 50 rpm).

FIG. 6 Tablet shape and dimensions for tensile strength calculation (from Pitt, K. G. and Heasley M. G. Determination of the tensile strength of elongated tablets. Powder Technology 238 (2013) 169-175).

FIG. 7 Impact of the anionic polymer carbomer (Carbopol 971P) level on the in vitro dissolution profiles of sustained release matrix tablets comprising dexpramipexole dihydrochloride monohydrate in 50 mM monobasic potassium phosphate buffer, pH 6.8 (P: Paddles at 50 rpm).

FIG. 8 Impact of the total amount of anionic polymer at a fixed anionic polymer ratio of 3:2 carbomer:sodium carboxymethylcellulose (NaCMC) on the in vitro dissolution profiles of sustained release matrix tablets comprising dexpramipexole dihydrochloride monohydrate in 50 mM monobasic potassium phosphate buffer, pH 6.8 (B: Baskets at 100 rpm).

FIG. 9 Impact of the anionic polymer ratio of carbomer:sodium carboxymethylcellulose (NaCMC) at a fixed total anionic polymer amount of 40% (w/w) on the in vitro dissolution profiles of sustained release matrix tablets comprising dexpramipexole dihydrochloride monohydrate in 50 mM monobasic potassium phosphate buffer, pH 6.8 (B: Baskets at 100 rpm).

FIG. 10 In vitro dissolution profiles of fast and slow prototypes of sustained release matrix tablets comprising dexpramipexole dihydrochloride monohydrate in 0.1 N HCl (B: Baskets at 100 rpm). NaCMC=sodium carboxymethylcellulose.

FIG. 11 In vitro dissolution profiles of fast and slow prototypes of sustained release matrix tablets comprising dexpramipexole dihydrochloride monohydrate in McIlvaine Buffer, pH 4.5 (B: Baskets at 100 rpm). NaCMC=sodium carboxymethylcellulose.

FIG. 12 In vitro dissolution profiles of fast and slow prototypes of sustained release matrix tablets comprising dexpramipexole dihydrochloride monohydrate in 50 mM monobasic potassium phosphate buffer, pH 6.8 (B: Baskets at 100 rpm). NaCMC=sodium carboxymethylcellulose.

FIG. 13 In vitro dissolution profiles of sustained release matrix tablets comprising dexpramipexole dihydrochloride monohydrate in 50 mM monobasic potassium phosphate buffer, pH 6.8 prepared at larger scale (1 kg blend) and with different processes (B: Baskets at 100 rpm). HSG=high sear wet-granulation, RC=roller compaction, NaCMC=sodium carboxymethylcellulose.

FIG. 14 Impact of type of manufacturing process and manufacturing scale on in vitro dissolution profiles of sustained release matrix tablets comprising dexpramipexole dihydrochloride monohydrate in 50 mM monobasic potassium phosphate buffer, pH 6.8 (B: Baskets at 100 rpm). HSG=high sear wet-granulation, DC=direct compression, RC=roller compaction, NaCMC=sodium carboxymethylcellulose; Feasibility scale=12.75 g blend, and scale of RC and HSG=1 kg blend).

FIG. 15 Particle diameter distribution (PSD) comparison of roller compacted batches.

FIG. 16 Compression study #1—weight variation plot with roller compaction (RC) parameters of 2.5 kN, 0.8 mm mesh screen and 25 rpm press speed.

FIG. 17 Compression study #2—weight variation plot with roller compaction (RC) parameters of 3.0 kN, 1.0 mm mesh screen and 20 rpm press speed.

FIG. 18 Evaluation of reproducibility between smaller (1 kg: R&D) and larger (10 kg; Eng batch) scale manufacturing. The graph shows the in vitro dissolution profiles of sustained release matrix tablets comprising dexpramipexole dihydrochloride monohydrate in 50 mM monobasic potassium phosphate buffer, pH 6.8 (B: Baskets at 100 rpm).

FIG. 19 Effect of tablet hardness on in vitro dissolution. The graph shows the in vitro dissolution profiles of sustained release matrix tablets comprising dexpramipexole dihydrochloride monohydrate in 50 mM monobasic potassium phosphate buffer, pH 6.8 (B: Baskets at 100 rpm).

FIG. 20 Weight variation plot for engineering batches with 20 rpm press speed.

FIG. 21 Evaluation of reproducibility between smaller (R&D) and larger (Eng batch and GMP clinical batch) scale manufacturing. The graph shows the in vitro dissolution profiles of sustained release matrix tablets comprising dexpramipexole dihydrochloride monohydrate in 50 mM monobasic potassium phosphate buffer, pH 6.8 (B: Baskets at 100 rpm).

FIG. 22 Weight variation plot for clinical trial batches with 20 rpm press speed.

FIG. 23 Coated tablets of clinical trial batch 23JM-107 (fast prototype).

FIG. 24 In vitro dissolution profiles of sustained release matrix tablets (batches 030444-36 and 030444-38) comprising dexpramipexole dihydrochloride monohydrate in 50 mM monobasic potassium phosphate buffer, pH 6.8 (B: Baskets at 100 rpm).

FIG. 25 In vitro dissolution profiles of sustained release matrix tablets comprising dexpramipexole dihydrochloride monohydrate (batch 030444-36; fast prototype) in 50 mM monobasic potassium phosphate buffer, pH 6.8 (B: Baskets at 100 rpm). Dissolution profiles are shown for tablets after manufacturing (initial), after 1 month storage under CRT conditions (1M), and after 11 weeks storage under CRT conditions (11 W).

FIG. 26 In vitro dissolution profiles of sustained release matrix tablets comprising dexpramipexole dihydrochloride monohydrate (batch 030444-38; slow prototype) in 50 mM monobasic potassium phosphate buffer, pH 6.8 (B: Baskets at 100 rpm). Dissolution profiles are shown for tablets after manufacturing (initial), after 1 month storage under CRT conditions (1M), and after 11 weeks storage under CRT conditions (11 W).

FIG. 27 Schematic outline of Phase I Clinical Trial EXHALE-6.

FIG. 28 In vitro dissolution profiles of sustained release matrix tablets comprising dexpramipexole 280 mg (equivalent to 376 mg Dexpramipexole Dihydrochloride) in 0.1 N HCl. The impact of total polymer level was evaluated. Polymers indicated in the legend include sodium carboxymethylcellulose and carbomer.

FIG. 29 In vitro dissolution profile of sustained release matrix tablet comprising dexpramipexole 223 mg (equivalent to 300 mg Dexpramipexole Dihydrochloride) in 0.1 N HCl.

FIG. 30 In vitro dissolution profiles of sustained release matrix tablets comprising dexpramipexole 223 mg (equivalent to 300 mg Dexpramipexole Dihydrochloride) in 0.1 N HCl. The impact of HPMC K100M level was evaluated.

FIG. 31 In vitro dissolution profiles of sustained release matrix tablets comprising dexpramipexole 223 mg (equivalent to 300 mg Dexpramipexole Dihydrochloride) in 0.1 N HCl. The impact of NaCMC 7HXF level was evaluated.

FIG. 32 In vitro dissolution profiles of sustained release matrix tablets comprising dexpramipexole 223 mg (equivalent to 300 mg Dexpramipexole Dihydrochloride) in 0.1 N HCl. The impact of NaCMC 7HXF level was evaluated.

FIG. 33 Process schematic chart of Dexpramipexole ER matrix 280 mg tablets (376 mg diHCl) confirmation batch lots 031466-17 and 031466-29.

FIG. 34 Weight variation plot with roller compaction (RC) at 40 rpm turret speed.

FIG. 35 Weight variation plot with roller compaction (RC) at 20 rpm turret speed.

FIG. 36 Coated tablets of Dexpramipexole ER matrix 280 mg tablets (376 mg diHCl) confirmation batch, lot 031466-29.

FIG. 37 In vitro dissolution profiles of sustained release matrix tablets comprising dexpramipexole 280 mg (equivalent to 376 mg Dexpramipexole Dihydrochloride) in 0.1 N HCl compared to ER2 clinical batch.

FIG. 38 Process schematic chart of Dexpramipexole ER matrix 223 mg tablets (300 mg diHCl) confirmation batch lots 031466-19 and 031466-24.

FIG. 39 Tabletability study of ER7H lot 031466-19.

FIG. 40 Weight variation plot of ER7H lot 031466-19.

FIG. 41 Tablet appearance of ER7H lot 031466-19.

FIG. 42 In vitro dissolution profiles of ER7H lots 031466-19A and 031466-19B.

FIG. 43 Tabletability study of ER7H lot 031466-24.

FIG. 44 Weight variation plot of ER7H lot 031466-24.

FIG. 45 Tablet appearance of ER7H lot 031466-24.

FIG. 46 In vitro dissolution profiles of ER7H lot 031466-24 second confirmation batch.

FIG. 47 Tabletability study of the impact of HPMC grade.

FIG. 48 In vitro dissolution profiles of ER7H lots 031466-24, 031466-31, and 031466-32 in 0.1 N HCL. The impact of HPMC grade was evaluated.

FIG. 49 Tabletability study of ER7H lot 031466-20.

FIG. 50 Weight variation plot of ER7H lot 031466-20.

FIG. 51 Tablet appearance of ER7H lot 031466-20.

FIG. 52 Weight variation plot of ER7H lot 031466-23.

FIG. 53 In vitro dissolution profiles of ER9 lot 031466-23.

FIG. 54 Particle size distribution of final blend from 2nd confirmation batch (with API co-mill process) vs. development batch (without API co-mill process).

FIG. 55 Tabletability study of ER9 lot 031466-26.

FIG. 56 Weight variation plot of ER7H lot 031466-26.

FIG. 57 Tablet appearance of ER7H lot 031466-26.

FIG. 58 In vitro dissolution profiles of ER9 lot 031466-26 confirmation batches.

FIG. 59 Process schematic chart of Dexpramipexole ER5 matrix 280 mg tablets (376 mg diHCl) Clinical Trial Material (CTM) batch.

FIG. 60 In-process tablet weight of Dexpramipexole ER5 matrix 280 mg tablets (376 mg diHCl) CTM batch.

FIG. 61 In-process tablet thickness of Dexpramipexole ER5 matrix 280 mg tablets (376 mg diHCl) CTM batch.

FIG. 62 In-process tablet hardness of Dexpramipexole ER5 matrix 280 mg tablets (376 mg diHCl) CTM batch.

FIG. 63 Tablet appearance of Dexpramipexole ER5 matrix 280 mg tablets (376 mg diHCl) CTM batch.

FIG. 64 In vitro dissolution profile of ER5 CTM batch.

FIG. 65 Process schematic chart of Dexpramipexole ER7H matrix 223 mg tablets (300 mg diHCl) CTM batch.

FIG. 66 In-process tablet weight of Dexpramipexole ER7H matrix 223 mg tablets (300 mg diHCl) CTM batch.

FIG. 67 In-process tablet thickness of Dexpramipexole ER7H matrix 223 mg tablets (300 mg diHCl) CTM batch.

FIG. 68 In-process tablet hardness of Dexpramipexole ER7H matrix 223 mg tablets (300 mg diHCl) CTM batch.

FIG. 69 Tablet appearance of Dexpramipexole ER7H matrix 223 mg tablets (300 mg diHCl) CTM batch.

FIG. 70 In vitro dissolution profile of ER7H CTM batch.

FIG. 71 Process schematic chart of Dexpramipexole ER9 matrix 223 mg tablets (300 mg diHCl) CTM batch.

FIG. 72 In-process tablet weight of Dexpramipexole ER9 matrix 223 mg tablets (300 mg diHCl) CTM batch.

FIG. 73 In-process tablet thickness of Dexpramipexole ER9 matrix 223 mg tablets (300 mg diHCl) CTM batch.

FIG. 74 In-process tablet hardness of Dexpramipexole ER9 matrix 223 mg tablets (300 mg diHCl) CTM batch.

FIG. 75 Tablet appearance of Dexpramipexole ER9 matrix 223 mg tablets (300 mg diHCl) CTM batch.

FIG. 76 In vitro dissolution profile of ER9 CTM batch.

FIG. 77 Process schematic chart of lab scale batch using milled Dexpramipexole Dihydrochloride Monohydrate.

FIG. 78 Tabletability study of (A) Lot 031466-33 milled-API and (B) Lot 031466-24 ER7H 2nd confirmation batch compressed at 20 rpm turret speed.

FIG. 79 In vitro dissolution profile of batch 031466-33 vs. ER7H CTM batch 24JM-083.

FIG. 80 Tabletability of Lot 031466-34 with 0.75% Magnesium Stearate and Lot 031466-34 with 0.50% Magnesium Stearate.

FIG. 81 In vitro dissolution profile of batch 031466-34 vs. ER7H CTM batch 24JM-083.

### DETAILED DESCRIPTION OF THE DISCLOSURE

Unless defined otherwise, all technical and scientific terms used herein have the same meaning as commonly understood by one of ordinary skill in the art to which this disclosure belongs. In case of conflict, the present application including the definitions will control. Unless otherwise required by context, singular terms shall include pluralities and plural terms shall include the singular. In general, the headings provided herein are not to be understood as limiting the various aspects and embodiments of the present disclosure. All publications, patents and other references mentioned herein are incorporated by reference in their entireties for all purposes as if each individual publication or patent application were specifically and individually indicated to be incorporated by reference.

Although methods and materials similar or equivalent to those described herein can be used in practice or testing of the present disclosure, suitable methods and materials are described below. The materials, methods and examples are illustrative only and are not intended to be limiting. Other features and advantages of the disclosure will be apparent from the detailed description and from the claims.

### I—Definitions

In the following, some definitions are provided to further define the present disclosure. Additional definitions may be found throughout the specification.

The singular forms “a,” “an,” and “the” include plural referents unless the context clearly dictates otherwise. The terms “a” (or “an”), as well as the terms “one or more” and “at least one” can be used interchangeably herein. In certain aspects, the term “a” or “an” means “single.” In other aspects, the term “a” or “an” includes “two or more” or “multiple.”

The term “about” is used herein to mean approximately, roughly, around, or in the regions of. When the term “about” is used in conjunction with a numerical value or range, it modifies that value or range by extending the boundaries above and below the numerical value(s) set forth by a variance of 10 percent, up or down (higher or lower). For example, within the meaning of the present disclosure, “about 50%” means in the range of 45%-55%, and “about 50% to about 60%” means in the range of 45%-66%. In addition, when the term “about” is used in conjunction with a “%” value, the recited value or range cannot exceed 100%. For example, within the meaning of the present disclosure, “about 99%” means in the range of 89.1%-100%.

The term “and/or,” where used herein, is to be taken as specific disclosure of each of the specified features or components with or without the other. Thus, the term “and/or” as used in a phrase such as “A and/or B” herein is intended to include “A and B”, “A or B”, “A” (alone), and “B” (alone). Likewise, the term “and/or” as used in a phrase such as “A, B, and/or C” is intended to encompass each of the following aspects: “A, B, and C”, “A, B, or C”, “A or C”, “A or B”, “B or C”, “A and C”, “A and B”, “B and C”, “A” (alone), “B” (alone), and “C” (alone).

As used herein, the term “comprising” means “including, but not limited to”.

As used herein, the term “consisting essentially of” means the method, composition or mixture includes the steps or components specifically recited, and may also include those that do not materially affect the basic and novel characteristics of the method, composition, or mixture. The basic and novel characteristics of the compositions of the present disclosure (such as of the tablets described herein) are that the compositions are suitable for oral administration to a human (i.e., the compositions described herein do not exceed a weight of about 1500 mg) once daily and thereby provide for an amount of dexpramipexole, or a pharmaceutically acceptable salt thereof, as specified herein (i.e., for an amount of about 50 mg to about 400 mg dexpramipexole dihydrochloride equivalent). Accordingly, the basic and novel characteristics of the methods of manufacturing of the present disclosure are that the methods result in the compositions (such as the tablets) as described in this paragraph or in intermediate products of the compositions (such as densified granules or blends). Further, the basic and novel characteristics of the mixture of the present disclosure are that with this mixture, the preparation of the compositions (such as tablets) as described in this paragraph is possible.

As used herein, the term “consisting of” means the method, composition, or mixture includes only the steps or components specifically recited thereafter.

As used herein, the term “any combination thereof” or “a combination thereof” covers any combination of the components or steps recited before the term. For example, the expression “a composition further comprising A, B, C, D, or any combination thereof” includes inter alia “a composition further comprising A,” “a composition further comprising A and B,” “a composition further comprising B and D,” “a composition further comprising A, B and C,” and “a composition further comprising A, B, C, and D”.

The term “homogeneous mixture,” as used herein, refers to a mixture wherein all of the ingredients have been thoroughly mixed such that the composition of the mixture is substantially the same throughout different portions of the mixture.

As used herein, the terms “pre-blend” and “blend” refer to homogeneous mixtures of the corresponding ingredients in the pre-blend or blend, respectively. A “blend” refers to a homogeneous mixture that comprises additional ingredients compared to a corresponding “pre-blend”.

The term “orally deliverable,” as used herein, means that a corresponding pharmaceutical composition (such as a tablet) is suitable for oral administration to a human by swallowing the composition as a whole.

The term “tablet core,” as used herein, refers to the part of the orally deliverable tablets described herein that provides for sustained release of dexpramipexole, or a pharmaceutically acceptable salt thereof. The tablet core comprises the amounts of dexpramipexole, or a pharmaceutically acceptable salt as specified herein and the amounts of one or more anionic polymers as specified herein and may comprise additional components such as a lubricant, a diluent, and/or a glidant. The tablet core might be coated (in particular with a non-functional film coating), but not necessarily has to be coated. In embodiments wherein the tablet core is not coated, the weight of the tablet (as defined further below) corresponds to the weight of the tablet core. The tablet core may be, for example, a single-layer tablet core or a multi-layer tablet core (e.g., bi-layer, tri-layer, etc.). In some embodiments, the tablet core is a single-layer tablet core (also referred to as a unitary core that is not divided into two or more layers).

The term “dexpramipexole,” as used herein, refers to (6R)-2-amino-4,5,6,7-tetrahydro-6-(propylamino)benzothiazole. The chemical structure of dexpramipexole is given above in formula (I). The term “dexpramipexole” refers to the “free base” (i.e., the neutral form of dexpramipexole as shown above in formula (I)) unless the context clearly dictates otherwise.

The term “pharmaceutically acceptable salt” is meant to indicate those salts that are suitable for use in contact with the tissues of a human without undue toxicity, irritation, allergic response, and the like, and are commensurate with a reasonable benefit/risk ratio. For example, Remington's Pharmaceutical Sciences, 23^{th} ed. (2020) and Berge et al. (1977), J. Pharm. Sciences, Vol 6., 1-19 describe pharmaceutically acceptable salts in detail. Pharmaceutically acceptable salts can generally also be in the form of hydrates (such as in the form of a monohydrate). In some embodiments, the pharmaceutically acceptable salt of dexpramipexole is dexpramipexole dihydrochloride or a hydrate thereof (such as the monohydrate). Examples of suitable pharmaceutically acceptable salts of dexpramipexole within the scope of the present disclosure include, but are not limited to, dexpramipexole dihydrochloride and dexpramipexole dihydrochloride monohydrate. Further suitable “pharmaceutically acceptable salts” of dexpramipexole within the scope of the present disclosure are described throughout the present specification.

The term “equivalent” is used herein to specify a certain amount of an ingredient with reference to a more specific form (such as a specific salt) of the ingredient. For example, the term “dexpramipexole dihydrochloride equivalent” is used to specify the amount of dexpramipexole, or a pharmaceutically acceptable salt thereof, in a pharmaceutical composition that corresponds the same quantity as dexpramipexole dihydrochloride. For instance, an amount of 319 mg dexpramipexole dihydrochloride monohydrate corresponds to 300 mg dexpramipexole dihydrochloride equivalent, and an amount of 223 mg dexpramipexole corresponds to 300 mg dexpramipexole dihydrochloride equivalent. As another example, an amount of 160 mg dexpramipexole dihydrochloride monohydrate corresponds to 150 mg dexpramipexole dihydrochloride equivalent, and an amount of 112 mg dexpramipexole corresponds to 150 mg dexpramipexole dihydrochloride equivalent.

The term “weight of the tablet,” as used herein, refers to the weight of the entire tablet including the weight of any potential coating surrounding the tablet core. For example, in embodiments wherein the tablet core is surrounded by a film coating, the term “weight of the tablet” also includes the weight of the film coating. However, as explained in this chapter (“I—Definitions”) above in the context of “tablet core”, the tablet core does not necessarily have to be coated. In embodiments wherein the tablet core is not surrounded by a coating, the “weight of the tablet” equals the “weight of the tablet core”.

“Immediate release,” as used herein, means that no effort is taken either through formulation or processing to modify the release of the drug from the dosage form. Typically, greater than 80% of drug is dissolved within 1 hour when analyzed with a USP method.

“Sustained release,” as used herein, refers to products where the time period of release is extended through formulation and/or processing of the drug product. Sustained release of an active agent to an environment occurs over a period of about eight hours, about 12 hours, about 16 hours, about 18 hours, about 20 hours, about 24 hours, or more than about 24 hours. A sustained release can begin within a few minutes after administration (such as within about 5 min or about 10 min after administration); the release can also begin after an expiration of a delay period (lag time) after administration. In some aspects, the active agent is released to an environment over a period of about 5 min to about eight hours, about 5 min to about 12 hours, about 5 min to about 16 hours, about 5 min to about 18 hours, about 5 min to about 20 hours, about 5 min to about 24 hours, or about 5 min to more than about 24 hours after administration. In some aspects, the active agent is released to an environment over a period of about 10 min to about eight hours, about 10 min to about 12 hours, about 10 min to about 16 hours, about 10 min to about 18 hours, about 10 min to about 20 hours, about 10 min to about 24 hours, or about 10 min to more than about 24 hours after administration. In certain embodiments, the drug is released at a substantially constant rate or pulsatile rate over an extended period of time. In some aspects, the sustained release provides for a constant drug level in the blood or target tissue of a human to which the sustained release device (such as a tablet) is administered.

“Extended release,” as used herein, refers to products where either the rate of release is controlled/reduced or the duration of release is extended through formulation and/or processing of the drug product.

“Controlled release,” as used herein, refers to products where the rate of drug release has been intentionally modified through formulation and/or processing of the drug product.

The terms “controlled release,” “sustained release,” or “extended release” or “ER” are used interchangeably herein.

The term “treating” refers to alleviating of the signs or symptoms associated with a specific disorder, disease, or condition, and/or removing of the signs or symptoms associated with a specific disorder, disease, or condition, and/or preventing of the worsening of the signs or symptoms associated with a specific disorder, disease, or condition. In some aspects, the treatment alleviates signs or symptoms associated with a specific disorder, disease, or condition. In other aspects, the treatment removes signs or symptoms associated with a specific disorder, disease, or condition. In other aspects, the treatment prevents worsening of the signs or symptoms associated with a specific disorder, disease, or condition.

The term “preventing” refers to prophylaxis of a specific disorder, disease, or condition in a human. In certain aspects, the human may be predisposed to the specific disorder, disease, or condition but does not yet experience or display the pathology, signs, or symptoms of the specific disorder, disease, or condition.

The term “in need thereof,” as used herein, means that the human subject has a need for the particular treatment or prevention and that the treatment or prevention is being given to the subject for that particular purpose.

### II—Pharmaceutical Composition (Orally Deliverable Tablet)

In certain aspects, the present disclosure is directed to a sustained release pharmaceutical composition in the form of an orally deliverable tablet comprising a tablet core and dexpramipexole, or a pharmaceutically acceptable salt thereof.

In some aspects, the sustained release pharmaceutical composition comprises dexpramipexole, or a pharmaceutically acceptable salt thereof, at an amount of about 50 mg to about 400 mg of dexpramipexole dihydrochloride equivalent. In some aspects, the tablet core comprises a homogeneous mixture of the amount of dexpramipexole, or a pharmaceutically acceptable salt thereof, and one or more anionic polymers. In some aspects, the one or more anionic polymers constitute about 30% to about 60% by weight of the tablet core. In some aspects, the weight of the tablet is about 1500 mg or less.

In some aspects, the sustained release pharmaceutical composition comprises dexpramipexole, or a pharmaceutically acceptable salt thereof, at an amount of about 50 mg to about 500 mg of dexpramipexole dihydrochloride equivalent. In some aspects, the weight of the tablet is about 1500 mg or less.

In certain aspects, the present disclosure is directed to a sustained release pharmaceutical composition in the form of an orally deliverable tablet comprising a tablet core and dexpramipexole, or a pharmaceutically acceptable salt thereof, in an amount of about 50 mg to about 400 mg of dexpramipexole dihydrochloride equivalent, wherein the tablet core comprises a homogeneous mixture of the amount of dexpramipexole, or a pharmaceutically acceptable salt thereof, and one or more anionic polymers, wherein the one or more anionic polymers constitute about 30% to about 60% by weight of the tablet core, and wherein the weight of the tablet is about 1500 mg or less.

In certain aspects, the present disclosure is directed to a sustained release pharmaceutical composition in the form of an orally deliverable tablet comprising a tablet core and dexpramipexole, or a pharmaceutically acceptable salt thereof, in an amount of about 50 mg to about 500 mg of dexpramipexole dihydrochloride equivalent, wherein the tablet core comprises a homogeneous mixture of the amount of dexpramipexole, or a pharmaceutically acceptable salt thereof, and one or more polymers, wherein the one or more polymers constitute about 5% to about 60% by weight of the tablet core, and wherein the weight of the tablet is about 1500 mg or less.

In some aspects, the one or more polymers are anionic polymers or neutral polymers. In some aspects, the one or more polymers are anionic polymers. In some aspects, the one or more anionic polymers constitute about 30% to about 60% by weight of the tablet core.

In some aspects, the one or more polymers are neutral polymers. In some aspects, the one or more neutral polymers constitute about 5% to about 25% by weight of the tablet core. In some aspects, the one or more neutral polymers constitute about 5% to about 60% by weight of the tablet core.

In some embodiments, the one or more neutral polymers constitute between about 5% and about 60% by weight of the tablet core. In some embodiments, the one or more neutral polymers constitute between about 10% and about 60% by weight of the tablet core. In some embodiments, the one or more neutral polymers constitute about 15% to about 60% by weight of the tablet core. In some embodiments, the one or more neutral polymers constitute between about 20% and about 60% by weight of the tablet core. In some embodiments, the one or more neutral polymers constitute about 25% to about 60% by weight of the tablet core. In some embodiments, the one or more neutral polymers constitute between about 30% and about 60% by weight of the tablet core. In some embodiments, the one or more neutral polymers constitute about 35% to about 60% by weight of the tablet core. In some embodiments, the one or more neutral polymers constitute between about 40% and about 60% by weight of the tablet core. In some embodiments, the one or more neutral polymers constitute about 45% to about 60% by weight of the tablet core. In some embodiments, the one or more neutral polymers constitute between about 50% and about 60% by weight of the tablet core. In some embodiments, the one or more neutral polymers constitute about 55% to about 60% by weight of the tablet core.

In some embodiments, the one or more neutral polymers constitute about 5% to about 55% by weight of the tablet core. In some embodiments, the one or more neutral polymers constitute about 5% to about 50% by weight of the tablet core. In some embodiments, the one or more neutral polymers constitute about 5% to about 45% by weight of the tablet core. In some embodiments, the one or more neutral polymers constitute about 5% to about 40% by weight of the tablet core. In some embodiments, the one or more neutral polymers constitute about 5% to about 35% by weight of the tablet core. In some embodiments, the one or more neutral polymers constitute about 5% to about 30% by weight of the tablet core.

In some embodiments, the one or more neutral polymers constitute about 20% by weight of the tablet core. In some embodiments, the one or more neutral polymers constitute about 25% by weight of the tablet core. In some embodiments, the one or more neutral polymers constitute about 30% by weight of the tablet core. In some embodiments, the one or more neutral polymers constitute about 35% by weight of the tablet core. In some embodiments, the one or more neutral polymers constitute about 40% by weight of the tablet core. In some embodiments, the one or more neutral polymers constitute about 45% by weight of the tablet core. In some embodiments, the one or more neutral polymers constitute about 50% by weight of the tablet core. In some embodiments, the one or more neutral polymers constitute about 55% by weight of the tablet core. In some embodiments, the one or more neutral polymers constitute about 60% by weight of the tablet core.

The pharmaceutical composition in the form of an orally deliverable tablet according to the present disclosure is further described in the sections below within this chapter (“II—Pharmaceutical Composition (Orally Deliverable Tablet)”). As acknowledged by a person of ordinary skill in the art, the embodiments described below may be combined to further embodiments. For example, one or more embodiments described under section “3. Tablet Core” may be combined with one or more embodiments described under section “4. Film coating” to a further embodiment.

### 1. Pharmaceutically Acceptable Salts Of Dexpramipexole

In some embodiments, the pharmaceutically acceptable salt of dexpramipexole is a halogenic acid salt, an inorganic acid salt, an organic acid salt, or an amino acid salt.

In some embodiments, the halogenic acid salt is a hydrobromic, hydrochloric, hydrofluoric, or hydroiodic acid salt.

In some embodiments, the inorganic acid salt is a nitric, perchloric, sulfuric, or phosphoric acid salt.

In some embodiments, the organic acid salt is a sulfonic, tartaric acid, acetic, malic, fumaric, succinic, citric, benzoic, gluconic, lactic, mandelic, mucic, pamoic, pantothenic, exalic or maleic acid salt. In some embodiments, the sulfonic acid salt is a methane sulfonic, trifluoromethane sulfonic, ethane sulfonic, benzene sulfonic or p-toluene sulfonic acid salt.

In some embodiments, the amino acid salt is aspartic or glutamic acid salt.

In some embodiments, the pharmaceutically acceptable salt is a methane sulfonic acid salt (“mesylate salt”), sulfuric acid salt (“sulfate salt”), tartaric acid salt (“tartrate salt”), p-toluene sulfonic acid salt (“tosylate salt”), phosphoric acid salt (“phosphate salt”), maleic acid salt (“maleate salt”), fumaric acid salt (“fumarate salt”), malic acid salt (“malate salt”), citric acid salt (“citrate salt”), succinic acid salt (“succinate salt”), or any combination thereof.

In some specific embodiments, the pharmaceutically acceptable salt is a methane sulfonic acid salt, phosphoric acid salt, fumaric acid salt, or any combination thereof.

The acid addition salt may be a mono- or di-acid addition salt, such as dihydrobromic, dihydrochloric, dihydrofluoric, dihydroiodic, disulfuric, diphosphoric, or diorganic acid salt. The acid addition salt may additionally be in the form of a hydrate, such as a dihydrochloride monohydrate.

In some embodiments, the pharmaceutically acceptable salt of dexpramipexole is dexpramipexole dihydrochloride or a hydrate thereof (such as the monohydrate).

In some specific embodiments, the pharmaceutically acceptable salt of dexpramipexole is dexpramipexole dihydrochloride.

In some specific embodiments, the pharmaceutically acceptable salt of dexpramipexole is dexpramipexole dihydrochloride monohydrate.

### 2. Amount of Dexpramipexole, or a Pharmaceutically Acceptable Salt Thereof

As outlined above, in some aspects, the sustained release pharmaceutical composition in the form of an orally deliverable tablet comprises dexpramipexole, or a pharmaceutically acceptable salt thereof, in an amount of about 50 mg to about 400 mg of dexpramipexole dihydrochloride equivalent. In some aspects, the sustained release pharmaceutical composition in the form of an orally deliverable tablet comprises dexpramipexole, or a pharmaceutically acceptable salt thereof, in an amount of about 50 mg to about 500 mg of dexpramipexole dihydrochloride equivalent.

In some embodiments, the amount of dexpramipexole, or a pharmaceutically acceptable salt thereof, in the pharmaceutical composition is about 50 mg to about 350 mg of dexpramipexole dihydrochloride equivalent. In some embodiments, the amount of dexpramipexole, or a pharmaceutically acceptable salt thereof, in the pharmaceutical composition is about 55 mg to about 340 mg, about 60 mg to about 330 mg, about 65 mg to about 320 mg, or about 70 mg to about 310 mg of dexpramipexole dihydrochloride equivalent.

In some embodiments, the amount of dexpramipexole, or a pharmaceutically acceptable salt thereof, in the pharmaceutical composition is about 50 mg to about 400 mg of dexpramipexole dihydrochloride equivalent. In some embodiments, the amount of dexpramipexole, or a pharmaceutically acceptable salt thereof, in the pharmaceutical composition is about 55 mg to about 395 mg, about 60 mg to about 390 mg, about 65 mg to about 385 mg, or about 70 mg to about 380 mg of dexpramipexole dihydrochloride equivalent.

In some embodiments, the amount of dexpramipexole, or a pharmaceutically acceptable salt thereof, in the pharmaceutical composition is about 75 mg to about 300 mg (such as about 75 mg, about 150 mg, or about 300 mg) of dexpramipexole dihydrochloride equivalent. In some embodiments, the amount of dexpramipexole, or a pharmaceutically acceptable salt thereof, in the pharmaceutical composition is about 300 mg to about 400 mg (such as about 300 mg or about 376 mg) of dexpramipexole dihydrochloride equivalent.

In specific embodiments, the amount of dexpramipexole, or a pharmaceutically acceptable salt thereof, in the pharmaceutical composition is about 75 mg of dexpramipexole dihydrochloride equivalent. In other specific embodiments, the amount of dexpramipexole, or a pharmaceutically acceptable salt thereof, in the pharmaceutical composition is about 150 mg of dexpramipexole dihydrochloride equivalent. In yet other specific embodiments, the amount of dexpramipexole, or a pharmaceutically acceptable salt thereof, in the pharmaceutical composition is about 300 mg of dexpramipexole dihydrochloride equivalent. In yet other specific embodiments, the amount of dexpramipexole, or a pharmaceutically acceptable salt thereof, in the pharmaceutical composition is about 376 mg of dexpramipexole dihydrochloride equivalent.

In some embodiments, the amount of dexpramipexole, or a pharmaceutically acceptable salt thereof, in the pharmaceutical composition is about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, or about 400 mg of dexpramipexole dihydrochloride equivalent. In some embodiments, the amount of dexpramipexole, or a pharmaceutically acceptable salt thereof, in the pharmaceutical composition is about 75 mg, about 150 mg, about 300 mg, or about 376 mg of dexpramipexole dihydrochloride equivalent.

In some embodiments, the amount of dexpramipexole, or a pharmaceutically acceptable salt thereof, in the pharmaceutical composition is about 50 mg to about 100 mg of dexpramipexole dihydrochloride equivalent. In some embodiments, the amount of dexpramipexole, or a pharmaceutically acceptable salt thereof, in the pharmaceutical composition is about 55 mg to about 95 mg of dexpramipexole dihydrochloride equivalent. In some embodiments, the amount of dexpramipexole, or a pharmaceutically acceptable salt thereof, in the pharmaceutical composition is about 60 mg to about 90 mg of dexpramipexole dihydrochloride equivalent. In some embodiments, the amount of dexpramipexole, or a pharmaceutically acceptable salt thereof, in the pharmaceutical composition is about 65 mg to about 85 mg of dexpramipexole dihydrochloride equivalent. In some embodiments, the amount of dexpramipexole, or a pharmaceutically acceptable salt thereof, in the pharmaceutical composition is about 70 mg to about 80 mg of dexpramipexole dihydrochloride equivalent. In specific embodiments, the amount of dexpramipexole, or a pharmaceutically acceptable salt thereof, in the pharmaceutical composition is about 75 mg of dexpramipexole dihydrochloride equivalent.

In some embodiments, the amount of dexpramipexole, or a pharmaceutically acceptable salt thereof, in the pharmaceutical composition is about 100 mg to about 350 mg of dexpramipexole dihydrochloride equivalent. In some embodiments, the amount of dexpramipexole, or a pharmaceutically acceptable salt thereof, is about 105 mg to about 345 mg of dexpramipexole dihydrochloride equivalent. In some embodiments, the amount of dexpramipexole, or a pharmaceutically acceptable salt thereof, is about 110 mg to about 340 mg of dexpramipexole dihydrochloride equivalent. In some embodiments, the amount of dexpramipexole, or a pharmaceutically acceptable salt thereof, is about 115 mg to about 335 mg of dexpramipexole dihydrochloride equivalent. In some embodiments, the amount of dexpramipexole, or a pharmaceutically acceptable salt thereof, is about 120 mg to about 335 mg of dexpramipexole dihydrochloride equivalent. In some embodiments, the amount of dexpramipexole, or a pharmaceutically acceptable salt thereof, is about 120 mg to about 330 mg of dexpramipexole dihydrochloride equivalent. In some embodiments, the amount of dexpramipexole, or a pharmaceutically acceptable salt thereof, in the pharmaceutical composition is about 125 mg to about 325 mg of dexpramipexole dihydrochloride equivalent. In some embodiments, the amount of dexpramipexole, or a pharmaceutically acceptable salt thereof, in the pharmaceutical composition is about 130 mg to about 320 mg of dexpramipexole dihydrochloride equivalent. In some embodiments, the amount of dexpramipexole, or a pharmaceutically acceptable salt thereof, in the pharmaceutical composition is about 135 mg to about 315 mg of dexpramipexole dihydrochloride equivalent. In some embodiments, the amount of dexpramipexole, or a pharmaceutically acceptable salt thereof, in the pharmaceutical composition is about 140 mg to about 310 mg of dexpramipexole dihydrochloride equivalent. In some embodiments, the amount of dexpramipexole, or a pharmaceutically acceptable salt thereof, in the pharmaceutical composition is about 145 mg to about 305 mg of dexpramipexole dihydrochloride equivalent. In some embodiments, the amount of dexpramipexole, or a pharmaceutically acceptable salt thereof, in the pharmaceutical composition is about 150 mg to about 300 mg of dexpramipexole dihydrochloride equivalent.

In some embodiments, the amount of dexpramipexole, or a pharmaceutically acceptable salt thereof, in the pharmaceutical composition is about 200 mg to about 400 mg, about 205 mg to about 395 mg, about 210 mg to about 390 mg, about 215 mg to about 385 mg, about 220 mg to about 380 mg, about 225 mg to about 375 mg, about 230 mg to about 370 mg, about 235 mg to about 365 mg, about 240 mg to about 360 mg, or about 245 mg to about 355 mg of dexpramipexole dihydrochloride equivalent. In some embodiments, the amount of dexpramipexole, or a pharmaceutically acceptable salt thereof, is about 250 mg to about 350 mg, about 255 mg to about 345 mg, about 260 mg to about 340 mg, about 265 mg to about 335 mg, or about 270 mg to about 330 mg of dexpramipexole dihydrochloride equivalent. In some embodiments, the amount of dexpramipexole, or a pharmaceutically acceptable salt thereof, in the pharmaceutical composition is about 275 mg to about 325 mg of dexpramipexole dihydrochloride equivalent. In some embodiments, the amount of dexpramipexole, or a pharmaceutically acceptable salt thereof, in the pharmaceutical composition is about 280 mg to about 320 mg of dexpramipexole dihydrochloride equivalent. In some embodiments, the amount of dexpramipexole, or a pharmaceutically acceptable salt thereof, in the pharmaceutical composition is about 285 mg to about 315 mg of dexpramipexole dihydrochloride equivalent. In some embodiments, the amount of dexpramipexole, or a pharmaceutically acceptable salt thereof, in the pharmaceutical composition is about 290 mg to about 310 mg of dexpramipexole dihydrochloride equivalent. In some embodiments, the amount of dexpramipexole, or a pharmaceutically acceptable salt thereof, in the pharmaceutical composition is about 295 mg to about 305 mg of dexpramipexole dihydrochloride equivalent. In specific embodiments, the amount of dexpramipexole, or a pharmaceutically acceptable salt thereof, in the pharmaceutical composition is about 300 mg of dexpramipexole dihydrochloride equivalent.

In some embodiments, the amount of dexpramipexole, or a pharmaceutically acceptable salt thereof, in the pharmaceutical composition is about 300 mg to about 450 mg of dexpramipexole dihydrochloride equivalent. In some embodiments, the amount of dexpramipexole, or a pharmaceutically acceptable salt thereof, in the pharmaceutical composition is about 320 mg to about 440 mg of dexpramipexole dihydrochloride equivalent. In some embodiments, the amount of dexpramipexole, or a pharmaceutically acceptable salt thereof, in the pharmaceutical composition is about 330 mg to about 430 mg of dexpramipexole dihydrochloride equivalent. In some embodiments, the amount of dexpramipexole, or a pharmaceutically acceptable salt thereof, in the pharmaceutical composition is about 340 mg to about 420 mg of dexpramipexole dihydrochloride equivalent. In some embodiments, the amount of dexpramipexole, or a pharmaceutically acceptable salt thereof, in the pharmaceutical composition is about 350 mg to about 410 mg of dexpramipexole dihydrochloride equivalent. In some embodiments, the amount of dexpramipexole, or a pharmaceutically acceptable salt thereof, in the pharmaceutical composition is about 360 mg to about 400 mg of dexpramipexole dihydrochloride equivalent. In some embodiments, the amount of dexpramipexole, or a pharmaceutically acceptable salt thereof, in the pharmaceutical composition is about 370 mg to about 390 mg of dexpramipexole dihydrochloride equivalent. In specific embodiments, the amount of dexpramipexole, or a pharmaceutically acceptable salt thereof, in the pharmaceutical composition is about 376 mg of dexpramipexole dihydrochloride equivalent.

In some embodiments, the amount of dexpramipexole, or a pharmaceutically acceptable salt thereof, in the pharmaceutical composition is about 50 mg to about 250 mg, about 55 mg to about 245 mg, about 60 mg to about 240 mg, about 65 mg to about 235 mg, about 70 mg to about 230 mg about 75 mg to about 225 mg, about 80 mg to about 220 mg, about 85 mg to about 215 mg, about 90 mg to about 210 mg, about 95 mg to about 205 mg, about 100 mg to about 200 mg, about 105 mg to about 195 mg, about 110 mg to about 190 mg, about 115 mg to about 185 mg, or about 120 mg to about 180 mg of dexpramipexole dihydrochloride equivalent. In some embodiments, the amount of dexpramipexole, or a pharmaceutically acceptable salt thereof, in the pharmaceutical composition is about 125 mg to about 175 mg of dexpramipexole dihydrochloride equivalent. In some embodiments, the amount of dexpramipexole, or a pharmaceutically acceptable salt thereof, in the pharmaceutical composition is about 130 mg to about 170 mg of dexpramipexole dihydrochloride equivalent. In some embodiments, the amount of dexpramipexole, or a pharmaceutically acceptable salt thereof, in the pharmaceutical composition is about 135 mg to about 165 mg of dexpramipexole dihydrochloride equivalent. In some embodiments, the amount of dexpramipexole, or a pharmaceutically acceptable salt thereof, in the pharmaceutical composition is about 140 mg to about 160 mg of dexpramipexole dihydrochloride equivalent. In some embodiments, the amount of dexpramipexole, or a pharmaceutically acceptable salt thereof, in the pharmaceutical composition is about 145 mg to about 165 mg of dexpramipexole dihydrochloride equivalent. In specific embodiments, the amount of dexpramipexole, or a pharmaceutically acceptable salt thereof, in the pharmaceutical composition is about 150 mg of dexpramipexole dihydrochloride equivalent.

In some embodiments, the pharmaceutical composition comprises about 60 mg to about 340 mg dexpramipexole dihydrochloride monohydrate.

In some embodiments, the pharmaceutical composition comprises about 60 mg to about 100 mg dexpramipexole dihydrochloride monohydrate. In some embodiments, the pharmaceutical composition comprises about 65 mg to about 95 mg dexpramipexole dihydrochloride monohydrate. In some embodiments, the pharmaceutical composition comprises about 70 mg to about 90 mg dexpramipexole dihydrochloride monohydrate. In some embodiments, the pharmaceutical composition comprises about 75 mg to about 85 mg dexpramipexole dihydrochloride monohydrate. In specific embodiments, the pharmaceutical composition comprises about 80 mg dexpramipexole dihydrochloride monohydrate.

In some embodiments, the pharmaceutical composition comprises about 140 mg to about 180 mg dexpramipexole dihydrochloride monohydrate. In some embodiments, the pharmaceutical composition comprises about 145 mg to about 175 mg dexpramipexole dihydrochloride monohydrate. In some embodiments, the pharmaceutical composition comprises about 150 mg to about 170 mg dexpramipexole dihydrochloride monohydrate. In some embodiments, the pharmaceutical composition comprises about 155 mg to about 165 mg dexpramipexole dihydrochloride monohydrate. In specific embodiments, the pharmaceutical composition comprises about 160 mg dexpramipexole dihydrochloride monohydrate.

In some embodiments, the pharmaceutical composition comprises about 300 mg to about 340 mg dexpramipexole dihydrochloride monohydrate. In some embodiments, the pharmaceutical composition comprises about 305 mg to about 335 mg dexpramipexole dihydrochloride monohydrate. In some embodiments, the pharmaceutical composition comprises about 310 mg to about 330 mg dexpramipexole dihydrochloride monohydrate. In some embodiments, the pharmaceutical composition comprises about 315 mg to about 325 mg dexpramipexole dihydrochloride monohydrate. In specific embodiments, the pharmaceutical composition comprises about 319 mg dexpramipexole dihydrochloride monohydrate.

In some embodiments, the pharmaceutical composition comprises about 300 mg to about 450 mg dexpramipexole dihydrochloride monohydrate.

In some embodiments, the pharmaceutical composition comprises about 320 mg to about 440 mg dexpramipexole dihydrochloride monohydrate. In some embodiments, the pharmaceutical composition comprises about 340 mg to about 430 mg dexpramipexole dihydrochloride monohydrate. In some embodiments, the pharmaceutical composition comprises about 360 mg to about 420 mg dexpramipexole dihydrochloride monohydrate. In some embodiments, the pharmaceutical composition comprises about 380 mg to about 410 mg dexpramipexole dihydrochloride monohydrate. In specific embodiments, the pharmaceutical composition comprises about 400 mg dexpramipexole dihydrochloride monohydrate.

In some embodiments, the pharmaceutical composition comprises about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 319 mg, about 320 mg, about 325 mg, about 330 mg, about 335 mg, about 340 mg, about 350 mg, about 360 mg, about 370 mg, about 380 mg, about 390 mg, about 400 mg, about 410 mg, about 420 mg, about 430 mg, about 440 mg, or about 450 mg dexpramipexole dihydrochloride monohydrate.

In some embodiments, the amount of dexpramipexole, or a pharmaceutically acceptable salt thereof, constitutes about 20% to about 50%, about 21% to about 49%, about 22% to about 48%, about 23% to about 47%, or about 24% to about 46% of dexpramipexole dihydrochloride monohydrate equivalent by weight of the tablet core. In some embodiments, the amount of dexpramipexole, or a pharmaceutically acceptable salt thereof, constitutes about 25% to about 45%, about 26% to about 44%, about 27% to about 43%, about 28% to about 42%, or about 29% to about 41% of dexpramipexole dihydrochloride monohydrate equivalent by weight of the tablet core. In some embodiments, the amount of dexpramipexole, or a pharmaceutically acceptable salt thereof, constitutes about 30% to about 40% of dexpramipexole dihydrochloride monohydrate equivalent by weight of the tablet core. In some embodiments, the amount of dexpramipexole, or a pharmaceutically acceptable salt thereof, constitutes about 34% to about 39% of dexpramipexole dihydrochloride monohydrate equivalent by weight of the tablet core. In some embodiments, the amount of dexpramipexole, or a pharmaceutically acceptable salt thereof, constitutes about 31% to about 39%, about 32% to about 38%, about 33% to about 37%, or about 34% to about 36% of dexpramipexole dihydrochloride monohydrate equivalent by weight of the tablet core. In specific embodiments, the amount of dexpramipexole, or a pharmaceutically acceptable salt thereof, constitutes about 35% of dexpramipexole dihydrochloride monohydrate equivalent by weight of the tablet core. In some embodiments, the amount of dexpramipexole, or a pharmaceutically acceptable salt thereof, constitutes about 33% to about 43%, about 34% to about 42%, about 35% to about 41%, about 36% to about 40%, or about 37% to about 39% of dexpramipexole dihydrochloride monohydrate equivalent by weight of the tablet core. In specific embodiments, the amount of dexpramipexole, or a pharmaceutically acceptable salt thereof, constitutes about 38% of dexpramipexole dihydrochloride monohydrate equivalent by weight of the tablet core. In some embodiments, the amount of dexpramipexole, or a pharmaceutically acceptable salt thereof, constitutes about 38% to about 48%, about 39% to about 47%, about 40% to about 46%, about 41% to about 45%, or about 42% to about 44% of dexpramipexole dihydrochloride monohydrate equivalent by weight of the tablet core. In specific embodiments, the amount of dexpramipexole, or a pharmaceutically acceptable salt thereof, constitutes about 43% of dexpramipexole dihydrochloride monohydrate equivalent by weight of the tablet core. In some embodiments, the pharmaceutically acceptable salt of dexpramipexole is dexpramipexole dihydrochloride monohydrate.

In specific embodiments, the amount of dexpramipexole, or a pharmaceutically acceptable salt thereof, constitutes about 30% to about 40% of dexpramipexole dihydrochloride monohydrate equivalent by weight of the tablet core.

In some embodiments, the amount of dexpramipexole, or a pharmaceutically acceptable salt thereof, constitutes about 20%, about 22%, about 24%, about 26%, about 30%, about 31%, about 32%, about 33%, about 34%, about 35%, about 36%, about 37%, about 38%, about 39%, about 40%, about 42%, about 44%, about 46%, about 48%, or about 50% of dexpramipexole dihydrochloride monohydrate equivalent by weight of the tablet core.

In some embodiments, the amount of dexpramipexole, or a pharmaceutically acceptable salt thereof, constitutes about 20% to about 50%, about 21% to about 49%, about 22% to about 48%, about 23% to about 47%, or about 24% to about 46% of dexpramipexole dihydrochloride monohydrate equivalent by weight of the tablet. In some embodiments, the amount of dexpramipexole, or a pharmaceutically acceptable salt thereof, constitutes about 25% to about 45%, about 26% to about 44%, about 27% to about 43%, about 28% to about 42%, or about 29% to about 41% of dexpramipexole dihydrochloride monohydrate equivalent by weight of the tablet. In some embodiments, the amount of dexpramipexole, or a pharmaceutically acceptable salt thereof, constitutes about 30% to about 40% of dexpramipexole dihydrochloride monohydrate equivalent by weight of the tablet. In some embodiments, the amount of dexpramipexole, or a pharmaceutically acceptable salt thereof, constitutes about 31% to about 39%, about 32% to about 38%, about 33% to about 37%, or about 34% to about 36% of dexpramipexole dihydrochloride monohydrate equivalent by weight of the tablet. In some embodiments, the amount of dexpramipexole, or a pharmaceutically acceptable salt thereof, constitutes about 32% to about 36% of dexpramipexole dihydrochloride monohydrate equivalent by weight of the tablet. In specific embodiments, the amount of dexpramipexole, or a pharmaceutically acceptable salt thereof, constitutes about 34% of dexpramipexole dihydrochloride monohydrate equivalent by weight of the tablet. In some embodiments, the amount of dexpramipexole, or a pharmaceutically acceptable salt thereof, constitutes about 30% to about 40% of dexpramipexole dihydrochloride monohydrate equivalent by weight of the tablet. In some embodiments, the amount of dexpramipexole, or a pharmaceutically acceptable salt thereof, constitutes about 31% to about 39%, about 32% to about 38%, about 33% to about 37%, or about 34% to about 36% of dexpramipexole dihydrochloride monohydrate equivalent by weight of the tablet. In specific embodiments, the amount of dexpramipexole, or a pharmaceutically acceptable salt thereof, constitutes about 35% of dexpramipexole dihydrochloride monohydrate equivalent by weight of the tablet. In some embodiments, the amount of dexpramipexole, or a pharmaceutically acceptable salt thereof, constitutes about 34% to about 38% of dexpramipexole dihydrochloride monohydrate equivalent by weight of the tablet. In specific embodiments, the amount of dexpramipexole, or a pharmaceutically acceptable salt thereof, constitutes about 36% of dexpramipexole dihydrochloride monohydrate equivalent by weight of the tablet. In some embodiments, the amount of dexpramipexole, or a pharmaceutically acceptable salt thereof, constitutes about 37% to about 47% of dexpramipexole dihydrochloride monohydrate equivalent by weight of the tablet. In some embodiments, the amount of dexpramipexole, or a pharmaceutically acceptable salt thereof, constitutes about 38% to about 46%, about 39% to about 45%, about 40% to about 44%, or about 41% to about 43% of dexpramipexole dihydrochloride monohydrate equivalent by weight of the tablet. In specific embodiments, the amount of dexpramipexole, or a pharmaceutically acceptable salt thereof, constitutes about 42% of dexpramipexole dihydrochloride monohydrate equivalent by weight of the tablet. In some embodiments, the pharmaceutically acceptable salt of dexpramipexole is dexpramipexole dihydrochloride monohydrate.

In specific embodiments, the amount of dexpramipexole, or a pharmaceutically acceptable salt thereof, constitutes about 30% to about 40% of dexpramipexole dihydrochloride monohydrate equivalent by weight of the tablet.

In some embodiments, the amount of dexpramipexole, or a pharmaceutically acceptable salt thereof, constitutes about 20%, about 22%, about 24%, about 26%, about 30%, about 31%, about 32%, about 33%, about 34%, about 35%, about 36%, about 37%, about 38%, about 39%, about 40%, about 42%, about 44%, about 46%, about 48%, or about 50% of dexpramipexole dihydrochloride monohydrate equivalent by weight of the tablet.

In some embodiments, the weight ratio of dexpramipexole dihydrochloride equivalent to the one or more anionic polymers is about 0.2:1 to about 1.3:1, about 0.3:1 to about 1.2:1, about 0.4:1 to about 1.1:1, or about 0.5:1 to about 1:1.

In some embodiments, the weight ratio of dexpramipexole dihydrochloride equivalent to the one or more polymers is about 0.2:1 to about 4.0:1, about 0.3:1 to about 3.8:1, about 0.4:1 to about 3.6:1, about 0.5:1 to about 3.5:1, about 0.6:1 to about 3.4:1, about 0.7:1 to about 3.3:1, about 0.8:1 to about 3.2:1, about 0.9:1 to about 3.1:1, about 1.0:1 to about 3.0:1, about 1.1:1 to about 2.9:1, or about 1.2:1 to about 2.8:1.

In some embodiments, the weight ratio of dexpramipexole dihydrochloride equivalent to the one or more anionic polymers is about 0.7:1 to about 0.9:1. In some specific embodiments, the weight ratio of dexpramipexole dihydrochloride equivalent to the one or more anionic polymers is about 0.7:1. In some other specific embodiments, the weight ratio of dexpramipexole dihydrochloride equivalent to the one or more anionic polymers is about 0.9:1.

In some embodiments, the weight ratio of dexpramipexole dihydrochloride equivalent to the one or more polymers is about 0.7:1 to about 0.9:1. In some specific embodiments, the weight ratio of dexpramipexole dihydrochloride equivalent to the one or more polymers is about 0.7:1. In some specific embodiments, the weight ratio of dexpramipexole dihydrochloride equivalent to the one or more polymers is about 0.8:1. In some other specific embodiments, the weight ratio of dexpramipexole dihydrochloride equivalent to the one or more polymers is about 0.9:1.

In some embodiments, the weight ratio of dexpramipexole dihydrochloride equivalent to the one or more polymers is about 1.0:1 to about 2.0:1 or about 1.25:1 to about 1.75:1. In some specific embodiments, the weight ratio of dexpramipexole dihydrochloride equivalent to the one or more polymers is about 1.5:1.

In some embodiments, the weight ratio of dexpramipexole dihydrochloride equivalent to the one or more polymers is about 2.0:1 to about 3.0:1 or about 2.5:1 to about 2.9:1. In some specific embodiments, the weight ratio of dexpramipexole dihydrochloride equivalent to the one or more polymers is about 2.8:1.

In some embodiments, the weight ratio of dexpramipexole dihydrochloride equivalent to the one or more anionic polymers is about 0.2:1, about 0.3:1, about 0.4:1, about 0.5:1, about 0.6:1, about 0.7:1, about 0.8:1, about 0.9:1, about 1:1, about 1.1:1, about 1.2:1, about 1.3:1, about 1.4:1, about 1.5:1, about 1.6:1, about 1.7:1, about 1.8:1, about 1.9:1, about 2.0:1, about 2.1:1, about 2.2:1, about 2.3:1, about 2.4:1, about 2.5:1, about 2.6:1, about 2.7:1, about 2.8:1, about 2.9:1, about 3.0:1, about 3.1:1, about 3.2:1, about 3.3:1, about 3.4:1, about 3.5:1, about 3.6:1, about 3.7:1, about 3.8:1, about 3.9:1, or about 4.0:1.

In some embodiments, the weight ratio of dexpramipexole dihydrochloride equivalent to the one or more polymers is about 0.2:1, about 0.3:1, about 0.4:1, about 0.5:1, about 0.6:1, about 0.7:1, about 0.8:1, about 0.9:1, about 1:1, about 1.1:1, about 1.2:1, about 1.3:1, about 1.4:1, about 1.5:1, about 1.6:1, about 1.7:1, about 1.8:1, about 1.9:1, about 2.0:1, about 2.1:1, about 2.2:1, about 2.3:1, about 2.4:1, about 2.5:1, about 2.6:1, about 2.7:1, about 2.8:1, about 2.9:1, about 3.0:1, about 3.1:1, about 3.2:1, about 3.3:1, about 3.4:1, about 3.5:1, about 3.6:1, about 3.7:1, about 3.8:1, about 3.9:1, or about 4.0:1.

In some embodiments, dexpramipexole, or a pharmaceutically acceptable salt thereof, in the pharmaceutical composition has a chiral purity for dexpramipexole, or the pharmaceutically acceptable salt thereof, of 99.95% or more. “Having a chiral purity for dexpramipexole, or the pharmaceutically acceptable salt thereof, of 99.95% or more” means that the portion of pramipexole, or a pharmaceutically acceptable salt of pramipexole (see formula (II) above showing the chemical structure of pramipexole), respectively, within the amount of dexpramipexole, or a pharmaceutically acceptable salt of dexpramipexole in the pharmaceutical composition is 0.05% or less. In some embodiments, dexpramipexole, or a pharmaceutically acceptable salt thereof, in the pharmaceutical composition has a chiral purity for dexpramipexole, or the pharmaceutically acceptable salt thereof, of 99.96% or more. In some embodiments, dexpramipexole, or a pharmaceutically acceptable salt thereof, in the pharmaceutical composition has a chiral purity for dexpramipexole, or the pharmaceutically acceptable salt thereof, of 99.97% or more. In some embodiments, dexpramipexole, or a pharmaceutically acceptable salt thereof, in the pharmaceutical composition has a chiral purity for dexpramipexole, or the pharmaceutically acceptable salt thereof, of 99.98% or more. In some embodiments, dexpramipexole, or a pharmaceutically acceptable salt thereof, in the pharmaceutical composition has a chiral purity for dexpramipexole, or the pharmaceutically acceptable salt thereof, of 99.99% or more. In some embodiments, dexpramipexole, or a pharmaceutically acceptable salt thereof, in the pharmaceutical composition has a chiral purity for dexpramipexole, or the pharmaceutically acceptable salt thereof, of 100%. The chiral purity might be determined by analyzing a sample of dexpramipexole, or a pharmaceutically acceptable salt thereof, by high performance liquid chromatography (HPLC).

In specific embodiments, dexpramipexole, or a pharmaceutically acceptable salt thereof, in the pharmaceutical composition has a chiral purity for dexpramipexole, or the pharmaceutically acceptable salt thereof, of 99.96% or more.

### 3. Tablet Core

As outlined above, the pharmaceutical composition in the form of an orally deliverable tablet according to the present disclosure comprises a tablet core.

In some aspects, the tablet core comprises a homogeneous mixture of the amount of dexpramipexole, or a pharmaceutically acceptable salt thereof, and one or more anionic polymers. In some aspects, the one or more anionic polymers constitute about 30% to about 60% by weight of the tablet core.

In some aspects, the tablet core comprises a homogeneous mixture of the amount of dexpramipexole, or a pharmaceutically acceptable salt thereof, and one or more polymers. In some aspects, the one or more polymers constitute about 5% to about 60% by weight of the tablet core.

In some embodiments, the one or more polymers are anionic polymers. In some embodiments, the one or more polymers are neutral polymers. In some embodiments, no more than one anionic polymer is included in the homogeneous mixture. In some embodiments, no more than one neutral polymer is included in the homogeneous mixture.

In some embodiments, the one or more anionic polymers are selected from carboxymethylcellulose, an acrylic acid polymer, a methyl methacrylic acid polymer, a methyl methacrylic acid copolymer, a methacrylic acid polymer, a methacrylic acid copolymer, a copolymer of methyl methacrylic acid and methacrylic acid, alignate, carrageenan, xanthan gum, or arabic gum. In some embodiments, the one or more anionic polymers are hydrophilic anionic polymers. A hydrophilic anionic polymer is readily soluble in water. In contrast thereto, a hydrophobic anionic polymer is not readily soluble in water, but instead is soluble in lipids and/or organic solvents. In some embodiments, the one or more anionic polymers are selected from carboxymethylcellulose and an acrylic acid polymer.

In some embodiments, carboxymethylcellulose is sodium carboxymethylcellulose or potassium carboxymethylcellulose. In specific embodiments, carboxymethylcellulose is sodium carboxymethylcellulose. An example of a sodium carboxymethylcellulose is Aqualon CMC 7HXF PH. In more specific embodiments, the viscosity of the sodium carboxymethylcellulose is about 1,500 to about 3,000 cP (centipoise; 1% in water), which corresponds to about 1,500 to about 3,000 mPa·s.

In specific embodiments, the acrylic acid polymer is a crosslinked acrylic acid polymer. An example of a crosslinked acrylic acid polymer is Carbopol 971P. In more specific embodiments, the viscosity of the crosslinked acrylic acid polymer is about 4,000 to about 11,000 cP (centipoise; 0.5% at pH 7.5), which corresponds to about 4,000 to about 11,000 mPa·s.

In some embodiments, the one or more anionic polymers constitute about 25% to about 55% by weight of the tablet core. In some embodiments, the one or more anionic polymers constitute about 30% to about 50% by weight of the tablet core. In some embodiments, the one or more anionic polymers constitute about 35% to about 55% by weight of the tablet core. In some embodiments, the one or more anionic polymers constitute about 37% to about 52% by weight of the tablet core. In some embodiments, the one or more anionic polymers constitute about 40% to about 50% by weight of the tablet core.

In some embodiments, the one or more anionic polymers constitute about 28% to about 48% by weight of the tablet core. In some embodiments, the one or more anionic polymers constitute about 20% to about 36%, about 22% to about 34%, about 24% to about 32%, or about 26% to about 30% by weight of the tablet core. In some embodiments, the one or more anionic polymers constitute about 27% to about 29% by weight of the tablet core. In specific embodiments, the one or more anionic polymers constitute about 28% by weight of the tablet core.

In some embodiments, the one or more anionic polymers constitute about 30% to about 48% by weight of the tablet core. In some embodiments, the one or more anionic polymers constitute about 35% to about 45%, about 36% to about 44%, about 37% to about 43%, or about 38% to about 42% by weight of the tablet core. In some embodiments, the one or more anionic polymers constitute about 39% to about 41% by weight of the tablet core. In specific embodiments, the one or more anionic polymers constitute about 40% by weight of the tablet core.

In some embodiments, the one or more anionic polymers constitute about 36% to about 48% by weight of the tablet core. In some embodiments, the one or more anionic polymers constitute about 37% to about 47%, about 38% to about 46%, about 39% to about 45%, or about 40% to about 44% by weight of the tablet core. In some embodiments, the one or more anionic polymers constitute about 41% to about 43% by weight of the tablet core. In specific embodiments, the one or more anionic polymers constitute about 42% by weight of the tablet core.

In some embodiments, the one or more anionic polymers constitute about 42% to about 52%, about 43% to about 51%, about 44% to about 50%, or about 45% to about 49% by weight of the tablet core. In some embodiments, the one or more anionic polymers constitute about 46% to about 48% by weight of the tablet core. In specific embodiments, the one or more anionic polymers constitute about 47% by weight of the tablet core.

In specific embodiments, the one or more anionic polymers constitute about 30% to about 50% by weight of the tablet core. In more specific embodiments, the one or more anionic polymers constitute about 40% to about 50% by weight of the tablet core.

In some embodiments, the one or more anionic polymers constitute about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, or about 60% by weight of the tablet core.

In some embodiments, the homogeneous mixture comprises the one or more anionic polymers at about 250 mg to about 550 mg, about 260 mg to about 540 mg, about 270 mg to about 530 mg, about 280 mg to about 520 mg, or about 290 mg to about 510 mg. In some embodiments, the homogeneous mixture comprises the one or more anionic polymers at about 150 mg to about 550 mg, about 160 mg to about 540 mg, about 170 mg to about 530 mg, about 180 mg to about 520 mg, or about 190 mg to about 510 mg. In some embodiments, the homogeneous mixture comprises the one or more anionic polymers at about 200 mg to about 500 mg. In some embodiments, the homogeneous mixture comprises the one or more anionic polymers at about 210 mg.

In some embodiments, the homogeneous mixture comprises the one or more anionic polymers at about 300 mg to about 500 mg. In some embodiments, the homogeneous mixture comprises the one or more anionic polymers at about 475 mg.

In some embodiments, the homogeneous mixture comprises the one or more anionic polymers at about 300 mg to about 450 mg. In some embodiments, the homogeneous mixture comprises the one or more anionic polymers at about 290 mg to about 390 mg, about 300 mg to about 380 mg, about 310 mg to about 370 mg, about 320 mg to about 360 mg, or about 330 mg to about 350 mg. In some embodiments, the homogeneous mixture comprises the one or more anionic polymers at about 315 mg to about 365 mg. In some embodiments, the homogeneous mixture comprises the one or more anionic polymers at about 340 mg.

In some embodiments, the homogeneous mixture comprises the one or more anionic polymers at about 375 mg to about 475 mg, about 385 mg to about 465 mg, about 395 mg to about 455 mg, about 405 mg to about 445 mg, or about 415 mg to about 435 mg. In some embodiments, the homogeneous mixture comprises the one or more anionic polymers at about 400 mg to about 450 mg. In some embodiments, the homogeneous mixture comprises the one or more anionic polymers at about 425 mg.

In some embodiments, the homogeneous mixture comprises the one or more anionic polymers at about 250 mg, about 260 mg, about 270 mg, about 280 mg, about 290 mg, about 300 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 405 mg, about 410 mg, about 415 mg, about 420 mg, about 425 mg, about 430 mg, about 435 mg, about 440 mg, about 445 mg, about 450 mg, about 460 mg, about 470 mg, about 480 mg, about 490 mg, about 500 mg, about 510 mg, about 520 mg, about 530 mg, about 540 mg, or about 550 mg.

In some embodiments, the tablet core comprises the homogeneous mixture of dexpramipexole, or a pharmaceutically acceptable salt thereof, and the one or more polymers in the form of densified granules. In some embodiments, the tablet core comprises the homogeneous mixture of dexpramipexole, or a pharmaceutically acceptable salt thereof, and the one or more anionic polymers in the form of densified granules. In some embodiments, the tablet core comprises the homogeneous mixture of dexpramipexole, or a pharmaceutically acceptable salt thereof, and the one or more neutral polymers in the form of densified granules. Other ingredients such as a filler or a lubricant may be part of the densified granules. The densified granules may be obtained by roller compaction. A suitable roller compaction process is described, for example, further below in the specification in Chapter “III-Methods of Manufacturing,” section “3. Further embodiments of the methods of manufacturing”.

In some embodiments, the tablet core comprises the homogeneous mixture of dexpramipexole, or a pharmaceutically acceptable salt thereof, and the one or more polymers. In some embodiments, the homogeneous mixture of dexpramipexole, or a pharmaceutically acceptable salt thereof, and the one or more polymers is not in the form of densified granules. Other ingredients such as a filler or a lubricant may be part of the homogenous mixture. Suitable processes for preparing and processing the homogenous mixture are described, for example, further below in the specification in Chapter “III-Methods of Manufacturing,” section “3. Further embodiments of the methods of manufacturing”.

In some embodiments, the homogeneous mixture comprises one, but no more than one anionic polymer. In specific embodiments, the homogeneous mixture comprises one, but no more than one anionic polymer and the anionic polymer is carboxymethylcellulose (such as sodium carboxymethylcellulose). An example of a sodium carboxymethylcellulose is Aqualon CMC 7HXF PH and an example of a crosslinked acrylic acid polymer is Carbopol 971P. In more specific embodiments, the viscosity of the sodium carboxymethylcellulose is about 1,500 to about 3,000 cP (centipoise; 1% in water) and/or the viscosity of the crosslinked acrylic acid polymer is about 4,000 to about 11,000 cP (centipoise; 0.5% at pH 7.5). In specific embodiments, the homogeneous mixture comprises one, but no more than one anionic polymer and the anionic polymer is an acrylic acid polymer, such as crosslinked acrylic acid polymer. An example of a crosslinked acrylic acid polymer is Carbopol 971P. In more specific embodiments, the viscosity of the crosslinked acrylic acid polymer is about 4,000 to about 11,000 cP (centipoise; 0.5% at pH 7.5).

In some embodiments, the homogeneous mixture comprises more than one anionic polymer, such as two, three, four, or five anionic polymers. Within the meaning of the present disclosure, if the homogeneous mixture comprises more than one anionic polymer, such as a first and a second anionic polymer, the anionic polymers are different anionic polymers. For example, a homogeneous mixture comprising two anionic polymers might comprise sodium carboxymethylcellulose as the first anionic polymer and an acrylic acid polymer as the second anionic polymer.

In some embodiments, the homogeneous mixture comprises two, but no more than two anionic polymers. In specific embodiments, the homogeneous mixture comprises two, but no more than two anionic polymers and the two anionic polymers are carboxymethylcellulose (such as sodium carboxymethylcellulose) and an acrylic acid polymer (such as a crosslinked acrylic acid polymer). An example of a sodium carboxymethylcellulose is Aqualon CMC 7HXF PH and an example of a crosslinked acrylic acid polymer is Carbopol 971P. In more specific embodiments, the viscosity of the sodium carboxymethylcellulose is about 1,500 to about 3,000 cP (centipoise; 1% in water) and/or the viscosity of the crosslinked acrylic acid polymer is about 4,000 to about 11,000 cP (centipoise; 0.5% at pH 7.5).

In some embodiments, the homogeneous mixture comprises three, but no more than three anionic polymers. In some embodiments, the homogeneous mixture comprises four, but no more than four anionic polymers. In some embodiments, the homogeneous mixture comprises five, but no more than five anionic polymers.

In some embodiments, the homogeneous mixture comprises one or more, but no more than five anionic polymers. In some embodiments, the homogeneous mixture comprises one or more, but no more than four anionic polymers. In some embodiments, the homogeneous mixture comprises one or more, but no more than three anionic polymers. In some embodiments, the homogeneous mixture comprises one or more, but no more than two anionic polymers.

In specific embodiments, the homogeneous mixture comprises two, but no more than two anionic polymers.

In specific embodiments, the homogeneous mixture comprises one or more, but no more than two anionic polymers.

In some embodiments, the homogeneous mixture consists essentially of dexpramipexole, or a pharmaceutically acceptable salt thereof, at the amounts specified herein (such as at about 75 mg to about 376 mg of dexpramipexole dihydrochloride equivalent) and one, but no more than one anionic polymers at the amounts specified herein (such as at about 30% to about 50% by weight of the tablet core). In some embodiments, the homogeneous mixture consists essentially of dexpramipexole, or a pharmaceutically acceptable salt thereof, at the amounts specified herein (such as at about 75 mg to about 300 mg of dexpramipexole dihydrochloride equivalent) and one or more, but no more than two anionic polymers at the amounts specified herein (such as at about 30% to about 50% by weight of the tablet core). If two anionic polymers are present, the two anionic polymers may be carboxymethylcellulose (such as sodium carboxymethylcellulose) and an acrylic acid polymer (such as a crosslinked acrylic acid polymer).

In specific embodiments, the homogeneous mixture consists essentially of dexpramipexole, or a pharmaceutically acceptable salt thereof, at the amounts specified herein (such as at about 75 mg to about 300 mg of dexpramipexole dihydrochloride equivalent) and two anionic polymers at the amounts specified herein (such as at about 30% to about 50% by weight of the tablet core). The two anionic polymers may be carboxymethylcellulose (such as sodium carboxymethylcellulose) and an acrylic acid polymer (such as a crosslinked acrylic acid polymer).

In some embodiments, the homogeneous mixture comprises a first anionic polymer and a second anionic polymer and the first anionic polymer is carboxymethylcellulose and the second anionic polymer is an acrylic acid polymer. In specific embodiments, carboxymethylcellulose is sodium carboxymethylcellulose and/or the acrylic acid polymer is a crosslinked acrylic acid polymer. An example of a sodium carboxymethylcellulose is Aqualon CMC 7HXF PH and an example of a crosslinked acrylic acid polymer is Carbopol 971P. The viscosity of the sodium carboxymethylcellulose may be about 1,500 to about 3,000 cP (centipoise; 1% in water) and/or the viscosity of the crosslinked acrylic acid polymer may be about 4,000 to about 11,000 cP (centipoise; 0.5% at pH 7.5).

In some embodiments, the amount of carboxymethylcellulose is about 23% to about 38% of sodium carboxymethylcellulose equivalent by weight of the tablet core. In some embodiments, the amount of carboxymethylcellulose is about 25% to about 30% of sodium carboxymethylcellulose equivalent by weight of the tablet core. In some embodiments, the amount of carboxymethylcellulose is about 28% of sodium carboxymethylcellulose equivalent by weight of the tablet core.

In some embodiments, the amount of carboxymethylcellulose is about 100 mg to about 300 mg of sodium carboxymethylcellulose equivalent. In some embodiments, the amount of carboxymethylcellulose is about 200 mg to about 250 mg of sodium carboxymethylcellulose equivalent. In some embodiments, the amount of carboxymethylcellulose is about 210 mg of sodium carboxymethylcellulose equivalent.

In some embodiments, the amount of carboxymethylcellulose is about 23% to about 38% of sodium carboxymethylcellulose equivalent by weight of the tablet core and the amount of the acrylic acid polymer is about 7% to about 22% by weight of the tablet core. In some embodiments, the amount of carboxymethylcellulose is about 23% to about 33% of sodium carboxymethylcellulose equivalent by weight of the tablet core and the amount of the acrylic acid polymer is about 7% to about 17%, about 8% by weight of the tablet core. In some embodiments, the amount of carboxymethylcellulose is about 24% to about 32% of sodium carboxymethylcellulose equivalent by weight of the tablet core and the amount of the acrylic acid polymer is about 8% to about 16% by weight of the tablet core. In some embodiments, the amount of carboxymethylcellulose is about 25% to about 31% of sodium carboxymethylcellulose equivalent by weight of the tablet core and the amount of the acrylic acid polymer is about 9% to about 15% by weight of the tablet core. In some embodiments, the amount of carboxymethylcellulose is about 26% to about 30% of sodium carboxymethylcellulose equivalent by weight of the tablet core and the amount of the acrylic acid polymer is about 10% to about 14% by weight of the tablet core. In some embodiments, the amount of carboxymethylcellulose is about 27% to about 29% of sodium carboxymethylcellulose equivalent by weight of the tablet core and the amount of the acrylic acid polymer is about 11% to about 13% by weight of the tablet core. In specific embodiments, the amount of carboxymethylcellulose is about 28% of sodium carboxymethylcellulose equivalent by weight of the tablet core and the amount of the acrylic acid polymer is about 12% by weight of the tablet core. In some embodiments, the amount of carboxymethylcellulose may also be about 28% to about 38% of sodium carboxymethylcellulose equivalent by weight of the tablet core and the amount of the acrylic acid polymer may be about 9% to about 19% by weight of the tablet core. In some embodiments, the amount of carboxymethylcellulose may also be about 29% to about 37% of sodium carboxymethylcellulose equivalent by weight of the tablet core and the amount of the acrylic acid polymer may be about 10% to about 18% by weight of the tablet core. In some embodiments, the amount of carboxymethylcellulose may also be about 30% to about 36% of sodium carboxymethylcellulose equivalent by weight of the tablet core and the amount of the acrylic acid polymer may be about 11% to about 17% by weight of the tablet core. In some embodiments, the amount of carboxymethylcellulose is about 25% to about 35% of sodium carboxymethylcellulose equivalent by weight of the tablet core and the amount of the acrylic acid polymer is about 8% to about 18% by weight of the tablet core. In some embodiments, the amount of carboxymethylcellulose is about 26% to about 34% of sodium carboxymethylcellulose equivalent by weight of the tablet core and the amount of the acrylic acid polymer is about 9% to about 17% by weight of the tablet core. In some embodiments, the amount of carboxymethylcellulose is about 27% to about 33% of sodium carboxymethylcellulose equivalent by weight of the tablet core and the amount of the acrylic acid polymer is about 10% to about 16% by weight of the tablet core. In some embodiments, the amount of carboxymethylcellulose is about 28% to about 32% of sodium carboxymethylcellulose equivalent by weight of the tablet core and the amount of the acrylic acid polymer is about 11% to about 15% by weight of the tablet core. In specific embodiments, the amount of carboxymethylcellulose is about 30% of sodium carboxymethylcellulose equivalent by weight of the tablet core and the amount of the acrylic acid polymer is about 13% by weight of the tablet core. In some embodiments, the amount of carboxymethylcellulose may also be about 31% to about 35% of sodium carboxymethylcellulose equivalent by weight of the tablet core and the amount of the acrylic acid polymer may be about 12% to about 16% by weight of the tablet core. In some embodiments, the amount of carboxymethylcellulose may also be about 32% to about 34% of sodium carboxymethylcellulose equivalent by weight of the tablet core and the amount of the acrylic acid polymer may be about 13% to about 15% by weight of the tablet core. In specific embodiments, the amount of carboxymethylcellulose is about 33% of sodium carboxymethylcellulose equivalent by weight of the tablet core and the amount of the acrylic acid polymer is about 14% by weight of the tablet core.

In some embodiments, the amount of carboxymethylcellulose is about 23%, about 24%, about 25%, about 26%, about 27%, about 28%, about 29%, about 30%, about 31%, about 32%, about 33%, about 34%, about 35%, about 36%, about 37%, or about 38% of sodium carboxymethylcellulose equivalent by weight of the tablet core and the amount of the acrylic acid polymer is about 7%, about 8%, about 9%, about 10%, about 11%, about 12%, about 13%, about 14%, about 15%, about 16%, about 17%, about 18%, about 19%, about 20%, about 21%, or about 22% by weight of the tablet core.

In some embodiments, the amount of carboxymethylcellulose is about 190 mg to about 350 mg of sodium carboxymethylcellulose equivalent and the amount of the acrylic acid polymer is about 60 mg to about 200 mg. In some embodiments, the amount of carboxymethylcellulose is about 205 mg to about 335 mg of sodium carboxymethylcellulose equivalent and the amount of the acrylic acid polymer is about 70 mg to about 145 mg. In even more specific embodiments, the amount of carboxymethylcellulose is about 333 mg of sodium carboxymethylcellulose equivalent and the amount of the acrylic acid polymer is about 143 mg. In some embodiments, the amount of carboxymethylcellulose is about 200 mg to about 250 mg of sodium carboxymethylcellulose equivalent and the amount of the acrylic acid polymer is about 75 mg to about 125 mg. In some embodiments, the amount of carboxymethylcellulose is about 220 mg to about 260 mg of sodium carboxymethylcellulose equivalent and the amount of the acrylic acid polymer is about 80 mg to about 120 mg. In some embodiments, the amount of carboxymethylcellulose is about 230 mg to about 250 mg of sodium carboxymethylcellulose equivalent and the amount of the acrylic acid polymer is about 90 mg to about 110 mg. In some embodiments, the amount of carboxymethylcellulose is about 235 mg to about 245 mg of sodium carboxymethylcellulose equivalent and the amount of the acrylic acid polymer is about 95 mg to about 105 mg. In specific embodiments, the amount of carboxymethylcellulose is about 235 mg to about 240 mg of sodium carboxymethylcellulose equivalent and the amount of the acrylic acid polymer is about 100 mg to about 105 mg. In even more specific embodiments, the amount of carboxymethylcellulose is about 238 mg of sodium carboxymethylcellulose equivalent and the amount of the acrylic acid polymer is about 102 mg. In some embodiments, the amount of carboxymethylcellulose is about 275 mg to about 325 mg of sodium carboxymethylcellulose equivalent and the amount of the acrylic acid polymer is about 100 mg to about 150 mg. In some embodiments, the amount of carboxymethylcellulose is about 280 mg to about 320 mg of sodium carboxymethylcellulose equivalent and the amount of the acrylic acid polymer is about 110 mg to about 150 mg. In some embodiments, the amount of carboxymethylcellulose is about 290 mg to about 310 mg of sodium carboxymethylcellulose equivalent and the amount of the acrylic acid polymer is about 120 mg to about 140 mg. In specific embodiments, the amount of carboxymethylcellulose is about 295 mg to about 305 mg of sodium carboxymethylcellulose equivalent and the amount of the acrylic acid polymer is about 125 mg to about 135 mg. In specific embodiments, the amount of carboxymethylcellulose is about 295 mg to about 300 mg of sodium carboxymethylcellulose equivalent and the amount of the acrylic acid polymer is about 125 mg to about 130 mg. In even more specific embodiments, the amount of carboxymethylcellulose is about 298 mg of sodium carboxymethylcellulose equivalent and the amount of the acrylic acid polymer is about 128 mg.

In some embodiments, the amount of carboxymethylcellulose is about 190 mg, about 200 mg, about 205 mg, about 210 mg, about 215 mg, about 220 mg, about 225 mg, about 230 mg, about 235 mg, about 238 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, or about 350 mg of sodium carboxymethylcellulose equivalent and the amount of the acrylic acid polymer is about 60 mg, about 65 mg, about 70 mg, about 75 mg, about 80 mg, about 85 mg, about 90 mg, about 95 mg, about 100 mg, about 102 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, or about 200 mg.

In some embodiments, the weight ratio of the acrylic acid polymer to sodium carboxymethylcellulose equivalent is about 3:2 to about 2:8. In some embodiments, the weight ratio of the acrylic acid polymer to sodium carboxymethylcellulose equivalent is about 3:3 (1:1) to about 2:7. In some embodiments, the weight ratio of the acrylic acid polymer to sodium carboxymethylcellulose equivalent is about 3:4 to about 2:6. In some embodiments, the weight ratio of the acrylic acid polymer to sodium carboxymethylcellulose equivalent is about 2:3 to about 2:8. In specific embodiments, the weight ratio of the acrylic acid polymer to sodium carboxymethylcellulose equivalent is about 3:5 to about 2:5. In even more specific embodiments, the weight ratio of the acrylic acid polymer to sodium carboxymethylcellulose equivalent is about 3:7.

In some embodiments, the weight ratio of the acrylic acid polymer to sodium carboxymethylcellulose equivalent is about 3:2, about 3:3 (1:1), about 3:4, about 3:5, about 3:6, about 3:7, about 3:8, about 3:9, about 2:3, about 2:4, about 2:5, about 2:6, about 2:7, or about 2:8.

In some embodiments, the homogeneous mixture comprises the first and the second anionic polymer, but no more other anionic polymers than the first and the second anionic polymer.

In some embodiments, the homogeneous mixture consists essentially of dexpramipexole, or a pharmaceutically acceptable salt thereof, and the first and the second anionic polymer.

In some embodiments, the homogeneous mixture in the tablet core in addition to the amount of dexpramipexole, or the pharmaceutically acceptable salt and the one or more anionic polymers at the amounts specified herein further comprises one or more hydrophilic neutral polymers. In some specific embodiments, the one or more hydrophilic neutral polymers are hypromellose (hydroxypropyl methylcellulose, HPMC), silicified hydroxypropyl methylcellulose, hydroxypropyl cellulose (HPC), and/or poly(ethylene oxide). Non-limiting examples of a HPMC are Methocel® K15M, Methocel® K100M, and Methocel® K200M. A non-limiting example of a HPC is Klucel® Xtend HXF. A non-limiting example of poly(ethylene oxide) is Sentry Polyox® WSR-303.

In some embodiments, the homogeneous mixture comprises one or more hydrophilic neutral polymers. In some embodiments, the homogeneous mixture comprises one, but no more than one neutral polymer. In some specific embodiments, the one or more hydrophilic neutral polymers are hypromellose (hydroxypropyl methylcellulose, HPMC), silicified hydroxypropyl methylcellulose, hydroxypropyl cellulose (HPC), and/or poly(ethylene oxide). Non-limiting examples of a HPMC are Methocel® K15M, Methocel® K100M, and Methocel® K200M. A non-limiting example of a HPC is Klucel® Xtend HXF. A non-limiting example of poly(ethylene oxide) is Sentry Polyox® WSR-303.

In some embodiments, the amount of hydroxypropyl methylcellulose or silicified hydroxypropyl methylcellulose is about 5% to about 25% by weight of the tablet core. In some embodiments, the amount of hydroxypropyl methylcellulose or silicified hydroxypropyl methylcellulose is about 10% to about 20% by weight of the tablet core. In some embodiments, the amount of hydroxypropyl methylcellulose or silicified hydroxypropyl methylcellulose is about 15% by weight of the tablet core.

In some embodiments, the one or more neutral polymers constitute between about 5% and about 60% by weight of the tablet core. In some embodiments, the one or more neutral polymers constitute between about 10% and about 60% by weight of the tablet core. In some embodiments, the one or more neutral polymers constitute about 15% to about 60% by weight of the tablet core. In some embodiments, the one or more neutral polymers constitute between about 20% and about 60% by weight of the tablet core. In some embodiments, the one or more neutral polymers constitute about 25% to about 60% by weight of the tablet core. In some embodiments, the one or more neutral polymers constitute between about 30% and about 60% by weight of the tablet core. In some embodiments, the one or more neutral polymers constitute about 35% to about 60% by weight of the tablet core. In some embodiments, the one or more neutral polymers constitute between about 40% and about 60% by weight of the tablet core. In some embodiments, the one or more neutral polymers constitute about 45% to about 60% by weight of the tablet core. In some embodiments, the one or more neutral polymers constitute between about 50% and about 60% by weight of the tablet core. In some embodiments, the one or more neutral polymers constitute about 55% to about 60% by weight of the tablet core.

In some embodiments, the one or more neutral polymers constitute about 5% to about 55% by weight of the tablet core. In some embodiments, the one or more neutral polymers constitute about 5% to about 50% by weight of the tablet core. In some embodiments, the one or more neutral polymers constitute about 5% to about 45% by weight of the tablet core. In some embodiments, the one or more neutral polymers constitute about 5% to about 40% by weight of the tablet core. In some embodiments, the one or more neutral polymers constitute about 5% to about 35% by weight of the tablet core. In some embodiments, the one or more neutral polymers constitute about 5% to about 30% by weight of the tablet core.

In some embodiments, the one or more neutral polymers constitute about 20% by weight of the tablet core. In some embodiments, the one or more neutral polymers constitute about 25% by weight of the tablet core. In some embodiments, the one or more neutral polymers constitute about 30% by weight of the tablet core. In some embodiments, the one or more neutral polymers constitute about 35% by weight of the tablet core. In some embodiments, the one or more neutral polymers constitute about 40% by weight of the tablet core. In some embodiments, the one or more neutral polymers constitute about 45% by weight of the tablet core. In some embodiments, the one or more neutral polymers constitute about 50% by weight of the tablet core. In some embodiments, the one or more neutral polymers constitute about 55% by weight of the tablet core. In some embodiments, the one or more neutral polymers constitute about 60% by weight of the tablet core.

In some embodiments, the amount of hydroxypropyl methylcellulose or silicified hydroxypropyl methylcellulose is about 50 mg to about 150 mg. In some embodiments, the amount of hydroxypropyl methylcellulose or silicified hydroxypropyl methylcellulose is about 80 mg to about 140 mg. In some embodiments, the amount of hydroxypropyl methylcellulose or silicified hydroxypropyl methylcellulose is about 113 mg.

In some embodiments, the homogeneous mixture in the tablet core in addition to the amount of dexpramipexole, or the pharmaceutically acceptable salt and the one or more anionic polymers at the amounts specified herein further comprises one or more lipophilic materials. In some specific embodiments, the one or more lipophilic materials are glycerol dibehenate and/or carnuba wax. A non-limiting example of a glycerol dibehenate is Compritol® 888 ATO.

In some embodiments, the homogeneous mixture in the tablet core in addition to the amount of dexpramipexole, or the pharmaceutically acceptable salt and the one or more polymers at the amounts specified herein further comprises a diluent (in the Examples also referred to as “filler”). In some embodiments, the homogeneous mixture in the tablet core in addition to the amount of dexpramipexole, or the pharmaceutically acceptable salt and the one or more anionic polymers at the amounts specified herein further comprises a diluent (in the Examples also referred to as “filler”). In some embodiments, the homogeneous mixture in the tablet core in addition to the amount of dexpramipexole, or the pharmaceutically acceptable salt and the one or more neutral polymers at the amounts specified herein further comprises a diluent (in the Examples also referred to as “filler”). As used herein, the term “diluent” is intended to mean an inert substance used as filler to create the desired bulk, flow properties, and compression characteristics in the preparation of tablets. Examples of suitable diluents are microcrystalline cellulose (such as silicified microcrystalline cellulose), powdered cellulose, precipitated calcium carbonate, sorbitol, starch, mannitol, anhydrous lactose, lactose monohydrate, and the like.

In some embodiments, the diluent constitutes about 10% to about 30%, about 11% to about 29%, about 12% to about 28%, about 13% to about 27%, about 14% to about 26%, or about 15% to about 25% by weight of the tablet core. In some embodiments, the diluent constitutes about 10% to about 50%, about 11% to about 49%, about 12% to about 48%, about 13% to about 47%, about 14% to about 46%, or about 15% to about 45% by weight of the tablet core. In some embodiments, the diluent constitutes about 42% by weight of the tablet core. In some embodiments, the diluent constitutes about 15% to about 40% by weight of the tablet core. In some embodiments, the diluent constitutes about 20% to about 35% by weight of the tablet core. In some embodiments, the diluent constitutes about 29% by weight of the tablet core. In some embodiments, the diluent constitutes about 21% by weight of the tablet core. In some embodiments, the diluent constitutes about 15% to about 20% by weight of the tablet core. In some embodiments, the diluent constitutes about 20% to about 25% by weight of the tablet core. In specific embodiments, the diluent constitutes about 16% by weight of the tablet core. In other specific embodiments, the diluent constitutes about 21% by weight of the tablet core. In some embodiments, the diluent constitutes about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, or about 45%. In some embodiments, the tablet core comprises about 50 mg to about 350 mg, about 75 mg to about 325 mg, about 100 mg to about 200 mg, about 110 mg to about 190 mg, about 120 mg to about 180 mg, about 130 mg to about 170 mg, or about 140 mg to about 160 mg diluent. In some embodiments, the tablet core comprises about 100 mg to about 150 mg diluent. In some embodiments, the tablet core comprises about 150 mg to about 200 mg diluent. In some embodiments, the tablet core comprises about 135 mg to about 155 mg diluent. In some embodiments, the tablet core comprises about 170 mg to about 190 mg diluent. In specific embodiments, the tablet core comprises about 145 mg diluent. In other specific embodiments, the tablet core comprises about 180 mg diluent. In other specific embodiments, the tablet core comprises about 214 mg diluent. In other specific embodiments, the tablet core comprises about 236 mg diluent. In other specific embodiments, the tablet core comprises about 312 mg diluent. In some embodiments, the tablet core comprises about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, or about 350 mg diluent.

In specific embodiments, the diluent is silicified microcrystalline cellulose. The silicified microcrystalline cellulose may be obtained by co-processing colloidal silicon dioxide and microcrystalline cellulose. In some embodiments, silicified microcrystalline cellulose constitutes about 10% to about 30%, about 11% to about 29%, about 12% to about 28%, about 13% to about 27%, about 14% to about 26%, or about 15% to about 25% by weight of the tablet core. In some embodiments, silicified microcrystalline cellulose constitutes about 10% to about 50%, about 11% to about 49%, about 12% to about 48%, about 13% to about 47%, about 14% to about 46%, or about 15% to about 45% by weight of the tablet core. In some embodiments, silicified microcrystalline cellulose constitutes about 42% by weight of the tablet core. In some embodiments, silicified microcrystalline cellulose constitutes about 15% to about 40% by weight of the tablet core. In some embodiments, silicified microcrystalline cellulose constitutes about 20% to about 35% by weight of the tablet core. In some embodiments, silicified microcrystalline cellulose constitutes about 29% by weight of the tablet core. In some embodiments, silicified microcrystalline cellulose constitutes about 21% by weight of the tablet core. In some embodiments, silicified microcrystalline cellulose constitutes about 15% to about 20% by weight of the tablet core. In some embodiments, silicified microcrystalline cellulose constitutes about 20% to about 25% by weight of the tablet core. In specific embodiments, silicified microcrystalline cellulose constitutes about 16% by weight of the tablet core. In other specific embodiments, silicified microcrystalline cellulose constitutes about 21% by weight of the tablet core. In some embodiments, silicified microcrystalline cellulose constitutes about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, or about 45%. In some embodiments, the tablet core comprises about 50 mg to about 350 mg, about 75 mg to about 325 mg, about 100 mg to about 200 mg, about 110 mg to about 190 mg, about 120 mg to about 180 mg, about 130 mg to about 170 mg, or about 140 mg to about 160 mg silicified microcrystalline cellulose. In some embodiments, the tablet core comprises about 100 mg to about 150 mg silicified microcrystalline cellulose. In some embodiments, the tablet core comprises about 150 mg to about 200 mg silicified microcrystalline cellulose. In some embodiments, the tablet core comprises about 135 mg to about 155 mg silicified microcrystalline cellulose. In some embodiments, the tablet core comprises about 170 mg to about 190 mg silicified microcrystalline cellulose. In specific embodiments, the tablet core comprises about 145 mg silicified microcrystalline cellulose. In other specific embodiments, the tablet core comprises about 180 mg silicified microcrystalline cellulose. In other specific embodiments, the tablet core comprises about 214 mg silicified microcrystalline cellulose. In other specific embodiments, the tablet core comprises about 236 mg silicified microcrystalline cellulose. In other specific embodiments, the tablet core comprises about 312 mg silicified microcrystalline cellulose. In some embodiments, the tablet core comprises about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, or about 350 mg silicified microcrystalline cellulose.

In specific embodiments, the diluent is microcrystalline cellulose. In some embodiments, microcrystalline cellulose constitutes about 10% to about 30%, about 11% to about 29%, about 12% to about 28%, about 13% to about 27%, about 14% to about 26%, or about 15% to about 25% by weight of the tablet core. In some embodiments, microcrystalline cellulose constitutes about 10% to about 50%, about 11% to about 49%, about 12% to about 48%, about 13% to about 47%, about 14% to about 46%, or about 15% to about 45% by weight of the tablet core. In some embodiments, microcrystalline cellulose constitutes about 42% by weight of the tablet core. In some embodiments, microcrystalline cellulose constitutes about 15% to about 40% by weight of the tablet core. In some embodiments, microcrystalline cellulose constitutes about 20% to about 35% by weight of the tablet core. In some embodiments, microcrystalline cellulose constitutes about 29% by weight of the tablet core. In some embodiments, microcrystalline cellulose constitutes about 21% by weight of the tablet core. In some embodiments, microcrystalline cellulose constitutes about 15% to about 20% by weight of the tablet core. In some embodiments, microcrystalline cellulose constitutes about 20% to about 25% by weight of the tablet core. In specific embodiments, microcrystalline cellulose constitutes about 16% by weight of the tablet core. In other specific embodiments, microcrystalline cellulose constitutes about 21% by weight of the tablet core. In some embodiments, microcrystalline cellulose constitutes about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, or about 45%. In some embodiments, the tablet core comprises about 50 mg to about 350 mg, about 75 mg to about 325 mg, about 100 mg to about 200 mg, about 110 mg to about 190 mg, about 120 mg to about 180 mg, about 130 mg to about 170 mg, or about 140 mg to about 160 mg microcrystalline cellulose. In some embodiments, the tablet core comprises about 100 mg to about 150 mg microcrystalline cellulose. In some embodiments, the tablet core comprises about 150 mg to about 200 mg microcrystalline cellulose. In some embodiments, the tablet core comprises about 135 mg to about 155 mg microcrystalline cellulose. In some embodiments, the tablet core comprises about 170 mg to about 190 mg microcrystalline cellulose. In specific embodiments, the tablet core comprises about 145 mg microcrystalline cellulose. In other specific embodiments, the tablet core comprises about 180 mg microcrystalline cellulose. In other specific embodiments, the tablet core comprises about 214 mg microcrystalline cellulose. In other specific embodiments, the tablet core comprises about 236 mg microcrystalline cellulose. In other specific embodiments, the tablet core comprises about 312 mg microcrystalline cellulose. In some embodiments, the tablet core comprises about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, or about 350 mg microcrystalline cellulose.

In some embodiments, the homogeneous mixture in the tablet core in addition to the amount of dexpramipexole, or the pharmaceutically acceptable salt and the one or more polymers at the amounts specified herein further comprises a lubricant. In some embodiments, the homogeneous mixture in the tablet core in addition to the amount of dexpramipexole, or the pharmaceutically acceptable salt and the one or more anionic polymers at the amounts specified herein further comprises a lubricant. In some embodiments, the homogeneous mixture in the tablet core in addition to the amount of dexpramipexole, or the pharmaceutically acceptable salt and the one or more neutral polymers at the amounts specified herein further comprises a lubricant. As used herein, the term “lubricant” is intended to mean substances used in tablet formulations to reduce friction during blending, (dry) granulation, and/or tablet compression. Examples of suitable lubricants are magnesium stearate, calcium stearate, sodium stearyl fumarate, zinc stearate, and the like. Also glycerol dibehenate and carnuba wax might be applied.

In some embodiments, the lubricant constitutes about 0.25% to about 1.25%, about 0.3% to about 1.2%, about 0.35% to about 1.15%, about 0.4% to about 1.10%, about 0.45% to about 1.05%, or about 0.5% to about 1% by weight of the tablet core. In some embodiments, the lubricant constitutes about 0.55% to about 0.95% by weight of the tablet core. In some embodiments, the lubricant constitutes about 0.6% to about 0.9% by weight of the tablet core. In some embodiments, the lubricant constitutes about 0.65% to about 0.85% by weight of the tablet core. In some embodiments, the lubricant constitutes about 0.7% to about 0.8% by weight of the tablet core. In specific embodiments, the lubricant constitutes about 0.75% by weight of the tablet core. In some embodiments, the lubricant constitutes about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, about 0.9%, about 0.95%, about 1%, about 1.05%, about 1.1%, about 1.15%, or about 1.2% by weight of the tablet core. In some embodiments, the tablet core comprises about 1 mg to about 12 mg, about 2 mg to about 11 mg, about 3 mg to about 10 mg, or about 4 mg to about 9 mg lubricant. In some embodiments, the tablet core comprises about 5 mg to about 8 mg lubricant. In some embodiments, the tablet core comprises about 6 mg to about 7 mg lubricant. In some embodiments, the tablet core comprises about 6 mg lubricant. In some embodiments, the tablet core comprises about 7 mg lubricant. In some embodiments, the tablet core comprises about 1 mg, about 2 mg, about 3 mg, about 4 mg, about 5 mg, about 6 mg, about 7 mg, about 8 mg, about 9 mg, about 10 mg, about 11 mg, or about 12 mg lubricant.

In specific embodiments, the lubricant is magnesium stearate. In some embodiments, magnesium stearate constitutes about 0.25% to about 1.25%, about 0.3% to about 1.2%, about 0.35% to about 1.15%, about 0.4% to about 1.1%, about 0.45% to about 1.05%, or about 0.5% to about 1% by weight of the tablet core. In some embodiments, magnesium stearate constitutes about 0.55% to about 0.95% by weight of the tablet core. In some embodiments, magnesium stearate constitutes about 0.6% to about 0.9% by weight of the tablet core. In some embodiments, magnesium stearate constitutes about 0.65% to about 0.85% by weight of the tablet core. In some embodiments, magnesium stearate constitutes about 0.7% to about 0.8% by weight of the tablet core. In specific embodiments, magnesium stearate constitutes about 0.75% by weight of the tablet core. In some embodiments, magnesium stearate constitutes about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, about 0.9%, about 0.95%, about 1%, about 1.05%, about 1.1%, about 1.15%, or about 1.2% by weight of the tablet core. In some embodiments, the tablet core comprises about 1 mg to about 12 mg, about 2 mg to about 11 mg, about 3 mg to about 10 mg, or about 4 mg to about 9 mg magnesium stearate. In some embodiments, the tablet core comprises about 5 mg to about 8 mg magnesium stearate. In some embodiments, the tablet core comprises about 6 mg to about 7 mg magnesium stearate. In some embodiments, the tablet core comprises about 6 mg magnesium stearate. In some embodiments, the tablet core comprises about 7 mg magnesium stearate. In some embodiments, the tablet core comprises about 1 mg, about 2 mg, about 3 mg, about 4 mg, about 5 mg, about 6 mg, about 7 mg, about 8 mg, about 9 mg, about 10 mg, about 11 mg, or about 12 mg magnesium stearate.

In some embodiments, the homogeneous mixture in the tablet core in addition to the amount of dexpramipexole, or the pharmaceutically acceptable salt and the one or more polymers at the amounts specified herein further comprises a glidant. In some embodiments, the homogeneous mixture in the tablet core in addition to the amount of dexpramipexole, or the pharmaceutically acceptable salt and the one or more anionic polymers at the amounts specified herein further comprises a glidant. In some embodiments, the homogeneous mixture in the tablet core in addition to the amount of dexpramipexole, or the pharmaceutically acceptable salt and the one or more neutral polymers at the amounts specified herein further comprises a glidant. As used herein, the term “glidant” is intended to mean substances used in tablet formulations to improve flowability during blending, roller compaction, and/or tablet compression. Examples of suitable glidants are silicon dioxide, colloidal silicon dioxide, ascorbyl palmitate, calcium palmitate, starch, talc, and the like.

In some embodiments, the glidant constitutes about 0.1% to about 0.9%, about 0.15% to about 0.85%, about 0.2% to about 0.8%, or about 0.25% to about 0.75% by weight of the tablet core. In some embodiments, the glidant constitutes about 0.3% to about 0.7% by weight of the tablet core. In some embodiments, the glidant constitutes about 0.35% to about 0.65% by weight of the tablet core. In some embodiments, the glidant constitutes about 0.4% to about 0.6% by weight of the tablet core. In some embodiments, the glidant constitutes about 0.45% to about 0.55% by weight of the tablet core. In specific embodiments, the glidant constitutes about 0.5% by weight of the tablet core. In some embodiments, the glidant constitutes about 0.1%, about 0.15%, about 0.2%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, or about 0.9% by weight of the tablet core. In some embodiments, the tablet core comprises about 1 mg to about 8 mg glidant. In some embodiments, the tablet core comprises about 2 mg to about 7 mg glidant. In some embodiments, the tablet core comprises about 3 mg to about 6 mg glidant. In some embodiments, the tablet core comprises about 4 mg to about 5 mg glidant. In some embodiments, the tablet core comprises about 4 mg glidant. In some embodiments, the tablet core comprises about 5 mg glidant. In some embodiments, the tablet core comprises about 1 mg, about 2 mg, about 3 mg, about 4 mg, about 5 mg, about 6 mg, about 7 mg, or about 8 mg glidant.

In specific embodiments, the glidant is colloidal silicon dioxide. In some embodiments, colloidal silicon dioxide constitutes about 0.1% to about 0.9%, about 0.15% to about 0.85%, about 0.2% to about 0.8%, or about 0.25% to about 0.75% by weight of the tablet core. In some embodiments, colloidal silicon dioxide constitutes about 0.3% to about 0.7% by weight of the tablet core. In some embodiments, colloidal silicon dioxide constitutes about 0.35% to about 0.65% by weight of the tablet core. In some embodiments, colloidal silicon dioxide constitutes about 0.4% to about 0.6% by weight of the tablet core. In some embodiments, colloidal silicon dioxide constitutes about 0.45% to about 0.55% by weight of the tablet core. In specific embodiments, colloidal silicon dioxide constitutes about 0.5% by weight of the tablet core. In some embodiments, colloidal silicon dioxide constitutes about 0.1%, about 0.15%, about 0.2%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, or about 0.9% by weight of the tablet core. In some embodiments, the tablet core comprises about 1 mg to about 8 mg colloidal silicon dioxide. In some embodiments, the tablet core comprises about 2 mg to about 7 mg colloidal silicon dioxide. In some embodiments, the tablet core comprises about 3 mg to about 6 mg colloidal silicon dioxide. In some embodiments, the tablet core comprises about 4 mg to about 5 mg colloidal silicon dioxide. In some embodiments, the tablet core comprises about 4 mg colloidal silicon dioxide. In some embodiments, the tablet core comprises about 5 mg colloidal silicon dioxide. In some embodiments, the tablet core comprises about 1 mg, about 2 mg, about 3 mg, about 4 mg, about 5 mg, about 6 mg, about 7 mg, or about 8 mg colloidal silicon dioxide.

In some embodiments, the homogeneous mixture in addition to the amount of dexpramipexole, or the pharmaceutically acceptable salt (such as at about 30% to about 40% dexpramipexole dihydrochloride monohydrate equivalent by weight of the tablet core) and the one or more polymers at the amounts specified herein (such as at about 10% to about 55% by weight of the tablet core) further comprises about 15% to about 25% diluent (such as silicified microcrystalline cellulose) by weight of the tablet core, about 0.5% to about 1% lubricant (such as magnesium stearate) by weight of the tablet core, and about 0.25% to about 0.75% glidant (such as colloidal silicon dioxide) by weight of the tablet core. In some embodiments, the homogeneous mixture in addition to the amount of dexpramipexole, or the pharmaceutically acceptable salt (such as at about 30% to about 40% dexpramipexole dihydrochloride monohydrate equivalent by weight of the tablet core) and the one or more anionic polymers at the amounts specified herein (such as at about 40% to about 50% by weight of the tablet core) further comprises about 15% to about 25% diluent (such as silicified microcrystalline cellulose) by weight of the tablet core, about 0.5% to about 1% lubricant (such as magnesium stearate) by weight of the tablet core, and about 0.25% to about 0.75% glidant (such as colloidal silicon dioxide) by weight of the tablet core. In some embodiments, the homogeneous mixture in addition to the amount of dexpramipexole, or the pharmaceutically acceptable salt (such as at about 30% to about 40% dexpramipexole dihydrochloride monohydrate equivalent by weight of the tablet core) and the one or more neutral polymers at the amounts specified herein (such as at about 5% to about 25% by weight of the tablet core) further comprises about 15% to about 25% diluent (such as silicified microcrystalline cellulose) by weight of the tablet core, about 0.5% to about 1% lubricant (such as magnesium stearate) by weight of the tablet core, and about 0.25% to about 0.75% glidant (such as colloidal silicon dioxide) by weight of the tablet core.

In some embodiments, the homogeneous mixture in addition to the amount of dexpramipexole, or the pharmaceutically acceptable salt and the one or more polymers at the amounts specified herein further comprises a microcrystalline cellulose, magnesium stearate, colloidal silicon dioxide, or any combination thereof. In some embodiments, the homogeneous mixture in addition to the amount of dexpramipexole, or the pharmaceutically acceptable salt and the one or more anionic polymers at the amounts specified herein further comprises a microcrystalline cellulose, magnesium stearate, colloidal silicon dioxide, or any combination thereof. In some embodiments, the homogeneous mixture in addition to the amount of dexpramipexole, or the pharmaceutically acceptable salt and the one or more neutral polymers at the amounts specified herein further comprises a microcrystalline cellulose, magnesium stearate, colloidal silicon dioxide, or any combination thereof. In specific embodiments, the homogeneous mixture further comprises a microcrystalline cellulose, magnesium stearate and colloidal silicon dioxide. The microcrystalline cellulose may be silicified microcrystalline cellulose.

In some embodiments, the homogeneous mixture in the tablet core consists essentially of:
(a) about 315 mg to about 325 mg dexpramipexole dihydrochloride monohydrate;
(b) about 235 mg to about 245 mg sodium carboxymethylcellulose;
(c) about 95 mg to about 105 mg crosslinked acrylic acid polymer;
(d) about 175 mg to about 185 mg silicified microcrystalline cellulose;
(e) about 5 mg to about 7 mg magnesium stearate; and
(f) about 3 mg to about 5 mg colloidal silicon dioxide.

In more specific embodiments, the homogeneous mixture in the tablet core consists essentially of:
(a) about 319 mg dexpramipexole dihydrochloride monohydrate;
(b) about 238 mg sodium carboxymethylcellulose;
(c) about 102 mg crosslinked acrylic acid polymer;
(d) about 180 mg silicified microcrystalline cellulose;
(e) about 6 mg magnesium stearate; and
(f) about 4 mg colloidal silicon dioxide.

In some embodiments, the homogeneous mixture in the tablet core consists essentially of:
(a) about 315 mg to about 325 mg dexpramipexole dihydrochloride monohydrate;
(b) about 295 mg to about 305 mg sodium carboxymethylcellulose;
(c) about 125 mg to about 135 mg crosslinked acrylic acid polymer;
(d) about 140 mg to about 150 mg silicified microcrystalline cellulose;
(e) about 6 mg to about 8 mg magnesium stearate; and
(f) about 4 mg to about 6 mg colloidal silicon dioxide.

In more specific embodiments, the homogeneous mixture consists essentially of:
(a) about 319 mg dexpramipexole dihydrochloride monohydrate;
(b) about 298 mg sodium carboxymethylcellulose;
(c) about 128 mg crosslinked acrylic acid polymer;
(d) about 145 mg silicified microcrystalline cellulose;
(e) about 7 mg magnesium stearate; and
(f) about 5 mg colloidal silicon dioxide.

In some embodiments, the homogeneous mixture in the tablet core consists essentially of:
(a) about 390 mg to about 410 mg dexpramipexole dihydrochloride monohydrate;
(b) about 325 mg to about 341 mg sodium carboxymethylcellulose;
(c) about 135 mg to about 145 mg crosslinked acrylic acid polymer;
(d) about 230 mg to about 240 mg microcrystalline cellulose or silicified microcrystalline cellulose;
(e) about 5 mg to about 10 mg magnesium stearate; and
(f) about 4 mg to about 7 mg colloidal silicon dioxide.

In more specific embodiments, the homogeneous mixture consists essentially of:
(a) about 400 mg dexpramipexole dihydrochloride monohydrate;
(b) about 333 mg sodium carboxymethylcellulose;
(c) about 143 mg crosslinked acrylic acid polymer;
(d) about 236 mg microcrystalline cellulose or silicified microcrystalline cellulose;
(e) about 8 mg magnesium stearate; and
(f) about 6 mg colloidal silicon dioxide.

In some embodiments, the homogeneous mixture in the tablet core consists essentially of:
(a) about 315 mg to about 325 mg dexpramipexole dihydrochloride monohydrate;
(b) about 110 mg to about 120 mg hydroxypropyl methylcellulose or silicified hydroxypropyl methylcellulose;
(c) about 310 mg to about 320 mg microcrystalline cellulose or silicified microcrystalline cellulose;
(d) about 3 mg to about 5 mg magnesium stearate; and
(e) about 3 mg to about 5 mg colloidal silicon dioxide.

In more specific embodiments, the homogeneous mixture consists essentially of:
(a) about 319 mg dexpramipexole dihydrochloride monohydrate;
(b) about 113 mg hydroxypropyl methylcellulose or silicified hydroxypropyl methylcellulose;
(c) about 313 mg microcrystalline cellulose or silicified microcrystalline cellulose;
(d) about 4 mg magnesium stearate; and
(e) about 4 mg colloidal silicon dioxide.

In some embodiments, the homogeneous mixture in the tablet core consists essentially of:
(a) about 315 mg to about 325 mg dexpramipexole dihydrochloride monohydrate;
(b) about 110 mg to about 120 mg hydroxypropyl methylcellulose or silicified hydroxypropyl methylcellulose;
(c) about 305 mg to about 315 mg microcrystalline cellulose or silicified microcrystalline cellulose;
(d) about 5 mg to about 7 mg magnesium stearate; and
(e) about 3 mg to about 5 mg colloidal silicon dioxide.

In more specific embodiments, the homogeneous mixture consists essentially of:
(a) about 319 mg dexpramipexole dihydrochloride monohydrate;
(b) about 113 mg hydroxypropyl methylcellulose or silicified hydroxypropyl methylcellulose;
(c) about 310 mg microcrystalline cellulose or silicified microcrystalline cellulose;
(d) about 6 mg magnesium stearate; and
(e) about 4 mg colloidal silicon dioxide.

In some embodiments, the homogeneous mixture in the tablet core consists essentially of:
(a) about 315 mg to about 325 mg dexpramipexole dihydrochloride monohydrate;
(b) about 110 mg to about 120 mg hydroxypropyl methylcellulose or silicified hydroxypropyl methylcellulose;
(c) about 305 mg to about 315 mg microcrystalline cellulose;
(d) about 5 mg to about 7 mg magnesium stearate; and
(e) about 3 mg to about 5 mg colloidal silicon dioxide.

In more specific embodiments, the homogeneous mixture consists essentially of:
(a) about 319 mg dexpramipexole dihydrochloride monohydrate;
(b) about 113 mg hydroxypropyl methylcellulose or silicified hydroxypropyl methylcellulose;
(c) about 310 mg microcrystalline cellulose;
(d) about 6 mg magnesium stearate; and
(e) about 4 mg colloidal silicon dioxide.

In some embodiments, the homogeneous mixture in the tablet core consists essentially of:
(a) about 315 mg to about 325 mg dexpramipexole dihydrochloride monohydrate;
(b) about 200 mg to about 220 mg sodium carboxymethylcellulose;
(c) about 210 mg to about 220 mg microcrystalline cellulose or silicified microcrystalline cellulose;
(d) about 3 mg to about 5 mg magnesium stearate; and
(e) about 3 mg to about 5 mg colloidal silicon dioxide.

In more specific embodiments, the homogeneous mixture consists essentially of:
(a) about 319 mg dexpramipexole dihydrochloride monohydrate;
(b) about 210 mg sodium carboxymethylcellulose;
(c) about 214 mg microcrystalline cellulose or silicified microcrystalline cellulose;
(d) about 4 mg magnesium stearate; and
(e) about 4 mg colloidal silicon dioxide.

In some embodiments, the homogeneous mixture in the tablet core consists essentially of:
(a) about 30% to about 40% dexpramipexole dihydrochloride monohydrate by weight of the tablet core;
(b) about 25% to about 35% sodium carboxymethylcellulose by weight of the tablet core;
(c) about 10% to about 20% crosslinked acrylic acid polymer by weight of the tablet core;
(d) about 15% to about 25% silicified microcrystalline cellulose by weight of the tablet core;
(e) about 0.5% to about 1.0% magnesium stearate by weight of the tablet core; and
(f) about 0.25% to about 0.75% colloidal silicon dioxide by weight of the tablet core.

In some embodiments, the homogeneous mixture in the tablet core consists essentially of:
(a) about 35% to about 40% dexpramipexole dihydrochloride monohydrate by weight of the tablet core;
(b) about 28% to about 33% sodium carboxymethylcellulose by weight of the tablet core;
(c) about 10% to about 15% crosslinked acrylic acid polymer by weight of the tablet core;
(d) about 16% to about 21% silicified microcrystalline cellulose by weight of the tablet core;
(e) about 0.5% to about 1.0% magnesium stearate by weight of the tablet core; and
(f) about 0.25% to about 0.75% colloidal silicon dioxide by weight of the tablet core.

In more specific embodiments, the homogeneous mixture in the tablet core consists essentially of:
(a) about 38% dexpramipexole dihydrochloride monohydrate by weight of the tablet core;
(b) about 28% sodium carboxymethylcellulose by weight of the tablet core;
(c) about 12% crosslinked acrylic acid polymer by weight of the tablet core;
(d) about 21% silicified microcrystalline cellulose by weight of the tablet core;
(e) about 0.75% magnesium stearate by weight of the tablet core; and
(f) about 0.5% colloidal silicon dioxide by weight of the tablet core.

In other more specific embodiments, the homogeneous mixture in the tablet core consists essentially of:
(a) about 35% dexpramipexole dihydrochloride monohydrate by weight of the tablet core;
(b) about 33% sodium carboxymethylcellulose by weight of the tablet core;
(c) about 14% crosslinked acrylic acid polymer by weight of the tablet core;
(d) about 16% silicified microcrystalline cellulose by weight of the tablet core;
(e) about 0.75% magnesium stearate by weight of the tablet core; and
(f) about 0.5% colloidal silicon dioxide by weight of the tablet core.

In more specific embodiments, the homogeneous mixture in the tablet core consists essentially of:
(a) about 36% dexpramipexole dihydrochloride monohydrate by weight of the tablet core;
(b) about 30% sodium carboxymethylcellulose by weight of the tablet core;
(c) about 13% crosslinked acrylic acid polymer by weight of the tablet core;
(d) about 21% microcrystalline cellulose or silicified microcrystalline cellulose by weight of the tablet core;
(e) about 0.75% magnesium stearate by weight of the tablet core; and
(f) about 0.5% colloidal silicon dioxide by weight of the tablet core.

In other more specific embodiments, the homogeneous mixture in the tablet core consists essentially of:
(a) about 43% dexpramipexole dihydrochloride monohydrate by weight of the tablet core;
(b) about 15% hydroxypropyl methylcellulose or silicified hydroxypropyl methylcellulose by weight of the tablet core;
(c) about 42% microcrystalline cellulose or silicified microcrystalline cellulose by weight of the tablet core;
(d) about 0.5% magnesium stearate by weight of the tablet core; and
(e) about 0.5% colloidal silicon dioxide by weight of the tablet core.

In other more specific embodiments, the homogeneous mixture in the tablet core consists essentially of:
(a) about 43% dexpramipexole dihydrochloride monohydrate by weight of the tablet core;
(b) about 15% hydroxypropyl methylcellulose or silicified hydroxypropyl methylcellulose by weight of the tablet core;
(c) about 41% microcrystalline cellulose or silicified microcrystalline cellulose by weight of the tablet core;
(d) about 0.75% magnesium stearate by weight of the tablet core; and
(e) about 0.5% colloidal silicon dioxide by weight of the tablet core.

In other more specific embodiments, the homogeneous mixture in the tablet core consists essentially of:
(a) about 43% dexpramipexole dihydrochloride monohydrate by weight of the tablet core;
(b) about 15% hydroxypropyl methylcellulose or silicified hydroxypropyl methylcellulose by weight of the tablet core;
(c) about 41% microcrystalline cellulose by weight of the tablet core;
(d) about 0.75% magnesium stearate by weight of the tablet core; and
(e) about 0.5% colloidal silicon dioxide by weight of the tablet core.

In more specific embodiments, the homogeneous mixture in the tablet core consists essentially of:
(a) about 43% dexpramipexole dihydrochloride monohydrate by weight of the tablet core;
(b) about 28% sodium carboxymethylcellulose by weight of the tablet core
(c) about 29% microcrystalline cellulose or silicified microcrystalline cellulose by weight of the tablet core;
(d) about 0.5% magnesium stearate by weight of the tablet core; and
(e) about 0.5% colloidal silicon dioxide by weight of the tablet core.

In some embodiments, the amount of dexpramipexole dihydrochloride monohydrate, the amount of sodium carboxymethylcellulose, the amount of crosslinked acrylic acid polymer, the amount of silicified microcrystalline cellulose, and one third of the amount of magnesium stearate are in the form of densified granules. The densified granules may be obtained by roller compaction.

In some embodiments, the weight of the tablet core is about 400 mg to about 1000 mg, about 450 mg to about 950 mg, about 500 mg to about 900 mg, about 550 mg to about 850 mg, or about 600 mg to about 800 mg. In some embodiments, the weight of the tablet core is about 400 mg to about 1400 mg, about 450 mg to about 1350 mg, about 500 mg to about 1300 mg, about 550 mg to about 1250 mg, or about 600 mg to about 1200 mg. In some embodiments, the weight of the tablet core is about 800 mg to about 950 mg. In some embodiments, the weight of the tablet core is about 800 mg to about 900 mg. In some embodiments, the weight of the tablet core is about 810 mg to about 890 mg, about 820 mg to about 880 mg, or about 830 mg to about 870 mg. In some embodiments, the weight of the tablet core is about 840 mg to about 860 mg. In specific embodiments, the weight of the tablet core is about 850 mg. In some embodiments, the weight of the tablet core is about 850 mg to about 950 mg. In some embodiments, the weight of the tablet core is about 860 mg to about 940 mg, about 870 mg to about 930 mg, or about 880 mg to about 920 mg. In some embodiments, the weight of the tablet core is about 890 mg to about 910 mg. In specific embodiments, the weight of the tablet core is about 900 mg. In some embodiments, the weight of the tablet core is about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, or about 1400 mg. In some embodiments, the weight of the tablet core is about 1300 mg or less. In some embodiments, the weight of the tablet core is about 1000 mg or less. In some embodiments, the weight of the tablet core is about 950 mg or less. In some embodiments, the weight of the tablet core is about 900 mg or less. In some embodiments, the weight of the tablet core is about 850 mg or less. In some embodiments, the weight of the tablet core is about 800 mg or less. In some embodiments, the weight of the tablet core is at least about 500 mg, at least about 550 mg, at least about 600 mg, at least about 650 mg, at least about 700 mg, at least about 750 mg, or at least about 800 mg.

### 4. Film Coating

In some embodiments, the pharmaceutical composition further comprises a film surrounding the tablet core. In general, the film coating is applied in an amount sufficient to provide complete coverage of the tablet core.

In some embodiments, the film coating is a nonfunctional coating. The term “nonfunctional” in this context means having no substantial effect on drug release properties of the pharmaceutical composition, but does not imply that the film coating serves no useful purpose. For example, such film coating can impart a distinctive appearance to the tablet (such as providing for a specific color of the tablets), provide protection against attrition during packaging and transportation, and improve ease of swallowing. In some embodiments, the film coating provides a moisture-barrier.

In general, a tablet can comprise more than one film coating.

In some embodiments, the film coating comprises polyvinyl alcohol, hydroxypropyl methylcellulose (HPMC), hydroxypropyl cellulose (HPC), methacrylate, methacrylic acid copolymers, ethylcellulose, or any combination thereof.

In some embodiments, the film coating comprises polyvinyl alcohol. In some specific embodiments, the film coating comprises polyvinyl alcohol, titanium dioxide, polyethylene glycol, and talc. In other specific embodiments, the film coating consists of polyvinyl alcohol, titanium dioxide, polyethylene glycol, and talc. Examples of suitable film coatings are Opadry® II 85F18422 White and Opadry® II 85F130089 Pink.

In some embodiments, the film coating further comprises a color additive. A color additive, as defined by regulation, is any dye, pigment, or substance that can provide color to a food, drug, or cosmetic. Examples of suitable color additives include but are not limited to iron oxide (e.g., ferric oxide red), lead oxide, and copper sulfate.

In some embodiments, the pharmaceutical composition comprises about 10 mg to about 40 mg film coating. In some embodiments, the pharmaceutical composition comprises about 15 mg to about 35 mg film coating. In some embodiments, the pharmaceutical composition comprises about 20 mg to about 30 mg film coating. In some embodiments, the pharmaceutical composition comprises about 24 mg to about 28 mg film coating. In some embodiments, the pharmaceutical composition comprises about 26 mg film coating. In some embodiments, the pharmaceutical composition comprises about 25 mg to about 29 mg film coating. In some embodiments, the pharmaceutical composition comprises about 27 mg film coating. In some embodiments, the pharmaceutical composition comprises about 10 mg, about 15 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, or about 40 mg film coating.

In some embodiments, the weight ratio of the film coating to the tablet core is about 0.01:1 to about 0.05:1. In some embodiments, the weight ratio of the film coating to the tablet core is about 0.02:1 to about 0.04:1. In some embodiments, the weight ratio of the film coating to the tablet core is about 0.025:1 to about 0.035:1. In specific embodiments, the weight ratio of the film coating to the tablet core is about 0.03:1. In some embodiments, the weight ratio of the film coating to the tablet core is about 0.01:1, about 0.015:1, about 0.02:1, about 0.025:1, about 0.03:1, about 0.035:1, about 0.04:1, about 0.045:1, or about 0.05:1.

In some embodiments, the film coating constitutes about 1% to about 5% by weight of the tablet. In some embodiments, the film coating constitutes about 2% to about 4% by weight of the tablet. In some embodiments, the film coating constitutes about 3% by weight of the tablet. In some embodiments, the film coating constitutes about 1%, about 2%, about 3%, about 4%, or about 5% by weight of the tablet.

### 5. Further Embodiments of the Pharmaceutical Composition

In some embodiments, the weight of the tablet is about 1500 mg or less. In some embodiments, the weight of the tablet is about 1400 mg or less. In some embodiments, the weight of the tablet is about 1300 mg or less. In some embodiments, the weight of the tablet is about 1200 mg or less. In some embodiments, the weight of the tablet is about 1100 mg or less. In some embodiments, the weight of the tablet is about 1000 mg or less. In some embodiments, the weight of the tablet is about 950 mg or less. In some embodiments, the weight of the tablet is about 900 mg or less.

In specific embodiments, the weight of the tablet is about 1000 mg or less.

In some embodiments, the weight of the tablet is at least about 100 mg, at least about 150 mg, at least about 200 mg, at least about 250 mg, at least about 300 mg, at least about 350 mg, at least about 400 mg, at least about 450 mg, at least about 500 mg, at least about 550 mg, at least about 600 mg, at least about 650 mg, at least about 700 mg, at least about 750 mg, at least about 800 mg, at least about 850 mg, or at least about 900 mg. In specific embodiments, the weight of the tablet is at least about 800 mg. In other specific embodiments, the weight of the tablet is at least about 850 mg. In yet other specific embodiments, the weight of the tablet is at least about 900 mg.

In some embodiments, the weight of the tablet is about 100 mg to about 1500 mg, about 150 mg to about 1500 mg, about 200 mg to about 1500 mg, about 250 mg to about 1500 mg, about 300 mg to about 1500 mg, about 350 mg to about 1500 mg, about 400 mg to about 1500 mg, about 450 mg to about 1500 mg, or about 500 mg to about 1500 mg. In some embodiments, the weight of the tablet is about 550 mg to about 1500 mg, about 600 mg to about 1500 mg, about 650 mg to about 1500 mg, about 700 mg to about 1500 mg, about 750 mg to about 1500 mg, about 800 mg to about 1500 mg, about 850 mg to about 1500 mg, or about 900 mg to about 1500 mg.

In some embodiments, the weight of the tablet is about 100 mg to about 1300 mg, about 150 mg to about 1300 mg, about 200 mg to about 1300 mg, about 250 mg to about 1300 mg, about 300 mg to about 1300 mg, about 350 mg to about 1300 mg, about 400 mg to about 1300 mg, about 450 mg to about 1300 mg, or about 500 mg to about 1300 mg. In some embodiments, the weight of the tablet is about 550 mg to about 1300 mg, about 600 mg to about 1300 mg, about 650 mg to about 1300 mg, about 700 mg to about 1300 mg, about 750 mg to about 1300 mg, about 800 mg to about 1300 mg, about 850 mg to about 1300 mg, or about 900 mg to about 1300 mg.

In some embodiments, the weight of the tablet is about 100 mg to about 1000 mg, about 150 mg to about 1000 mg, about 200 mg to about 1000 mg, about 250 mg to about 1000 mg, about 300 mg to about 1000 mg, about 350 mg to about 1000 mg, about 400 mg to about 1000 mg, about 450 mg to about 1000 mg, or about 500 mg to about 1000 mg. In some embodiments, the weight of the tablet is about 550 mg to about 1000 mg, about 600 mg to about 1000 mg, about 650 mg to about 1000 mg, about 700 mg to about 1000 mg, about 750 mg to about 1000 mg, about 800 mg to about 1000 mg, about 850 mg to about 1000 mg, or about 900 mg to about 1000 mg.

In some embodiments, the weight of the tablet is about 700 mg to about 1000 mg. In some embodiments, the weight of the tablet is about 800 mg to about 1000 mg. In some embodiments, the weight of the tablet is about 850 mg to about 950 mg.

In some embodiments, the weight of the tablet is about 750 mg to about 800 mg. In some embodiments, the weight of the tablet is about 760 mg to about 790 mg or about 770 mg to about 780 mg. In some embodiments, the weight of the tablet is about 773 mg.

In some embodiments, the weight of the tablet is about 850 mg to about 900 mg. In some embodiments, the weight of the tablet is about 860 mg to about 890 mg or about 870 mg to about 880 mg. In some embodiments, the weight of the tablet is about 876 mg.

In some embodiments, the weight of the tablet is about 900 mg to about 950 mg. In some embodiments, the weight of the tablet is about 910 mg to about 940 mg or about 920 mg to about 930 mg. In some embodiments, the weight of the tablet is about 927 mg.

In some embodiments, the weight of the tablet is about 1130 mg to about 1190 mg. In some embodiments, the weight of the tablet is about 1140 mg to about 1180 mg or about 1150 mg to about 1170 mg. In some embodiments, the weight of the tablet is about 1159 mg.

In specific embodiments, the weight of the tablet is about 1000 mg or less. In other specific embodiments, the weight of the tablet is less than about 1000 mg. In some embodiments, the weight of the tablet is at least about 850 mg.

In some embodiments, the weight of the tablet is about 800 mg, about 805 mg, about 810 mg, about 815 mg, about 820 mg, about 825 mg, about 830 mg, about 835 mg, about 840 mg, about 845 mg, about 850 mg, about 855 mg, about 860 mg, about 865 mg, about 870 mg, about 875 mg, about 876 mg, about 880 mg, about 885 mg, about 890 mg, about 895 mg, about 900 mg, about 905 mg, about 910 mg, about 915 mg, about 920 mg, about 925 mg, about 927 mg, about 930 mg, about 935 mg, about 940 mg, about 945 mg, about 950 mg, about 955 mg, about 960 mg, about 965 mg, about 970 mg, about 975 mg, about 980 mg, about 985 mg, about 990 mg, about 995 mg, or about 1000 mg.

Tablets can be of any suitable shape, for example round, oval, elliptic, capsule, cylindrical, square, rectangle, or triangle. In some embodiments, the tablet is an oval shaped tablet. An “oval shaped” tablet has a long axis and a short axis with no flat edged sides. The ratio of the long and short axis can vary, thereby resulting in different shaped oval tablets. In other embodiments, the tablet is a capsule shaped tablet. As used herein, the term “capsule shaped tablet” also includes “modified capsule shaped tablet”. In some embodiments, the tablet is a modified capsule shaped tablet. As known by one skilled in the art, in a “modified capsule shaped tablet”, the long edges of the tablet when viewed in top view provide a slight radius. See, for example, Natoli et al., 2017, Developing Solid Oral Dosage Forms (Second Edition), Chapter 33—Development, Optimization, and Scale-Up of Process Parameters: Tablet Compression, Academic Press, Pages 917-951 (herewith incorporated by reference).

In some embodiments, the length of the short axis of the tablet is about 7.5 mm to about 9.5 mm, the length of the long axis of the tablet is about 18 mm to about 20 mm, and the thickness of the tablet is about 5 mm to about 7 mm. In some embodiments, the length of the short axis of the tablet is about 8 mm to about 9 mm, the length of the long axis of the tablet is about 18.5 mm to about 19.5 mm, and the thickness of the tablet is about 5.5 mm to about 6.5 mm. In specific embodiments, the length of the short axis of the tablet is about 8.5 mm, the length of the long axis of the tablet is about 19 mm, and/or the thickness of the tablet is about 6 mm.

In some embodiments, about 20% to about 40%, about 25% to about 30%, or about 30% to about 40% of dexpramipexole, or the pharmaceutically acceptable salt thereof, is released at about 4 hours of incubation of the tablet in about 900 mL of 50 mM monobasic potassium phosphate buffer, pH 6.8, at a temperature of 37±0.5° C. as measured using an USP type I apparatus operated at a spindle rotation speed of about 100 rpm. In some embodiments, about 30% to about 60%, about 35% to about 45%, or about 45% to about 55% of dexpramipexole, or the pharmaceutically acceptable salt thereof, is released at about 8 hours of incubation of the tablet in about 900 mL of 50 mM monobasic potassium phosphate buffer, pH 6.8, at a temperature of 37±0.5° C. as measured using an USP type I apparatus operated at a spindle rotation speed of about 100 rpm. In some embodiments, about 40% to about 70%, about 45% to about 55%, or about 60% to about 65% of dexpramipexole, or the pharmaceutically acceptable salt thereof, is released at about 12 hours of incubation of the tablet in about 900 mL of 50 mM monobasic potassium phosphate buffer, pH 6.8, at a temperature of 37±0.5° C. as measured using an USP type I apparatus operated at a spindle rotation speed of about 100 rpm. In some embodiments, about 50% to about 70%, about 50% to about 60%, or about 60% to about 70% of dexpramipexole, or the pharmaceutically acceptable salt thereof, is released at about 16 hours of incubation of the tablet in about 900 mL of 50 mM monobasic potassium phosphate buffer, pH 6.8, at a temperature of 37±0.5° C. as measured using an USP type I apparatus operated at a spindle rotation speed of about 100 rpm. In some embodiments, about 55% to about 75%, about 55% to about 65%, or about 65% to about 75% of dexpramipexole, or the pharmaceutically acceptable salt thereof, is released at about 20 hours of incubation of the tablet in about 900 mL of 50 mM monobasic potassium phosphate buffer, pH 6.8, at a temperature of 37±0.5° C. as measured using an USP type I apparatus operated at a spindle rotation speed of about 100 rpm. In some embodiments, about 60% to about 80%, about 60% to about 70%, or about 70% to about 80% of dexpramipexole, or the pharmaceutically acceptable salt thereof, is released at about 24 hours of incubation of the tablet in about 900 mL of 50 mM monobasic potassium phosphate buffer, pH 6.8, at a temperature of 37±0.5° C. as measured using an USP type I apparatus operated at a spindle rotation speed of about 100 rpm.

In some embodiments, at least about 40% but no more than about 50% of dexpramipexole, or the pharmaceutically acceptable salt thereof, is released at about 12 hours of incubation of the tablet in about 900 mL of 50 mM monobasic potassium phosphate buffer, pH 6.8, at a temperature of 37±0.5° C. as measured using an USP type I apparatus operated at a spindle rotation speed of about 100 rpm. In some embodiments, at least about 50% but no more than about 60% of dexpramipexole, or the pharmaceutically acceptable salt thereof, is released at about 12 hours of incubation of the tablet in about 900 mL of 50 mM monobasic potassium phosphate buffer, pH 6.8, at a temperature of 37±0.5° C. as measured using an USP type I apparatus operated at a spindle rotation speed of about 100 rpm. In some embodiments, at least about 50% but no more than about 60% of dexpramipexole, or the pharmaceutically acceptable salt thereof, is released at about 16 hours of incubation of the tablet in about 900 mL of 50 mM monobasic potassium phosphate buffer, pH 6.8, at a temperature of 37±0.5° C. as measured using an USP type I apparatus operated at a spindle rotation speed of about 100 rpm. In some embodiments, at least about 60% but no more than about 70% of dexpramipexole, or the pharmaceutically acceptable salt thereof, is released at about 16 hours of incubation of the tablet in about 900 mL of 50 mM monobasic potassium phosphate buffer, pH 6.8, at a temperature of 37±0.5° C. as measured using an USP type I apparatus operated at a spindle rotation speed of about 100 rpm. In some embodiments, at least about 60% but no more than about 70% of dexpramipexole, or the pharmaceutically acceptable salt thereof, is released at about 24 hours of incubation of the tablet in about 900 mL of 50 mM monobasic potassium phosphate buffer, pH 6.8, at a temperature of 37±0.5° C. as measured using an USP type I apparatus operated at a spindle rotation speed of about 100 rpm. In some embodiments, at least about 70% but no more than about 80% of dexpramipexole, or the pharmaceutically acceptable salt thereof, is released at about 24 hours of incubation of the tablet in about 900 mL of 50 mM monobasic potassium phosphate buffer, pH 6.8, at a temperature of 37±0.5° C. as measured using an USP type I apparatus operated at a spindle rotation speed of about 100 rpm.

In some embodiments, about 20% to about 40% or about 30% to about 40% of dexpramipexole, or the pharmaceutically acceptable salt thereof, is released at about 4 hours of incubation of the tablet in about 900 mL of McIlvaine buffer, pH 4.5, at a temperature of 37±0.5° C. as measured using an USP type I apparatus operated at a spindle rotation speed of about 100 rpm. In some embodiments, about 40% to about 60%, or about 40% to about 45%, or about 45% to about 55% of dexpramipexole, or the pharmaceutically acceptable salt thereof, is released at about 8 hours of incubation of the tablet in about 900 mL of McIlvaine buffer, pH 4.5, at a temperature of 37±0.5° C. as measured using an USP type I apparatus operated at a spindle rotation speed of about 100 rpm. In some embodiments, about 50% to about 70%, or about 50% to about 60%, or about 60% to about 70% of dexpramipexole, or the pharmaceutically acceptable salt thereof, is released at about 12 hours of incubation of the tablet in about 900 mL of McIlvaine buffer, pH 4.5, at a temperature of 37±0.5° C. as measured using an USP type I apparatus operated at a spindle rotation speed of about 100 rpm. In some embodiments, about 60% to about 80%, or about 60% to about 70%, or about 65% to about 75% of dexpramipexole, or the pharmaceutically acceptable salt thereof, is released at about 16 hours of incubation of the tablet in about 900 mL of McIlvaine buffer, pH 4.5, at a temperature of 37±0.5° C. as measured using an USP type I apparatus operated at a spindle rotation speed of about 100 rpm. In some embodiments, about 60% to about 80%, or about 65% to about 75%, or about 75% to about 85% of dexpramipexole, or the pharmaceutically acceptable salt thereof, is released at about 20 hours of incubation of the tablet in about 900 mL of McIlvaine buffer, pH 4.5, at a temperature of 37±0.5° C. as measured using an USP type I apparatus operated at a spindle rotation speed of about 100 rpm. In some embodiments, about 70% to about 90%, or about 70% to about 80%, or about 80% to about 85% of dexpramipexole, or the pharmaceutically acceptable salt thereof, is released at about 24 hours of incubation of the tablet in about 900 mL of McIlvaine buffer, pH 4.5, at a temperature of 37±0.5° C. as measured using an USP type I apparatus operated at a spindle rotation speed of about 100 rpm.

In some embodiments, at least about 50% but no more than about 60% of dexpramipexole, or the pharmaceutically acceptable salt thereof, is released at about 12 hours of incubation of the tablet in about 900 mL of McIlvaine buffer, pH 4.5, at a temperature of 37±0.5° C. as measured using an USP type I apparatus operated at a spindle rotation speed of about 100 rpm. In some embodiments, at least about 60% but no more than about 70% of dexpramipexole, or the pharmaceutically acceptable salt thereof, is released at about 12 hours of incubation of the tablet in about 900 mL of McIlvaine buffer, pH 4.5, at a temperature of 37±0.5° C. as measured using an USP type I apparatus operated at a spindle rotation speed of about 100 rpm. In some embodiments, at least about 60% but no more than about 70% of dexpramipexole, or the pharmaceutically acceptable salt thereof, is released at about 16 hours of incubation of the tablet in about 900 mL of McIlvaine buffer, pH 4.5, at a temperature of 37±0.5° C. as measured using an USP type I apparatus operated at a spindle rotation speed of about 100 rpm. In some embodiments, at least about 70% but no more than about 80% of dexpramipexole, or the pharmaceutically acceptable salt thereof, is released at about 16 hours of incubation of the tablet in about 900 mL of McIlvaine buffer, pH 4.5, at a temperature of 37±0.5° C. as measured using an USP type I apparatus operated at a spindle rotation speed of about 100 rpm. In some embodiments, at least about 70% but no more than about 80% of dexpramipexole, or the pharmaceutically acceptable salt thereof, is released at about 24 hours of incubation of the tablet in about 900 mL of McIlvaine buffer, pH 4.5, at a temperature of 37±0.5° C. as measured using an USP type I apparatus operated at a spindle rotation speed of about 100 rpm. In some embodiments, at least about 80% but no more than about 90% of dexpramipexole, or the pharmaceutically acceptable salt thereof, is released at about 24 hours of incubation of the tablet in about 900 mL of McIlvaine buffer, pH 4.5, at a temperature of 37±0.5° C. as measured using an USP type I apparatus operated at a spindle rotation speed of about 100 rpm.

In some embodiments, about 50% to about 70% or about 55% to about 65% of dexpramipexole, or the pharmaceutically acceptable salt thereof, is released at about 4 hours of incubation of the tablet in about 900 mL of 0.1 N HCl at a temperature of 37±0.5° C. as measured using an USP type I apparatus operated at a spindle rotation speed of about 100 rpm. In some embodiments, about 75% to about 95%, about 75% to about 85%, or about 85% to about 95% of dexpramipexole, or the pharmaceutically acceptable salt thereof, is released at about 8 hours of incubation of the tablet in about 900 mL of 0.1 N HCl at a temperature of 37±0.5° C. as measured using an USP type I apparatus operated at a spindle rotation speed of about 100 rpm. In some embodiments, about 90% to about 100% of dexpramipexole, or the pharmaceutically acceptable salt thereof, is released at about 12 hours of incubation of the tablet in about 900 mL of 0.1 N HCl at a temperature of 37±0.5° C. as measured using an USP type I apparatus operated at a spindle rotation speed of about 100 rpm. In some embodiments, about 90% to about 100% of dexpramipexole, or the pharmaceutically acceptable salt thereof, is released at about 16 hours of incubation of the tablet in about 900 mL of 0.1 N HCl at a temperature of 37±0.5° C. as measured using an USP type I apparatus operated at a spindle rotation speed of about 100 rpm. In some embodiments, about 90% to about 100% of dexpramipexole, or the pharmaceutically acceptable salt thereof, is released at about 20 hours of incubation of the tablet in about 900 mL of 0.1 N HCl at a temperature of 37±0.5° C. as measured using an USP type I apparatus operated at a spindle rotation speed of about 100 rpm. In some embodiments, about 90% to about 100% of dexpramipexole, or the pharmaceutically acceptable salt thereof, is released at about 24 hours of incubation of the tablet in about 900 mL of 0.1 N HCl at a temperature of 37±0.5° C. as measured using an USP type I apparatus operated at a spindle rotation speed of about 100 rpm.

In some embodiments, at least about 90% of dexpramipexole, or the pharmaceutically acceptable salt thereof, is released at about 12 hours of incubation of the tablet in about 900 mL of 0.1 N HCl at a temperature of 37±0.5° C. as measured using an USP type I apparatus operated at a spindle rotation speed of about 100 rpm. In some embodiments, at least about 90% of dexpramipexole, or the pharmaceutically acceptable salt thereof, is released at about 16 hours of incubation of the tablet in about 900 mL of 0.1 N HCl at a temperature of 37±0.5° C. as measured using an USP type I apparatus operated at a spindle rotation speed of about 100 rpm. In some embodiments, at least about 90% of dexpramipexole, or the pharmaceutically acceptable salt thereof, is released at about 24 hours of incubation of the tablet in about 900 mL of 0.1 N HCl at a temperature of 37±0.5° C. as measured using an USP type I apparatus operated at a spindle rotation speed of about 100 rpm.

In some embodiments, the in vitro dissolution profile measured using an USP type I apparatus operated at a spindle rotation speed of about 100 rpm and incubating the tablet in about 900 mL of 50 mM monobasic potassium phosphate buffer, pH 6.8, at a temperature of 37±0.5° C., is substantially the same after storing the tablet under controlled room temperature (CRT) conditions (i.e., at a temperature of 20° C. to 25° C.) for about 4 weeks, about 5 weeks, about 6 weeks, about 7 weeks, about 8 weeks, about 9 weeks, about 10 weeks, about 11 weeks, or about 12 weeks. In some embodiments, the in vitro dissolution profile measured using an USP type I apparatus operated at a spindle rotation speed of about 100 rpm and incubating the tablet in about 900 mL of 50 mM monobasic potassium phosphate buffer, pH 6.8, at a temperature of 37±0.5° C., is substantially the same after storing the tablet under controlled room temperature (CRT) conditions (i.e., at a temperature of 20° C. to 25° C.) for at least about 4 weeks (such as for about 4 to about 12 weeks, or about 4 to about 11 weeks), at least about 5 weeks, at least about 6 weeks, at least about 7 weeks, at least about 8 weeks, at least about 9 weeks, at least about 10 weeks, at least about 11 weeks, or at least about 12 weeks.

In some embodiments, the pharmaceutical composition is capable of providing a sigmoidal, pseudo-zero order, or zero order release of dexpramipexole, or a pharmaceutically acceptable salt thereof. In specific embodiments, the release is a zero order release.

In some embodiments, the pharmaceutical composition comprises 0.05% or less pramipexole, or a pharmaceutically acceptable salt thereof, by weight of the tablet. In some embodiments, the pharmaceutical composition comprises 0.04% or less pramipexole, or a pharmaceutically acceptable salt thereof, by weight of the tablet. In some embodiments, the pharmaceutical composition comprises 0.03% or less pramipexole, or a pharmaceutically acceptable salt thereof, by weight of the tablet. In some embodiments, the pharmaceutical composition comprises 0.02% or less pramipexole, or a pharmaceutically acceptable salt thereof, by weight of the tablet. In some embodiments, the pharmaceutical composition comprises 0.015% or less pramipexole, or a pharmaceutically acceptable salt thereof, by weight of the tablet. In some embodiments, the pharmaceutical composition comprises 0.014% or less pramipexole, or a pharmaceutically acceptable salt thereof, by weight of the tablet. In some embodiments, the pharmaceutical composition comprises 0.01% or less pramipexole, or a pharmaceutically acceptable salt thereof, by weight of the tablet.

In specific embodiments, the pharmaceutical composition comprises 0.015% or less pramipexole, or a pharmaceutically acceptable salt thereof, by weight of the tablet.

In some embodiments, the pharmaceutical composition does not comprise pramipexole, or a pharmaceutically acceptable salt thereof, at the detection limit when analyzed with high performance liquid chromatography (HPLC).

In some embodiments, the one or more polymers in the compositions described herein are anionic polymers or neutral polymers. In some embodiments, the one or more polymers are anionic polymers. In some embodiments, the one or more anionic polymers constitute about 30% to about 60% by weight of the tablet. In some embodiments, the one or more anionic polymers constitute about 30% to about 50% by weight of the tablet. In specific embodiments, the one or more anionic polymers constitute about 38% to about 48% (such as about 39% or about 46%) by weight of the tablet.

In specific embodiments of the present disclosure, the amount of dexpramipexole, or a pharmaceutically acceptable salt thereof, in the pharmaceutical composition is about 50 mg to about 350 mg (such as about 75 mg, about 150 mg, or about 300 mg) dexpramipexole dihydrochloride equivalent and the one or more anionic polymers constitute about 30% to about 55% (such as about 30% to about 50%) by weight of the tablet core. In these specific embodiments, the one or more anionic polymers may be a carboxymethylcellulose and/or a crosslinked acrylic acid polymer. In the specific embodiments described in this paragraph, the pharmaceutical composition may comprise 0.015% or less pramipexole, or a pharmaceutically acceptable salt thereof, by weight of the tablet. The weight of the tablet may be about 1000 mg or less (such as about 800 mg to about 1000 mg).

In specific embodiments of the present disclosure, the amount of dexpramipexole, or a pharmaceutically acceptable salt thereof, in the pharmaceutical composition is about 75 mg to about 300 mg (such as about 75 mg, about 150 mg, or about 300 mg) dexpramipexole dihydrochloride equivalent and the one or more anionic polymers constitute about 30% to about 50% (such as about 40% to about 50%) by weight of the tablet core. In specific embodiments of the present disclosure, the amount of dexpramipexole, or a pharmaceutically acceptable salt thereof, in the pharmaceutical composition is about 75 mg to about 300 mg (such as about 75 mg, about 150 mg, or about 300 mg) dexpramipexole dihydrochloride equivalent and the one or more anionic polymers constitute about 30% to about 60% (such as about 40% to about 50%) by weight of the tablet core. In these specific embodiments, the one or more anionic polymers may be a carboxymethylcellulose and/or a crosslinked acrylic acid polymer. In the specific embodiments described in this paragraph, the pharmaceutical composition may comprise 0.015% or less pramipexole, or a pharmaceutically acceptable salt thereof, by weight of the tablet. The weight of the tablet may be about 1000 mg or less (such as about 800 mg to about 1000 mg).

In some embodiments, the one or more polymers are neutral polymers. In some embodiments, the one or more neutral polymers constitute about 5% to about 25% by weight of the tablet. In some embodiments, the one or more neutral polymers constitute about 10% to about 20% by weight of the tablet. In specific embodiments, the one or more neutral polymers constitute about 13% to about 17% (such as about 15%) by weight of the tablet.

In some embodiments, the one or more neutral polymers constitute between about 5% and about 60% by weight of the tablet core. In some embodiments, the one or more neutral polymers constitute between about 10% and about 60% by weight of the tablet core. In some embodiments, the one or more neutral polymers constitute about 15% to about 60% by weight of the tablet core. In some embodiments, the one or more neutral polymers constitute between about 20% and about 60% by weight of the tablet core. In some embodiments, the one or more neutral polymers constitute about 25% to about 60% by weight of the tablet core. In some embodiments, the one or more neutral polymers constitute between about 30% and about 60% by weight of the tablet core. In some embodiments, the one or more neutral polymers constitute about 35% to about 60% by weight of the tablet core. In some embodiments, the one or more neutral polymers constitute between about 40% and about 60% by weight of the tablet core. In some embodiments, the one or more neutral polymers constitute about 45% to about 60% by weight of the tablet core. In some embodiments, the one or more neutral polymers constitute between about 50% and about 60% by weight of the tablet core. In some embodiments, the one or more neutral polymers constitute about 55% to about 60% by weight of the tablet core.

In some embodiments, the one or more neutral polymers constitute about 5% to about 55% by weight of the tablet core. In some embodiments, the one or more neutral polymers constitute about 5% to about 50% by weight of the tablet core. In some embodiments, the one or more neutral polymers constitute about 5% to about 45% by weight of the tablet core. In some embodiments, the one or more neutral polymers constitute about 5% to about 40% by weight of the tablet core. In some embodiments, the one or more neutral polymers constitute about 5% to about 35% by weight of the tablet core. In some embodiments, the one or more neutral polymers constitute about 5% to about 30% by weight of the tablet core.

In some embodiments, the one or more neutral polymers constitute about 20% by weight of the tablet core. In some embodiments, the one or more neutral polymers constitute about 25% by weight of the tablet core. In some embodiments, the one or more neutral polymers constitute about 30% by weight of the tablet core. In some embodiments, the one or more neutral polymers constitute about 35% by weight of the tablet core. In some embodiments, the one or more neutral polymers constitute about 40% by weight of the tablet core. In some embodiments, the one or more neutral polymers constitute about 45% by weight of the tablet core. In some embodiments, the one or more neutral polymers constitute about 50% by weight of the tablet core. In some embodiments, the one or more neutral polymers constitute about 55% by weight of the tablet core. In some embodiments, the one or more neutral polymers constitute about 60% by weight of the tablet core.

In specific embodiments of the present disclosure, the amount of dexpramipexole, or a pharmaceutically acceptable salt thereof, in the pharmaceutical composition is about 50 mg to about 350 mg (such as about 75 mg, about 150 mg, or about 300 mg) dexpramipexole dihydrochloride equivalent and the one or more neutral polymers constitute about 5% to about 25% (such as about 10% to about 20%) by weight of the tablet core. In these specific embodiments, the one or more neutral polymers may be a hydroxypropyl methylcellulose or silicified hydroxypropyl methylcellulose. In the specific embodiments described in this paragraph, the pharmaceutical composition may comprise 0.015% or less pramipexole, or a pharmaceutically acceptable salt thereof, by weight of the tablet. The weight of the tablet may be about 1000 mg or less (such as about 800 mg to about 1000 mg).

In specific embodiments of the present disclosure, the amount of dexpramipexole, or a pharmaceutically acceptable salt thereof, in the pharmaceutical composition is about 75 mg to about 300 mg (such as about 75 mg, about 150 mg, or about 300 mg) dexpramipexole dihydrochloride equivalent and the one or more neutral polymers constitute about 5% to about 25% (such as about 10% to about 20%) by weight of the tablet core. In specific embodiments of the present disclosure, the amount of dexpramipexole, or a pharmaceutically acceptable salt thereof, in the pharmaceutical composition is about 75 mg to about 300 mg (such as about 75 mg, about 150 mg, or about 300 mg) dexpramipexole dihydrochloride equivalent and the one or more neutral polymers constitute about 5% to about 25% (such as about 10% to about 20%) by weight of the tablet core. In these specific embodiments, the one or more neutral polymers may be a hydroxypropyl methylcellulose or silicified hydroxypropyl methylcellulose. In the specific embodiments described in this paragraph, the pharmaceutical composition may comprise 0.015% or less pramipexole, or a pharmaceutically acceptable salt thereof, by weight of the tablet. The weight of the tablet may be about 1000 mg or less (such as about 800 mg to about 1000 mg).

In some embodiments, the tensile strength of the orally deliverable tablet is about 1.7 MPa or more. In specific embodiments, the tensile strength of the orally deliverable tablet is about 1.7 MPa to about 2.5 MPa.

In some embodiments, the hardness of the orally deliverable tablet is about 15 to about 30 kilopond (kp). In some embodiments, the hardness of the orally deliverable tablet is about 20 to about 25 kp, such as about 22 kp. In some embodiments, the hardness of the orally deliverable tablet is about 15 kp, about 16 kp, about 17 kp, about 18 kp, about 19 kp, about 20 kp, about 21 kp, about 22 kp, about 23 kp, about 24 kp, about 25 kp, about 26 kp, about 27 kp, about 28 kp, about 29 kp, or about 30 kp.

In specific embodiments of the present disclosure, the homogeneous mixture consists essentially of about 50 mg to about 350 mg (such as about 75 mg, about 150 mg, or about 300 mg) dexpramipexole dihydrochloride equivalent and the one or more anionic polymers, wherein the one or more anionic polymers constitute about 30% to about 55% (such as about 30% to about 50%) by weight of the tablet core. In these specific embodiments, the one or more anionic polymers may be a carboxymethylcellulose and/or a crosslinked acrylic acid polymer. In the specific embodiments described in this paragraph, the pharmaceutical composition may comprise 0.015% or less pramipexole, or a pharmaceutically acceptable salt thereof, by weight of the tablet. The weight of the tablet may be about 1000 mg or less (such as about 800 mg to about 1000 mg).

In specific embodiments of the present disclosure, the homogeneous mixture consists essentially of about 75 mg to about 300 mg (such as about 75 mg, about 150 mg, or about 300 mg) dexpramipexole dihydrochloride equivalent and the one or more anionic polymers, wherein the one or more anionic polymers constitute about 30% to about 50% (such as about 40% to about 50%) by weight of the tablet core. In these specific embodiments, the one or more anionic polymers may be a carboxymethylcellulose and/or a crosslinked acrylic acid polymer. In the specific embodiments described in this paragraph, the pharmaceutical composition may comprise 0.015% or less pramipexole, or a pharmaceutically acceptable salt thereof, by weight of the tablet. The weight of the tablet may be about 1000 mg or less (such as about 800 mg to about 1000 mg).

In specific embodiments of the present disclosure, the homogeneous mixture consists essentially of about 50 mg to about 350 mg (such as about 75 mg, about 150 mg, or about 300 mg) dexpramipexole dihydrochloride equivalent and the one or more neutral polymers, wherein the one or more neutral polymers constitute about 5% to about 25% (such as about 10% to about 20%) by weight of the tablet core. In these specific embodiments, the one or more neutral polymers may be a hydroxypropyl methylcellulose or silicified hydroxypropyl methylcellulose. In the specific embodiments described in this paragraph, the pharmaceutical composition may comprise 0.015% or less pramipexole, or a pharmaceutically acceptable salt thereof, by weight of the tablet. The weight of the tablet may be about 1000 mg or less (such as about 800 mg to about 1000 mg).

In specific embodiments of the present disclosure, the homogeneous mixture consists essentially of about 75 mg to about 300 mg (such as about 75 mg, about 150 mg, or about 300 mg) dexpramipexole dihydrochloride equivalent and the one or more neutral polymers, wherein the one or more neutral polymers constitute about 5% to about 25% (such as about 10% to about 20%) by weight of the tablet core. In these specific embodiments, the one or more neutral polymers may be a hydroxypropyl methylcellulose or silicified hydroxypropyl methylcellulose. In the specific embodiments described in this paragraph, the pharmaceutical composition may comprise 0.015% or less pramipexole, or a pharmaceutically acceptable salt thereof, by weight of the tablet. The weight of the tablet may be about 1000 mg or less (such as about 800 mg to about 1000 mg).

In some embodiments, one or more of the ingredients of the pharmaceutical composition other than dexpramipexole, or the pharmaceutically acceptable salt thereof, such as one or more of an (anionic) polymer, a lubricant, a glidant, and/or a diluent, are “generally recognized as safe” (in short: “GRAS”) by the United States Food and Drug Administration (FDA) and/or are recognized as safe by the provisions of the International Council for Harmonisation of Technical Requirements for Registration of Pharmaceuticals for Human Use (ICH). In some embodiments, one or more of the ingredients of the pharmaceutical composition other than dexpramipexole, or the pharmaceutically acceptable salt thereof, such as one or more of an (anionic) polymer, a lubricant, a glidant, and/or a diluent, are such ingredients that are listed in a Pharmacopoeia, such as the European and/or US Pharmacopoeia.

In some embodiments, the pharmaceutical composition can be part of a kit comprising the pharmaceutical composition packaged into a container (which might be, for example, a blister package or a bottle), the container being accompanied by a package insert providing pertinent information such as, for example, dosage and administration information, contraindications, precautions, drug interactions and adverse reactions. In some embodiments, the kit further comprises a desiccant. A desiccant is a hygroscopic substance that is used to induce or sustain a state of dryness (desiccation) in its vicinity. Examples of desiccants are silica gel, activated charcoal, calcium sulfate, calcium chloride, and molecular sieves (typically, zeolites).

### III—Methods of Manufacturing

In certain aspects, the present disclosure further relates to methods of manufacturing as described in this chapter below.

### 1. Method of Manufacturing Densified Granules

The present disclosure relates to a method of manufacturing densified granules for preparing a sustained release pharmaceutical composition comprising dexpramipexole, or a pharmaceutically acceptable salt thereof, the method comprising:
preparing a pre-blend by mixing dexpramipexole, or a pharmaceutically acceptable salt thereof, and one or more polymers; and
preparing densified granules from the pre-blend;
wherein dexpramipexole, or the pharmaceutically acceptable salt thereof, constitutes about 30% to about 40% of dexpramipexole dihydrochloride monohydrate equivalent by weight of the densified granules and the one or more polymers constitute about 5% to about 60% by weight of the densified granules.

The present disclosure relates to a method of manufacturing densified granules for preparing a sustained release pharmaceutical composition comprising dexpramipexole, or a pharmaceutically acceptable salt thereof, the method comprising:
preparing a pre-blend by mixing dexpramipexole, or a pharmaceutically acceptable salt thereof, and one or more anionic polymers; and
preparing densified granules from the pre-blend;
wherein dexpramipexole, or the pharmaceutically acceptable salt thereof, constitutes about 30% to about 40% of dexpramipexole dihydrochloride monohydrate equivalent by weight of the densified granules and the one or more anionic polymers constitute about 30% to about 55% by weight of the densified granules. The densified granules prepared by the method described in the above paragraph can be used in the production of a sustained release orally deliverable pharmaceutical composition, for example, in the form of a tablet or capsule.

The present disclosure relates to a method of manufacturing densified granules for preparing a sustained release pharmaceutical composition comprising dexpramipexole, or a pharmaceutically acceptable salt thereof, the method comprising:
preparing a pre-blend by mixing dexpramipexole, or a pharmaceutically acceptable salt thereof, and one or more anionic polymers; and
preparing densified granules from the pre-blend;
wherein dexpramipexole, or the pharmaceutically acceptable salt thereof, constitutes about 30% to about 40% of dexpramipexole dihydrochloride monohydrate equivalent by weight of the densified granules and the one or more anionic polymers constitute about 30% to about 55% by weight of the densified granules.

In some embodiments, the pre-blend further comprises additional excipients. In some embodiments, the pre-blend further comprises a microcrystalline cellulose, magnesium stearate, or a combination thereof. In some embodiments, the pre-blend further comprises a microcrystalline cellulose and magnesium stearate. In some embodiments, the microcrystalline cellulose is silicified microcrystalline cellulose.

In some embodiments, the pre-blend comprises an antioxidant. In some embodiments, the antioxidant is capable of acting as a nitrite scavenger. Non-limiting examples of nitrite scavengers include, but are not limited to, ascorbic acid, L-cysteine, caffeic acid, cysteine HCL, methionine, tartaric acid, gallic acid, uric acid, and sodium sulphite. In some embodiments, the pre-blend comprises one nitrite scavenger. In another embodiment, the pre-blend comprises multiple nitrite scavengers. In some embodiments, the nitrite scavengers constitute about 0.1% to about 1.5% by weight of the tablet core. In some embodiments, the nitrite scavenger constitutes about 0.1% to about 1.0% by weight of the tablet core. In some embodiments, the nitrite scavenger constitutes about 0.1% to about 0.5% by weight of the tablet core. In some embodiments, the nitrite scavenger constitutes about 0.5% to about 1.5% by weight of the tablet core. In some embodiments, the nitrite scavenger constitutes about 1% to about 1.5% by weight of the tablet core. In some embodiments, the nitrite scavenger constitutes about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, or about 1.5% by weight of the tablet core.

In some embodiments, dexpramipexole, or the pharmaceutically acceptable salt thereof, constitutes about 35% to about 40% of dexpramipexole dihydrochloride monohydrate equivalent by weight of the densified granules. In some embodiments, dexpramipexole, or the pharmaceutically acceptable salt thereof, constitutes about 35% to about 44% of dexpramipexole dihydrochloride monohydrate equivalent by weight of the densified granules. In some embodiments, dexpramipexole, or the pharmaceutically acceptable salt thereof, constitutes about 36% to about 38% of dexpramipexole dihydrochloride monohydrate equivalent by weight of the densified granules. In some embodiments, dexpramipexole, or the pharmaceutically acceptable salt thereof, constitutes about 36% of dexpramipexole dihydrochloride monohydrate equivalent by weight of the densified granules. In some embodiments, dexpramipexole, or the pharmaceutically acceptable salt thereof, constitutes about 38% of dexpramipexole dihydrochloride monohydrate equivalent by weight of the densified granules. In some embodiments, dexpramipexole, or the pharmaceutically acceptable salt thereof, constitutes about 30%, about 31%, about 32%, about 33%, about 34%, about 35%, about 36%, about 37%, about 38%, about 39%, or about 40% of dexpramipexole dihydrochloride monohydrate equivalent by weight of the densified granules. In some embodiments, the weight ratio of dexpramipexole dihydrochloride equivalent to the one or more anionic polymers is about 0.2:1 to about 1.3:1, about 0.3:1 to about 1.2:1, about 0.4:1 to about 1.1:1, or about 0.5:1 to about 1:1. In some embodiments, the weight ratio of dexpramipexole dihydrochloride equivalent to the one or more polymers is about 0.2:1 to about 4.0:1, about 0.3:1 to about 3.8:1, about 0.4:1 to about 3.6:1, about 0.5:1 to about 3.5:1, about 0.6:1 to about 3.4:1, about 0.7:1 to about 3.3:1, about 0.8:1 to about 3.2:1, about 0.9:1 to about 3.1:1, about 1.0:1 to about 3.0:1, about 1.1:1 to about 2.9:1, or about 1.2:1 to about 2.8:1. In some embodiments, the weight ratio of dexpramipexole dihydrochloride equivalent to the one or more anionic polymers is about 0.7:1 to about 0.9:1. In some embodiments, the weight ratio of dexpramipexole dihydrochloride equivalent to the one or more anionic polymers is about 0.2:1, about 0.3:1, about 0.4:1, about 0.5:1, about 0.6:1, about 0.7:1, about 0.8:1, about 0.9:1, about 1:1, about 1.1:1, about 1.2:1, or about 1.3:1. In some embodiments, the weight ratio of dexpramipexole dihydrochloride equivalent to the one or more polymers is about 0.7:1 to about 0.9:1. In some embodiments, the weight ratio of dexpramipexole dihydrochloride equivalent to the one or more polymers is about 0.2:1, about 0.3:1, about 0.4:1, about 0.5:1, about 0.6:1, about 0.7:1, about 0.8:1, about 0.9:1, about 1:1, about 1.1:1, about 1.2:1, about 1.3:1, about 1.4:1, about 1.5:1, about 1.6:1, about 1.7:1, about 1.8:1, about 1.9:1, about 2.0:1, about 2.1:1, about 2.2:1, about 2.3:1, about 2.4:1, about 2.5:1, about 2.6:1, about 2.7:1, about 2.8:1, about 2.9:1, about 3.0:1, about 3.1:1, about 3.2:1, about 3.3:1, about 3.4:1, about 3.5:1, about 3.6:1, about 3.7:1, about 3.8:1, about 3.9:1, or about 4.0:1.

In some embodiments, the one or more polymers are anionic polymers or neutral polymers. In some embodiments, the one or more polymers are anionic polymers.

In some embodiments, the one or more anionic polymers constitute about 20% to about 55% by weight of the densified granules. In some embodiments, the one or more anionic polymers constitute about 35% to about 50% by weight of the densified granules. In some embodiments, the one or more anionic polymers constitute about 40% to about 50% by weight of the densified granules. In some embodiments, the one or more anionic polymers constitute about 35% to about 45%, about 36% to about 44%, about 37% to about 43%, or about 38% to about 42% by weight of the densified granules. In some embodiments, the one or more anionic polymers constitute about 39% to about 41% by weight of the densified granules. In some embodiments, the one or more anionic polymers constitute about 40% by weight of the densified granules. In some embodiments, the one or more anionic polymers constitute about 43% to about 53%, about 44%, to about 52%, about 45% to about 51%, or about 46% to about 50% by weight of the densified granules. In some embodiments, the one or more anionic polymers constitute about 47% to about 49% by weight of the densified granules. In some embodiments, the one or more anionic polymers constitute about 48% by weight of the densified granules. In some embodiments, the one or more anionic polymers constitute about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, or about 55% by weight of the densified granules. In some embodiments, the one or more anionic polymers constitute about 48% by weight of the densified granules. In some embodiments, the one or more anionic polymers constitute about 43% by weight of the densified granules. In some embodiments, the one or more anionic polymers constitute about 40% by weight of the densified granules.

In some embodiments, the one or more anionic polymers are selected from carboxymethylcellulose, an acrylic acid polymer, a methacrylic acid polymer, a methacrylic acid copolymer, alignate, carrageenan, xanthan gum, or arabic gum. In some embodiments, the one or more anionic polymers are hydrophilic anionic polymers. In some embodiments, the one or more anionic polymers are selected from carboxymethylcellulose and an acrylic acid polymer. The carboxymethylcellulose may be sodium carboxymethylcellulose. An example of a sodium carboxymethylcellulose is Aqualon CMC 7HXF PH. In more specific embodiments, the viscosity of the sodium carboxymethylcellulose is about 1,500 to about 3,000 cP (centipoise; 1% in water). The acrylic acid polymer may be crosslinked acrylic acid polymer. An example of a crosslinked acrylic acid polymer is Carbopol 971P. In more specific embodiments, the viscosity of the crosslinked acrylic acid polymer is about 4,000 to about 11,000 cP (centipoise; 0.5% at pH 7.5).

In some embodiments, the pre-blend comprises one, but no more than one anionic polymer. In specific embodiments, the pre-blend comprises one, but no more than one anionic polymer and the anionic polymer is carboxymethylcellulose, such as sodium carboxymethylcellulose. In specific embodiments, the pre-blend comprises one, but no more than one anionic polymer and the anionic polymer is an acrylic acid polymer, such as crosslinked acrylic acid polymer.

In some embodiments, the pre-blend comprises more than one anionic polymer, such as two, three, four, or five anionic polymers. Within the meaning of the present disclosure, if the pre-blend comprises more than one anionic polymer, such as a first and a second anionic polymer, the anionic polymers are different anionic polymers. For example, a pre-blend comprising two anionic polymers might comprise sodium carboxymethylcellulose as the first anionic polymer and an acrylic acid polymer as the second anionic polymer.

In some embodiments, the pre-blend comprises one or more, but no more than five anionic polymers. In some embodiments, the pre-blend comprises one or more, but no more than four anionic polymers. In some embodiments, the pre-blend comprises one or more, but no more than three anionic polymers. In some embodiments, the pre-blend comprises one or more, but no more than two anionic polymers.

In some embodiments, the pre-blend comprises two, but no more than two anionic polymers. In specific embodiments, the pre-blend comprises two, but no more than two anionic polymers and the two anionic polymers are carboxymethylcellulose (such as sodium carboxymethylcellulose) and an acrylic acid polymer (such as a crosslinked acrylic acid polymer).

In some embodiments, the densified granules consists essentially of dexpramipexole, or a pharmaceutically acceptable salt thereof, constituting about 30% to about 40% of dexpramipexole dihydrochloride monohydrate equivalent by weight of the densified granules and one or more, but no more than two anionic polymers at the amounts specified herein (such as at about 30% to about 50% by weight of the densified granules). If two anionic polymers are present, the two anionic polymers may be carboxymethylcellulose (such as sodium carboxymethylcellulose) and an acrylic acid polymer (such as a crosslinked acrylic acid polymer). In some embodiments, carboxymethylcellulose constitutes about 20% to about 36% of sodium carboxymethylcellulose equivalent by weight of the densified granules. In some embodiments, carboxymethylcellulose constitutes about 21% to about 35%, about 22% to about 34%, about 23% to about 33%, about 24% to about 32%, about 25% to about 31%, about 26% to about 30%, or about 27% to about 29% of sodium carboxymethylcellulose equivalent by weight of the densified granules. In specific embodiments, carboxymethylcellulose constitutes about 28% of sodium carboxymethylcellulose equivalent by weight of the densified granules.

In some embodiments, the densified granules consists essentially of dexpramipexole, or a pharmaceutically acceptable salt thereof, constituting about 30% to about 40% of dexpramipexole dihydrochloride monohydrate equivalent by weight of the densified granules and two anionic polymers at the amounts specified herein (such as at about 30% to about 50% by weight of the densified granules). The two anionic polymers may be carboxymethylcellulose (such as sodium carboxymethylcellulose) and an acrylic acid polymer (such as a crosslinked acrylic acid polymer).

In some embodiments, the pre-blend comprises a first anionic polymer and a second anionic polymer and the first anionic polymer is carboxymethylcellulose and the second anionic polymer is an acrylic acid polymer. The acrylic acid polymer may be crosslinked acrylic acid polymer and/or the carboxymethylcellulose may be sodium carboxymethylcellulose. In some embodiments, carboxymethylcellulose constitutes about 23% to about 37% of sodium carboxymethylcellulose equivalent by weight of the densified granules and the acrylic acid polymer constitutes about 7% to about 18% by weight of the densified granules. In some embodiments, carboxymethylcellulose constitutes about 23% to about 33%, about 24% to about 32%, about 25% to about 31%, about 26% to about 30%, or about 27% to about 29% of sodium carboxymethylcellulose equivalent by weight of the densified granules and the acrylic acid polymer constitutes about 7% to about 16%, about 8% to about 15%, about 10% to about 14%, or about 11% to about 13% by weight of the densified granules. In specific embodiments, carboxymethylcellulose constitutes about 28% of sodium carboxymethylcellulose equivalent by weight of the densified granules and the acrylic acid polymer constitutes about 12% by weight of the densified granules. In other embodiments, carboxymethylcellulose constitutes about 25% to about 35%, about 26% to about 34%, about 27% to about 33%, or about 28% to about 32% of sodium carboxymethylcellulose equivalent by weight of the densified granules and the acrylic acid polymer constitutes about 9% to about 17%, about 10% to about 16%, about 11% to about 15%, or about 12% to about 14% by weight of the densified granules. In other specific embodiments, carboxymethylcellulose constitutes about 30% of sodium carboxymethylcellulose equivalent by weight of the densified granules and the acrylic acid polymer constitutes about 13% by weight of the densified granules. In other embodiments, carboxymethylcellulose constitutes about 29% to about 37%, about 30% to about 36%, about 31% to about 35%, or about 32% to about 34% of sodium carboxymethylcellulose equivalent by weight of the densified granules and the acrylic acid polymer constitutes about 10% to about 18%, about 11% to about 17%, about 12% to about 16%, or about 13% to about 15% by weight of the densified granules. In other specific embodiments, carboxymethylcellulose constitutes about 33% of sodium carboxymethylcellulose equivalent by weight of the densified granules and the acrylic acid polymer constitutes about 14% by weight of the densified granules. In some embodiments, the amount of carboxymethylcellulose is about 23%, about 24%, about 25%, about 26%, about 27%, about 28%, about 29%, about 30%, about 31%, about 32%, about 33%, about 34%, about 35%, about 36%, or about 37% of sodium carboxymethylcellulose equivalent by weight of the tablet core and the amount of the acrylic acid polymer is about 7%, about 8%, about 9%, about 10%, about 11%, about 12%, about 13%, about 14%, about 15%, about 16%, about 17%, or about 18% by weight of the densified granules. In some embodiments, the weight ratio of the acrylic acid polymer to sodium carboxymethylcellulose equivalent is about 3:2, about 3:3 (1:1), about 3:4, about 3:5, about 3:6, about 3:7, about 3:8, about 3:9, about 2:3, about 2:4, about 2:5, about 2:6, about 2:7, or about 2:8. In specific embodiments, the weight ratio of the acrylic acid polymer to sodium carboxymethylcellulose equivalent is about 3:7. In some of the embodiments described in this paragraph, the pre-blend comprises the first and the second anionic polymer, but no more other anionic polymers than the first and the second anionic polymer. In some of the embodiments described in this paragraph, the pre-blend consists essentially of dexpramipexole, or a pharmaceutically acceptable salt thereof, and the first and the second anionic polymer.

In some embodiments, the one or more polymers are neutral polymers.

In some specific embodiments, the one or more neutral polymers are hypromellose (hydroxypropyl methylcellulose, HPMC), hydroxypropyl cellulose (HPC), silicified hydroxypropyl methylcellulose, and/or poly(ethylene oxide). Non-limiting examples of a HPMC are Methocel® K15M, Methocel® K100M, and Methocel® K200M. A non-limiting example of a HPC is Klucel® Xtend HXF. A non-limiting example of poly(ethylene oxide) is Sentry Polyox® WSR-303.

In some embodiments, the pre-blend comprises one, but no more than one neutral polymer. In specific embodiments, the pre-blend comprises one, but no more than one neutral polymer and the neutral polymer is hydroxypropyl methylcellulose or silicified hydroxypropyl methylcellulose.

In some embodiments, the pre-blend comprises more than one neutral polymer, such as two, three, four, or five neutral polymers. Within the meaning of the present disclosure, if the pre-blend comprises more than one neutral polymer, such as a first and a second neutral polymer, the neutral polymers are different neutral polymers. For example, a pre-blend comprising two neutral polymers might comprise hydroxypropyl methylcellulose or silicified hydroxypropyl methylcellulose.

In some embodiments, the pre-blend comprises one or more, but no more than five neutral polymers. In some embodiments, the pre-blend comprises one or more, but no more than four neutral polymers. In some embodiments, the pre-blend comprises one or more, but no more than three neutral polymers. In some embodiments, the pre-blend comprises one or more, but no more than two neutral polymers.

In some embodiments, the densified granules consists essentially of dexpramipexole, or a pharmaceutically acceptable salt thereof, constituting about 30% to about 40% of dexpramipexole dihydrochloride monohydrate equivalent by weight of the densified granules and one or more, but no more than two neutral polymers at the amounts specified herein (such as at about 5% to about 25% by weight of the densified granules). If two neutral polymers are present, the first neutral polymer may be hydroxypropyl methylcellulose or silicified hydroxypropyl methylcellulose. In some embodiments, hydroxypropyl methylcellulose or silicified hydroxypropyl methylcellulose constitutes about 5% to about 25% by weight of the densified granules. In some embodiments, hydroxypropyl methylcellulose or silicified hydroxypropyl methylcellulose constitutes about 10% to about 20%, about 11% to about 19%, about 12% to about 18%, about 13% to about 17%, or about 14% to about 16% by weight of the densified granules. In specific embodiments, hydroxypropyl methylcellulose or silicified hydroxypropyl methylcellulose constitutes about 15% by weight of the densified granules.

In some embodiments, the one or more neutral polymers constitute about 5% to about 25% by weight of the densified granules. In some embodiments, the one or more neutral polymers constitutes about 10% to about 20%, about 11% to about 19%, about 12% to about 18%, about 13% to about 17%, or about 14% to about 16% by weight of the densified granules. In specific embodiments, the one or more neutral polymers constitute about 15% by weight of the densified granules.

In some embodiments, the one or more neutral polymers are hydroxypropyl methylcellulose or silicified hydroxypropyl methylcellulose. In some embodiments, the hydroxypropyl methylcellulose or silicified hydroxypropyl methylcellulose constitutes about 5% to about 25% by weight of the densified granules. In some embodiments, the hydroxypropyl methylcellulose or silicified hydroxypropyl methylcellulose constitutes about 10% to about 20%, about 11% to about 19%, about 12% to about 18%, about 13% to about 17%, or about 14% to about 16% by weight of the densified granules. In specific embodiments, the hydroxypropyl methylcellulose or silicified hydroxypropyl methylcellulose constitutes about 15% by weight of the densified granules.

In some embodiments, the amount of hydroxypropyl methylcellulose or silicified hydroxypropyl methylcellulose is about 5% and about 60% by weight of the densified granules. In some embodiments, the amount of hydroxypropyl methylcellulose or silicified hydroxypropyl methylcellulose is about 10% and about 60% by weight of the densified granules. In some embodiments, the one or more neutral polymers constitute about 15% to about 60% by weight of the densified granules. In some embodiments, the amount of hydroxypropyl methylcellulose or silicified hydroxypropyl methylcellulose is about 20% and about 60% by weight of the densified granules. In some embodiments, the one or more neutral polymers constitute about 25% to about 60% by weight of the densified granules. In some embodiments, the amount of hydroxypropyl methylcellulose or silicified hydroxypropyl methylcellulose is about 30% and about 60% by weight of the densified granules. In some embodiments, the one or more neutral polymers constitute about 35% to about 60% by weight of the densified granules. In some embodiments, the amount of hydroxypropyl methylcellulose or silicified hydroxypropyl methylcellulose is about 40% and about 60% by weight of the densified granules. In some embodiments, the one or more neutral polymers constitute about 45% to about 60% by weight of the densified granules. In some embodiments, the amount of hydroxypropyl methylcellulose or silicified hydroxypropyl methylcellulose is about 50% and about 60% by weight of the densified granules. In some embodiments, the one or more neutral polymers constitute about 55% to about 60% by weight of the densified granules.

In some embodiments, the amount of hydroxypropyl methylcellulose or silicified hydroxypropyl methylcellulose is about 5% to about 55% by weight of the densified granules. In some embodiments, the amount of hydroxypropyl methylcellulose or silicified hydroxypropyl methylcellulose is about 5% to about 50% by weight of the densified granules. In some embodiments, the amount of hydroxypropyl methylcellulose or silicified hydroxypropyl methylcellulose is about 5% to about 45% by weight of the densified granules. In some embodiments, the amount of hydroxypropyl methylcellulose or silicified hydroxypropyl methylcellulose is about 5% to about 40% by weight of the densified granules. In some embodiments, the amount of hydroxypropyl methylcellulose or silicified hydroxypropyl methylcellulose is about 5% to about 35% by weight of the densified granules. In some embodiments, the amount of hydroxypropyl methylcellulose or silicified hydroxypropyl methylcellulose is about 5% to about 30% by weight of the densified granules.

In some embodiments, the amount of hydroxypropyl methylcellulose or silicified hydroxypropyl methylcellulose is about 20% by weight of the densified granules. In some embodiments, the amount of hydroxypropyl methylcellulose or silicified hydroxypropyl methylcellulose is about 25% by weight of the densified granules. In some embodiments, the amount of hydroxypropyl methylcellulose or silicified hydroxypropyl methylcellulose is about 30% by weight of the densified granules. In some embodiments, the amount of hydroxypropyl methylcellulose or silicified hydroxypropyl methylcellulose is about 35% by weight of the densified granules. In some embodiments, the amount of hydroxypropyl methylcellulose or silicified hydroxypropyl methylcellulose is about 40% by weight of the densified granules. In some embodiments, the amount of hydroxypropyl methylcellulose or silicified hydroxypropyl methylcellulose is about 45% by weight of the densified granules. In some embodiments, the amount of hydroxypropyl methylcellulose or silicified hydroxypropyl methylcellulose is about 50% by weight of the densified granules. In some embodiments, the amount of hydroxypropyl methylcellulose or silicified hydroxypropyl methylcellulose is about 55% by weight of the densified granules. In some embodiments, the amount of hydroxypropyl methylcellulose or silicified hydroxypropyl methylcellulose is about 60% by weight of the densified granules.

In some embodiments, the pre-blend further comprises a diluent such as silicified microcrystalline cellulose. As regards further suitable diluents, reference is also made to chapter “II—Pharmaceutical Composition (Orally Deliverable Tablet),” section “3. Tablet Core” above. In some embodiments, the diluent constitutes about 10% to about 25% by weight of the densified granules. In some embodiments, the diluent constitutes about 10% to about 50% by weight of the densified granules. In some embodiments, the diluent constitutes about 15% to about 45% by weight of the densified granules. In some embodiments, the diluent constitutes about 18% to about 24% by weight of the densified granules. In some embodiments, the diluent constitutes about 20% to about 22% by weight of the densified granules. In some embodiments, the diluent constitutes about 21% by weight of the densified granules. In some embodiments, the diluent constitutes about 13% to about 19% by weight of the densified granules. In some embodiments, the diluent constitutes about 15% to about 17% by weight of the densified granules. In some embodiments, the diluent constitutes about 16% by weight of the densified granules. In some embodiments, the diluent constitutes about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, or about 45% by weight of the densified granules.

In some embodiments, the pre-blend further comprises a lubricant such as magnesium stearate. As regards further suitable lubricants, reference is also made to chapter “II—Pharmaceutical Composition (Orally Deliverable Tablet),” section “3. Tablet Core” above. In some embodiments, the lubricant constitutes about 0.1% to about 0.4% or about 0.15% to about 0.35% by weight of the densified granules. In some embodiments, the lubricant constitutes about 0.2% to about 0.3% by weight of the densified granules. In some embodiments, the lubricant constitutes about 0.25% by weight of the densified granules. In some embodiments, the lubricant constitutes about 0.1%, about 0.15%, about 0.2%, about 0.25%, about 0.3%, about 0.35%, or about 0.4% by weight of the densified granules.

### 2. Method of Manufacturing a Blend and Method of Manufacturing an Orally Deliverable Table

The present disclosure further relates to a method of manufacturing a blend for preparing a sustained release pharmaceutical composition comprising dexpramipexole, or a pharmaceutically acceptable salt thereof, the method comprising:
preparing a pre-blend by mixing dexpramipexole, or a pharmaceutically acceptable salt thereof, and one or more polymers;
preparing densified granules from the pre-blend; and
preparing a final blend by mixing the densified granules with a glidant and a lubricant;
wherein dexpramipexole, or the pharmaceutically acceptable salt thereof, constitutes about 30% to about 40% of dexpramipexole dihydrochloride monohydrate equivalent by weight of the blend and the one or more polymers constitute about 5% to about 60% by weight of the blend.

The present disclosure further relates to a method of manufacturing a blend for preparing a sustained release pharmaceutical composition comprising dexpramipexole, or a pharmaceutically acceptable salt thereof, the method comprising:
preparing a pre-blend by mixing dexpramipexole, or a pharmaceutically acceptable salt thereof, and one or more anionic polymers;
preparing densified granules from the pre-blend; and
preparing a blend by mixing the densified granules with a glidant and a lubricant;
wherein dexpramipexole, or the pharmaceutically acceptable salt thereof, constitutes about 30% to about 40% of dexpramipexole dihydrochloride monohydrate equivalent by weight of the blend and the one or more anionic polymers constitute about 30% to about 55% by weight of the blend.

The present disclosure further relates to a method of manufacturing a blend for preparing a sustained release pharmaceutical composition comprising dexpramipexole, or a pharmaceutically acceptable salt thereof, the method comprising:
preparing a pre-blend by mixing dexpramipexole, or a pharmaceutically acceptable salt thereof, and one or more anionic polymers; and
preparing a final blend by mixing the pre-blend with a glidant and a lubricant;
wherein dexpramipexole, or the pharmaceutically acceptable salt thereof, constitutes about 30% to about 40% of dexpramipexole dihydrochloride monohydrate equivalent by weight of the blend and the one or more anionic polymers constitute about 30% to about 55% by weight of the blend.

In some embodiments, the pre-blend further comprises additional excipients. In some embodiments, the final blend further comprises additional excipients.

In some embodiments, the final blend comprises an antioxidant. In some embodiments, the antioxidant is capable of acting as a nitrite scavenger. Non-limiting examples of nitrite scavengers include, but are not limited to, ascorbic acid, L-cysteine, caffeic acid, cysteine HCL, methionine, tartaric acid, gallic acid, uric acid, and sodium sulphite. In some embodiments, the pre-blend comprises one nitrite scavenger. In another embodiment, the final blend comprises multiple nitrite scavengers. In some embodiments, the nitrite scavengers constitute about 0.1% to about 1.5% by weight of the tablet core. In some embodiments, the nitrite scavenger constitutes about 0.1% to about 1.0% by weight of the tablet core. In some embodiments, the nitrite scavenger constitutes about 0.1% to about 0.5% by weight of the tablet core. In some embodiments, the nitrite scavenger constitutes about 0.5% to about 1.5% by weight of the tablet core. In some embodiments, the nitrite scavenger constitutes about 1% to about 1.5% by weight of the tablet core. In some embodiments, the nitrite scavenger constitutes about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, or about 1.5% by weight of the tablet core.

In some embodiments, the one or more polymers are anioic polymers or neutral polymers. In some embodiments, the one or more polymers are anionic polymers. In some embodiments, the one or more anionic polymers constitute about 20% to about 55% by weight of the densified granules.

In some embodiments, the one or more neutral polymers constitute between about 5% and about 60% by weight of the densified granules. In some embodiments, the one or more neutral polymers constitute between about 10% and about 60% by weight of the densified granules. In some embodiments, the one or more neutral polymers constitute about 15% to about 60% by weight of the densified granules. In some embodiments, the one or more neutral polymers constitute between about 20% and about 60% by weight of the densified granules. In some embodiments, the one or more neutral polymers constitute about 25% to about 60% by weight of the densified granules. In some embodiments, the one or more neutral polymers constitute between about 30% and about 60% by weight of the densified granules. In some embodiments, the one or more neutral polymers constitute about 35% to about 60% by weight of the densified granules. In some embodiments, the one or more neutral polymers constitute between about 40% and about 60% by weight of the densified granules. In some embodiments, the one or more neutral polymers constitute about 45% to about 60% by weight of the densified granules. In some embodiments, the one or more neutral polymers constitute between about 50% and about 60% by weight of the densified granules. In some embodiments, the one or more neutral polymers constitute about 55% to about 60% by weight of the densified granules.

In some embodiments, the one or more neutral polymers constitute about 5% to about 55% by weight of the densified granules. In some embodiments, the one or more neutral polymers constitute about 5% to about 50% by weight of the densified granules. In some embodiments, the one or more neutral polymers constitute about 5% to about 45% by weight of the densified granules. In some embodiments, the one or more neutral polymers constitute about 5% to about 40% by weight of the densified granules. In some embodiments, the one or more neutral polymers constitute about 5% to about 35% by weight of the densified granules. In some embodiments, the one or more neutral polymers constitute about 5% to about 30% by weight of the densified granules.

In some embodiments, the one or more neutral polymers constitute about 20% by weight of the densified granules. In some embodiments, the one or more neutral polymers constitute about 25% by weight of the densified granules. In some embodiments, the one or more neutral polymers constitute about 30% by weight of the densified granules. In some embodiments, the one or more neutral polymers constitute about 35% by weight of the densified granules. In some embodiments, the one or more neutral polymers constitute about 40% by weight of the densified granules. In some embodiments, the one or more neutral polymers constitute about 45% by weight of the densified granules. In some embodiments, the one or more neutral polymers constitute about 50% by weight of the densified granules. In some embodiments, the one or more neutral polymers constitute about 55% by weight of the densified granules. In some embodiments, the one or more neutral polymers constitute about 60% by weight of the densified granules.

The present disclosure yet further relates to a method of manufacturing a sustained release pharmaceutical composition in the form of an orally deliverable tablet comprising a tablet core with dexpramipexole, or a pharmaceutically acceptable salt thereof, the method comprising:
preparing a pre-blend by mixing dexpramipexole, or a pharmaceutically acceptable salt thereof, and one or more polymers;
preparing densified granules from the pre-blend;
preparing a blend by mixing the densified granules with a glidant and a lubricant; and
compressing the blend into a tablet core;
wherein dexpramipexole, or the pharmaceutically acceptable salt thereof, constitutes about 30% to about 40% of dexpramipexole dihydrochloride monohydrate equivalent by weight of the blend and the one or more polymers constitute about 5% to about 60% by weight of the blend.

The present disclosure yet further relates to a method of manufacturing a sustained release pharmaceutical composition in the form of an orally deliverable tablet comprising a tablet core with dexpramipexole, or a pharmaceutically acceptable salt thereof, the method comprising:
preparing a pre-blend by mixing dexpramipexole, or a pharmaceutically acceptable salt thereof, and one or more anionic polymers;
preparing densified granules from the pre-blend;
preparing a final blend by mixing the densified granules with a glidant and a lubricant; and
compressing the blend into a tablet core;
wherein dexpramipexole, or the pharmaceutically acceptable salt thereof, constitutes about 30% to about 40% of dexpramipexole dihydrochloride monohydrate equivalent by weight of the blend and the one or more anionic polymers constitute about 30% to about 55% by weight of the blend.

The present disclosure yet further relates to a method of manufacturing a sustained release pharmaceutical composition in the form of an orally deliverable tablet comprising a tablet core with dexpramipexole, or a pharmaceutically acceptable salt thereof, the method comprising:
preparing a pre-blend by mixing dexpramipexole, or a pharmaceutically acceptable salt thereof, and one or more anionic polymers;
preparing a final blend by mixing the pre-blend with a glidant and a lubricant; and
compressing the blend into a tablet core;
wherein dexpramipexole, or the pharmaceutically acceptable salt thereof, constitutes about 30% to about 40% of dexpramipexole dihydrochloride monohydrate equivalent by weight of the blend and the one or more anionic polymers constitute about 30% to about 55% by weight of the blend.

In some embodiments, the pre-blend further comprises additional excipients. In some embodiments, the final blend further comprises additional excipients.

In some embodiments, the one or more polymers are anioic polymers or neutral polymers.

In some embodiments, the one or more polymers are anionic polymers. In some embodiments, the one or more anionic polymers constitute about 20% to about 55% by weight of the densified granules.

In some embodiments, the one or more polymers are neutral polymers. In some embodiments, the one or more neutral polymers constitute about 5% to about 25% by weight of the tablet core.

In some embodiments, the press speed during compressing the blend is about 10 to about 30 rpm, such as about 15 to about 25 rpm. In some embodiments, the press speed during compressing the blend is about 18 rpm to about 22 rpm, such as about 20 rpm. In some embodiments, the feeder speed during compressing the blend is about 20 to about 40 rpm, such as about 25 to about 35 rpm. In some embodiments, the feeder speed during compressing the blend is about 28 rpm to about 32 rpm, such as about 30 rpm. In some embodiments, the blend is compressed into the tablet core using a Korsch XL-100.

In some embodiments of the method of manufacturing a sustained release pharmaceutical composition in the form of an orally deliverable tablet, the method further comprises coating the tablet core with a film coating.

In some embodiments, the film coating is applied through a film coating solution that is sprayed onto the tablet core. In some embodiments, the pan size for film coating is about 17 to about 21 in, such as about 19 in. In some embodiments, the nozzle spot opening is about 1.0 to about 1.4 mm, such as about 1.2 mm. In some embodiments, the drum speed is about 10 rpm to about 14 rpm, such as about 12 rpm. In some embodiments, the inlet air volume is about 178 to about 184 cfm, such as about 180 to about 182 cfm. In some embodiments, the inlet air temperature is about 60 to about 65° C., such as about 63 to about 64° C. or about 61 to about 63° C. In some embodiments, the exhaust temperature is about 48 to about 50° C., such as about 48° C. In some embodiments, the spray rate is about 18 to about 24 g/min. In some embodiments, the spray rate is about 20 to about 22 g/min, such as about 22 g/min. In some embodiments, the pattern air pressure is about 18 to about 22 psi, such as about 20 psi. In some embodiments, the atomizing air pressure is about 18 to about 22 psi, such as about 20 psi.

The solvent for the film coating solution may be purified water and/or the solids in the film coating solution may constitute about 20% by weight of the film coating solution.

In some embodiments, the film coating comprises polyvinyl alcohol. In specific embodiments, the film coating comprises polyvinyl alcohol, titanium dioxide, polyethylene glycol and talc. As regards further suitable coatings, reference is made to chapter “II—Pharmaceutical Composition (Orally Deliverable Tablet),” section “4. Film Coating” above.

In some embodiments, dexpramipexole, or the pharmaceutically acceptable salt thereof, constitutes about 30% to about 50% of dexpramipexole dihydrochloride monohydrate equivalent by weight of the blend. In some embodiments, dexpramipexole, or the pharmaceutically acceptable salt thereof, constitutes about 35% to about 40% of dexpramipexole dihydrochloride monohydrate equivalent by weight of the blend. In some embodiments, dexpramipexole, or the pharmaceutically acceptable salt thereof, constitutes about 35% to about 45% of dexpramipexole dihydrochloride monohydrate equivalent by weight of the blend. In some embodiments, dexpramipexole, or the pharmaceutically acceptable salt thereof, constitutes about 32% to about 38%, about 33% to about 37%, or about 34% to about 36% of dexpramipexole dihydrochloride monohydrate equivalent by weight of the blend. In some embodiments, dexpramipexole, or the pharmaceutically acceptable salt thereof, constitutes about 35% of dexpramipexole dihydrochloride monohydrate equivalent by weight of the blend. In some embodiments, dexpramipexole, or the pharmaceutically acceptable salt thereof, constitutes about 36% to about 40% or about 37% to about 39% of dexpramipexole dihydrochloride monohydrate equivalent by weight of the blend. In some embodiments, dexpramipexole, or the pharmaceutically acceptable salt thereof, constitutes about 38% of dexpramipexole dihydrochloride monohydrate equivalent by weight of the blend. In some embodiments, dexpramipexole, or the pharmaceutically acceptable salt thereof, constitutes about 30%, about 31%, about 32%, about 33%, about 34%, about 35%, about 36%, about 37%, about 38%, about 39%, about 40%, about 41%, about 42%, about 43%, about 44%, or about 45% of dexpramipexole dihydrochloride monohydrate equivalent by weight of the blend.

In some embodiments, the weight ratio of dexpramipexole dihydrochloride equivalent to the one or more anionic polymers is about 0.2:1 to about 1.3:1, about 0.3:1 to about 1.2:1, about 0.4:1 to about 1.1:1, or about 0.5:1 to about 1:1. In some embodiments, the weight ratio of dexpramipexole dihydrochloride equivalent to the one or more polymers is about 0.2:1 to about 4.0:1, about 0.3:1 to about 3.8:1, about 0.4:1 to about 3.6:1, about 0.5:1 to about 3.5:1, about 0.6:1 to about 3.4:1, about 0.7:1 to about 3.3:1, about 0.8:1 to about 3.2:1, about 0.9:1 to about 3.1:1, about 1.0:1 to about 3.0:1, about 1.1:1 to about 2.9:1, or about 1.2:1 to about 2.8:1. In some embodiments, the weight ratio of dexpramipexole dihydrochloride equivalent to the one or more anionic polymers is about 0.7:1 to about 0.9:1. In some embodiments, the weight ratio of dexpramipexole dihydrochloride equivalent to the one or more anionic polymers is about 0.2:1, about 0.3:1, about 0.4:1, about 0.5:1, about 0.6:1, about 0.7:1, about 0.8:1, about 0.9:1, about 1:1, about 1.1:1, about 1.2:1, or about 1.3:1. In some embodiments, the weight ratio of dexpramipexole dihydrochloride equivalent to the one or more polymers is about 0.7:1 to about 0.9:1. In some embodiments, the weight ratio of dexpramipexole dihydrochloride equivalent to the one or more anionic polymers is about 0.2:1, about 0.3:1, about 0.4:1, about 0.5:1, about 0.6:1, about 0.7:1, about 0.8:1, about 0.9:1, about 1:1, about 1.1:1, about 1.2:1, about 1.3:1, about 1.4:1, about 1.5:1, about 1.6:1, about 1.7:1, about 1.8:1, about 1.9:1, about 2.0:1, about 2.1:1, about 2.2:1, about 2.3:1, about 2.4:1, about 2.5:1, about 2.6:1, about 2.7:1, about 2.8:1, about 2.9:1, about 3.0:1, about 3.1:1, about 3.2:1, about 3.3:1, about 3.4:1, about 3.5:1, about 3.6:1, about 3.7:1, about 3.8:1, about 3.9:1, or about 4.0:1.

In some embodiments, the one or more anionic polymers constitute about 20% to about 55% by weight of the blend. In some embodiments, the one or more anionic polymers constitute about 30% to about 50% by weight of the blend. In some embodiments, the one or more anionic polymers constitute about 35% to about 55% by weight of the blend. In some embodiments, the one or more anionic polymers constitute about 35% to about 50% by weight of the blend. In some embodiments, the one or more anionic polymers constitute about 40% to about 50% by weight of the blend. In some embodiments, the one or more anionic polymers constitute about 35% to about 45%, about 36% to about 44%, about 37% to about 43%, about 38% to about 42%, or about 39% to about 41% by weight of the blend. In some embodiments, the one or more anionic polymers constitute about 40% by weight of the blend. In some embodiments, the one or more anionic polymers constitute about 42% to about 52%, about 43% to about 51%, about 44% to about 50%, about 45% to about 49%, or about 46% to about 48% by weight of the blend. In some embodiments, the one or more anionic polymers constitute about 47% by weight of the blend. In some embodiments, the one or more anionic polymers constitute about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, or about 55% by weight of the blend.

In some embodiments, the one or more anionic polymers are selected from carboxymethylcellulose, an acrylic acid polymer, a methacrylic acid polymer, a methacrylic acid copolymer, alignate, carrageenan, xanthan gum, or arabic gum. In some embodiments, the one or more anionic polymers are hydrophilic anionic polymers. In some embodiments, the one or more anionic polymers are selected from carboxymethylcellulose and an acrylic acid polymer. The carboxymethylcellulose may be sodium carboxymethylcellulose. An example of a sodium carboxymethylcellulose is Aqualon CMC 7HXF PH. In more specific embodiments, the viscosity of the sodium carboxymethylcellulose is about 1,500 to about 3,000 cP (centipoise; 1% in water). The acrylic acid polymer may be crosslinked acrylic acid polymer. An example of a crosslinked acrylic acid polymer is Carbopol 971P. In more specific embodiments, the viscosity of the crosslinked acrylic acid polymer is about 4,000 to about 11,000 cP (centipoise; 0.5% at pH 7.5).

In some embodiments, the (pre-)blend comprises one, but no more than one anionic polymer. In specific embodiments, the (pre-)blend comprises one but no more than one anionic polymer and the anionic polymer is carboxymethylcellulose, such as sodium carboxymethylcellulose. In specific embodiments, the (pre-)blend comprises one but no more than one anionic polymer and the anionic polymer is an acrylic acid polymer, such as crosslinked acrylic acid polymer.

In some embodiments, the (pre-)blend comprises more than one anionic polymer, such as two, three, four, or five anionic polymers. Within the meaning of the present disclosure, if the (pre-)blend comprises more than one anionic polymer, such as a first and a second anionic polymer, the anionic polymers are different anionic polymers. For example, a (pre-)blend comprising two anionic polymers might comprise sodium carboxymethylcellulose as the first anionic polymer and an acrylic acid polymer as the second anionic polymer.

In some embodiments, the (pre-)blend comprises one or more, but no more than five anionic polymers. In some embodiments, the (pre-)blend comprises one or more, but no more than four anionic polymers. In some embodiments, the (pre-)blend comprises one or more, but no more than three anionic polymers. In some embodiments, the (pre-)blend comprises one or more, but no more than two anionic polymers.

In some embodiments, the (pre-)blend comprises two, but no more than two anionic polymers. In specific embodiments, the (pre-)blend comprises two, but no more than two anionic polymers and the two anionic polymers are carboxymethylcellulose (such as sodium carboxymethylcellulose) and an acrylic acid polymer (such as a crosslinked acrylic acid polymer).

In some embodiments, the blend consists essentially of dexpramipexole, or a pharmaceutically acceptable salt thereof, constituting about 30% to about 40% of dexpramipexole dihydrochloride monohydrate equivalent by weight of the blend and one or more, but no more than two anionic polymers at the amounts specified herein (such as at about 30% to about 50% by weight of the blend). In some embodiments, the blend consists essentially of dexpramipexole, or a pharmaceutically acceptable salt thereof, constituting about 30% to about 50% of dexpramipexole dihydrochloride monohydrate equivalent by weight of the blend and one or more, but no more than two anionic polymers at the amounts specified herein (such as at about 30% to about 50% by weight of the blend). The two anionic polymers may be carboxymethylcellulose (such as sodium carboxymethylcellulose) and an acrylic acid polymer (such as a crosslinked acrylic acid polymer).

In some embodiments, the blend consists essentially of dexpramipexole, or a pharmaceutically acceptable salt thereof, constituting about 30% to about 40% of dexpramipexole dihydrochloride monohydrate equivalent by weight of the blend and two anionic polymers at the amounts specified herein (such as at about 30% to about 50% by weight of the blend). In some embodiments, the blend consists essentially of dexpramipexole, or a pharmaceutically acceptable salt thereof, constituting about 30% to about 50% of dexpramipexole dihydrochloride monohydrate equivalent by weight of the blend and two anionic polymers at the amounts specified herein (such as at about 30% to about 50% by weight of the blend). The two anionic polymers may be carboxymethylcellulose (such as sodium carboxymethylcellulose) and an acrylic acid polymer (such as a crosslinked acrylic acid polymer).

In some embodiments, the pre-blend comprises a first anionic polymer and a second anionic polymer and the first anionic polymer is carboxymethylcellulose and the second anionic polymer is an acrylic acid polymer. The acrylic acid polymer may be crosslinked acrylic acid polymer and/or the carboxymethylcellulose may be sodium carboxymethylcellulose. In some embodiments, carboxymethylcellulose constitutes about 23% to about 37% of sodium carboxymethylcellulose equivalent by weight of the blend and the acrylic acid polymer constitutes about 7% to about 18% by weight of the blend. In some embodiments, carboxymethylcellulose constitutes about 23% to about 33%, about 24% to about 32%, about 25% to about 31%, about 26% to about 30%, or about 27% to about 29% of sodium carboxymethylcellulose equivalent by weight of the blend and the acrylic acid polymer constitutes about 7% to about 16%, about 8% to about 15%, about 10% to about 14%, or about 11% to about 13% by weight of the blend. In specific embodiments, carboxymethylcellulose constitutes about 28% of sodium carboxymethylcellulose equivalent by weight of the blend and the acrylic acid polymer constitutes about 12% by weight of the blend. In other embodiments, carboxymethylcellulose constitutes about 29% to about 37%, about 30% to about 36%, about 31% to about 35%, or about 32% to about 34% of sodium carboxymethylcellulose equivalent by weight of the blend and the acrylic acid polymer constitutes about 10% to about 18%, about 11% to about 17%, about 12% to about 16%, or about 13% to about 15% by weight of the blend. In other specific embodiments, carboxymethylcellulose constitutes about 33% of sodium carboxymethylcellulose equivalent by weight of the blend and the acrylic acid polymer constitutes about 14% by weight of the blend. In some embodiments, the weight ratio of the acrylic acid polymer to sodium carboxymethylcellulose equivalent is about 3:2, about 3:3 (1:1), about 3:4, about 3:5, about 3:6, about 3:7, about 3:8, about 3:9, about 2:3, about 2:4, about 2:5, about 2:6, about 2:7, or about 2:8. In specific embodiments, the weight ratio of the acrylic acid polymer to sodium carboxymethylcellulose equivalent is about 3:7. In some of the embodiments described in this paragraph, the (pre-)blend comprises the first and the second anionic polymer, but no more other anionic polymers than the first and the second anionic polymer. In some of the embodiments described in this paragraph, the (pre-)blend consists essentially of dexpramipexole, or a pharmaceutically acceptable salt thereof, and the first and the second anionic polymer.

In some embodiments, the one or more polymers are neutral polymers.

In some embodiments, the one or more neutral polymers constitute about 5% to about 25% by weight of the densified granules. In some embodiments, the one or more neutral polymers constitutes about 10% to about 20%, about 11% to about 19%, about 12% to about 18%, about 13% to about 17%, or about 14% to about 16% by weight of the densified granules. In specific embodiments, the one or more neutral polymers constitute about 15% by weight of the densified granules.

In some specific embodiments, the one or more neutral polymers are hypromellose (hydroxypropyl methylcellulose, HPMC), silicified hydroxypropyl methylcellulose, hydroxypropyl cellulose (HPC), and/or poly(ethylene oxide). Non-limiting examples of a HPMC are Methocel® K15M, Methocel® K100M, and Methocel® K200M. A non-limiting example of a HPC is Klucel® Xtend HXF. A non-limiting example of poly(ethylene oxide) is Sentry Polyox® WSR-303.

In some embodiments, the one or more neutral polymers are hydroxypropyl methylcellulose or silicified hydroxypropyl methylcellulose. In some embodiments, the hydroxypropyl methylcellulose or silicified hydroxypropyl methylcellulose constitutes about 5% to about 25% by weight of the densified granules. In some embodiments, the hydroxypropyl methylcellulose or silicified hydroxypropyl methylcellulose constitutes about 10% to about 20%, about 11% to about 19%, about 12% to about 18%, about 13% to about 17%, or about 14% to about 16% by weight of the densified granules. In specific embodiments, the hydroxypropyl methylcellulose or silicified hydroxypropyl methylcellulose constitutes about 15% by weight of the densified granules.

In some embodiments, the amount of hydroxypropyl methylcellulose or silicified hydroxypropyl methylcellulose is about 5% and about 60% by weight of the densified granules. In some embodiments, the amount of hydroxypropyl methylcellulose or silicified hydroxypropyl methylcellulose is about 10% and about 60% by weight of the densified granules. In some embodiments, the one or more neutral polymers constitute about 15% to about 60% by weight of the densified granules. In some embodiments, the amount of hydroxypropyl methylcellulose or silicified hydroxypropyl methylcellulose is about 20% and about 60% by weight of the densified granules. In some embodiments, the one or more neutral polymers constitute about 25% to about 60% by weight of the densified granules. In some embodiments, the amount of hydroxypropyl methylcellulose or silicified hydroxypropyl methylcellulose is about 30% and about 60% by weight of the densified granules. In some embodiments, the one or more neutral polymers constitute about 35% to about 60% by weight of the densified granules. In some embodiments, the amount of hydroxypropyl methylcellulose or silicified hydroxypropyl methylcellulose is about 40% and about 60% by weight of the densified granules. In some embodiments, the one or more neutral polymers constitute about 45% to about 60% by weight of the densified granules. In some embodiments, the amount of hydroxypropyl methylcellulose or silicified hydroxypropyl methylcellulose is about 50% and about 60% by weight of the densified granules. In some embodiments, the one or more neutral polymers constitute about 55% to about 60% by weight of the densified granules.

In some embodiments, the amount of hydroxypropyl methylcellulose or silicified hydroxypropyl methylcellulose is about 5% to about 55% by weight of the densified granules. In some embodiments, the amount of hydroxypropyl methylcellulose or silicified hydroxypropyl methylcellulose is about 5% to about 50% by weight of the densified granules. In some embodiments, the amount of hydroxypropyl methylcellulose or silicified hydroxypropyl methylcellulose is about 5% to about 45% by weight of the densified granules. In some embodiments, the amount of hydroxypropyl methylcellulose or silicified hydroxypropyl methylcellulose is about 5% to about 40% by weight of the densified granules. In some embodiments, the amount of hydroxypropyl methylcellulose or silicified hydroxypropyl methylcellulose is about 5% to about 35% by weight of the densified granules. In some embodiments, the amount of hydroxypropyl methylcellulose or silicified hydroxypropyl methylcellulose is about 5% to about 30% by weight of the densified granules.

In some embodiments, the amount of hydroxypropyl methylcellulose or silicified hydroxypropyl methylcellulose is about 20% by weight of the densified granules. In some embodiments, the amount of hydroxypropyl methylcellulose or silicified hydroxypropyl methylcellulose is about 25% by weight of the densified granules. In some embodiments, the amount of hydroxypropyl methylcellulose or silicified hydroxypropyl methylcellulose is about 30% by weight of the densified granules. In some embodiments, the amount of hydroxypropyl methylcellulose or silicified hydroxypropyl methylcellulose is about 35% by weight of the densified granules. In some embodiments, the amount of hydroxypropyl methylcellulose or silicified hydroxypropyl methylcellulose is about 40% by weight of the densified granules. In some embodiments, the amount of hydroxypropyl methylcellulose or silicified hydroxypropyl methylcellulose is about 45% by weight of the densified granules. In some embodiments, the amount of hydroxypropyl methylcellulose or silicified hydroxypropyl methylcellulose is about 50% by weight of the densified granules. In some embodiments, the amount of hydroxypropyl methylcellulose or silicified hydroxypropyl methylcellulose is about 55% by weight of the densified granules. In some embodiments, the amount of hydroxypropyl methylcellulose or silicified hydroxypropyl methylcellulose is about 60% by weight of the densified granules.

In some embodiments, the pre-blend comprises one, but no more than one neutral polymer. In specific embodiments, the pre-blend comprises one, but no more than one neutral polymer and the neutral polymer is hydroxypropyl methylcellulose or silicified hydroxypropyl methylcellulose.

In some embodiments, the pre-blend comprises more than one neutral polymer, such as two, three, four, or five neutral polymers. Within the meaning of the present disclosure, if the pre-blend comprises more than one neutral polymer, such as a first and a second neutral polymer, the neutral polymers are different neutral polymers. For example, a pre-blend comprising two neutral polymers might comprise hydroxypropyl methylcellulose or silicified hydroxypropyl methylcellulose.

In some embodiments, the pre-blend comprises one or more, but no more than five neutral polymers. In some embodiments, the pre-blend comprises one or more, but no more than four neutral polymers. In some embodiments, the pre-blend comprises one or more, but no more than three neutral polymers. In some embodiments, the pre-blend comprises one or more, but no more than two neutral polymers.

In some embodiments, the densified granules consists essentially of dexpramipexole, or a pharmaceutically acceptable salt thereof, constituting about 30% to about 40% of dexpramipexole dihydrochloride monohydrate equivalent by weight of the densified granules and one or more, but no more than two neutral polymers at the amounts specified herein (such as at about 5% to about 25% by weight of the densified granules). If two neutral polymers are present, the first neutral polymer may be hydroxypropyl methylcellulose or silicified hydroxypropyl methylcellulose. In some embodiments, hydroxypropyl methylcellulose or silicified hydroxypropyl methylcellulose constitutes about 5% to about 25% by weight of the densified granules. In some embodiments, hydroxypropyl methylcellulose or silicified hydroxypropyl methylcellulose constitutes about 10% to about 20%, about 11% to about 19%, about 12% to about 18%, about 13% to about 17%, or about 14% to about 16% by weight of the densified granules. In specific embodiments, hydroxypropyl methylcellulose or silicified hydroxypropyl methylcellulose constitutes about 15% by weight of the densified granules.

In some embodiments, the glidant constitutes about 0.1% to about 0.9%, about 0.15% to about 0.85%, about 0.2% to about 0.8%, or about 0.25% to about 0.75% by weight of the blend. In specific embodiments, the glidant constitutes about 0.5% by weight of the blend. In some embodiments, the glidant constitutes about 0.1%, about 0.15%, about 0.2%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, or about 0.9% by weight of the blend. As regards suitable glidants, reference is generally made to chapter “II—Pharmaceutical Composition (Orally Deliverable Tablet),” section “3. Tablet Core” above. In some embodiments, the glidant is colloidal silicon dioxide. In some embodiments, the glidant is colloidal silicon dioxide and colloidal silicon dioxide constitutes about 0.25% to about 0.75% by weight of the blend. In specific embodiments, the glidant is colloidal silicon dioxide and colloidal silicon dioxide constitutes about 0.5% by weight of the blend.

In some embodiments, the lubricant constitutes about 0.1% to about 1.4%, about 0.15% to about 0.85%, about 0.2% to about 1.3%, or about 0.25% to about 0.75% by weight of the blend. In some embodiments, the lubricant constitutes about 0.5% to about 1% by weight of the blend. In some embodiments, the lubricant constitutes about 0.1%, about 0.15%, about 0.2%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, or about 0.9%, or about 0.95%, about 1%, about 1.1%, about 1.2%, about 1.3%, or about 1.4% by weight of the blend. As regards suitable lubricants, reference is generally made to chapter “II—Pharmaceutical Composition (Orally Deliverable Tablet),” section “3. Tablet Core” above. In some embodiments, the lubricant is magnesium stearate. In some embodiments, the lubricant is magnesium stearate and magnesium stearate constitutes about 0.25% to about 1.25% by weight of the blend. In some embodiments, the lubricant is magnesium stearate and magnesium stearate constitutes about 0.5% to about 1% by weight of the blend.

In some embodiments, the glidant is colloidal silicon dioxide and the lubricant is magnesium stearate. In specific embodiments, colloidal silicon dioxide constitutes about 0.25% to about 0.75% by weight of the blend and magnesium stearate constitutes about 0.5% to about 1% by weight of the blend.

In some embodiments, the pre-blend further comprises a diluent such as silicified microcrystalline cellulose. As regards further suitable diluents, reference is also made to chapter “II—Pharmaceutical Composition (Orally Deliverable Tablet),” section “3. Tablet Core” above. In some embodiments, the diluent constitutes about 10% to about 25% by weight of the blend. In some embodiments, the diluent constitutes about 18% to about 24% by weight of the blend. In some embodiments, the diluent constitutes about 20% to about 22% by weight of the blend. In some embodiments, the diluent constitutes about 21% by weight of the blend. In some embodiments, the diluent constitutes about 13% to about 19% by weight of the blend. In some embodiments, the diluent constitutes about 14% to about 18% by weight of the blend. In some embodiments, the diluent constitutes about 16% by weight of the blend. In some embodiments, the diluent constitutes about 10%, about 11%, about 12%, about 13%, about 14%, about 15%, about 16%, about 17%, about 18%, about 19%, about 20%, about 21%, about 22%, about 23%, about 24%, or about 25% by weight of the blend.

In some embodiments, also the pre-blend comprises an amount of lubricant. The lubricant may be magnesium stearate. As regards further suitable lubricants, reference is also made to chapter “II—Pharmaceutical Composition (Orally Deliverable Tablet),” section “3. Tablet Core” above. In some embodiments, the amount of lubricant with which the densified granules are mixed to prepare the blend may also be magnesium stearate. In specific embodiments, the pre-blend comprises about 0.25% magnesium stearate by weight of the pre-blend and the densified granules are mixed with an amount of magnesium stearate that constitutes about 0.5% by weight of the blend.

In some embodiments, the pre-blend further comprises a microcrystalline cellulose (such as silicified microcrystalline cellulose) and magnesium stearate.

In some embodiments, the densified granules constitute about 99% by weight of the blend. In some embodiments, the densified granules constitute about 99% by weight of the blend, the glidant constitutes about 0.5% by weight of the blend and the lubricant constitutes about 0.5% by weight of the blend.

In some embodiments, the blend consists essentially of:
(a) about 35% to about 40% dexpramipexole dihydrochloride monohydrate by weight of the blend;
(b) about 25% to about 30% sodium carboxymethylcellulose by weight of the blend;
(c) about 10% to about 15% crosslinked acrylic acid polymer by weight of the blend;
(d) about 18% to about 23% silicified microcrystalline cellulose by weight of the blend;
(e) about 0.5% to about 1.0% magnesium stearate by weight of the blend; and
(f) about 0.25% to about 0.75% colloidal silicon dioxide by weight of the blend.

In some embodiments, the blend consists essentially of:
(a) about 38% dexpramipexole dihydrochloride monohydrate by weight of the blend;
(b) about 28% sodium carboxymethylcellulose by weight of the blend;
(c) about 12% crosslinked acrylic acid polymer by weight of the blend;
(d) about 21% silicified microcrystalline cellulose by weight of the blend;
(e) about 0.75% magnesium stearate by weight of the blend; and
(f) about 0.5% colloidal silicon dioxide by weight of the blend.

In some embodiments, the blend consists essentially of:
(a) about 33% to about 38% dexpramipexole dihydrochloride monohydrate by weight of the blend;
(b) about 30% to about 35% sodium carboxymethylcellulose by weight of the blend;
(c) about 12% to about 17% crosslinked acrylic acid polymer by weight of the blend;
(d) about 13% to about 18% silicified microcrystalline cellulose by weight of the blend;
(e) about 0.5% to about 1.0% magnesium stearate by weight of the blend; and
(f) about 0.25% to about 0.75% colloidal silicon dioxide by weight of the blend.

In some embodiments, the blend consists essentially of:
(a) about 35% dexpramipexole dihydrochloride monohydrate by weight of the blend;
(b) about 33% sodium carboxymethylcellulose by weight of the blend;
(c) about 14% crosslinked acrylic acid polymer by weight of the blend;
(d) about 16% silicified microcrystalline cellulose by weight of the blend;
(e) about 0.75% magnesium stearate by weight of the blend; and
(f) about 0.5% colloidal silicon dioxide by weight of the blend.

In some embodiments, the blend consists essentially of:
(a) about 33% to about 39% dexpramipexole dihydrochloride monohydrate by weight of the blend;
(b) about 27% to about 33% sodium carboxymethylcellulose by weight of the blend;
(c) about 10% to about 16% crosslinked acrylic acid polymer by weight of the blend;
(d) about 18% to about 24% microcrystalline cellulose or silicified microcrystalline cellulose by weight of the blend;
(e) about 0.5% to about 1.0% magnesium stearate by weight of the blend; and
(f) about 0.25% to about 0.75% colloidal silicon dioxide by weight of the blend.

In some embodiments, the blend consists essentially of:
(a) about 36% dexpramipexole dihydrochloride monohydrate by weight of the blend;
(b) about 30% sodium carboxymethylcellulose by weight of the blend;
(c) about 13% crosslinked acrylic acid polymer by weight of the blend;
(d) about 21% microcrystalline cellulose or silicified microcrystalline cellulose by weight of the blend;
(e) about 0.75% magnesium stearate by weight of the blend; and
(f) about 0.5% colloidal silicon dioxide by weight of the blend.

In some embodiments, the blend consists essentially of:
(a) about 40% to about 46% dexpramipexole dihydrochloride monohydrate by weight of the blend;
(b) about 10% to about 20% hydroxypropyl methylcellulose or silicified hydroxypropyl methylcellulose by weight of the blend;
(c) about 38% to about 46% microcrystalline cellulose or silicified microcrystalline cellulose by weight of the blend;
(d) about 0.25% to about 0.75% magnesium stearate by weight of the blend; and
(e) about 0.25% to about 0.75% colloidal silicon dioxide by weight of the blend.

In some embodiments, the blend consists essentially of:
(a) about 43% dexpramipexole dihydrochloride monohydrate by weight of the blend;
(b) about 15% hydroxypropyl methylcellulose or silicified hydroxypropyl methylcellulose by weight of the blend;
(c) about 42% microcrystalline cellulose or silicified microcrystalline cellulose by weight of the blend;
(d) about 0.5% magnesium stearate by weight of the blend; and
(e) about 0.5% colloidal silicon dioxide by weight of the blend.

In some embodiments, the blend consists essentially of:
(a) about 40% to about 46% dexpramipexole dihydrochloride monohydrate by weight of the blend;
(b) about 10% to about 20% hydroxypropyl methylcellulose or silicified hydroxypropyl methylcellulose by weight of the blend;
(c) about 37% to about 45% microcrystalline cellulose or silicified microcrystalline cellulose by weight of the blend;
(d) about 0.5% to about 1.0% magnesium stearate by weight of the blend; and
(e) about 0.25% to about 0.75% colloidal silicon dioxide by weight of the blend.

In some embodiments, the blend consists essentially of:
(a) about 43% dexpramipexole dihydrochloride monohydrate by weight of the blend;
(b) about 15% hydroxypropyl methylcellulose or silicified hydroxypropyl methylcellulose by weight of the blend;
(c) about 41% microcrystalline cellulose or silicified microcrystalline cellulose by weight of the blend;
(d) about 0.75% magnesium stearate by weight of the blend; and
(e) about 0.5% colloidal silicon dioxide by weight of the blend.

In some embodiments, the blend consists essentially of:
(a) about 40% to about 46% dexpramipexole dihydrochloride monohydrate by weight of the blend;
(b) about 10% to about 20% hydroxypropyl methylcellulose or silicified hydroxypropyl methylcellulose by weight of the blend;
(c) about 37% to about 45% microcrystalline cellulose by weight of the blend;
(d) about 0.5% to about 1.0% magnesium stearate by weight of the blend; and
(e) about 0.25% to about 0.75% colloidal silicon dioxide by weight of the blend.

In some embodiments, the blend consists essentially of:
(a) about 43% dexpramipexole dihydrochloride monohydrate by weight of the blend;
(b) about 15% hydroxypropyl methylcellulose or silicified hydroxypropyl methylcellulose by weight of the blend;
(c) about 41% microcrystalline cellulose by weight of the blend;
(d) about 0.75% magnesium stearate by weight of the blend; and
(e) about 0.5% colloidal silicon dioxide by weight of the blend.

In some embodiments, the blend consists essentially of:
(a) about 40% to about 46% dexpramipexole dihydrochloride monohydrate by weight of the blend;
(b) about 24% to about 32% sodium carboxymethylcellulose by weight of the blend;
(c) about 25% to about 34% microcrystalline cellulose or silicified microcrystalline cellulose by weight of the blend;
(d) about 0.25% to about 0.75% magnesium stearate by weight of the blend; and
(e) about 0.25% to about 0.75% colloidal silicon dioxide by weight of the blend.

In some embodiments, the blend consists essentially of:
(a) about 43% dexpramipexole dihydrochloride monohydrate by weight of the blend;
(b) about 28% sodium carboxymethylcellulose by weight of the blend;
(c) about 29% microcrystalline cellulose or silicified microcrystalline cellulose by weight of the blend;
(d) about 0.5% magnesium stearate by weight of the blend; and
(e) about 0.5% colloidal silicon dioxide by weight of the blend.

In some embodiments, one third of the amount of magnesium stearate in the blend is already part of the pre-blend.

The amount of glidant and the amount of lubricant applied for preparing a blend may be added concomitantly. Alternatively, preparing a blend may comprise:
mixing the dry granules with an amount of glidant thereby providing a mixture of dry granules and the amount of glidant; and
mixing the mixture of dry granules and the amount of glidant with an amount of lubricant thereby providing a blend.

### 3. Further Embodiments of the Methods of Manufacturing

The embodiments described under this section (“3. Further embodiments of the methods of manufacturing”) below can be combined with any of the embodiments described above under sections “1. Method of manufacturing densified granules” and “2. Method of manufacturing a blend and method of manufacturing an orally deliverable tablet”.

In some embodiments, the pharmaceutically acceptable salt of dexpramipexole is a methane sulfonic acid salt, sulfuric acid salt, tartaric acid salt, p-toluene sulfonic acid salt, phosphoric acid salt, maleic acid salt, fumaric acid salt, malic acid salt, citric acid salt, succinic acid salt, or any combination thereof. In some embodiments, the pharmaceutically acceptable salt of dexpramipexole is dexpramipexole dihydrochloride or a hydrate thereof (such as the monohydrate). In some embodiments, the pharmaceutically acceptable salt of dexpramipexole is dexpramipexole dihydrochloride monohydrate. Further suitable salts for the methods described in this chapter can be found above in chapter “II—Pharmaceutical Composition (Orally Deliverable Tablet),” section “1. Pharmaceutically acceptable salts of dexpramipexole.”

In some embodiments, preparing a pre-blend comprises:
mixing dexpramipexole, or a pharmaceutically acceptable salt thereof, one or more polymers and microcrystalline cellulose (such as silicified microcrystalline cellulose) thereby providing a mixture of dexpramipexole, or a pharmaceutically acceptable salt thereof, the one or more polymers and the microcrystalline cellulose; and
mixing the mixture of dexpramipexole, or a pharmaceutically acceptable salt thereof, the one or more polymers and the microcrystalline cellulose (such as silicified microcrystalline cellulose) with magnesium stearate thereby providing a pre-blend.

In some embodiments, preparing a pre-blend comprises:
mixing dexpramipexole, or a pharmaceutically acceptable salt thereof, one or more anionic polymers and microcrystalline cellulose (such as silicified microcrystalline cellulose) thereby providing a mixture of dexpramipexole, or a pharmaceutically acceptable salt thereof, the one or more anionic polymers and the microcrystalline cellulose; and
mixing the mixture of dexpramipexole, or a pharmaceutically acceptable salt thereof, the one or more anionic polymers and the microcrystalline cellulose (such as silicified microcrystalline cellulose) with magnesium stearate thereby providing a pre-blend.

In some embodiments, the one or more polymers are anionic polymers or neutral polymers. In some embodiments, the one or more polymers are anionic polymers. In some embodiments, the one or more polymers are neutral polymers.

In some embodiments, the densified granules are prepared by dry granulation (such as slugging or roller compaction). In some embodiments, the densified granules are prepared by slugging. In specific embodiments, the densified granules are prepared by roller compaction. Preparing the densified granules by roller compaction was shown herein to be particular robust as it minimized variability and formulation sensitivity (see in particular Examples 4 and 5).

In some embodiments, the densified granules are prepared by roller compaction. In some embodiments, the compaction force applied during roller compaction is about 4 to about 6 kN, such as about 5 kN. In some embodiments, the roll gap applied during roller compaction is about 2 to about 3 mm, such as about 2.5 mm. In some embodiments, the roll speed applied during roller compaction is about 2 to about 4 rpm, such as about 3 rpm. In some embodiments, the roll type applied during roller compaction is a knurled roller. In some embodiments, the tamp/feed auger ratio applied during roller compaction is from about 160% to about 180%, such as about 170%. In some embodiments, the granulator rotor type applied for roller compaction is a star-type rotor. In some embodiments, the granulator rotor speed applied during roller compaction is about 60 rpm/60 rpm to about 80 rpm/80 rpm CW/CCW (clockwise/counter-clockwise), such as about 70 rpm/70 rpm CW/CCW. In some embodiments, the granulator angle applied during roller compaction is about 230°/330° to about 270°/370° C. W/CCW, such as about 250°/350° C. W/CCW. In some embodiments, the agitator speed applied during roller compaction is about 8 to about 12 rpm, such as about 10 rpm. In some embodiments, the roller compactor applied is a Gerteis MiniPactor.

In some embodiments, the densified granules are prepared by roller compaction, wherein (i) the compaction force applied during roller compaction is about 4 to about 6 kN, such as about 5 kN, (ii) the roll gap applied during roller compaction is about 2 to about 3 mm, such as about 2.5 mm, (iii) the roll speed applied during roller compaction is about 2 to about 4 rpm, such as about 3 rpm, (iv) the roll type applied during roller compaction is a knurled roller, (v) the tamp/feed auger ratio applied during roller compaction is from about 160% to about 180%, such as about 170%, (vi) the granulator rotor type applied for roller compaction is a star-type rotor, (vii) the granulator rotor speed applied during roller compaction is about 60 rpm/60 rpm to about 80 rpm/80 rpm CW/CCW (clockwise/counter-clockwise), such as about 70 rpm/70 rpm CW/CCW, (viii) the granulator angle applied during roller compaction is about 230°/330° to about 270°/370° C. W/CCW, such as about 250°/350° C. W/CCW, and (ix) the agitator speed applied during roller compaction is about 8 to about 12 rpm, such as about 10 rpm. The roller compactor applied may be a Gerteis MiniPactor.

In other embodiments, the densified granules are prepared by wet granulation. In some embodiments, the densified granules are prepared by twin screw wet granulation. In some embodiments, the densified granules are prepared by high shear wet granulation. In some embodiments, the densified granules are prepared by low shear wet granulation.

### 4. Products Obtained by the Methods Of Manufacturing

In some aspects, the present disclosure is also directed to densified granules obtained by the method of manufacturing densified granules described above. In some aspects, the present disclosure is also directed to a sustained release orally deliverable pharmaceutical composition (such as a tablet or a capsule) comprising the densified granules obtained by the method of manufacturing densified granules described above.

In some aspects, the present disclosure is also directed to a blend obtained by the method of manufacturing a blend described above.

In some aspects, the present disclosure is also directed to a sustained release pharmaceutical composition in the form of an orally deliverable tablet obtained by the method of manufacturing a sustained release composition in the form of an orally deliverable tablet described above.

### IV—Mixture

In certain aspects, the present disclosure further relates to a mixture comprising dexpramipexole, or a pharmaceutically acceptable salt thereof, constituting about 30% to about 40% of dexpramipexole dihydrochloride monohydrate equivalent by weight of the mixture and about 5% to about 60% of one or more anionic polymers by weight of the mixture.

In certain aspects, the present disclosure further relates to a mixture comprising dexpramipexole, or a pharmaceutically acceptable salt thereof, constituting about 30% to about 40% of dexpramipexole dihydrochloride monohydrate equivalent by weight of the mixture and about 30% to about 55% of one or more anionic polymers by weight of the mixture. In some embodiments, the one or more polymers are anionic polymers or neutral polymers. In some embodiments, the one or more polymers are anionic polymers. In some embodiments, the one or more polymers are neutral polymers.

With the mixture described herein, sustained release pharmaceutical compositions can be prepared (see section “Pharmaceutical composition comprising the mixture” in this chapter below) that are suitable to provide dexpramipexole, or a pharmaceutically acceptable salt thereof, in an amount sufficient to achieve therapeutic and/or prophylactic effects in a human when administered only once per day.

In some embodiments, the mixture as described herein is homogeneous.

In some embodiments, the weight ratio of dexpramipexole dihydrochloride equivalent to the one or more anionic polymers is about 0.2:1 to about 1.3:1, about 0.3:1 to about 1.2:1, about 0.4:1 to about 1.1:1, or about 0.5:1 to about 1:1. In some embodiments, the weight ratio of dexpramipexole dihydrochloride equivalent to the one or more polymers is about 0.2:1 to about 4.0:1, about 0.3:1 to about 3.8:1, about 0.4:1 to about 3.6:1, about 0.5:1 to about 3.5:1, about 0.6:1 to about 3.4:1, about 0.7:1 to about 3.3:1, about 0.8:1 to about 3.2:1, about 0.9:1 to about 3.1:1, about 1.0:1 to about 3.0:1, about 1.1:1 to about 2.9:1, or about 1.2:1 to about 2.8:1. In some embodiments, the weight ratio of dexpramipexole dihydrochloride equivalent to the one or more anionic polymers is about 0.7:1 to about 0.9:1. In some embodiments, the weight ratio of dexpramipexole dihydrochloride equivalent to the one or more polymers is about 0.7:1 to about 0.9:1.

In some embodiments, the weight ratio of dexpramipexole dihydrochloride equivalent to the one or more anionic polymers is about 0.2:1, about 0.3:1, about 0.4:1, about 0.5:1, about 0.6:1, about 0.7:1, about 0.8:1, about 0.9:1, about 1:1, about 1.1:1, about 1.2:1, or about 1.3:1. In some embodiments, the weight ratio of dexpramipexole dihydrochloride equivalent to the one or more polymers is about 0.2:1, about 0.3:1, about 0.4:1, about 0.5:1, about 0.6:1, about 0.7:1, about 0.8:1, about 0.9:1, about 1:1, about 1.1:1, about 1.2:1, about 1.3:1, about 1.4:1, about 1.5:1, about 1.6:1, about 1.7:1, about 1.8:1, about 1.9:1, about 2.0:1, about 2.1:1, about 2.2:1, about 2.3:1, about 2.4:1, about 2.5:1, about 2.6:1, about 2.7:1, about 2.8:1, about 2.9:1, about 3.0:1, about 3.1:1, about 3.2:1, about 3.3:1, about 3.4:1, about 3.5:1, about 3.6:1, about 3.7:1, about 3.8:1, about 3.9:1, or about 4.0:1.

In some embodiments, dexpramipexole, or a pharmaceutically acceptable salt thereof, constitutes about 30% to about 40% of dexpramipexole dihydrochloride monohydrate equivalent by weight of the mixture. In some embodiments, dexpramipexole, or a pharmaceutically acceptable salt thereof, constitutes about 30% to about 50% of dexpramipexole dihydrochloride monohydrate equivalent by weight of the mixture. In some embodiments, dexpramipexole, or a pharmaceutically acceptable salt thereof, constitutes about 35% to about 40% of dexpramipexole dihydrochloride monohydrate equivalent by weight of the mixture. In some embodiments, dexpramipexole, or a pharmaceutically acceptable salt thereof, constitutes about 35% or about 38% of dexpramipexole dihydrochloride monohydrate equivalent by weight of the mixture. In some embodiments, dexpramipexole, or a pharmaceutically acceptable salt thereof, constitutes about 30%, about 31%, about 32%, about 33%, about 34%, about 35%, about 36%, about 37%, about 38%, about 39%, about 40%, about 41%, about 42%, about 43%, about 44%, or about 45% of dexpramipexole dihydrochloride monohydrate equivalent by weight of the mixture.

In some embodiments, the pharmaceutically acceptable salt of dexpramipexole is a methane sulfonic acid salt, sulfuric acid salt, tartaric acid salt, p-toluene sulfonic acid salt, phosphoric acid salt, maleic acid salt, fumaric acid salt, malic acid salt, citric acid salt, succinic acid salt, or any combination thereof. In some embodiments, the pharmaceutically acceptable salt of dexpramipexole is dexpramipexole dihydrochloride or a hydrate thereof (such as the monohydrate). In some embodiments, the pharmaceutically acceptable salt of dexpramipexole is dexpramipexole dihydrochloride monohydrate. Further suitable salts for the mixture described in this chapter can be found above in chapter “II—Pharmaceutical Composition (Orally Deliverable Tablet)”, section “1. Pharmaceutically acceptable salts of dexpramipexole.”

In some embodiments, dexpramipexole, or the pharmaceutically acceptable salt thereof, has a chiral purity for dexpramipexole, or the pharmaceutically salt thereof, of 99.96% or more.

In some embodiments, the one or more anionic polymers constitute about 25% to about 55% by weight of the mixture. In some embodiments, the one or more anionic polymers constitute about 30% to about 50% by weight of the mixture. In some embodiments, the one or more anionic polymers constitute about 35% to about 55% by weight of the mixture. In some embodiments, the one or more anionic polymers constitute about 35% to about 50% by weight of the mixture. In some embodiments, the one or more anionic polymers constitute about 40% to about 50% by weight of the mixture. In some embodiments, the one or more anionic polymers constitute about 35% to about 45%, about 36% to about 44%, about 37% to about 43%, about 38% to about 42% or about 39% to about 41% by weight of the mixture. In some embodiments, the one or more anionic polymers constitute about 40% by weight of the mixture. In some embodiments, the one or more anionic polymers constitute about 42% to about 52%, about 43% to about 51%, about 44% to about 50%, about 45% to about 49%, or about 46% to about 48% by weight of the mixture. In some embodiments, the one or more anionic polymers constitute about 47% by weight of the mixture. In some embodiments, the one or more anionic polymers constitute about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, or about 55% by weight of the mixture.

In some embodiments, the one or more anionic polymers are selected from carboxymethylcellulose, an acrylic acid polymer, a methacrylic acid polymer, a methacrylic acid copolymer, alignate, carrageenan, xanthan gum, or arabic gum. In some embodiments, the one or more anionic polymers are hydrophilic anionic polymers. In some embodiments, the one or more anionic polymers are selected from carboxymethylcellulose and an acrylic acid polymer. The carboxymethylcellulose may be sodium carboxymethylcellulose. An example of a sodium carboxymethylcellulose is Aqualon CMC 7HXF PH. In more specific embodiments, the viscosity of the sodium carboxymethylcellulose is about 1,500 to about 3,000 cP (centipoise; 1% in water). The acrylic acid polymer may be a crosslinked acrylic acid polymer. An example of a crosslinked acrylic acid polymer is Carbopol 971P. In more specific embodiments, the viscosity of the crosslinked acrylic acid polymer is about 4,000 to about 11,000 cP (centipoise; 0.5% at pH 7.5).

In some embodiments, the mixture comprises one, but no more than one anionic polymer. In specific embodiments, the mixture comprises one, but no more than one anionic polymer and the anionic polymer is carboxymethylcellulose, such as sodium carboxymethylcellulose. In specific embodiments, the mixture comprises one, but no more than one anionic polymer and the anionic polymer is an acrylic acid polymer, such as crosslinked acrylic acid polymer.

In some embodiments, the amount of carboxymethylcellulose is about 23% to about 33%, about 24% to about 32%, about 25% to about 31%, about 26% to about 30%, about 27% to about 29% of sodium carboxymethylcellulose equivalent by weight of the mixture. In specific embodiments, the amount of carboxymethylcellulose is about 28% of sodium carboxymethylcellulose equivalent by weight of the mixture.

In some embodiments, the mixture comprises more than one anionic polymer, such as two, three, four, or five anionic polymers. Within the meaning of the present disclosure, if the mixture comprises more than one anionic polymer, such as a first and a second anionic polymer, the anionic polymers are different anionic polymers. For example, a mixture comprising two anionic polymers might comprise sodium carboxymethylcellulose as the first anionic polymer and an acrylic acid polymer as the second anionic polymer.

In some embodiments, the mixture comprises one or more, but no more than five anionic polymers. In some embodiments, the mixture comprises one or more, but no more than four anionic polymers. In some embodiments, the mixture comprises one or more, but no more than three anionic polymers. In some embodiments, the mixture comprises one or more, but no more than two anionic polymers.

In some embodiments, the mixture comprises two, but no more than two anionic polymers. In specific embodiments, the mixture comprises two, but no more than two anionic polymers and the two anionic polymers are carboxymethylcellulose (such as sodium carboxymethylcellulose) and an acrylic acid polymer (such as a crosslinked acrylic acid polymer).

In some embodiments, the mixture consists essentially of dexpramipexole, or a pharmaceutically acceptable salt thereof, constituting about 30% to about 40% of dexpramipexole dihydrochloride monohydrate equivalent by weight of the mixture and one or more, but no more than two anionic polymers at the amounts specified herein (such as at about 30% to about 50% by weight of the mixture). The two anionic polymers may be carboxymethylcellulose (such as sodium carboxymethylcellulose) and an acrylic acid polymer (such as a crosslinked acrylic acid polymer).

In some embodiments, the mixture consists essentially of dexpramipexole, or a pharmaceutically acceptable salt thereof, constituting about 30% to about 40% of dexpramipexole dihydrochloride monohydrate equivalent by weight of the mixture and two anionic polymers at the amounts specified herein (such as at about 30% to about 50% by weight of the mixture). The two anionic polymers may be carboxymethylcellulose (such as sodium carboxymethylcellulose) and an acrylic acid polymer (such as a crosslinked acrylic acid polymer).

In some embodiments, the mixture comprises a first anionic polymer and a second anionic polymer, wherein the first anionic polymer is carboxymethylcellulose and the second anionic polymer is an acrylic acid polymer. The carboxymethylcellulose may be sodium carboxymethylcellulose and/or the acrylic acid polymer may be a crosslinked acrylic acid polymer. In some embodiments, carboxymethylcellulose constitutes about 23% to about 37% of sodium carboxymethylcellulose equivalent by weight of the mixture and the acrylic acid polymer constitutes about 7% to about 18% by weight of the mixture. In some embodiments, the amount of carboxymethylcellulose is about 23% to about 33%, about 24% to about 32%, about 25% to about 31%, about 26% to about 30%, about 27% to about 29% of sodium carboxymethylcellulose equivalent by weight of the mixture and the amount of the acrylic acid polymer is about 7% to about 16%, about 8% to about 15%, about 10% to about 14%, or about 11% to about 13% by weight of the mixture. In specific embodiments, the amount of carboxymethylcellulose is about 28% of sodium carboxymethylcellulose equivalent by weight of the mixture and the amount of the acrylic acid polymer is about 12% by weight of the mixture. In some embodiments, the amount of carboxymethylcellulose is about 25% to about 35%, about 26% to about 34%, about 27% to about 33%, about 28% to about 32%, about 29% to about 31% of sodium carboxymethylcellulose equivalent by weight of the mixture and the amount of the acrylic acid polymer is about 8% to about 18%, about 9% to about 17%, about 10% to about 16%, or about 11% to about 15% by weight of the mixture. In specific embodiments, the amount of carboxymethylcellulose is about 30% of sodium carboxymethylcellulose equivalent by weight of the mixture and the amount of the acrylic acid polymer is about 13% by weight of the mixture. In other embodiments, the amount of carboxymethylcellulose is about 29% to about 37%, about 30% to about 36%, about 31% to about 35%, or about 32% to about 34% of sodium carboxymethylcellulose equivalent by weight of the mixture and the amount of the acrylic acid polymer is about 10% to about 18%, about 11% to about 17%, about 12% to about 16%, or about 13% to about 15% by weight of the mixture. In other specific embodiments, the amount of carboxymethylcellulose is about 33% of sodium carboxymethylcellulose equivalent by weight of the blend and the amount of the acrylic acid polymer is about 14% by weight of the mixture. In some embodiments, the weight ratio of the acrylic acid polymer to sodium carboxymethylcellulose equivalent in the mixture is about 3:2 to about 2:8. More specific, the weight ratio of the acrylic acid polymer to sodium carboxymethylcellulose equivalent is about 2:3 to about 2:8. In some embodiments, the weight ratio of the acrylic acid polymer to sodium carboxymethylcellulose equivalent is about 3:2, about 3:3 (1:1), about 3:4, about 3:5, about 3:6, about 3:7, about 3:8, about 3:9, about 2:3, about 2:4, about 2:5, about 2:6, about 2:7, or about 2:8. More specific, the weight ratio of the acrylic acid polymer to sodium carboxymethylcellulose equivalent is about 3:7. In some of the embodiments described in this paragraph, the mixture comprises the first and the second anionic polymer, but no more other anionic polymers than the first and the second anionic polymer. In some of the embodiments described in this paragraph, the mixture consists essentially of dexpramipexole, or a pharmaceutically acceptable salt and the first and the second anionic polymer.

In some embodiments, the mixture consists essentially of dexpramipexole, or a pharmaceutically acceptable salt thereof, and two anionic polymers.

In some embodiments, the mixture comprises the amount of dexpramipexole, or a pharmaceutically acceptable salt thereof, and the one or more anionic polymers in the form of densified granules. Other ingredients such as a diluent or a lubricant may be part of the densified granules. The densified granules may be obtained by roller compaction. A suitable roller compaction process is described, for example, above in the specification in Chapter “III-Methods of Manufacturing,” section “3. Further embodiments of the methods of manufacturing”.

In some embodiments, the mixture in addition to the amount of dexpramipexole, or the pharmaceutically acceptable salt and the one or more anionic polymers at the amounts specified herein further comprises one or more hydrophilic neutral polymers, and/or one or more lipophilic materials. In some embodiments, the mixture in addition to the amount of dexpramipexole, or the pharmaceutically acceptable salt at the amounts specified herein further comprises one or more hydrophilic neutral polymers, and/or one or more lipophilic materials. The one or more hydrophilic neutral polymers may be hypromellose (hydroxypropyl methylcellulose, HPMC), silicified hydroxypropyl methylcellulose, hydroxypropyl cellulose (HPC) and/or poly(ethylene oxide), and the one or more lipophilic materials may be glycerol dibehenate and/or carnuba wax.

In some embodiments, the one or more polymers are neutral polymers. In some embodiments, the one or more neutral polymers constitute about 5% to about 25% by weight of the mixture. In some embodiments, the one or more neutral polymers constitutes about 10% to about 20%, about 11% to about 19%, about 12% to about 18%, about 13% to about 17%, or about 14% to about 16% by weight of the mixture. In specific embodiments, the one or more neutral polymers constitute about 15% by weight of the mixture.

In some specific embodiments, the one or more neutral polymers are hypromellose (hydroxypropyl methylcellulose, HPMC), silicified hydroxypropyl methylcellulose, hydroxypropyl cellulose (HPC), and/or poly(ethylene oxide). Non-limiting examples of a HPMC are Methocel® K15M, Methocel® K100M, and Methocel® K200M. A non-limiting example of a HPC is Klucel® Xtend HXF. A non-limiting example of poly(ethylene oxide) is Sentry Polyox® WSR-303.

In some embodiments, the one or more neutral polymers are hydroxypropyl methylcellulose or silicified hydroxypropyl methylcellulose. In some embodiments, the hydroxypropyl methylcellulose or silicified hydroxypropyl methylcellulose constitutes about 5% to about 25% by weight of the mixture. In some embodiments, the hydroxypropyl methylcellulose constitutes or silicified hydroxypropyl methylcellulose about 10% to about 20%, about 11% to about 19%, about 12% to about 18%, about 13% to about 17%, or about 14% to about 16% by weight of the mixture. In specific embodiments, the hydroxypropyl methylcellulose or silicified hydroxypropyl methylcellulose constitutes about 15% by weight of the mixture.

In some embodiments, the amount of hydroxypropyl methylcellulose or silicified hydroxypropyl methylcellulose is about 5% and about 60% by weight of the densified granules. In some embodiments, the amount of hydroxypropyl methylcellulose or silicified hydroxypropyl methylcellulose is about 10% and about 60% by weight of the densified granules. In some embodiments, the one or more neutral polymers constitute about 15% to about 60% by weight of the densified granules. In some embodiments, the amount of hydroxypropyl methylcellulose or silicified hydroxypropyl methylcellulose is about 20% and about 60% by weight of the densified granules. In some embodiments, the one or more neutral polymers constitute about 25% to about 60% by weight of the densified granules. In some embodiments, the amount of hydroxypropyl methylcellulose or silicified hydroxypropyl methylcellulose is about 30% and about 60% by weight of the densified granules. In some embodiments, the one or more neutral polymers constitute about 35% to about 60% by weight of the densified granules. In some embodiments, the amount of hydroxypropyl methylcellulose or silicified hydroxypropyl methylcellulose is about 40% and about 60% by weight of the densified granules. In some embodiments, the one or more neutral polymers constitute about 45% to about 60% by weight of the densified granules. In some embodiments, the amount of hydroxypropyl methylcellulose or silicified hydroxypropyl methylcellulose is about 50% and about 60% by weight of the densified granules. In some embodiments, the one or more neutral polymers constitute about 55% to about 60% by weight of the densified granules.

In some embodiments, the amount of hydroxypropyl methylcellulose or silicified hydroxypropyl methylcellulose is about 5% to about 55% by weight of the densified granules. In some embodiments, the amount of hydroxypropyl methylcellulose or silicified hydroxypropyl methylcellulose is about 5% to about 50% by weight of the densified granules. In some embodiments, the amount of hydroxypropyl methylcellulose or silicified hydroxypropyl methylcellulose is about 5% to about 45% by weight of the densified granules. In some embodiments, the amount of hydroxypropyl methylcellulose or silicified hydroxypropyl methylcellulose is about 5% to about 40% by weight of the densified granules. In some embodiments, the amount of hydroxypropyl methylcellulose or silicified hydroxypropyl methylcellulose is about 5% to about 35% by weight of the densified granules. In some embodiments, the amount of hydroxypropyl methylcellulose or silicified hydroxypropyl methylcellulose is about 5% to about 30% by weight of the densified granules.

In some embodiments, the amount of hydroxypropyl methylcellulose or silicified hydroxypropyl methylcellulose is about 20% by weight of the densified granules. In some embodiments, the amount of hydroxypropyl methylcellulose or silicified hydroxypropyl methylcellulose is about 25% by weight of the densified granules. In some embodiments, the amount of hydroxypropyl methylcellulose or silicified hydroxypropyl methylcellulose is about 30% by weight of the densified granules. In some embodiments, the amount of hydroxypropyl methylcellulose or silicified hydroxypropyl methylcellulose is about 35% by weight of the densified granules. In some embodiments, the amount of hydroxypropyl methylcellulose or silicified hydroxypropyl methylcellulose is about 40% by weight of the densified granules. In some embodiments, the amount of hydroxypropyl methylcellulose or silicified hydroxypropyl methylcellulose is about 45% by weight of the densified granules. In some embodiments, the amount of hydroxypropyl methylcellulose or silicified hydroxypropyl methylcellulose is about 50% by weight of the densified granules. In some embodiments, the amount of hydroxypropyl methylcellulose or silicified hydroxypropyl methylcellulose is about 55% by weight of the densified granules. In some embodiments, the amount of hydroxypropyl methylcellulose or silicified hydroxypropyl methylcellulose is about 60% by weight of the densified granules.

In some embodiments, the mixture comprises one, but no more than one neutral polymer. In specific embodiments, the mixture comprises one, but no more than one neutral polymer and the neutral polymer is hydroxypropyl methylcellulose or silicified hydroxypropyl methylcellulose.

In some embodiments, the mixture comprises more than one neutral polymer, such as two, three, four, or five neutral polymers. Within the meaning of the present disclosure, if the mixture comprises more than one neutral polymer, such as a first and a second neutral polymer, the neutral polymers are different neutral polymers. For example, a mixture comprising two neutral polymers might comprise hydroxypropyl methylcellulose or silicified hydroxypropyl methylcellulose as the first polymer.

In some embodiments, the mixture comprises one or more, but no more than five neutral polymers. In some embodiments, the mixture comprises one or more, but no more than four neutral polymers. In some embodiments, the mixture comprises one or more, but no more than three neutral polymers. In some embodiments, the mixture comprises one or more, but no more than two neutral polymers.

In some embodiments, the mixture consists essentially of dexpramipexole, or a pharmaceutically acceptable salt thereof, constituting about 30% to about 40% of dexpramipexole dihydrochloride monohydrate equivalent by weight of the mixture and one or more, but no more than two neutral polymers at the amounts specified herein (such as at about 5% to about 25% by weight of the mixture). If two neutral polymers are present, the first neutral polymer may be hydroxypropyl methylcellulose or silicified hydroxypropyl methylcellulose. In some embodiments, hydroxypropyl methylcellulose or silicified hydroxypropyl methylcellulose constitutes about 5% to about 25% by weight of the mixture. In some embodiments, hydroxypropyl methylcellulose or silicified hydroxypropyl methylcellulose constitutes about 10% to about 20%, about 11% to about 19%, about 12% to about 18%, about 13% to about 17%, or about 14% to about 16% by weight of the mixture. In specific embodiments, hydroxypropyl methylcellulose or silicified hydroxypropyl methylcellulose constitutes about 15% by weight of the mixture.

In some embodiments, the mixture further comprises a diluent (such as silicified microcrystalline cellulose), a lubricant (such as magnesium stearate), and/or a glidant (such as colloidal silicon dioxide). In some embodiments, the diluent constitutes about 10% to about 25% (such as about 15% to about 20%) by weight of the mixture. In some embodiments, the lubricant constitutes about 0.5% to about 1% (such as about 0.75%) by weight of the mixture. In some embodiments, the glidant constitutes about 0.25% to about 0.75% (such as about 0.5%) by weight of the mixture.

In some embodiments, the mixture in addition to the amount of dexpramipexole, or the pharmaceutically acceptable salt (at about 30% to about 40% dexpramipexole dihydrochloride monohydrate equivalent by weight of the mixture) and the one or more anionic polymers at the amounts specified herein (such as at about 40% to about 50% by weight of the mixture) further comprises about 10% to about 25% diluent (such as silicified microcrystalline cellulose) by weight of the mixture, about 0.5% to about 1% lubricant (such as magnesium stearate) by weight of the mixture, and about 0.25% to about 0.75% glidant (such as colloidal silicon dioxide) by weight of the mixture.

In some embodiments, the mixture in addition to the amount of dexpramipexole, or the pharmaceutically acceptable salt (at about 30% to about 50% dexpramipexole dihydrochloride monohydrate equivalent by weight of the mixture) further comprises about 10% to about 25% diluent (such as silicified microcrystalline cellulose) by weight of the mixture, about 0.5% to about 1% lubricant (such as magnesium stearate) by weight of the mixture, and about 0.25% to about 0.75% glidant (such as colloidal silicon dioxide) by weight of the mixture.

In some embodiments, the mixture further comprises a microcrystalline cellulose, magnesium stearate, colloidal silicon dioxide, or any combination thereof. In specific embodiments, the mixture further comprises a microcrystalline cellulose, magnesium stearate, and colloidal silicon dioxide. The microcrystalline cellulose may be silicified microcrystalline cellulose.

In some embodiments, the mixture comprises 0.02% or less pramipexole, or a pharmaceutically acceptable salt thereof, by weight of the mixture. In specific embodiments, the mixture comprises 0.015% or less pramipexole, or a pharmaceutically acceptable salt thereof, by weight of the mixture.

In some embodiments, the mixture does not comprise pramipexole, or a pharmaceutically acceptable salt thereof, above the detection limit when analyzed with high performance liquid chromatography (HPLC).

### Pharmaceutical Composition Comprising the Mixture

The present disclosure further relates to a sustained release pharmaceutical composition comprising the mixture as described above in this chapter (“IV—Mixture”). In some embodiments, the sustained release pharmaceutical composition is orally deliverable. In some embodiments, the sustained release pharmaceutical composition is a composition that can be delivered sublingual or buccal.

In some embodiments, the composition comprises dexpramipexole, or a pharmaceutically acceptable salt thereof, in an amount of about 50 mg to about 400 mg of dexpramipexole dihydrochloride equivalent. In some embodiments, the composition comprises dexpramipexole, or a pharmaceutically acceptable salt thereof, in an amount of about 75 mg to about 300 mg of dexpramipexole dihydrochloride equivalent. In specific embodiments, the amount of dexpramipexole, or a pharmaceutically acceptable salt thereof, is about 75 mg of dexpramipexole dihydrochloride equivalent. In other specific embodiments, the amount of dexpramipexole, or a pharmaceutically acceptable salt thereof, is about 150 mg of dexpramipexole dihydrochloride equivalent. In yet other specific embodiments, the amount of dexpramipexole, or a pharmaceutically acceptable salt thereof, is about 300 mg of dexpramipexole dihydrochloride equivalent. In yet further specific embodiments, the amount of dexpramipexole, or a pharmaceutically acceptable salt thereof, is about 376 mg of dexpramipexole dihydrochloride equivalent. In some embodiments, the pharmaceutically acceptable salt of dexpramipexole is dexpramipexole dihydrochloride monohydrate.

In some embodiments, the amount of dexpramipexole, or a pharmaceutically acceptable salt thereof, constitutes about 25% to about 45% of dexpramipexole dihydrochloride monohydrate equivalent by weight of the composition. In some embodiments, the amount of dexpramipexole, or a pharmaceutically acceptable salt thereof, constitutes about 30% to about 40% of dexpramipexole dihydrochloride monohydrate equivalent by weight of the composition.

Further suitable amounts of dexpramipexole, or a pharmaceutically acceptable salt thereof, for the composition comprising the mixture described in this section are described above in chapter “II—Pharmaceutical Composition (Orally Deliverable Tablet),” section “2. Amount of dexpramipexole, or a pharmaceutically acceptable salt thereof.”

In some embodiments, the pharmaceutical composition comprises 0.02% or less pramipexole, or a pharmaceutically acceptable salt thereof, by weight of the composition. In specific embodiments, the composition comprises 0.015% or less pramipexole, or a pharmaceutically acceptable salt thereof, by weight of the composition.

In some embodiments, the pharmaceutical composition does not comprise pramipexole, or a pharmaceutically acceptable salt thereof, above the detection limit when analyzed with high performance liquid chromatography (HPLC).

In some embodiments, the weight of the composition is about 1500 mg or less. The “weight of the composition” refers to the weight of the entire composition (e.g., in case of a tablet, the weight also includes any (non-functional) coating that might be present). In some embodiments, the weight of the composition is about 1300 mg or less. In some embodiments, the weight of the composition is about 1000 mg or less. In some embodiments, the weight of the composition is about 800 mg to about 1000 mg. Further suitable weights for the composition comprising the mixture described in this section are described above in chapter “II—Pharmaceutical Composition (Orally Deliverable Tablet),” section “5. Further embodiments of the pharmaceutical composition” in the context of the orally deliverable tablet described therein.

In some embodiments, the pharmaceutical composition is capable of providing a sigmoidal, pseudo-zero order, or zero order release of dexpramipexole, or a pharmaceutically acceptable salt thereof. In specific embodiments, the release is a zero order release.

In specific embodiments, the amount of dexpramipexole, or a pharmaceutically acceptable salt thereof, in the pharmaceutical composition is about 50 mg to about 400 mg (such as about 75 mg, about 150 mg, about 300 mg, or about 376 mg) dexpramipexole dihydrochloride equivalent and the one or more polymers constitute about 5% to about 60% (such as about 10% to about 55%) by weight of the mixture. In other specific embodiments, the amount of dexpramipexole, or a pharmaceutically acceptable salt thereof, in the pharmaceutical composition is about 50 mg to about 350 mg (such as about 75 mg, about 150 mg, or about 300 mg) dexpramipexole dihydrochloride equivalent and the one or more anionic polymers constitute about 30% to about 55% (such as about 30% to about 50%) by weight of the mixture. In these specific embodiments, the one or more polymers are anionic polymers, and the one or more anionic polymers may be a carboxymethylcellulose and/or a crosslinked acrylic acid polymer. In other specific embodiments, the one or more polymers are neutral polymers, and the one or more neutral polymers may be hydroxypropyl methylcellulose or silicified hydroxypropyl methylcellulose. In the specific embodiments described in this paragraph, the pharmaceutical composition may comprise 0.015% or less pramipexole, or a pharmaceutically acceptable salt thereof, by weight of the mixture. The weight of the composition may be about 1000 mg or less (such as about 800 mg to about 1000 mg).

In specific embodiments, the amount of dexpramipexole, or a pharmaceutically acceptable salt thereof, in the pharmaceutical composition is about 75 mg to about 300 mg (such as about 75 mg, about 150 mg, or about 300 mg) dexpramipexole dihydrochloride equivalent and the one or more anionic polymers constitute about 30% to about 50% (such as about 40% to about 50%) by weight of the mixture. In these specific embodiments, the one or more anionic polymers may be a carboxymethylcellulose and/or a crosslinked acrylic acid polymer. In the specific embodiments described in this paragraph, the pharmaceutical composition may comprise 0.015% or less pramipexole, or a pharmaceutically acceptable salt thereof, by weight of the mixture. The weight of the composition may be about 1000 mg or less (such as about 800 mg to about 1000 mg).

In some embodiments, the composition is an orally deliverable capsule. In specific embodiments, the orally deliverable capsule comprises the mixture enclosed in one or more beads. The orally deliverable capsule comprises a number of beads that results in the amounts of dexpramipexole, or a pharmaceutically acceptable salt thereof, as described above (e.g., about 75 mg to about 300 mg, such as about 75 mg, about 150 mg, or about 300 mg, dexpramipexole dihydrochloride equivalent). The orally deliverable capsule comprises a number of beads that results in the amounts of dexpramipexole, or a pharmaceutically acceptable salt thereof, as described above (e.g., about 75 mg to about 400 mg, such as about 75 mg, about 150 mg, about 300 mg, or about 376 mg dexpramipexole dihydrochloride equivalent). For example, if each bead comprises about 10 mg dexpramipexole dihydrochloride equivalent, the capsule comprises 30 beads to result in a total dose of about 300 mg dexpramipexole dihydrochloride equivalent.

In some embodiments, the present disclosure is also directed to a pharmaceutical composition comprising the mixture as described herein in the form of a powder or a (granulation) sprinkle. Powders or (granulation) sprinkles are in particular intended for patients who have difficulties with swallowing (for example, patients with Parkinson's disease, multipole sclerosis, and/or epilepsy). In some embodiments, the composition is a capsule comprising the powder or the (granulation) sprinkle. Such a capsule can be designed to be easily opened by patients and care-givers, enabling them to dose the contents of the capsule in a consistent manner. The powder or (granulation) sprinkle can be, for example, applied on food or in beverages. See, for example, Han Sol et al., 2020, Asian Journal of Pharmaceutical Sciences, volume 15, issue 3, pages 292-310 (a review on sprinkle formulations).

In other embodiments, the composition is an orally deliverable tablet. The orally deliverable tablet may comprise the mixture in a tablet core. The tablet core may be surrounded by a film coating as described above in chapter “II—Pharmaceutical Composition (Orally Deliverable Tablet),” section “4. Film Coating.”

In addition to the embodiments expressly described above in this chapter (“IV—Mixture”) above, reference is generally also made to additional embodiments as described in chapter “II—Pharmaceutical Composition (Orally Deliverable Tablet)” above. As will be understood by a person of ordinary skill in the art, in embodiments described above in chapter “II—Pharmaceutical Composition (Orally Deliverable Tablet)” wherein reference is made to “by weight of the tablet core,” the corresponding embodiments in the context of the mixture described in this chapter above (“IV—Mixture”) are intended to refer to “by weight of the mixture”.

In some embodiments, the pharmaceutical composition can be part of a kit comprising the pharmaceutical composition packaged into a container (which might be, for example, a blister package or a bottle), the container being accompanied by a package insert providing pertinent information such as, for example, dosage and administration information, contraindications, precautions, drug interactions and adverse reactions. In some embodiments, the kit further comprises a desiccant. A desiccant is a hygroscopic substance that is used to induce or sustain a state of dryness (desiccation) in its vicinity. Examples of desiccants are silica gel, activated charcoal, calcium sulfate, calcium chloride, and molecular sieves (typically, zeolites).

### V—Methods of Treatment or Prevention

In certain aspects, the present disclosure further relates to methods of treating or preventing a disorder, disease, or condition in a human subject in need thereof, the methods comprising orally administering to the human subject the pharmaceutical composition of the present disclosure. Regarding the pharmaceutical composition as referred to in this chapter (“V—Methods of Treatment or Prevention”) below, reference is made to the pharmaceutical composition as described herein (in particular, as described in the chapter “II—Pharmaceutical Composition (Orally Deliverable Tablet)” above and in the chapter “IV—Mixture,” section “Pharmaceutical composition comprising the mixture” above) (briefly indicated below by reference to “the pharmaceutical composition described herein”). The term “orally administering” as used herein may include the act of self-administration by a human in need thereof or administration by another person such as a health care provider.

In some embodiments, the present disclosure is directed to a method of treating or preventing a respiratory disease in a human subject in need thereof, the method comprising orally administering to the human subject the pharmaceutical composition described herein.

In some embodiments, the present disclosure is directed to a method of treating or preventing asthma in a human subject in need thereof, the method comprising orally administering to the human subject the pharmaceutical composition described herein. Asthma is a heterogeneous disease, usually characterized by chronic airway inflammation. Asthma can be defined by the history of respiratory symptoms such as wheeze, shortness of breath, chest tightness, and cough that vary over time and in intensity, together with expiratory airflow limitation. Asthma is a condition in which the airways narrow due to airways smooth muscle constriction and mucosal swelling. The increase of mucus production in the airway lumen further contributes to obstructing airflow and worsening asthma symptoms. This can make breathing difficult and trigger coughing, wheezing and shortness of breath. Asthma may lead to life-threatening asthma attacks or exacerbations.

In some embodiments, treating asthma comprises a reduction in frequency of asthma exacerbations in the human subject.

In some embodiments, treating asthma comprises that the human subject shows improvement in a measurement selected from the group consisting of forced expiratory volume in 1 second (FEVI), forced vital capacity (FVC), annualized CompEx event rate, morning peak expiratory flow (PEF), score on Asthma Control Questionnaire (ACQ), score of Asthma Quality of Life Questionnaire (AQLQ), score on St. George's Respiratory Questionnaire, and any combinations thereof.

In some embodiments, the asthma is severe asthma. Severe asthma is defined as asthma that is uncontrolled on GINA Step 4 or 5 therapy. “GINA” refers to “Global Initiative For Asthma.”

In some embodiments, the asthma is eosinophilic asthma. Eosinophilic asthma (or “asthma of the eosinophilic phenotype”) is a form of asthma that involves abnormally high levels of eosinophils in the blood or in the lungs. A human subject having eosinophilic asthma has an eosinophil level of at least 150 cells per microliter in the peripheral blood. In some embodiments, the eosinophil level is at least 300 cells per microliter in the peripheral blood.

In some embodiments, the eosinophilic asthma is moderate eosinophilic asthma. In some embodiments, the eosinophilic asthma is severe eosinophilic asthma.

In some embodiments, the present disclosure is directed to a method of treating or preventing chronic obstructive pulmonary disease (COPD) in a human subject in need thereof, the method comprising orally administering to the human subject the pharmaceutical composition described herein. In some embodiments, the chronic obstructive pulmonary disease is emphysema or chronic bronchitis.

In some embodiments, the present disclosure is directed to a method of treating or preventing an eosinophilic disorder (i.e., disorders involving elevated levels of eosinophils) in a human subject in need thereof, the method comprising orally administering to the human subject the pharmaceutical composition described herein.

In some embodiments, the eosinophilic disorder is selected from the group consisting of hypereosinophilic syndrome, chronic rhinosinusitis with nasal polyps, nasal polyposis, atopic dermatitis, eosinophilic granulomatosis with polyangiitis, eosinophilic gastroenteritis, eosinophilic esophagitis, and any combination thereof.

In some embodiments, the present disclosure is directed to a method of treating or preventing eosinophilic granulomatosis with polyangiitis in a human subject in need thereof, the method comprising orally administering to the human subject the pharmaceutical composition described herein. “Eosinophilic granulomatosis with polyangiitis” is also known in the art as Churg-Strauss syndrome.

In some embodiments, the present disclosure is directed to a method of treating or preventing hypereosinophilic syndrome in a human subject in need thereof, the method comprising orally administering to the human subject the pharmaceutical composition described herein. In some embodiments, hypereosinophilic syndrome is characterized by levels of eosinophils at or above about 1,000 cells per microliter in the peripheral blood. In some embodiments, hypereosinophilic syndrome is characterized by levels of eosinophils at or above about 1,500 cells per microliter in the peripheral blood.

In some embodiments, the disclosure is directed to a method of treating or preventing an ear, nose, throat or eye disease in a human subject in need thereof, the method comprising orally administering to the human subject the pharmaceutical composition described herein.

In some embodiments, the disclosure is directed to a method of treating or preventing chronic rhinosinusitis with nasal polyps in a human subject in need thereof, the method comprising orally administering to the human subject the pharmaceutical composition described herein.

In some embodiments, the present disclosure as directed to a method of treating or preventing a gastrointestinal disease in a human subject in need thereof, the method comprising orally administering to the human subject the pharmaceutical composition described herein.

In some embodiments, the present disclosure as directed to a method of treating or preventing eosinophilic esophagitis in a human subject in need thereof, the method comprising orally administering to the human subject the pharmaceutical composition described herein.

In some embodiments, the present disclosure as directed to a method of treating or preventing eosinophilic gastroenteritis in a human subject in need thereof, the method comprising orally administering to the human subject the pharmaceutical composition described herein.

In specific embodiments, the pharmaceutical composition is administered once daily. Once daily is also referred to herein as “quaque die”/“QD” or “once per day”. “Once daily” administration as used herein refers to administration about every 24 hours.

In some embodiments, the pharmaceutical composition is administered about every 22 to 26 hours. In some embodiments, the pharmaceutical composition is administered about every 23 to 25 hours.

In some embodiments, the pharmaceutical composition is administered to the subject as maintenance therapy (e.g., administration might occur over a period of several years, or over the lifetime of the human subject).

In some embodiments, the pharmaceutical composition is administered about 2 hours after the human subject has consumed a meal. In some embodiments, the pharmaceutical composition is administered orally with about 200 mL to about 300 mL of water.

In some embodiments, the present disclosure is directed to the pharmaceutical composition described herein (in particular as described above in chapter “II—Pharmaceutical Composition (Orally Deliverable Tablet)” or chapter “IV—Mixture,” section “Pharmaceutical composition comprising the mixture”) for use as a medicament.

In some embodiments, the present disclosure is directed to the pharmaceutical composition described herein (in particular, described above in chapter “II—Pharmaceutical Composition (Orally Deliverable Tablet)” or chapter “IV—Mixture,” section “Pharmaceutical composition comprising the mixture”) for use in a method of treating or preventing as described herein (in particular, described above in this chapter (“V—Methods of Treatment or Prevention”)).

In some embodiments, the present disclosure is directed to the use of the pharmaceutical composition described herein (in particular, described above in chapter “II—Pharmaceutical Composition (Orally Deliverable Tablet)” or chapter “IV—Mixture,” section “Pharmaceutical composition comprising the mixture”) in the manufacture of a medicament for a method of treating or preventing as described herein (in particular, described above in this chapter (“V—Methods of Treatment or Prevention”)).

### EXAMPLES

The following examples are intended solely as an illustration and should not be regarded as restricting the disclosure in any way.

In the examples below, orally deliverable matrix tablets comprising dexpramipexole dihydrochloride monohydrate have been manufactured. In vitro dissolution profiles of the manufactured tablets were compared to Mirapex® extended release (ER) tablets, comprising 4.5 mg of pramipexole dihydrochloride monohydrate. Inactive ingredients of the pramipexole extended release tablets are, according to the manufacturer, hypromellose, corn starch, carbomer homopolymer, colloidal silicon dioxide, and magnesium stearate.

Dissolution profiles were evaluated using USP <711> Dissolution Apparatus I (Baskets) and II (Paddles) in 900 mL of solvent, equilibrated to 37.0±0.5° C., with agitation at 100 rpm (Baskets, B) or 50 rpm (Paddles, P), respectively, for 24 hours. Samples were collected after 1, 2, 4, 6, 8, 10, 12, 16, 20 and 24 hours. The analytical finish method employed isocratic reversed phase HPLC with UV detection at 265 nm to resolve the active peak. Following confirmation of system suitability, quantitation of the percentage released was achieved by comparing the dexpramipexole peak area in the sample solution chromatograms to the mean dexpramipexole peak area in standard solution chromatograms, correcting for the quantity of drug substance removed during sampling at earlier time points. Different solvents were used for dissolution studies, including 0.1 N HCl, McIlvaine Buffer, pH 4.5, and 50 mM monobasic potassium phosphate buffer, pH 6.8. As known in the art, McIlvaine Buffer is made from citric acid and disodium hydrogen phosphate.

In Examples 1 to 3, the different tablet batches were manufactured each at a smaller scale of 15 tablets (12.75 g blend) unless otherwise indicated with the objective to evaluate dissolution. The manufacturing process was scaled up in Examples 4 and 5 once a suitable sustained release matrix system was identified.

319 mg dexpramipexole dihydrochloride monohydrate is equivalent to 300 mg dexpramipexole dihydrochloride.

400 mg dexpramipexole dihydrochloride monohydrate is equivalent to 376 mg dexpramipexole dihydrochloride.

In the Examples below, dexpramipexole dihydrochloride monohydrate comprising 0.04% or less of pramipexole dihydrochloride monohydrate was applied (i.e., the chiral purity for dexpramipexole dihydrochloride monohydrate was at least 99.96%). Stability data indicated that the chiral purity did not change over time during tablet manufacture and storage.

The Examples below demonstrate that tablets formulated according to the present disclosure (Examples 3-9) are suitable for once daily oral administration to a human, in contrast to Examples 1 and 2 in which the tablets were not formulated according to the present disclosure.

### Example 1—Hydrophilic Neutral Polymers

First, the feasibility of hydrophilic neutral polymers as matrix components was evaluated. In particular, hypromellose (hydroxypropyl methylcellulose; K15M, K100M, K200M), hydroxypropyl cellulose (Klucel Xtend HXF), and poly(ethylene oxide) (Sentry PolyOx WSR-303) were evaluated. A comparison of these polymers is provided in Table 1. Also different manufacturing processes were evaluated, i.e., direct compression (DC) and twin screw wet-granulation (TSWG).

TABLE 1
Comparison of hydrophilic neutral polymers evaluated in matrix tablets.

| Polymer | Grade | Nominal Viscosity | Structure |
|---|---|---|---|
| Hypromellose/ Hydroxypropyl methylcellulose (HPMC) | Methocel K15M Methocel K100M Benecel K200M | ~15,000 cP (2% in water) ~100,000 cP (2% in water) ~200,000 cP (2% in water) | 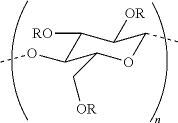 |
| | | | R = H or CH₃ or |
| | | | CH₂CH(OH)CH₃ |
| Hydroxypropyl cellulose (HPC) | Klucel Xtend HXF | ~2,250 cP (1% in water) | 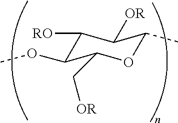 |
| | | | R = H or CH₂CH(OH)CH₃ |
| Poly(ethylene oxide) | Sentry Polyox WSR-303 | ~9,000 cP (1% in water) | 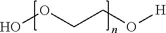 |

### Example 1.1—Hydrophilic Neutral Polymers and Direct Compression

Table 2 below shows the formulations studied in the form of tablets manufactured by direct compression. In each tablet, the total level of hydrophilic neutral polymer was kept constant at 50% w/w of the formulation. Because of the high drug loading that was required, only about 10% of filler (silicified microcrystalline cellulose) could be included in the formulations.

TABLE 2
Formulations used to study the impact of hydrophilic neutral polymers
on dexpramipexole dihydrochloride monohydrate dissolution.

| | 030443-01 | | 030443-03 | | 030444-06 | | 030443-05 | | 030443-07 | |
|---|---|---|---|---|---|---|---|---|---|---|
| | mg/ | % | mg/ | % | mg/ | % | mg/ | % | mg/ | % |
| Material | tab | w/w | tab | w/w | tab | w/w | tab | w/w | tab | w/w |
| Dexpramipexole | 319.00 | 37.53 | 319.00 | 37.53 | 319.00 | 37.53 | 319.00 | 37.53 | 319.00 | 37.53 |
| Dihydrochloride Monohydrate | | | | | | | | | | |
| Hypromellose | 425.00 | 50.00 | — | — | — | — | — | — | — | — |
| (Methocel K15M) | | | | | | | | | | |
| Hypromellose | — | — | 425.00 | 50.00 | — | — | — | — | — | — |
| (Methocel K100M) | | | | | | | | | | |
| Hypromellose | — | — | — | — | 425.00 | 50.00 | — | — | — | — |
| (Benecel K200M) | | | | | | | | | | |
| Hydroxypropyl Cellulose | — | — | — | — | — | — | 425.00 | 50.00 | — | — |
| (Klucel Xtend HXF) | | | | | | | | | | |
| Poly(ethylene oxide) | — | — | — | — | — | — | — | — | 425.00 | 50.00 |
| (Sentry Polyox WSR-303) | | | | | | | | | | |
| Silicified MCC | 91.13 | 10.72 | 91.13 | 10.72 | 97.50 | 11.47 | 91.13 | 10.72 | 91.13 | 10.72 |
| (Prosolv HD90) | | | | | | | | | | |
| Colloidal Silicon Dioxide | 6.38 | 0.75 | 6.38 | 0.75 | 4.25 | 0.50 | 6.38 | 0.75 | 6.38 | 0.75 |
| (Aerosil 200 P) | | | | | | | | | | |
| Magnesium Stearate | 8.50 | 1.00 | 8.50 | 1.00 | 4.25 | 0.50 | 8.50 | 1.00 | 8.50 | 1.00 |
| (Ligamed MF-2-V) | | | | | | | | | | |
| Total | 850.00 | 100.00 | 850.00 | 100.00 | 850.00 | 100.00 | 850.00 | 100.00 | 850.00 | 100.00 |

Hypromellose, HPC, and poly(ethylene oxide) - matrix polymer; silicified microcrystalline cellulose (MCC) = filler; colloidal silicon dioxide = glidant; magnesium stearate = lubricant.

The tablets were manufactured according to the following steps:
Screen excipients (except magnesium stearate) through #30 mesh screen;
Blend for 15 min at 25 rpm;
Screen magnesium stearate through #40 mesh screen;
Lubricate for 3 min at 25 rpm; and
Compress in a single station Globe Pharma MTCM manual hydraulic tablet press.

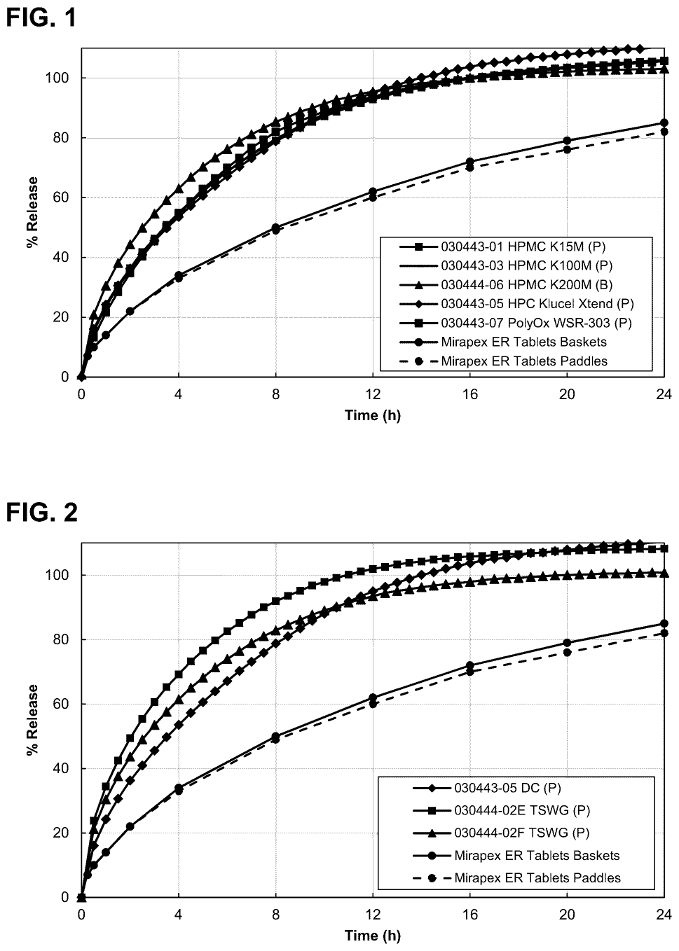

The dissolution results are provided in FIG. 1 and show that even with 50% w/w hydrophilic neutral polymer a significantly faster dissolution than with the comparison tablet with pramipexole was observed. Furthermore, there was no significant difference between the various types of hydrophilic neutral polymers studied.

### Example 1.2—Hydrophilic Neutral Polymers and Twin Screw Wet-Granulation

In addition to direct compression (DC), twin-screw wet-granulation (TSWG) was evaluated to increase the granular density and interaction between the drug substance and rate-controlling tablet ingredients. Twin-screw granulation is generally carried out as a continuous process and involves higher and more uniform shear rates as compared to traditional high shear granulation. For this trial, a Pharma 11 Twin Screw Extruder was used and the total level of hydrophilic neutral polymer was kept constant at 50% w/w of the formulation to enable comparison to the direct compression process. The formulations evaluated are shown in Table 3.

TABLE 3
Formulations for DC vs. TSWG manufacturing
method comparison.

| | 030443-05 | | 030444-02 | |
|---|---|---|---|---|
| | (DC) | | (TSWG) | |
| Material | mg/tab | % w/w | mg/tab | % w/w |
| Dexpramipexole Dihydrochloride | 319.00 | 37.53 | 319.00 | 37.53 |
| Monohydrate | | | | |
| Hydroxypropyl Cellulose (Klucel | 425.00 | 50.00 | 425.00 | 50.00 |
| Xtend HXF) | | | | |
| Silicified MCC (Prosolv HD90) | 91.13 | 10.72 | 97.50 | 11.47 |
| Colloidal Silicon Dioxide (Aerosil | 6.38 | 0.75 | 4.25 | 0.50 |
| 200 P) | | | | |
| Magnesium Stearate (Ligamed | 8.50 | 1.00 | 4.25 | 0.50 |
| MF-2-V) | | | | |
| Total | 850.00 | 100.00 | 850.00 | 100.00 |

HPC = matrix polymer; silicified microcrystalline cellulose (MCC) = filler; colloidal silicon dioxide = glidant; magnesium stearate = lubricant.

The TSWG tablets were manufactured according to the following steps:
Screen excipients (except magnesium stearate) through a #30 mesh screen;
Pre-blend the drug substance, polymer, and filler for 10 min at 25 rpm;
Perform twin-screw wet granulation (various sub-lots were collected at different feed rates, screw speeds, and water addition rates, see Table 4 below);
Dry granules in oven at 60° C.;
Co-mill the dried granules with 1.143 mm screen and 3300 rpm;
Screen colloidal silicon dioxide and magnesium stearate through a #30 mesh screen;
Perform final blending for 4 min at 25 rpm; and
Compress in a single station Globe Pharma MTCM manual hydraulic tablet press.

The TSWG process parameters under which the different batches were prepared are shown in Table 4.

TABLE 4
Twin-screw wet-granulation process parameters studied.
The granulation liquid used was purified water.

| | | Kneading | Screw | Solid (S) | Liquid (L) | | |
|---|---|---|---|---|---|---|---|
| | Kneading | Elements/ | Speed | Feed Rate | Feed Rate | | |
| Batch | Zones | Zone | (rpm) | (kg/h) | (kg/h) | L/S Ratio | Torque |
| 030444-02A | 3 | 4 × 60° | 150 | 1.0 | 0.16 | 0.16 | — |
| 030444-02B | 3 | 4 × 60° | 150 | 1.4 | 0.23 | 0.16 | 1.4 |
| 030444-02C | 3 | 4 × 60° | 300 | 1.4 | 0.31 | 0.22 | 1.3 |
| 030444-02D | 3 | 4 × 60° | 500 | 1.4 | 0.31 | 0.22 | 0.9 |
| 030444-02E | 3 | 8 × 60° | 300 | 0.8 | 0.16 | 0.20 | 1.6 |
| 030444-02F | 3 | 8 × 60° | 300 | 1.2 | 0.16 | 0.13 | 6.4 |

The dissolution results are provided in FIG. 2 (exemplary showing dissolution of batches 030444-02E and 030444-02F). As shown in FIG. 2, also the twin-screw wet-granulation process (with different parameters tested) did not result in tablets with a suitable dissolution profile for a sustained release tablet that can be given only once per day. Dissolution was too fast.

In conclusion, although a hydrophilic neutral polymer is used in the comparison tablet with pramipexole, the matrix tablets comprising hydrophilic neutral polymers evaluated above did not provide for suitable dissolution profiles.

### Example 2—Lipophilic Materials

In a next step, different lipophilic materials as matrix components were investigated. Lipophilic materials are non-swellable and hydrophobic and generally do not erode in aqueous media. The drug release from lipophilic matrices occurs via diffusion. However, due to the hydrophobic nature of the matrix, the water penetration into the tablet is generally slower than with hydrophilic matrices. The two lipophilic materials studied were glycerol behenate (Compritol 888 ATO) and Carnuba Wax. The properties of these materials are compared in Table 5.

TABLE 5
Comparison of lipophilic materials evaluated.

| | | Melt | |
|---|---|---|---|
| Material | Description | Temp | Structure |
| Glycerol Dibehenate (Compritol 888 ATO) | Consists of mono-, di-, and triesters of behenic acid (C₂₂), the diester fraction being predominant. | 71° C. (65- 77° C.) | 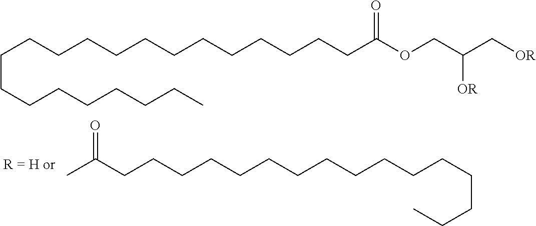 |
| Carnuba | Wax of leaves of | 80-86° C. | Mixture of different compounds |
| Wax | the carnuba palm | | |
| | Copernicia | | |
| | prunifera. It | | |
| | predominantly | | |
| | contains fatty acid | | |
| | esters and fatty | | |
| | alcohols in the C₂₆- | | |
| | C₃₀ range. | | |

Lipophilic materials alone or in combination with hydrophilic neutral polymers were evaluated in the following. Furthermore, based on the comparably lower melting temperature of these materials, melt granulation was also investigated and compared to direct compression.

### Example 2.1—Lipophilic Materials and Direct Compression

For the batches manufactured using direct compression, the following steps were employed:
Screen excipients (except magnesium stearate) through #30 mesh screen;
Blend for 15 min at 25 rpm;
Screen magnesium stearate through #40 mesh screen;
Lubricate for 3 min at 25 rpm; and
Compress in a single station Globe Pharma MTCM manual hydraulic tablet press.

Table 6 shows the compositions evaluated and manufactured with direct compression process. Tablet 030443-05 was discussed previously in Example 1 and was included to evaluate the relative impact of the lipophilic materials in the matrix.

TABLE 6
Formulations used to study the impact of lipophilic materials on dexpramipexole
dihydrochloride monohydrate dissolution (tablets produced by direct compression).

| | 030443-05 | | 030443-09 | | 030443-23 | |
|---|---|---|---|---|---|---|
| Material | mg/tab | % w/w | mg/tab | % w/w | mg/tab | % w/w |
| Dexpramipexole Dihydrochloride Monohydrate | 319.00 | 37.53 | 319.00 | 37.53 | 319.00 | 37.53 |
| Hydroxypropyl Cellulose (Klucel Xtend HXF) | 425.00 | 50.00 | — | — | 425.00 | 50.00 |
| Glycerol Dibehenate (Compritol 888 ATO) | — | — | 425.00 | 50.00 | 85.00 | 10.00 |
| Silicified MCC (Prosolv HD90) | 91.13 | 10.72 | 97.50 | 11.47 | 12.50 | 1.47 |
| Colloidal Silicon Dioxide (Aerosil 200 P) | 6.38 | 0.75 | 4.25 | 0.50 | 4.25 | 0.50 |
| Magnesium Stearate (Ligamed MF-2-V) | 8.50 | 1.00 | 4.25 | 0.50 | 4.25 | 0.50 |
| Total | 850.00 | 100.00 | 850.00 | 100.00 | 850.00 | 100.00 |

HPC = matrix polymer; glycerol dibehenate = lipophilic matrix; silicified microcrystalline cellulose (MCC) = filler; colloidal silicon dioxide = glidant; magnesium stearate = lubricant.

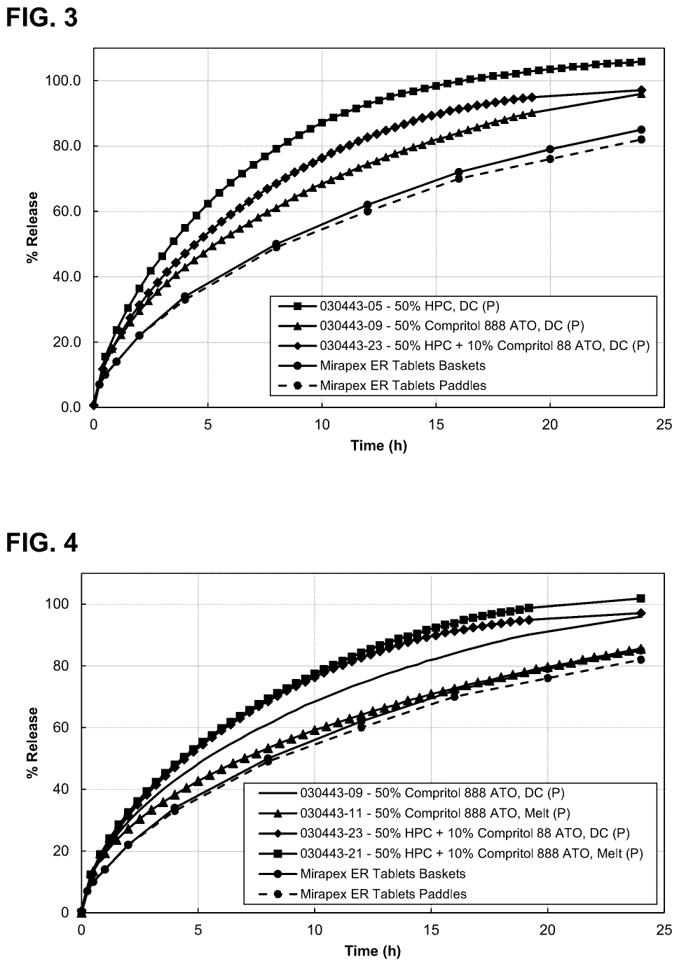

FIG. 3 shows the results from the dissolution studies. Although the results indicate that Compritol 888 ATO slows down the dissolution to a greater extent than the hydrophilic neutral polymer Hydroxypropyl cellulose, the dissolution profile was still too fast.

### Example 2.2—Lipophilic Materials and Melt Granulation

In addition to direct compression, melt granulation using various lipophilic materials was also evaluated. For batches manufactured using melt granulation, the following steps were employed:
Screen the drug substance, lipophilic material, and Silicified Microcrystalline Cellulose, SMCC, through a #30 mesh screen;
Blend for 15 min at 25 rpm;
Place the mixture in an oven at 75° C. (glycerol dibehenate) or 90° C. (carnuba wax) for 3 h;
Cool the material at room temperature and pass it through a #20 mesh screen;
Screen magnesium stearate through #40 mesh screen;
Lubricate for 3 min at 25 rpm; and
Compress in a single station Globe Pharma MTCM manual hydraulic tablet press.

The formulations manufactured using the melt granulation process and using direct compression outlined above are compared in Table 7 below.

TABLE 7
Formulations used to study the impact of lipophilic materials on dexpramipexole dihydrochloride monohydrate
dissolution (tablets produced by melt granulation or direct compression, respectively).

| | 030443-09 and -11 | | 030443-21 and -23 | | 030443-35 | |
|---|---|---|---|---|---|---|
| Material | mg/tab | % w/w | mg/tab | % w/w | mg/tab | % w/w |
| Dexpramipexole Dihydrochloride Monohydrate | 319.00 | 37.53 | 319.00 | 37.53 | 319.00 | 37.53 |
| Hydroxypropyl Cellulose (Klucel Xtend HXF) | — | — | 425.00 | 50.00 | — | — |
| Glycerol Dibehenate (Compritol 888 ATO) | 425.00 | 50.00 | 85.00 | 10.00 | — | — |
| Carnuba Wax | | | | | 425.00 | 50.00 |
| Silicified MCC (Prosolv HD90) | 97.50 | 11.47 | 12.50 | 1.47 | 97.50 | 11.47 |
| Colloidal Silicon Dioxide (Aerosil 200 P) | 4.25 | 0.50 | 4.25 | 0.50 | 4.25 | 0.50 |
| Magnesium Stearate (Ligamed MF-2-V) | 4.25 | 0.50 | 4.25 | 0.50 | 4.25 | 0.50 |
| Total | 850.00 | 100.00 | 850.00 | 100.00 | 850.00 | 100.00 |

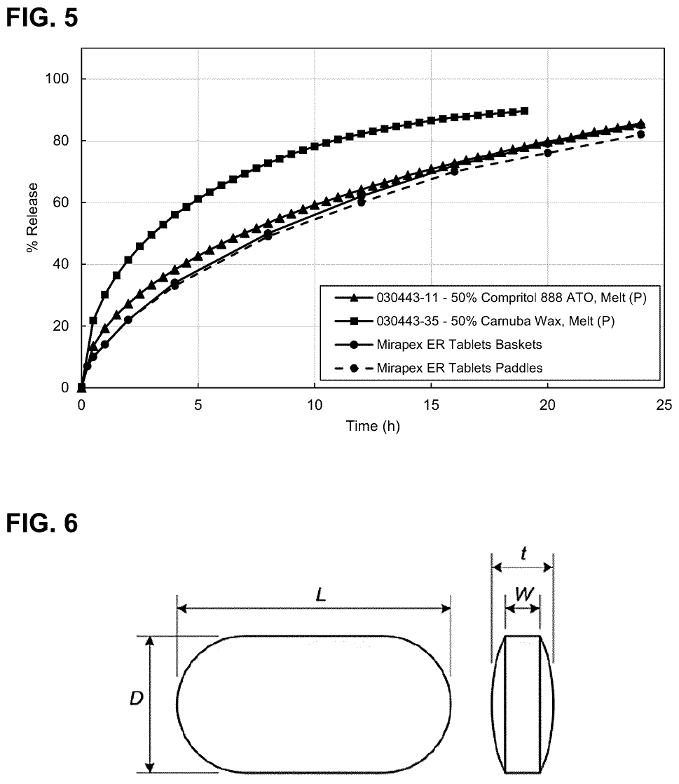

The dissolution results are provided in FIG. 4 (comparison of direct compression, DC, versus melt granulation) and FIG. 5 (comparison of glycerol dibehenate and carnuba wax; tablets prepared using melt granulation). The results indicate that when Compritol 888 ATO is used at a lower level (10%) in combination with HPC (50%), there was no significant difference between DC and melt granulation. However, when the Compritol 888 ATO content is high (50%), the melt granulation process slows down the dissolution significantly. Additionally, the results indicate the glycerol dibehenate is more effective at slowing the dissolution than carnuba wax.

Taken together, a suitable dissolution profile was obtained with batch 030443-11 (50% w/w Compritol 888 ATO; prepared by melt granulation).

### Example 2.3—Lipophilic Materials: Compressibility

In a next step, tablet hardness and tensile strength calculations were performed to better assess the practicality and processability of the formulations containing lipophilic materials. The tensile strength is a good measurement of the tablet strength since it is an intrinsic property, normalized by the tablet shape and size. Generally, a tensile strength of 1.7 MPa or more is targeted to ensure that the tablet is mechanically strong enough to withstand downstream manufacturing steps and distribution. See also Pitt, K. G. and Heasley M. G, 2013, “Determination of the tensile strength of elongated tablets”, Powder Technology 238: 169-175; herewith incorporated by reference in its entirety. The equation used for calculation of the tensile strength of an elongated tablet is:

$$σ = \frac{2}{3} \frac{10 F / ( π {D}^{2} )}{( 2.84 \frac{t}{D} ) - ( 0.126 \frac{t}{W} ) + ( 3.15 \frac{W}{D} ) + 0.01}$$

where F is the breaking force in Newton (i.e. the “hardness”), and D, t, and W for elongated tablets are shown in FIG. 6. As shown in FIG. 6, D is the length of the short axis of the tablet, L is the length of the long axis of the tablet, t is the overall thickness of the tablet, and W is the tablet wall height. For the sustained release matrix tablets comprising 319 mg dexpramipexole dihydrochloride monohydrate, the tablet tooling used is 8.51 mm×19.00 mm (D×L).

The hardness (i.e. breaking force) and tensile strength results are provided in Table 8.

TABLE 8
Tablet hardness (i.e. breaking force) and tensile strength for tablets
with hydrophilic neutral polymers and/or lipophilic materials.

| | | | Compaction | Tablet | Tensile |
|---|---|---|---|---|---|
| | | Pro- | Force | Hardness | Strength |
| Batch | Description | cess | (psi) | (kp) | (MPa) |
| 030443-05 | 50% HPC only | DC | 3000 | 16.3 | 1.47 |
| 030443-23 | 50% HPC, | DC | 3000 | 14.6 | 1.27 |
| | 10% Compritol | | | | |
| 030443-21 | 50% HPC, | Melt | 3000 | 15.7 | 1.40 |
| | 10% Compritol | | | | |
| 030443-15* | 25% HPC, | Melt | 3000 | 10.0 | 0.90 |
| | 25% Compritol | | | | |
| 030443-09 | 50% Compritol | DC | 3000 | 6.0 | 0.53 |
| | only | | | | |
| 030443-11 | 50% Compritol | Melt | 3000 | 7.3 | 0.65 |
| | only | | | | |
| 030443-35 | 50% Carnuba | Melt | 3000 | 14.5 | 1.25 |
| | Wax only | | | | |

*Dissolution results for batch 030443-15 are not shown in FIG. 4, but were even faster than batches 030443-21 and 030443-23.

The results show that the hardness and tensile strength decrease with increasing Compritol 888 ATO content. Batch 030443-11, which had a dissolution profile close to the comparison tablet with pramipexole, has a tensile strength <1.0 MPa, which is too low for downstream processing. In general, the batches with the lipophilic materials yield low tensile strengths. Also the tablet with HPC only (batch 030443-05) revealed an insufficient tensile strength.

Thus, despite a promising dissolution profile observed for Batch 030443-11, the matrix tablets with lipophilic materials prepared were overall mechanically weak and unsuitable for manufacturing (in particular on a routine and large-scale basis).

### Example 3—Hydrophilic Anionic Polymers

In the following, the impact of anionic polymers on the dissolution was evaluated. Anionic polymers have negatively charged ions, generally located on functional groups of the main polymer backbone. The drug release from anionic polymer matrices occurs via diffusion and erosion. However, due to the ionic nature of the matrix, interactions between the drug substance and matrix can occur that increase the diffusional resistance or decrease it, depending on the ionic charges involved. Two types of anionic polymers were evaluated: carbomer (Carbopol 971P) and sodium carboxymethylcellulose (Aqualon CMC 7HXF PH). The properties of these materials are compared in Table 9.

TABLE 9
Comparison of anionic polymers evaluated. The pKa value of carbomer
(Carbopol 971P) is 6.0 ± 0.5 and the pKa value of sodium carboxymethylcellulose
(Aqualon CMC 7HXF PH) is ~4.0.

| Material | Description | Viscosity | Structure |
|---|---|---|---|
| Carbomer (Carbopol 971P) | Crosslinked acrylic acid polymer | 4,000- 11,000 cP (0.5% at pH 7.5) | 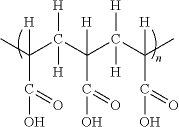 |
| Sodium carboxymethyl- cellulose (Aqualon CMC 7HXF PH) | Sodium salt of a cellulose derivative with carboxymethyl groups | 1,500- 3,000 cP (1% in water) | 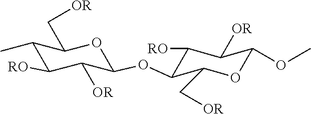 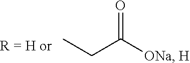 |

The tablets with anionic polymers were manufactured using direct compression employing the following steps (unless indicated otherwise):
Screen excipients (except magnesium stearate) through #30 mesh screen;
Blend for 15 min at 25 rpm;
Screen magnesium stearate through #40 mesh screen;
Lubricate for 3 min at 25 rpm;
Compress in a single station Globe Pharma MTCM manual hydraulic tablet press.

### Example 3.1—Single Anionic Polymer (Carbomer)

First, the impact of increasing quantities of carbomer on the dissolution of dexpramipexole was evaluated. The formulations studied are provided in Table 10.

TABLE 10
Formulations used to study the impact of the anionic polymer carbomer
on dexpramipexole dihydrochloride monohydrate dissolution.

| | 030443-36 | | 030444-04 | | 030444-05 | | 030443-37 | |
|---|---|---|---|---|---|---|---|---|
| Material | mg/tab | % w/w | mg/tab | % w/w | mg/tab | % w/w | mg/tab | % w/w |
| Dexpramipexole | 319.00 | 37.53 | 319.00 | 37.53 | 319.00 | 37.53 | 319.00 | 37.53 |
| Dihydrochloride Monohydrate | | | | | | | | |
| Carbomer (Carbopol 971P) | 85.00 | 10.00 | 255.00 | 30.00 | 340.00 | 40.00 | 425.00 | 50.00 |
| Silicified MCC (Prosolv HD90) | 437.50 | 51.47 | 267.50 | 31.47 | 182.50 | 21.47 | 97.50 | 11.47 |
| Colloidal Silicon Dioxide | 4.25 | 0.50 | 4.25 | 0.50 | 4.25 | 0.50 | 4.25 | 0.50 |
| (Aerosil 200 P) | | | | | | | | |
| Magnesium Stearate | 4.25 | 0.50 | 4.25 | 0.50 | 4.25 | 0.50 | 4.25 | 0.50 |
| (Ligamed MF-2-V) | | | | | | | | |
| Total | 850.00 | 100.00 | 850.00 | 100.00 | 850.00 | 100.00 | 850.00 | 100.00 |

Carbomer = matrix polymer; silicified MCC = filler; colloidal silicon dioxide = glidant; magnesium stearate = lubricant.

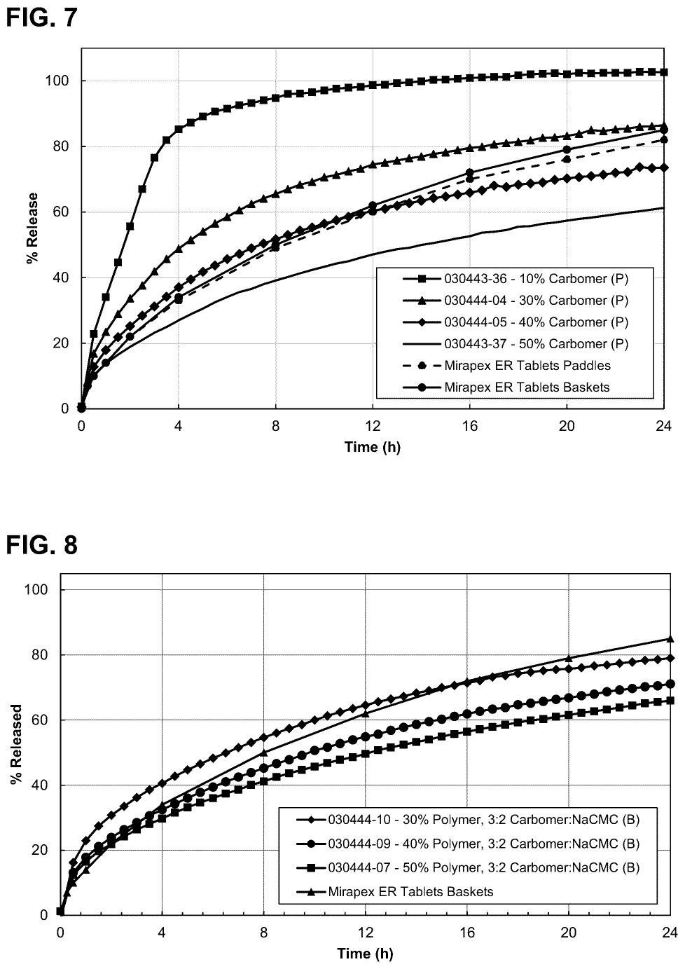

The dissolution profiles of the different tablets are provided in FIG. 7. It can be seen that carbomer had a strong impact on the level of dissolution, with increasing carbomer resulting in slower dissolution. While a carbomer amount of 10% w/w did not result in a suitable in vitro dissolution profile, the higher amounts evaluated provided for suitable release.

Additionally, the hardness and tensile strength of the tablets containing carbomer were evaluated. The results are presented in Table 11 and show that carbomer resulted in good compressibility and yielded strong tablets.

TABLE 11
Tablet hardness (i.e. breaking force) and tensile strength results.

| | | | Compaction | | Tensile |
|---|---|---|---|---|---|
| | | Pro- | Force | Hardness | Strength |
| Batch | Description | cess | (psi) | (kp) | (MPa)* |
| 030443-36 | 10% Carbomer | DC | 4000 | 25.9 | 2.34 |
| 030444-04 | 30% Carbomer | DC | 4000 | 24.3 | 2.20 |
| 030444-05 | 40% Carbomer | DC | 4000 | 23.4 | 2.11 |
| 030443-37 | 50% Carbomer | DC | 4000 | 20.1 | 1.82 |

*Tablet thickness was not captured during these batches and a value of 6.50 mm was assumed.

### Example 3.2 Combination of Anionic Polymers (Carbomer and Sodium Carboxymethylcellulose)

In addition to evaluating the impact of one anionic polymer, also a combination of two anionic polymers was evaluated. The effect of varying the total anionic polymer amount, as well as the ratio of anionic polymers within the formulations on dissolution profiles was studied.

In particular, an exemplary combination of carbomer and sodium carboxymethylcellulose was evaluated herein. The impact of the total anionic polymer amount (% w/w) was evaluated while maintaining a fixed polymer ratio of 3:2 carbomer:sodium carboxymethylcellulose. In addition, the impact of the polymer ratio was evaluated while maintaining a fixed total anionic polymer amount of 40% w/w. The formulations studied are presented in Table 12 (impact of total anionic polymer amount) and Table 13 (impact of % polymer ratio).

TABLE 12
Formulations for evaluating the impact of the total anionic polymer
amount at a fixed ratio (3:2 Carbomer:Sodium Carboxymethylcellulose,
CMC) on dexpramipexole dihydrochloride monohydrate dissolution.

| | 030444-10 | | 030444-09 | | 030444-07 | |
|---|---|---|---|---|---|---|
| | (30% polymer) | | (40% polymer) | | (50% polymer) | |
| Material | mg/tab | % w/w | mg/tab | % w/w | mg/tab | % w/w |
| Dexpramipexole Dihydrochloride | 319.00 | 37.53 | 319.00 | 37.53 | 319.00 | 37.53 |
| Monohydrate | | | | | | |
| Carbomer (Carbopol 971P) | 153.00 | 18.00 | 204.00 | 24.00 | 255.00 | 30.00 |
| Sodium Carboxymethylcellulose | 102.00 | 12.00 | 136.00 | 16.00 | 170.00 | 20.00 |
| (Aqualon CMC 7HXF PH) | | | | | | |
| Silicified MCC (Prosolv HD90) | 267.50 | 31.47 | 182.50 | 21.47 | 97.50 | 11.47 |
| Colloidal Silicon Dioxide (Aerosil 200 P) | 4.25 | 0.50 | 4.25 | 0.50 | 4.25 | 0.50 |
| Magnesium Stearate (Ligamed MF-2-V) | 4.25 | 0.50 | 4.25 | 0.50 | 4.25 | 0.50 |
| Total | 850.00 | 100.00 | 850.00 | 100.00 | 850.00 | 100.00 |

TABLE 13
Formulations for evaluating the impact of the ratio of anionic polymers at a fixed total anionic
polymer amount of 40% w/w on dexpramipexole dihydrochloride monohydrate dissolution.

| | 030444-09 | | 030444-11 | | 030444-12 | | 030444-13 | | 030444-21 | |
|---|---|---|---|---|---|---|---|---|---|---|
| | (3:2) | | (1:1) | | (2:3) | | (3:7) | | (2:8) | |
| Material | mg/tab | % w/w | mg/tab | % w/w | mg/tab | % w/w | mg/tab | % w/w | mg/tab | % w/w |
| Dexpramipexole | 319.00 | 37.53 | 319.00 | 37.53 | 319.00 | 37.53 | 319.00 | 37.53 | 319.00 | 37.53 |
| Dihydrochloride | | | | | | | | | | |
| Monohydrate | | | | | | | | | | |
| Carbomer | 204.00 | 24.00 | 170.00 | 20.00 | 136.00 | 16.00 | 102.00 | 12.00 | 68.00 | 8.00 |
| (Carbopol 971P) | | | | | | | | | | |
| Sodium Carboxy- | 136.00 | 16.00 | 170.00 | 20.00 | 204.00 | 24.00 | 238.00 | 28.00 | 272.00 | 32.00 |
| methylcellulose (Aqualon | | | | | | | | | | |
| CMC 7HXF PH) | | | | | | | | | | |
| Silicified MCC | 182.50 | 21.47 | 182.50 | 21.47 | 182.50 | 21.47 | 182.50 | 21.47 | 182.50 | 21.47 |
| (Prosolv HD90) | | | | | | | | | | |
| Colloidal Silicon Dioxide | 4.25 | 0.50 | 4.25 | 0.50 | 4.25 | 0.50 | 4.25 | 0.50 | 4.25 | 0.50 |
| (Aerosil 200 P) | | | | | | | | | | |
| Magnesium Stearate | 4.25 | 0.50 | 4.25 | 0.50 | 4.25 | 0.50 | 4.25 | 0.50 | 4.25 | 0.50 |
| (Ligamed MF-2-V) | | | | | | | | | | |
| Total | 850.00 | 100.00 | 850.00 | 100.00 | 850.00 | 100.00 | 850.00 | 100.00 | 850.00 | 100.00 |

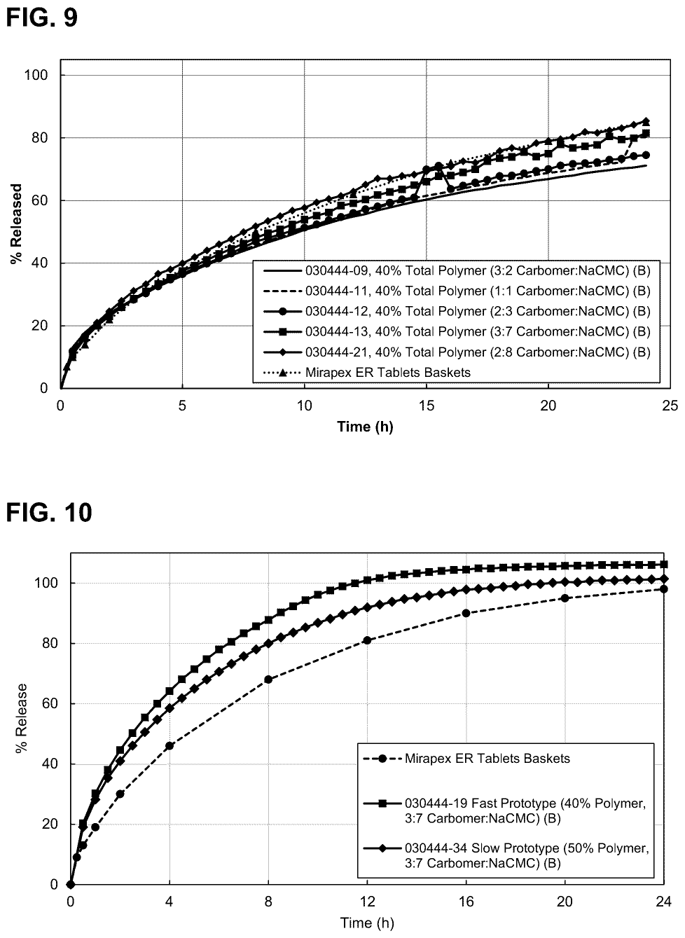

The dissolution results are provided in FIG. 8 (impact of total anionic polymer amount at a fixed polymer ratio) and FIG. 9 (impact of polymer ratio at a fixed total anionic polymer amount). The results show that, overall, for all evaluated formulations, desirable dissolution profiles were achieved over a wide ratio range of the two anionic polymers and over a wide range of total anionic polymer amount.

Additionally, the hardness and tensile strength of the tested batches were evaluated to further confirm the suitability of these formulations for manufacturing. The results are presented in Table 14 and show that the all formulations had good compressibility and yielded strong tablets.

TABLE 14
Tablet hardness (i.e. breaking force) and tensile strength results.

| | | | Compaction | | Tensile |
|---|---|---|---|---|---|
| | | Pro- | Force | Hardness | Strength |
| Batch | Description | cess | (psi) | (kp) | (MPa) |
| 030444-09 | 40% polymer, 3:2 | DC | 4800 | 21.5 | 2.06 |
| 030444-11 | 40% polymer, 1:1 | DC | 4800 | 21.3 | 2.07 |
| 030444-12 | 40% polymer, 2:3 | DC | 4800 | 17.8 | 1.71 |
| 030444-13 | 40% polymer, 3:7 | DC | 4800 | 18.9 | 1.84 |
| 030444-21 | 40% polymer, 2:8 | DC | 4800 | 18.3 | 1.81 |

### Example 3.3—Dissolution in Different Media

To further study dissolution in different media, two prototype tablets with two anionic polymers were selected. In these prototypes, the carbomer:sodium carboxymethylcellulose ratio was fixed at 3:7 and the total anionic polymer amount was varied from 40% w/w total anionic polymer (fast prototype) to 50% w/w total anionic polymer (slow prototype). When the total anionic polymer amount was increased to 50% w/w, the tablet weight was also increased from 850 mg to 900 mg by adding an additional 50 mg of filler (silicified microcrystalline cellulose). The slow prototype contained 425 mg of total anionic polymers, which constitutes 50% w/w of 850 mg (the tablet core weight of the fast prototype). However, with the slow prototype tablet weight increased up to 900 mg for the addition of more filler (silicified microcrystalline cellulose), the slow prototype actually contained 47.23% w/w polymer. However, for ease of discussion, reference is made to 50% w/w total anionic polymer in the context of the slow prototype in the following. The formulations tested are shown in Table 15.

TABLE 15
Formulation of fast and slow prototype for
dissolution studies in different media.

| | 030444-19* | | 030444-34 | |
|---|---|---|---|---|
| | Fast Prototype | | Slow Prototype | |
| | (40% total polymer) | | (50% total polymer) | |
| Material | mg/tab | % w/w | mg/tab | % w/w |
| Dexpramipexole | 319.00 | 37.53 | 319.00 | 35.44 |
| Dihydrochloride Monohydrate | | | | |
| Carbomer (Carbopol 971P) | 102.00 | 12.00 | 127.50 | 14.17 |
| Sodium | 238.00 | 28.00 | 297.50 | 33.06 |
| Carboxymethylcellulose | | | | |
| (Aqualon CMC 7HXF PH) | | | | |
| Silicified MCC (Prosolv | 182.50 | 21.47 | 147.00 | 16.33 |
| HD90) | | | | |
| Colloidal Silicon Dioxide | 4.25 | 0.50 | 4.50 | 0.50 |
| (Aerosil 200 P) | | | | |
| Magnesium Stearate (Ligamed | 4.25 | 0.50 | 4.50 | 0.50 |
| MF-2-V) | | | | |
| Total | 850.00 | 100.00 | 900.00 | 100.00 |

*Batch 030444-19 further comprises a coating as shown in Table 18. The manufacture of batch 030444-19 is described in Example 4.

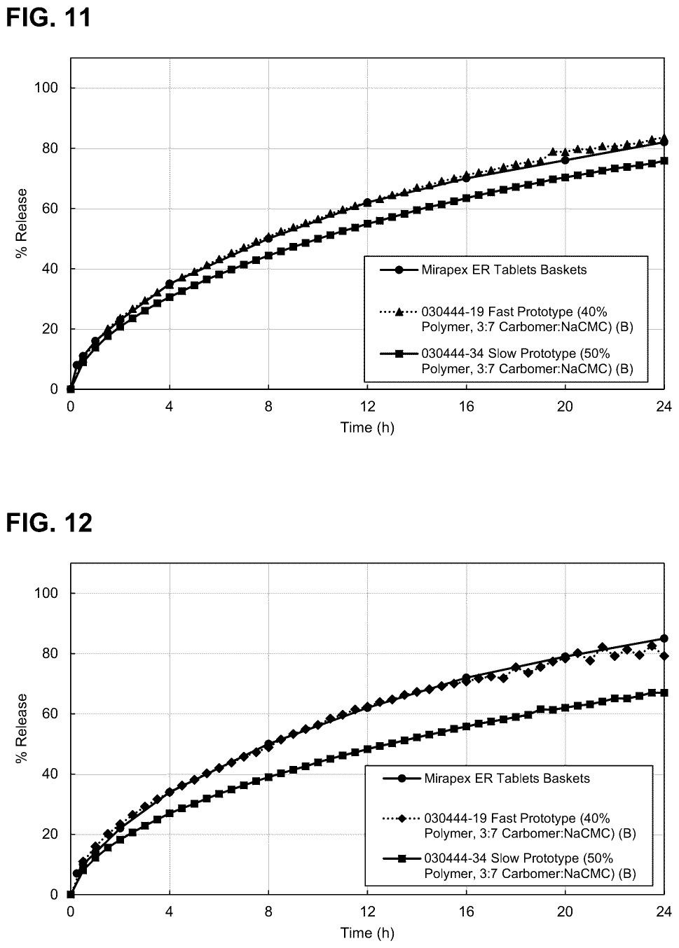

Dissolution results are presented in FIG. 10 (0.1 N HCl), FIG. 11 (McIlvaine Buffer, pH 4.5), and FIG. 12 (Sodium Phosphate Buffer, pH 6.8). In addition to dissolution, the slow prototype with the increased tablet weight was also evaluated for compressibility by measuring the tablet hardness and tensile strength. The results are presented in Table 16 and show that the resultant tensile strength is adequate for manufacturing.

TABLE 16
Tablet hardness (i.e. breaking force) and tensile strength results.

| | | | Compaction | | Tensile |
|---|---|---|---|---|---|
| | | | Force | Hardness | Strength |
| Batch | Description | Process | (psi) | (kp) | (MPa) |
| 030444-34 | Slow: 50% | DC | 5000 | 20.7 | 1.84 |
| | polymer, 3:7 | | | | |

### Example 4—Process Evaluation

The tablets comprising anionic polymers as evaluated under Example 3 above were prepared by direct compression (DC) followed by manual compaction using a scale of 15 tablets per batch (i.e., 12.75 g blend) unless otherwise indicated. After identification of tablets resulting in desirable dissolution profiles (i.e., tablets with the amounts of anionic polymer specified herein), further process evaluation was performed with those matrices. In particular, two different processes (high shear wet granulation, HSG, and roller compaction, RC) were performed at a higher scale per batch, i.e. 1 kg blend.

Two different formulations were manufactured each using the two different processes (HSG and RC). Table 17 gives an overview of the process evaluation and formulations, whereas Table 18 gives the details on the tablet formulations investigated. The different tablet batches evaluated in this Example were coated with a HPMC:HPC (1:1) system. However, the tablet dissolution is controlled by the functional matrix-forming polymers in the tablet core.

TABLE 17
Summary of process evaluation and formulations.

| | | Carbomer:NaCMC | Total Anionic |
|---|---|---|---|
| Batch Number | Process | Polymer Ratio | Polymer Amount |
| 030444-15 | HSG | 3:7* | 40% (12%:28%)* |
| 030444-17 | HSG | 2:8 | 40% (8%:32%) |
| 030444-19 | RC | 3:7* | 40% (12%:28%)* |
| 030444-21 | RC | 2:8 | 40% (8%:32%) |

CMC = Carboxymethylcellulose.
*This formulation represents the fast prototype of Example 3.3.

TABLE 18
Batches evaluated by different manufacturing processes (HSG =
high shear granulation; RC = roller compaction).

| | 030444-15 | | 030444-17 | | 030444-19 | | 030444-21 | |
|---|---|---|---|---|---|---|---|---|
| | (HSG, 3:7)* | | (HSG, 2:8) | | (RC, 3:7)* | | (RC, 2:8) | |
| Material | mg/tab | % w/w | mg/tab | % w/W | mg/tab | % w/w | mg/tab | % w/w |
| Dexpramipexole | 319.00 | 37.53 | 319.00 | 37.53 | 319.00 | 37.53 | 319.00 | 37.53 |
| Dihydrochloride Monohydrate | | | | | | | | |
| Carbomer (Carbopol 971P) | 102.00 | 12.00 | 68.00 | 8.00 | 102.00 | 12.00 | 68.00 | 8.00 |
| Sodium Carboxymethyl- | 238.00 | 28.00 | 272.00 | 32.00 | 238.00 | 28.00 | 272.00 | 32.00 |
| cellulose (Aqualon CMC 7HXF | | | | | | | | |
| PH) | | | | | | | | |
| Silicified MCC (Prosolv HD90) | 182.50 | 21.47 | 182.50 | 21.47 | 182.50 | 21.47 | 182.50 | 21.47 |
| Colloidal Silicon Dioxide | 4.25 | 0.50 | 4.25 | 0.50 | 4.25 | 0.50 | 4.25 | 0.50 |
| (Aerosil 200 P) | | | | | | | | |
| Magnesium Stearate | 4.25 | 0.50 | 4.25 | 0.50 | 4.25 | 0.50 | 4.25 | 0.50 |
| (Ligamed MF-2-V) | | | | | | | | |
| Tablet Core Total | 850.00 | 100.00 | 850.00 | 100.00 | 850.00 | 100.00 | 850.00 | 100.00 |
| Hypomellose (Methocel E3) | 12.75 | 1.50 | 12.75 | 1.50 | 12.75 | 1.50 | 12.75 | 1.50 |
| Hydroxypropyl Cellulose | 12.75 | 1.50 | 12.75 | 1.50 | 12.75 | 1.50 | 12.75 | 1.50 |
| (Klucel EXF) | | | | | | | | |
| Coated Tablet Total | 875.50 | 103.00 | 875.50 | 103.00 | 875.50 | 103.00 | 875.50 | 103.00 |

*This formulation represents the fast prototype of Example 3.3.

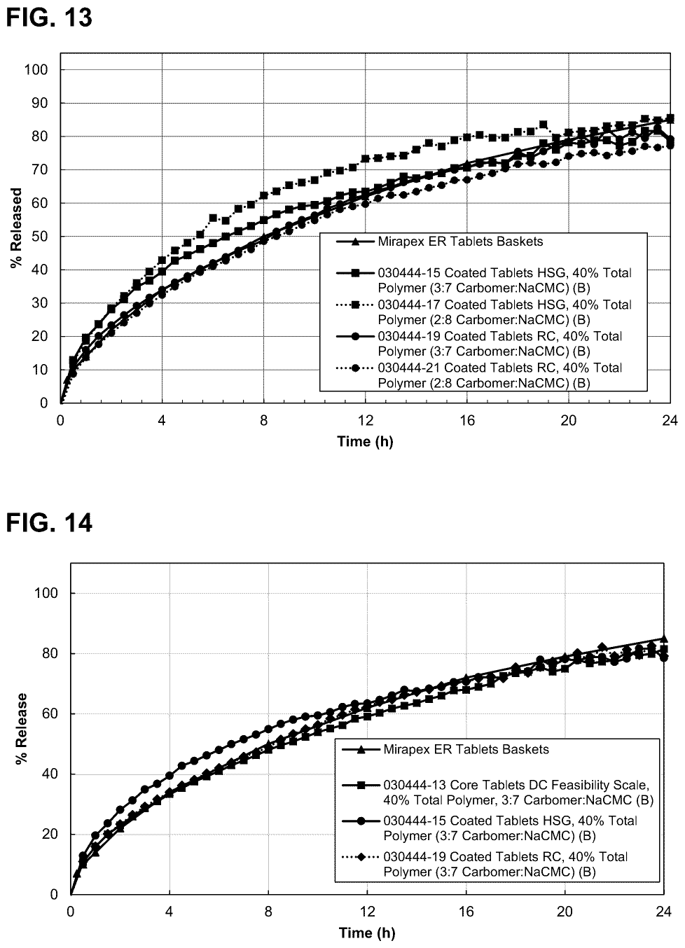

The dissolution results of the four batches evaluated are shown in FIG. 13. Overall, the data demonstrate that the different manufacturing processes generally resulted in tablets that all provide for a beneficial in vitro dissolution profile (i.e., an in vitro dissolution profile that renders the matrix tablets suitable for once daily administration to a human). However, when evaluated more closely, the data show there was less spread between the two formulations when manufactured using the roller compaction process suggesting that it is a more robust process with the potential to reduce variability and formulation sensitivity.

The impact of process and scale is further compared in FIG. 14, which shows 3 different batches all with the same formulation (40% polymer, 3:7 ratio) manufactured under 3 different conditions: direct compression (scale: 12.75 g blend), high shear wet-granulation (scale: 1 kg blend), and roller compaction (scale: 1 kg blend). In general, the formulation is reproducible with respect to any process and scale.

### Example 5—Process Development

Although the dissolution profiles of the tablets with one or more anionic polymer were not strongly impacted by different production processes, based on the results in Example 4, a manufacturing process including preparation of densified granules by roller compaction seemed to be particularly favorable. Roller compaction was shown to be even more robust than other evaluated processes as it reduces variability and formulation sensitivity (FIG. 13).

In general, the manufacturing process involving roller compaction is carried out by: preparing a pre-blend comprising the drug substance (dexpramipexole, or a pharmaceutically acceptable salt thereof) and further excipients such as the anionic polymer(s); preparing densified granules from the pre-blend by roller compaction; preparing a blend by mixing the densified granules with a glidant and a lubricant; compressing the blend into a tablet core; and optionally coating the tablet core with a film coating. Preparing a blend comprises pre-lubrication, in which 0.5% w/w glidant is added, and lubrication, in which 0.5% w/w lubricant is added.

The roller compaction process is a dry process that did not incorporate water or heat. Also during preparation of the blend, no water was added or heat applied. The compression process did not introduce any new excipients or used water. Water was introduced only during the coating process by applying a film coating solution, but the water was subsequently removed again.

Specific roller compaction and compression parameters were evaluated in the following.

### Example 5.1—Roller Compaction Process Development

Smaller scale development (1 kg scale) of the roller compaction process was carried out using a Gerteis MiniPolygran, whereas larger scale manufacturing (Examples 5.3 and 5.4) was carried out using a Gerteis MiniPactor, which is a larger roller compactor. A comparison of both roller compactors is given in Table 19.

TABLE 19
Comparison of Gerteis MiniPolygran
and MiniPactor roller compactors.

| | | |
|---|---|---|
| Characteristic | MiniPolygran | MiniPactor |

| | | | | |
|---|---|---|---|---|
| Roll Diameter | 150 | mm | 250 | mm |

| | | |
|---|---|---|
| Throughput | max. 50 kg/h | max. 100 kg/h |

| | | | | |
|---|---|---|---|---|
| Compaction Force | 1-15 | kN/cm | 1-20 | kN/cm |
| Roll Gap | 1-4 | mm | 1-6 | mm |
| Roll Speed | 1-10 | rpm | 1-30 | rpm |

During smaller scale development, four batches (scale: 1 kg blend) were manufactured using the Gerteis MiniPolygran. Two batches were manufactured using a compaction force of 2.5 kN and milled with 0.8 mm mesh screen and two batches were manufactured with a higher force of 3.0 kN and a larger 1.0 mm mesh screen. The process parameters studied are summarized in Table 20 and the characterization results of the final blend (which contains 99% w/w densified granules) are provided in Table 21. The remaining 1% w/w of the blend are made up by 0.5% w/w each of colloidal silicon dioxide and magnesium stearate. See also Table 18 providing the composition of batches 030444-19 and -21, and Table 22 providing the composition of batches 030444-36 and -38.

TABLE 20
Summary of roller compaction (RC) process parameters studied.

| | Lab Scale-Up Batches | | | | Stability Batches | | | |
|---|---|---|---|---|---|---|---|---|
| Parameter | 030444-19 | | 030444-21 | | 030444-36 | | 030444-38 | |
| Compaction Force | 2.5 | kN | 2.5 | kN | 3.0 | kN | 3.0 | kN |
| Roll Gap | 1.5 | mm | 1.5 | mm | 1.5 | mm | 1.5 | mm |
| Roll Speed | 5 | rpm | 5 | rpm | 5 | rpm | 5 | rpm |
| Screen Size/Type | 0.8 | mm mesh | 0.8 | mm mesh | 1.0 | mm mesh | 1.0 | mm mesh |

| | | | | |
|---|---|---|---|---|
| Granulator Type | Star | Star | Star | Star |
| Granulator Speed (CW/CCW) | 45 rpm/45 rpm | 45 rpm/45 rpm | 45 rpm/45 rpm | 45 rpm/45 rpm |
| Granulator Angle (CW/CCW) | 250°/350° | 250°/350° | 250°/350° | 250°/350° |

TABLE 21
Characterization of pre-blend and final blend (final blend comprising
99% w/w densified granules prepared by roller compaction).

| | | | Blend (comprising 99% w/w densified granules)\hat{ }\hat{ } | | | |
|---|---|---|---|---|---|---|
| | | Pre-Blend\hat{ } | 030444-19* | 030444-21 | 030444-36 | 030444-38 |
| Characteristic | | 030444-36 | (2.5 kN, 0.8 mm) | (2.5 kN, 0.8 mm) | (3.0 kN, 1.0 mm) | (3.0 kN, 1.0 mm) |
| Bulk Density | | 0.49 | 0.52 | 0.53 | 0.55 | 0.59 |
| Tapped Density | | 0.76 | — | 0.73 | 0.77 | 0.75 |
| Angle of Repose | | 25.6* | — | 24.90 | 22.80 | 19.50 |
| PSD | Mesh | % Retained | % Retained | % Retained | % Retained | % Retained |
| | #30 | 1.93 | — | 3.02 | 17.47 | 14.24 |
| | #40 | 4.15 | — | 7.40 | 11.43 | 11.24 |
| | #60 | 12.68 | — | 12.97 | 13.07 | 12.59 |
| | #80 | 10.85 | — | 11.94 | 9.22 | 9.22 |
| | #100 | 10.81 | — | 7.03 | 5.38 | 5.68 |
| | #140 | 9.54 | — | 9.60 | 8.53 | 8.04 |
| | Pan | 48.70 | — | 46.92 | 32.49 | 34.07 |

\hat{ }pre-blend: mixture of dexpramipexole dihydrochloride monohydrate, anionic polymer, and silicified microcrystalline cellulose before roller compaction.
\hat{ }\hat{ }blend: mixture of densified granules, colloidal silicon dioxide (glidant), and magnesium stearate (lubricant).
*A final blend sample from batch 030444-19 was not collected and further testing as for bulk density was not performed.

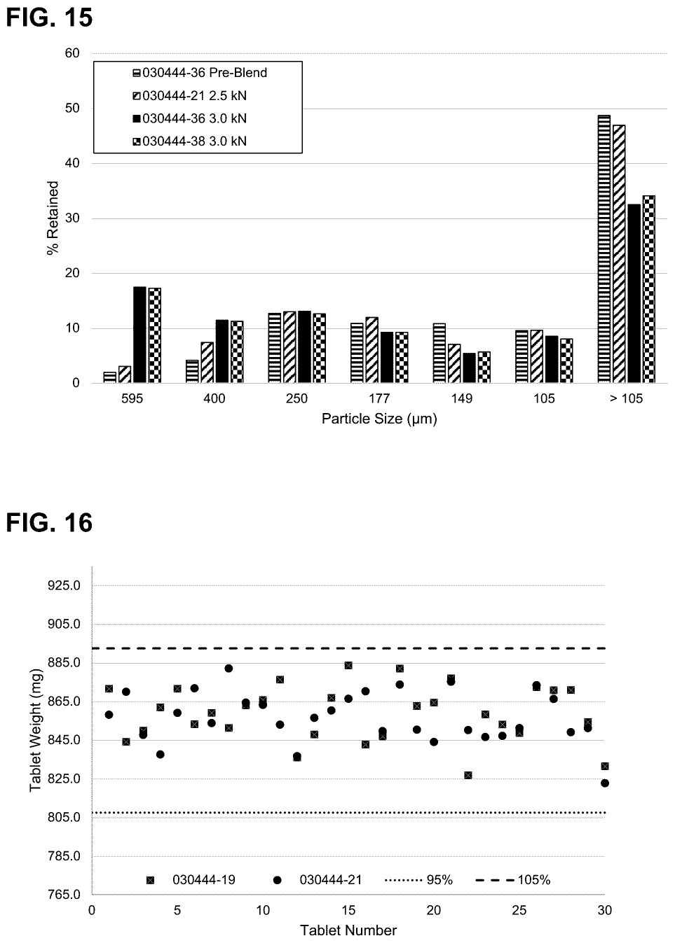

From the data in Table 21, it is evident the roller compaction process increases the bulk/tapped density, improves the flow (i.e. lower angle of repose), and increases the particle size distribution (PSD). See also FIG. 15.

TABLE 22
Formulation of batches 030444-36 and 030444-38.

| | 030444-36 | | 030444-38 | |
|---|---|---|---|---|
| | (Fast Profile) | | (Slow Profile) | |
| Material | mg/tab | % w/w | mg/tab | % w/w |
| Dexpramipexole Dihydrochloride Monohydrate | 319.01 | 37.53 | 319.01 | 35.44 |
| Carbomer (Carbopol 971P) | 102.00 | 12.00 | 127.50 | 14.17 |
| Sodium Carboxymethylcellulose (Aqualon CMC 7HXF | 238.00 | 28.00 | 297.50 | 33.06 |
| PH) | | | | |
| Silicified MCC (Prosolv HD90) | 180.36 | 21.22 | 144.74 | 16.08 |
| Colloidal Silicon Dioxide (Aerosil 200 P) | 4.25 | 0.50 | 4.50 | 0.50 |
| Magnesium Stearate (Ligamed MF-2-V) | 6.38 | 0.75 | 6.75 | 0.75 |
| Core Tablet Total | 850.00 | 100.00 | 900.00 | 100.00 |
| Opadry II White 85F18422 | 25.50 | 3.00 | 27.00 | 3.00 |
| Coated Tablet Total | 875.50 | 103.00 | 927.00 | 103.00 |

### Example 5.2—Compression Process Development

Smaller scale compression studies were performed using a Korsch XL-100 rotary tablet press with the final blends from the four roller compacted batches discussed above (Example 5.1). Larger scale batches (Examples 5.3 and 5.4) were also compressed using a Korsch XL-100.

A summary of the process parameters used is provided in Table 23. Start, middle, and end of batch samples were collected for weight, thickness, and hardness measurements and the results are summarized in Table 24 (030444-19, RC at 2.5 kN, 0.8 mm mesh screen), Table 25 (030444-21, RC at 2.5 kN, 0.8 mm mesh screen), Table 26 (030444-36, RC at 3.0 kN, 1.0 mm mesh screen), and Table 27 (030444-38, RC at 3.0 kN, 1.0 mm mesh screen).

TABLE 23
Summary of compression process parameters studied.

| | Compression study #1 | | Compression study #2 | |
|---|---|---|---|---|
| | (RC: 2.5 kN, 0.8 mm mesh) | | (RC: 3.0 kN, 1.0 mm mesh) | |
| Parameter | 030444-19 | 030444-21 | 030444-36 | 030444-38 |

Press Set-Up

| Tooling | 8.51 mm × 19 mm | | 8.51 mm × 19 mm | | 8.51 mm × 19 mm | | 8.51 mm × 19 mm | |
|---|---|---|---|---|---|---|---|---|
| Turret used | 8 station D | | 8 station D | | 8 station D | | 8 station D | |
| Number of punches used | 4 stations | | 4 stations | | 4 stations | | 4 stations | |
| Fill cam used | 8-14 | mm | 8-14 | mm | 8-14 | mm | 8-14 | mm |

Parameters

| | | | | | | | | |
|---|---|---|---|---|---|---|---|---|
| Pre-compression force | 0.5-0.7 | kN | 0.9-1.1 | kN | 0.5-0.6 | kN | 0.5-0.7 | kN |
| Main compression force | 21.8-27.2 | kN | 20.8-23.3 | kN | 22.5-23.8 | kN | 19.3-20.7 | kN |
| Feeder speed | 25 | rpm | 25 | rpm | 30 | rpm | 30 | rpm |
| Press speed | 25 | rpm | 28 | rpm | 20 | rpm | 20 | rpm |
| Fill depth | 11.4-11.9 | mm | 9.9 | mm | 8.7-8.9 | mm | 9.5-9.7 | mm |

TABLE 24
Summary of in-process compression checks for batch 030444-19.

| | Beginning | | | Middle | | | End | | |
|---|---|---|---|---|---|---|---|---|---|
| Tablet | Weight | Thickness | Hardness | Weight | Thickness | Hardness | Weight | Thickness | Hardness |
| No. | (mg) | (mm) | (kp) | (mg) | (mm) | (kp) | (mg) | (mm) | (kp) |
| 1 | 871.6 | 6.08 | 21.6 | 876.3 | 6.12 | 22.9 | 877.0 | 6.13 | 22.3 |
| 2 | 844.1 | 6.00 | 20.7 | 836.0 | 6.01 | 18.0 | 826.8 | 5.99 | 16.5 |
| 3 | 849.9 | 6.01 | 21.0 | 847.9 | 6.03 | 19.5 | 858.3 | 6.05 | 21.6 |
| 4 | 861.9 | 6.04 | 24.4 | 866.9 | 6.11 | 22.9 | 853.1 | 6.06 | 19.0 |
| 5 | 871.6 | 6.09 | 23.9 | 883.7 | 6.16 | 24.7 | 848.7 | 6.04 | 18.3 |
| 6 | 853.2 | 6.01 | 23.2 | 842.7 | 6.03 | 18.1 | 872.4 | 6.12 | 22.6 |
| 7 | 859.1 | 6.04 | 21.9 | 846.9 | 6.06 | 18.2 | 870.8 | 6.11 | 20.9 |
| 8 | 851.3 | 6.01 | 21.1 | 882.0 | 6.13 | 22.9 | 870.9 | 6.10 | 20.5 |
| 9 | 863.0 | 6.06 | 21.9 | 862.6 | 6.09 | 21.7 | 854.4 | 6.08 | 19.3 |
| 10 | 865.7 | 6.07 | 23.0 | 864.4 | 6.08 | 19.8 | 831.5 | 6.03 | 16.2 |
| Avg. | 859.1 | 6.04 | 22.3 | 860.9 | 6.08 | 20.9 | 856.4 | 6.07 | 19.7 |
| Min. | 844.1 | 6.00 | 20.7 | 836.0 | 6.01 | 18.0 | 826.8 | 5.99 | 16.2 |
| Max | 871.6 | 6.09 | 24.4 | 883.7 | 6.16 | 24.7 | 877.0 | 6.13 | 22.6 |

TABLE 25
Summary of in-process compression checks for batch 030444-21.

| | Beginning | | | Middle | | | End | | |
|---|---|---|---|---|---|---|---|---|---|
| Tablet | Weight | Thickness | Hardness | Weight | Thickness | Hardness | Weight | Thickness | Hardness |
| No. | (mg) | (mm) | (kp) | (mg) | (mm) | (kp) | (mg) | (mm) | (kp) |
| 1 | 858.1 | 6.08 | 23.2 | 853.0 | 6.04 | 19.1 | 875.2 | 6.12 | 19.2 |
| 2 | 870.0 | 6.11 | 18.9 | 836.7 | 6.02 | 16.2 | 850.2 | 6.06 | 17.8 |
| 3 | 847.7 | 6.08 | 17.2 | 856.5 | 6.03 | 17.9 | 846.6 | 6.01 | 16.7 |
| 4 | 837.6 | 6.04 | 15.7 | 860.3 | 6.07 | 19.2 | 847.2 | 6.04 | 16.9 |
| 5 | 859.1 | 6.10 | 17.6 | 866.4 | 6.08 | 19.1 | 851.2 | 6.03 | 17.0 |
| 6 | 871.8 | 6.12 | 19.6 | 870.3 | 6.10 | 20.5 | 873.4 | 6.14 | 19.2 |
| 7 | 853.8 | 6.10 | 17.7 | 849.7 | 6.02 | 17.4 | 866.3 | 6.09 | 18.3 |
| 8 | 882.1 | 6.16 | 20.1 | 873.7 | 6.11 | 19.9 | 849.1 | 6.01 | 16.7 |
| 9 | 864.4 | 6.12 | 17.0 | 850.4 | 6.06 | 17.7 | 851.2 | 6.04 | 17.1 |
| 10 | 863.2 | 6.10 | 20.0 | 844.0 | 6.02 | 15.5 | 822.7 | 5.98 | 14.1 |
| Avg. | 860.8 | 6.10 | 18.7 | 856.1 | 6.06 | 18.3 | 853.3 | 6.05 | 17.3 |
| Min. | 837.6 | 6.04 | 15.7 | 836.7 | 6.02 | 15.5 | 822.7 | 5.98 | 14.1 |
| Max | 882.1 | 6.16 | 23.2 | 873.7 | 6.11 | 20.5 | 875.2 | 6.14 | 19.2 |

TABLE 26
Summary of in-process compression checks for batch 030444-36 (fast prototype).

| | Beginning | | | Middle | | | End | | |
|---|---|---|---|---|---|---|---|---|---|
| Tablet | Weight | Thickness | Hardness | Weight | Thickness | Hardness | Weight | Thickness | Hardness |
| No. | (mg) | (mm) | (kp) | (mg) | (mm) | (kp) | (mg) | (mm) | (kp) |
| 1 | 843.2 | 5.99 | 20.0 | 848.0 | 5.99 | 22.1 | 847.8 | 5.97 | 21.0 |
| 2 | 840.5 | 5.98 | 18.7 | 853.2 | 6.01 | 22.3 | 853.7 | 5.99 | 22.1 |
| 3 | 849.9 | 5.99 | 20.6 | 849.5 | 5.99 | 22.2 | 846.6 | 5.98 | 21.3 |
| 4 | 849.7 | 5.98 | 21.2 | 856.6 | 6.00 | 22.0 | 849.4 | 5.98 | 22.9 |
| 5 | 857.8 | 6.00 | 21.0 | 857.3 | 6.01 | 20.4 | 836.1 | 5.95 | 20.0 |
| 6 | 848.8 | 5.97 | 21.6 | 850.4 | 6.00 | 21.0 | 856.1 | 5.99 | 22.3 |
| 7 | 850.9 | 6.00 | 20.0 | 844.5 | 5.98 | 19.8 | 859.7 | 6.01 | 22.3 |
| 8 | 851.8 | 6.00 | 20.6 | 851.4 | 6.00 | 21.8 | 852.2 | 5.98 | 20.5 |
| 9 | 848.0 | 6.00 | 21.5 | 841.3 | 5.96 | 19.9 | 856.9 | 5.99 | 23.4 |
| 10 | 845.7 | 6.00 | 19.9 | 850.7 | 5.99 | 20.6 | 847.8 | 5.97 | 20.3 |
| Avg. | 848.6 | 5.99 | 20.5 | 850.3 | 5.99 | 21.2 | 850.6 | 5.98 | 21.6 |
| Min. | 840.5 | 5.97 | 18.7 | 841.3 | 5.96 | 19.8 | 836.1 | 5.95 | 20.0 |
| Max | 857.8 | 6.00 | 21.6 | 857.3 | 6.01 | 22.3 | 859.7 | 6.01 | 23.4 |

TABLE 27
Summary of in-process compression checks for batch 030444-38 (slow prototype).

| | Beginning | | | Middle | | | End | | |
|---|---|---|---|---|---|---|---|---|---|
| Tablet | Weight | Thickness | Hardness | Weight | Thickness | Hardness | Weight | Thickness | Hardness |
| No. | (mg) | (mm) | (kp) | (mg) | (mm) | (kp) | (mg) | (mm) | (kp) |
| 1 | 899.3 | 6.38 | 19.5 | 896.2 | 6.33 | 19.5 | 890.1 | 6.33 | 18.8 |
| 2 | 898.4 | 6.36 | 17.5 | 890.0 | 6.33 | 20.1 | 892.7 | 6.33 | 19.8 |
| 3 | 895.6 | 6.37 | 19.3 | 900.2 | 6.33 | 19.6 | 903.6 | 6.36 | 19.9 |
| 4 | 902.3 | 6.38 | 20.1 | 898.4 | 6.33 | 19.2 | 903.5 | 6.35 | 18.9 |
| 5 | 910.4 | 6.41 | 20.1 | 906.1 | 6.34 | 20.3 | 889.2 | 6.34 | 17.6 |
| 6 | 868.3 | 6.30 | 15.3 | 899.2 | 6.34 | 19.4 | 899.0 | 6.34 | 19.6 |
| 7 | 895.7 | 6.37 | 18.8 | 888.2 | 6.30 | 18.4 | 890.1 | 6.34 | 19.8 |
| 8 | 899.0 | 6.38 | 19.5 | 896.5 | 6.33 | 19.1 | 907.6 | 6.38 | 21.1 |
| 9 | 908.5 | 6.40 | 19.6 | 886.0 | 6.30 | 19.3 | 907.4 | 6.40 | 20.0 |
| 10 | 896.2 | 6.14 | 26.9 | 890.3 | 6.33 | 18.6 | 901.8 | 6.35 | 20.2 |
| Avg. | 897.4 | 6.35 | 19.7 | 895.1 | 6.33 | 19.4 | 898.5 | 6.35 | 19.6 |
| Min. | 868.3 | 6.14 | 15.3 | 886.0 | 6.30 | 18.4 | 889.2 | 6.33 | 17.6 |
| Max | 910.4 | 6.41 | 26.9 | 906.1 | 6.34 | 20.3 | 907.6 | 6.40 | 21.1 |

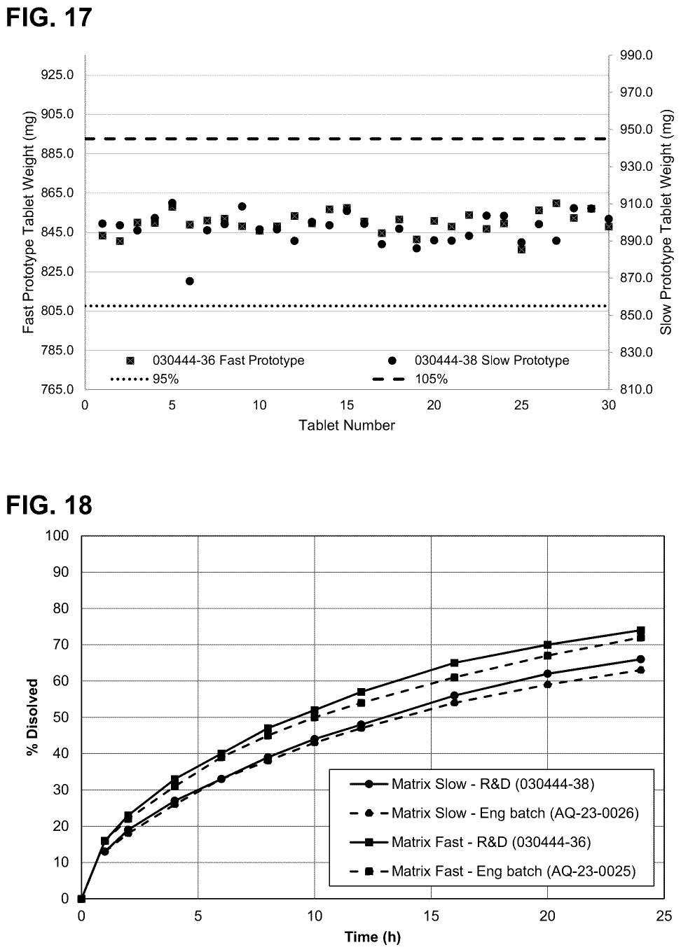

The results indicated that the process was overall robust. Weight variation between the final tablets was generally low (FIG. 16 and FIG. 17).

### Example 5.3 Scale-Up Batches (10 kg)

Two further batches (engineering batches) were manufactured at a scale of 10 kg. The formulations are provided in Table 28 and were identical to the formulations in Table 22 (which were manufactured at a scale of 1 kg; supra). A summary of the scale-up process parameters is shown in Table 29.

TABLE 28
Formulation of engineering batches AQ-23-0025 and AQ-23-0026.

| | AQ-23-0025 | | AQ-23-0026 | |
|---|---|---|---|---|
| | (Fast Profile) | | (Slow Profile) | |
| Material | mg/tab | % w/w | mg/tab | % w/w |
| Dexpramipexole Dihydrochloride Monohydrate | 319.01 | 37.53 | 319.01 | 35.44 |
| Carbomer (Carbopol 971P) | 102.00 | 12.00 | 127.50 | 14.17 |
| Sodium Carboxymethyl-cellulose (Aqualon CMC 7HXF PH) | 238.00 | 28.00 | 297.50 | 33.06 |
| Silicified MCC (Prosolv HD90) | 180.36 | 21.22 | 144.74 | 16.08 |
| Colloidal Silicon Dioxide (Aerosil 200 P) | 4.25 | 0.50 | 4.50 | 0.50 |
| Magnesium Stearate (Ligamed MF-2-V) | 6.38 | 0.75 | 6.75 | 0.75 |
| Core Tablet Total | 850.00 | 100.00 | 900.00 | 100.00 |
| Opadry II White 85F18422 | 25.50 | 3.00 | 27.00 | 3.00 |
| Coated Tablet Total | 875.50 | 103.00 | 927.00 | 103.00 |

TABLE 29
Summary of process parameters for engineering batches AQ-23-0025 and AQ-23-0026.

| Process Parameters | | AQ-23-0025 (Fast) | | AQ-23-0026 (Slow) | |
|---|---|---|---|---|---|
| Roller | Roller Pressure (kN) | 5 | | 5 | |
| Compaction | Roller Gap (mm) | 2.5 | | 2.5 | |
| | Roller Speed (rpm) | 3 | | 3 | |
| | Agitator Speed (rpm) | 10 | | 10 | |
| | Tamp/Feed Ratio | 170% | | 170% | |
| | Granulator Speed CW/CCW | 70 rpm/70 rpm | | 70 rpm/70 rpm | |
| | Granulator Angle CW/CCW | 250°/350° | | 250°/350° | |
| Compression | Target Weight (mg) | 850.00 | (786.25-913.75) | 900.00 | (832.50-967.50) |
| | Target Composite (mg) (10 tabs) | 8500 | (8075-8925) | 9000 | (8550-9450) |
| | Target Thickness (mm) | Report Results | | Report Results | |
| | Target Hardness (kP) | 22 | (15-30) | 22 | (15-30) |
| | Press Speed (rpm) | 20 | | 20 | |
| | Feeder Speed (rpm) | 30 | | 30 | |
| Coating | Pan size (in) | 19 | | 19 | |
| | Nozzle port opening (mm) | 1.2 | | 1.2 | |
| | Drum Speed (rpm) | 10-14 | | 12 | |
| | Inlet Air Volume (cfm) | 179-184 | | 178-185 | |
| | Inlet Air Temperature (° C.) | 60-64 | | 63-66 | |
| | Exhaust Air Temperature (° C.) | 45-49 | | 49-50 | |
| | Target Weight Gain | 3% | (2%-4%) | 3% | (2%-4%) |

| | | |
|---|---|---|
| Spray Rate (g/min) | 21-25 | 19-20 |
| Pattern Air Pressure (psi) | 20 | 20 |
| Atomizing Air Pressure (psi) | 15-20 | 20 |

FIG. 18 shows a comparison of in vitro dissolution of the larger-scale tablets (10 kg) to the in vitro dissolution of the smaller-scale tablets (1 kg) and indicates good reproducibility.

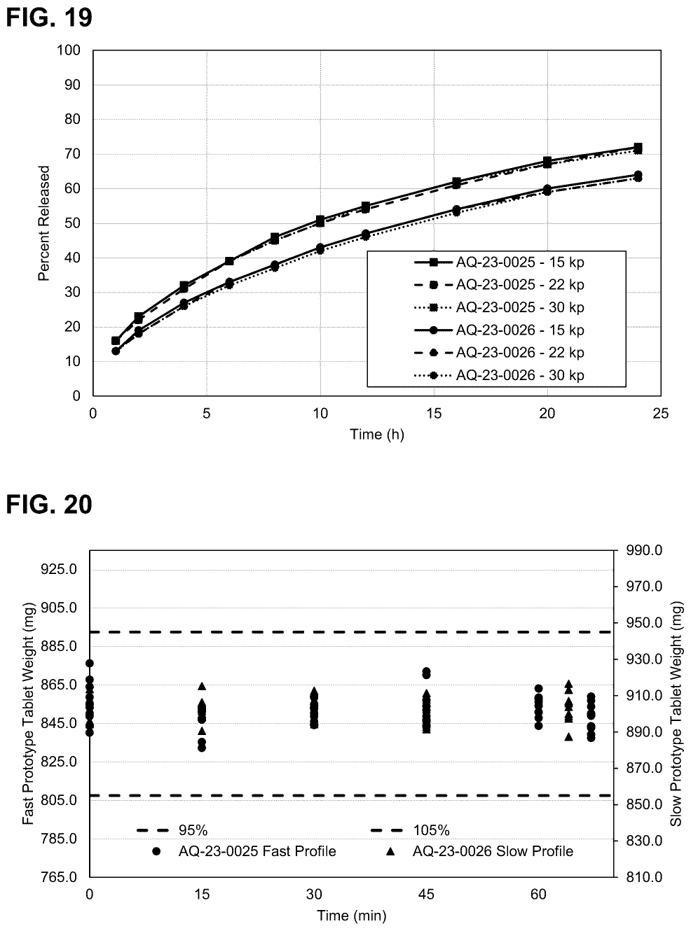

The larger-scale tablets were also produced with a varying tablet hardness to study the effect of tablet hardness on dissolution. The in vitro dissolution profiles are presented in FIG. 19 and show that the dissolution was independent of tablet hardness. The compression was proceeded with a target tablet hardness of 22 kp.

Moreover, in-process samples were collected every 15 min during the compression process. The tablet weight variation is presented in FIG. 20 and shows that the tablets were controlled well within +/−5%.

### Example 5.4—Clinical Trial Material Batches

Phase I clinical trial material batches were manufactured at a similar scale as the batches in Example 5.3 (engineering batches manufactured at a scale of 10 kg). The formulations are provided in Table 30 and the process parameters are presented in Table 31.

TABLE 30
Formulation of batches 23JM-107 and 23JM-108 (clinical trial batches). See also Table 32.

| | 23JM-107 (Fast) | | 23JM-108 (Slow) | |
|---|---|---|---|---|
| Material | mg/tab | % w/w | mg/tab | % w/w |
| Dexpramipexole Dihydrochloride Monohydrate | 319.01 | 37.53 | 319.01 | 35.44 |
| Carbomer (Carbopol 971P) | 102.00 | 12.00 | 127.50 | 14.17 |
| Sodium Carboxymethyl-cellulose (Aqualon CMC 7HXF PH) | 238.00 | 28.00 | 297.50 | 33.06 |
| Silicified MCC (Prosolv HD90) | 180.36 | 21.22 | 144.74 | 16.08 |
| Colloidal Silicon Dioxide (Aerosil 200 P) | 4.25 | 0.50 | 4.50 | 0.50 |
| Magnesium Stearate (Ligamed MF-2-V) | 6.38 | 0.75 | 6.75 | 0.75 |
| Core Tablet Total | 850.00 | 100.00 | 900.00 | 100.00 |
| Opadry II White 85F18422 | 25.50 | 3.00 | 27.00 | 3.00 |
| Coated Tablet Total | 875.50 | 103.00 | 927.00 | 103.00 |

TABLE 31
Summary of process parameters for batches 23JM-107 and 23JM-108.

| Process Parameters | | 23JM-107 (Fast) | | 23JM-108 (Slow) | |
|---|---|---|---|---|---|
| Roller | Force (kN) | 5 | | 5 | |
| Compaction | Gap (mm) | 2.5 | | 2.5 | |
| | Speed (rpm) | 3 | | 3 | |
| | Agitator Speed (rpm) | 10 | | 10 | |
| | Tamp/Feed Ratio | 170% | | 170% | |
| | Granulator Speed CW/CCW | 70 rpm/70 rpm | | 70 rpm/70 rpm | |
| | Granulator Angle CW/CCW | 250°/350° | | 250°/350° | |
| Compression | Target Weight (mg) | 850.00 | (786.25-913.75) | 900.00 | (832.50-967.50) |
| | Target Composite (mg) | 8500 | (8075-8925) | 9000 | (8550-9450) |
| | Target Thickness (mm) | Report Results | | Report Results | |
| | Target Hardness (kP) | 22 | (15-30) | 22 | (15-30) |
| | Press Speed (rpm) | 20 | | 20 | |
| | Feeder Speed (rpm) | 30 | | 30 | |
| Coating | Pan size (in) | 19 | | 19 | |
| | Nozzle port opening (mm) | 1.2 | | 1.2 | |
| | Drum Speed (rpm) | 12 | | 12 | |
| | Inlet Air Volume (cfm) | 180-182 | | 180-182 | |
| | Inlet Air Temperature (° C.) | 63-64 | | 61-63 | |
| | Exhaust Air Temperature(° C.) | 48-50 | | 48 | |
| | Target Weight Gain | 3% | (2%-4%) | 3% | (2%-4%) |

| | | |
|---|---|---|
| Spray Rate (g/min) | 22 | 20-22 |
| Pattern Air Pressure (psi) | 20 | 20 |
| Atomizing Air Pressure (psi) | 20 | 20 |

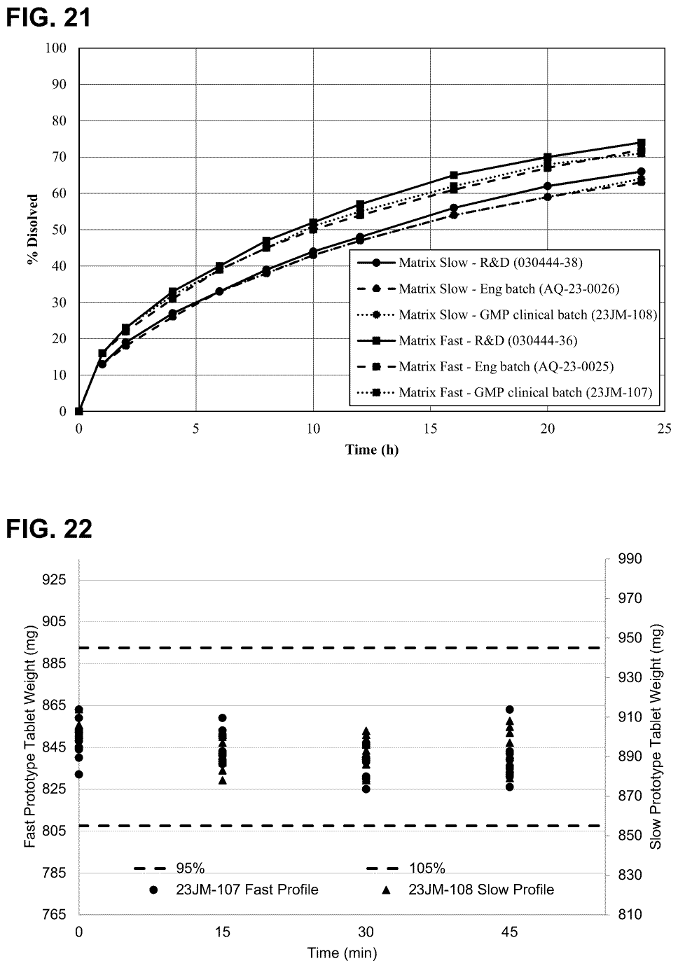

Dissolution of the clinical batches are compared to the lab-scale stability batches (1 kg) and engineering batches (10 kg); the results are presented in FIG. 21 and indicate good reproducibility.

In-process samples were collected at 15 min intervals, and the tablet weight variations are presented in FIG. 22. Tablet weights were consistent throughout the process for both batches and well controlled within the limits.

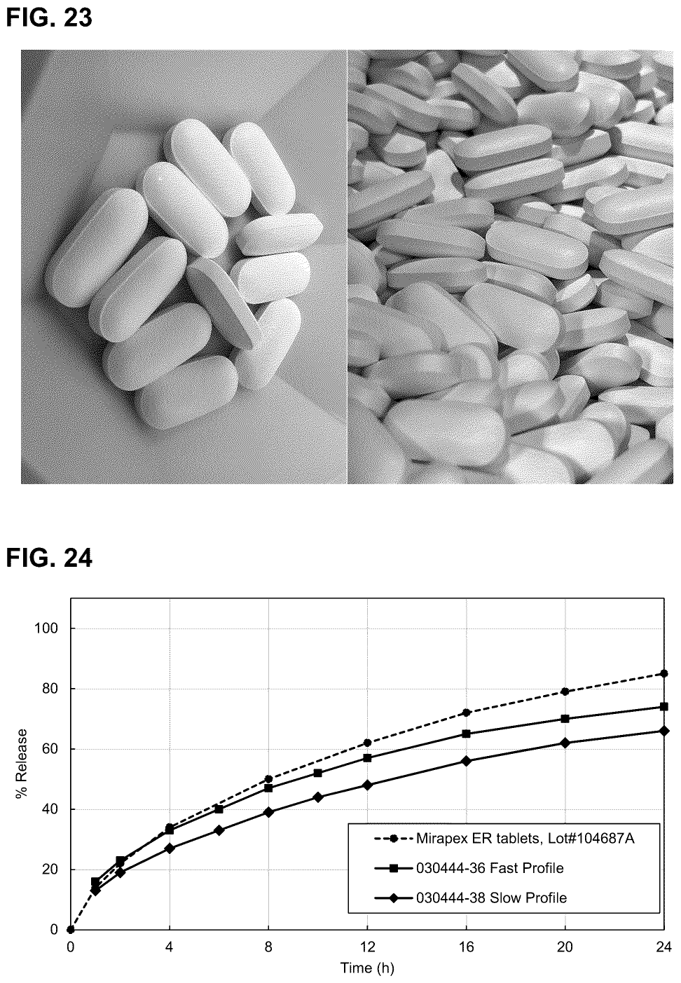

The resulting tablets were free of scuff marks and had no observable defects, as shown in FIG. 23.

### Example 6—Stability Evaluation

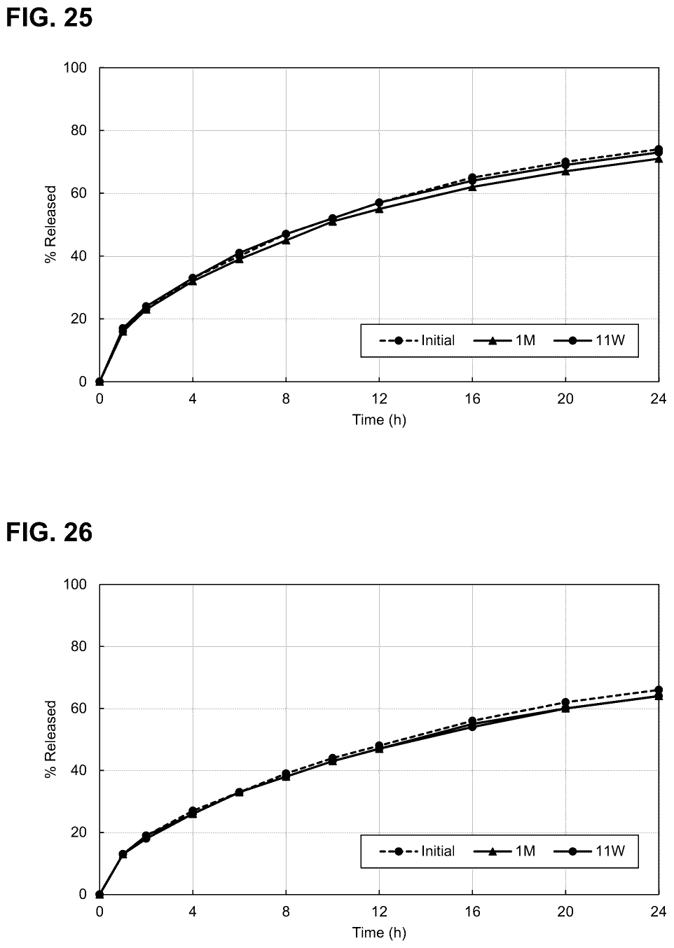

The tablet formulations shown in Table 22 (030444-36 and 030444-38) manufactured at a 1 kg scale and comprising an Opadry film coating system were evaluated for their stability under controlled room temperature (CRT) conditions (temperature range of 20° C. to 25° C.). FIG. 24 shows the in vitro dissolution of the tablet formulations shown in Table 22 compared to the pramipexole ER comparison tablet. As can be taken from FIGS. 25 and 26, the tablet formulations were stable when stored under CRT conditions for 1 month, as well as 11 weeks.

### Example 7—Clinical Evaluation

For evaluation in clinical trials, two matrix tablet formulations (“slow” and “fast”) as shown in Table 32 were prepared (see also Example 5.4).

TABLE 32
Formulations of dexpramipexole dihydrochloride monohydrate sustained
release matrix tablets (“slow” and “fast”) for
evaluation in clinical trials. N/A = not applicable. See also Table 30.

| | Matrix Tablet, Fast | | | Matrix Tablet, Slow | | |
|---|---|---|---|---|---|---|
| | % w/w | % w/w | | % w/w | % w/w | |
| Ingredient | (core) | (total) | mg/tab | (core) | (total) | mg/tab |

Intra-Granular

| | | | | | | |
|---|---|---|---|---|---|---|
| Dexpramipexole Dihydrochloride | 37.53 | 36.44 | 319.01* | 35.44 | 34.41 | 319.01* |
| Monohydrate | | | | | | |
| Sodium Carboxymethylcellulose | 28.00 | 27.18 | 238.00 | 33.06 | 32.09 | 297.50 |
| (Aqualon CMC 7HXF PH) | | | | | | |
| Carbomer (Carbopol 971P) | 12.00 | 11.65 | 102.00 | 14.17 | 13.75 | 127.50 |
| Silicified Microcrystalline | 21.22 | 20.60 | 180.36 | 16.08 | 15.61 | 144.74 |
| Cellulose (Prosolv HD90) | | | | | | |
| Magnesium Stearate | 0.25 | 0.24 | 2.13 | 0.25 | 0.24 | 2.25 |
| (Ligamed MF-2-V) | | | | | | |

Extra-Granular

| | | | | | | |
|---|---|---|---|---|---|---|
| Colloidal Silicon Dioxide | 0.50 | 0.49 | 4.25 | 0.50 | 0.49 | 4.50 |
| (Aerosil 200P) | | | | | | |
| Magnesium Stearate | 0.50 | 0.49 | 4.25 | 0.50 | 0.49 | 4.50 |
| (Ligamed MF-2-V) | | | | | | |
| Total (Tablet Core) | 100.00 | 97.09 | 850.00 | 100.00 | 97.09 | 900.00 |

Film Coating

| | | | | | | |
|---|---|---|---|---|---|---|
| Opadry II White 85F18422 | N/A | 2.91 | 25.50 | N/A | 2.91 | 27.00 |
| (Polyvinyl Alcohol, Titanium | | | | | | |
| Dioxide, Polyethylene Glycol, | | | | | | |
| Talc) | | | | | | |
| Purified water\hat{ } | N/A | N/A | 102.00 | N/A | N/A | 108.00 |
| Total (Tablet) | N/A | 100.00 | 875.50 | N/A | 100.00 | 927.00 |

*319.01 mg of dexpramipexole dihydrochloride monohydrate is equivalent to 300.00 mg dexpramipexole dihydrochloride.
\hat{ }Removed during processing; solids in coating solution = 20% w/w.

As described above, the tablets were prepared using roller compaction. More particular, the intra-granular ingredients shown in Table 32 were mixed into a pre-blend from which subsequently densified granules were prepared by roller compaction. The densified granules were subsequently blended with glidant (colloidal silicon dioxide, pre-lubrication) and lubricant (magnesium stearate, lubrication). See Table 32.

### Example 8—Phase I Clinical Trial in Healthy Participants (EXHALE-6)

The matrix tablet formulations shown in Table 32 are given to healthy human volunteers once a day (QD; quaque die) to assess bioavailability of dexpramipexole. The clinical study is designed to compare four ER tablet formulations (QD tablet formulations), including the two matrix tablet formulations of Table 32, to an immediate release (IR) tablet formulation (bis in die (BID) tablet formulation). The ER tablet formulations provide a dose of 300 mg dexpramipexole dihydrochloride equivalent (which corresponds to a daily dose of 300 mg dexpramipexole dihydrochloride equivalent, as the ER tablet formulations are given once a day). The IR tablet formulation provides a dose of 150 mg dexpramipexole dihydrochloride equivalent (which also corresponds to a daily dose of 300 mg dexpramipexole dihydrochloride equivalent, as the IR tablet formulation is given twice a day). The composition of the IR tablet formulation is given in Table 33.

TABLE 33
Composition of BID IR tablet formulation.

| Component | Function | Quantity per Tablet | |
|---|---|---|---|
| Dexpramipexole | Drug Substance | 159.50 | mg* |
| dihydrochloride monohydrate | | | |
| Microcrystalline cellulose | Diluent | 132.19 | mg |
| Mannitol | Diluent | 65.50 | mg |
| Crospovidone | Disintegrant | 15.00 | mg |
| Magnesium stearate | Glidant | 2.81 | mg |

| | | |
|---|---|---|
| Core Total | 375.0 | mg |

Tablet Coat

| | | | |
|---|---|---|---|
| Opadry II white | Film former | 11.25 | mg |

| | | |
|---|---|---|
| Coated tablet Total | 386.25 | mg |

*159.50 mg of dexpramipexole dihydrochloride monohydrate is equivalent to 150.0 mg dexpramipexole dihydrochloride based on molecular weight ratio of monohydrate to anhydrous API.

### Study Design

A Phase I, open-label, single-center, five-period, randomized, crossover study in 15 healthy adult male and female participants is conducted to evaluate the relative bioavailability of dexpramipexole extended release (ER) formulations to an immediate release (IR) tablet formulation. Participants provide informed consent and complete screening procedures within 21 days prior to Day −1. All participants eligible for enrollment must meet all inclusion criteria and none of the exclusion criteria. Participants are randomly assigned to one of five sequences (i.e., study arms or study groups) and receive single oral doses of dexpramipexole ER formulations on four dosing days and two oral doses of dexpramipexole IR formulation given every 12 hours (q12h) on one dosing day for a total of five dosing days/periods. Each participant receives a different formulation in each period. Participants are receiving dexpramipexole ER and IR formulations on Days 1, 4, 8, 11, and 15, according to random assignment. Drug plasma exposure is expected to occur within the first 24 hours following administration. The washout period before the next treatment period is expected to be sufficient given the study design and drug properties. Participants are required to be domiciled at the study site for the duration of the study (Day −1 to Day 17) and return for a follow-up visit on Day 18. The entire duration of the study, including Screening, is approximately 6 weeks.

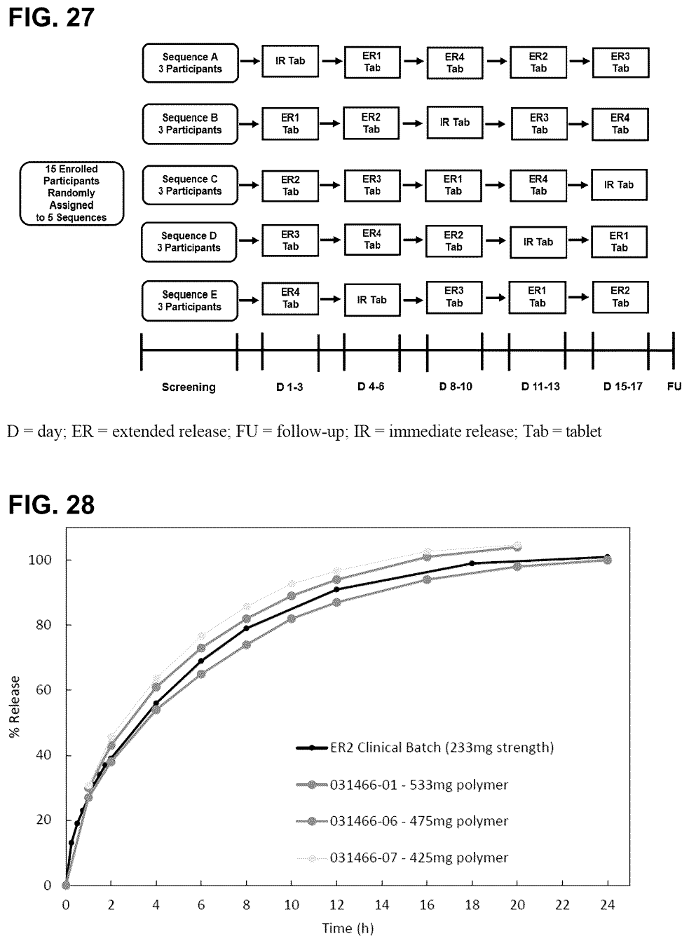

A schematic outline of the clinical study is given in FIG. 27 (wherein ER1 and ER2 correspond to the two matrix tablet formulations of Table 32).

### Study Assessments—Pharmacokinetics (PK)

Blood samples (three mL each; up to 22 samples per period) for the preparation of plasma are collected at specified time points for 48 hours after oral administration of dexpramipexole (relative to first dose for IR formulation) for the determination of dexpramipexole concentrations in plasma. Thus, blood samples are collected for plasma dexpramipexole concentration at predose (0), 0.5, 1, 1.5, 2, 2.5, 3, 4, 5, 6, 8, 10, 12 (predose for second dose of IR formulation), 12.5, 13, 14, 15, 16, 20, 24, 36, and 48 hours postdose (relative to first dose for IR formulation). When a PK sample collection time point corresponds with study drug dosing, the PK sample is collected before study drug dosing. Table 34 provides the schedule for pharmacokinetic blood sample collection.

TABLE 34
Pharmacokinetic blood sample collection schedule.

| | | Collection Window | | PK Blood Sample |
|---|---|---|---|---|
| Days | Time (h) | Relative to Time | | Collection |
| Days 1, 4, 8, | 0 | Up to 60 min predose | | X |
| 11, and 15 | 0.5 | ±5 | min | X |
| | 1 | ±5 | min | X |
| | 1.5 | ±5 | min | X |
| | 2 | ±5 | min | X |
| | 2.5 | ±5 | min | X |
| | 3 | ±5 | min | X |
| | 4 | ±5 | min | X |
| | 5 | ±5 | min | X |
| | 6 | ±5 | min | X |
| | 8 | ±5 | min | X |
| | 10 | ±5 | min | X |
| | 12 | ±5 | min^{a} | X |
| | 12.5 | ±5 | min | X |
| | 13 | ±5 | min | X |
| | 14 | ±5 | min | X |
| | 15 | ±5 | min | X |
| | 16 | ±5 | min | X |
| | 20 | ±15 | min | X |
| Days 2, 5, 9, | 24 | ±15 | min | X |
| 12, and 16 | 36 | ±15 | min | X |
| Days 3, 6, 10, | 48 | ±15 | min | X^{b} |

13, and 17

Abbreviation: CRU = clinical research unit; h = hour; min = minute; PK = pharmacokinetic.
^{a}Must be collected before the second IR dose.
^{b}Participants will be discharged from the CRU after the 48-hour PK blood sample collection on Day 17 (Period 5).

Plasma dexpramipexole concentrations are analyzed using noncompartmental methods for the determination of the following PK parameters:
maximum observed plasma concentration (Cₘₐₓ; for IR tablet, calculated after each dose [Cₘₐₓ over the 0- to 12-hour interval, C_{max,0-12}; and Cₘₐₓ over the 12- to 24-hour interval, C_{max,12-24}] and overall),
time to the maximum observed plasma concentration (tₘₐₓ; for IR tablet, calculated after each dose [tₘₐₓ over the 0- to 12-hour interval, t_{max,0-12}; and tₘₐₓ over the 12- to 24-hour interval, t_{max,12-24}] and overall),
area under the concentration-time curve (AUC) from time 0 to the time of 24 hours postdose (AUC₀₋₂₄),
AUC from time 0 to the time of the last measurable concentration (AUC₀₋ₜ),
AUC from time 0 extrapolated to infinity (AUC_{0-inf}; for ER formulations only),
time prior to the first measurable (non-zero) concentration (t_{lag}; for IR tablet, calculated for the first dose only),
apparent terminal elimination rate constant (λ_{Z}; for IR tablet, calculated after the second dose only), and
apparent terminal half-life (t_{1/2}; for IR tablet, calculated after the second dose only).

Additional PK parameters that are determined to fully characterize PK of dexpramipexole are described in the statistical analysis plan (SAP).

### Study Assessments—Safety

Safety and tolerability are monitored throughout the study by observed and reported adverse events (AEs; serious and non-serious AEs), clinical laboratory assessments (including serum chemistry, hematology, and urinalysis; see Table 35 showing clinical laboratory assessment tests that are performed), vital signs (blood pressure, heart rate, respiratory rate, and oral body temperature), electrocardiogram (ECG) measurements, and physical examinations. If an ECG is scheduled at the same time point as blood sample collection, the ECG is performed first.

TABLE 35
Clinical Laboratory Tests

| Hematology | Serum Chemistry | Urinalysis |
|---|---|---|
| Complete Blood Count (CBC) | Alanine aminotransferase (ALT) | Appearance |
| Hematocrit | Albumin | Bilirubin |
| Hemoglobin | Alkaline phosphatase | Blood |
| Platelet count | Aspartate aminotransferase (AST) | Color |
| Red blood cell count | Bicarbonate | Glucose |
| White blood cell count | Blood urea nitrogen (BUN) | Ketones |
| (with automated | Calcium | Leukocyte esterase |
| differential) | Chloride | Nitrite |
| | Creatinine | pH |
| | Direct bilirubin | Protein |
| | Indirect bilirubin | Specific gravity |
| | Gamma-glutamyl transferase | Urobilinogen |
| | (GGT) | Microscopic examination: |
| | Glucose | red blood cells; white |
| | Estimated glomerular filtration | blood cells; epithelial |
| | rate (eGFR)^{a} | cells; bacteria, crystals, |
| | Lactic dehydrogenase (LDH) | casts, etc. (if present) |
| | Magnesium | |
| | Phosphorus | |
| | Potassium | |
| | Sodium | |
| | Total bilirubin | |
| | Total protein | |
| | Uric acid | |

Diagnostic Screening Tests:

| Blood | Urine Drug Screen and Cotinine |
|---|---|
| Serology: hepatitis panel (hepatitis C antibody, | Urine drug screen: amphetamines, barbiturates, |
| hepatitis B surface antigen, hepatitis B core | benzodiazepines, cannabinoids, cocaine, alcohol, |
| antibody), and HIV antibody (Screening) | and opiates (Screening and Check-in [Day −1]). |
| Serum pregnancy test (all female participants at | Cotinine: |
| Screening, Check-in [Day −1], and before | (Screening and Check-in [Day −1]). |
| Discharge/Early Termination) | Additional urine may be collected as needed per |
| FSH (female participants not surgically sterile | the schedule of assessments. |
| who have had spontaneous amenorrhea for at least | |
| 1 year) at Screening | |

^{a}eGFR is calculated using the Chronic Kidney Disease Epidemiology Collaboration formula at Screening.

An AE is any untoward medical occurrence associated with the use of a drug in humans and which does not necessarily have to have a causal relationship with this treatment. An AE can be any unfavorable and unintended sign, symptom, or disease temporally associated with the use of a medicinal product, whether or not considered related to the medicinal product [ICH E2A Guideline, 1994].

A serious adverse event (SAE) is any untoward medical occurrence that at any dose:
Results in death
Is life threatening, i.e., the participant was, in the opinion of the investigator, at immediate risk of death from the event as it occurred (it does not include an event that, had it occurred in a more severe form, might have caused death)
Results in a significant, persistent, or permanent change, impairment, damage or disruption in the participant's body function/structure, physical activities and/or quality of life
Requires in-participant hospitalization or prolongs hospitalization
Is a congenital anomaly/birth defect, or
Is an important medical event.

It is determined whether there is a reasonable possibility or not that the investigational product caused an AE, based on whether there is evidence that suggests a causal relationship between the investigational product and the AE.

Each AE is rated for severity (intensity) according to the following definitions:
Mild: No interference with daily activities
Moderate: Some interference with activity not requiring medical intervention
Severe: Prevents daily activity and requires medical intervention
Potentially Life Threatening: ER visit or hospitalization

Adverse events of any intensity are reported as SAEs if at least 1 criteria established above is met. The occurrence of a life-threatening adverse event results in the individual being discontinued from the study drug. The event is followed until it resolves or stabilizes.

The schedule of assessments for Periods 1, 2, 3, 4, and 5 is given in Table 36 below.

TABLE 36
Schedule of assessments for Periods 1, 2, 3, 4, and 5.

| | Day-21 | Day-1 | | | | | | | | | | | | |
|---|---|---|---|---|---|---|---|---|---|---|---|---|---|---|
| | Screen- | Check- | Day 1, 4, 8, 11, and 15 | | | | | | | | | | | |
| Event | ing^{b} | In | 0 h | 0.5 h | 1 h | 1.5 h | 2 h | 2.5 h | 3 h | 4 h | 5 h | 6 h | 8 h | 10 h |
| Informed | x | | | | | | | | | | | | | |
| consent | | | | | | | | | | | | | | |
| Medical | x | | | | | | | | | | | | | |
| history | | | | | | | | | | | | | | |
| Physical | x | | | | | | | | | | | | | |
| examination | | | | | | | | | | | | | | |
| (including | | | | | | | | | | | | | | |
| weight and | | | | | | | | | | | | | | |
| height)^{c} | | | | | | | | | | | | | | |
| BMI | x | | | | | | | | | | | | | |
| Vital signs^{d} | x | x | x | | | | x | | | x | | | x | |
| ECG | x | x | | | | | | | | | | | | |
| (12-lead) | | | | | | | | | | | | | | |
| Hematology | x | x | | | | | | | | | | | | |
| Clinical | x | x | | | | | | | | | | | | |
| chemistry | | | | | | | | | | | | | | |
| Urinalysis | x | x | | | | | | | | | | | | |
| HIV antibody | x | | | | | | | | | | | | | |
| Hepatitis | x | | | | | | | | | | | | | |
| serology^{e} | | | | | | | | | | | | | | |
| Serum | x | x^{g} | | | | | | | | | | | | |
| pregnancy | | | | | | | | | | | | | | |
| test^{f} | | | | | | | | | | | | | | |
| Urine | x | x^{g} | | | | | | | | | | | | |
| cotinine | | | | | | | | | | | | | | |
| screen | | | | | | | | | | | | | | |
| Urine | x | x^{g} | | | | | | | | | | | | |
| drug screen^{h} | | | | | | | | | | | | | | |
| PK sampling | | | x | x | x | x | x | x | x | x | x | x | x | x |
| ER tablet | | | x | | | | | | | | | | | |
| study drug | | | | | | | | | | | | | | |
| admini- | | | | | | | | | | | | | | |
| stration^{j} | | | | | | | | | | | | | | |
| IR tablet | | | x | | | | | | | | | | | |
| study drug | | | | | | | | | | | | | | |
| admini- | | | | | | | | | | | | | | |
| stration^{j} | | | | | | | | | | | | | | |
| Concomitant | x | x | [symbol] | | | | | | | | | | | |
| Medications | | | | | | | | | | | | | | |

| Adverse | x | x | | [symbol] | | | | | | | |
|---|---|---|---|---|---|---|---|---|---|---|---|
| Events | | | | | | | | | | | |
| Clinic Visit^{k} | x | [symbol] | | | | | | | | | |
| | | | | | | | | Days 2, 5, | | Days 3, 6, | |
| | Day 1, 4, 8, 11, and 15 | | | | | | | 9, 12, and 16 | | 10, 13, and 17 | |
| Event | 12 h | 12.5 h | 13 h | 14 h | 15 h | 16 h | 20 h | 24 h | 36 h | 48 h | 18^{a} |
| Informed | | | | | | | | | | | |
| consent | | | | | | | | | | | |
| Medical | | | | | | | | | | | |
| history | | | | | | | | | | | |
| Physical | | | | | | | | | | x | x |
| examination | | | | | | | | | | | |
| (including | | | | | | | | | | | |
| weight and | | | | | | | | | | | |
| height)^{c} | | | | | | | | | | | |
| BMI | | | | | | | | | | | |
| Vital signs^{d} | | | | | | | | x | | x | x |
| ECG | | | | | | | | | | x | x |
| (12-lead) | | | | | | | | | | | |
| Hematology | | | | | | | | | | x | x |
| Clinical | | | | | | | | | | x | x |
| chemistry | | | | | | | | | | | |
| Urinalysis | | | | | | | | | | x | x |
| HIV antibody | | | | | | | | | | | |
| Hepatitis | | | | | | | | | | | |
| serology^{e} | | | | | | | | | | | |
| Serum | | | | | | | | | | | x |
| pregnancy | | | | | | | | | | | |
| test^{f} | | | | | | | | | | | |
| Urine | | | | | | | | | | | |
| cotinine | | | | | | | | | | | |
| screen | | | | | | | | | | | |
| Urine | | | | | | | | | | | |
| drug screen^{h} | | | | | | | | | | | |
| PK sampling | x | x | x | x | x | x | x | x | x | x | xⁱ |
| ER tablet | | | | | | | | | | | |
| study drug | | | | | | | | | | | |
| admini- | | | | | | | | | | | |
| stration^{j} | | | | | | | | | | | |
| IR tablet | x | | | | | | | | | | |
| study drug | | | | | | | | | | | |
| admini- | | | | | | | | | | | |
| stration^{j} | | | | | | | | | | | |
| Concomitant | [symbol] | | | | | | | | | | |
| Medications | | | | | | | | | | | |

| | |
|---|---|
| Adverse | [symbol] |
| Events | |
| Clinic Visit^{k} | [symbol] |

ECG = electrocardiogram;
ER = extended release;
FU = follow-up;
IR = immediate release;
PK = pharmacokinetic
^{a}Day 18 procedures should be conducted within 72-96 hrs after last dose. Day 18 study evaluations are also to be performed as early termination procedures.
^{b}All screening procedures are to be completed within 21 days prior to Day −1.
^{c}Height will be measured only at screening.
^{d}Vital signs will be assessed within 1 hour of dosing.
^{e}Hepatitis C antibody, hepatitis B surface antigen, hepatitis B core antibody.
^{f}Serum pregnancy testing for all female participants at screening, check-in [Day −1], and before discharge/early termination. Follicle-stimulating hormone testing at screening only for female participants not surgically sterile and who have had spontaneous amenorrhea for at least 12 months.
^{g}Negative pregnancy test, drug screen and cotinine results were to be obtained prior to first dose.
^{h}Amphetamines, benzodiazepines, cocaine, barbiturates, cannabis, opiates, and alcohol.
ⁱPK sample to be collected only as a part of early termination procedures.
^{j}Participant will be administered dose based on sequence assignment.
^{k}Participants will be confined on Day −1 until completion of the 48-hour postdose evaluations on Day 17.

### Study Objectives

Primary Objective: To compare the relative bioavailability of a single dose of each ER formulation to two doses of the IR formulation given every 12 hours (q12h).

Secondary Objectives: To evaluate the safety and tolerability of each formulation and to describe the pharmacokinetics (PK) of the IR dosage formulation and each ER dosage formulation.

### Study Endpoints

Primary Endpoints: Cₘₐₓ and AUC₀₋₂₄

Key Secondary Endpoints: Safety endpoints: AEs, vital signs (systolic and diastolic blood pressure, respiratory rate, pulse, and temperature), clinical laboratory assessments, ECGs, and physical examinations

Other Secondary Endpoints: AUC₀₋ₜ, AUC_{0-inf}, tₘₐₓ, t_{lag}, and other parameters, as appropriate

### Inclusion Criteria

A participant may be included in the study only if all of the following inclusion criteria are met:
1. Is a healthy male or female at least 18 and not greater than 55 years old at screening
2. Is a non-smoker defined by a history of non-smoking or smoking cessation and tobacco (including nicotine replacement) abstinence for over 1 year from screening
3. Has a body mass index (BMI) of ≥18 and ≤32 kg/m²: (BMI=weight (kg)±[height (m)]²) at screening
4. Has normal findings upon physical examination (i.e., no clinically significant abnormal physical findings)
5. Has normal or not clinically significant 12-lead electrocardiogram (ECG) findings at screening and Day −1
6. Has a normal systemic blood pressure defined as a systolic pressure of 90-140 mmHg and a diastolic pressure of 50-90 mmHg, may repeat at discretion of the Investigator at screening.
7. Has a normal resting heart rate between 50 and 100 beats per minute (bpm) on vitals, may repeat at discretion of Investigator at screening.
8. Is willing and able to communicate with the study team and abide by all study requirements and restrictions.
9. Is willing to provide signed, written informed consent.

### Exclusion Criteria

A participant meeting any of the following criteria is not eligible to participate in the study:
1. Has a history or current evidence of any significant acute or chronic medical illness.
2. Has a history of any gastrointestinal issues or surgery (except simple appendectomy, cholecystectomy, or hernia repair) that could possibly impact drug absorption.
3. Has any major surgery within 12 weeks prior to (Day −1).
4. Has a history of recent (within 6 months) drug or alcohol abuse.
5. Has evidence of organ dysfunction or any clinically significant deviation from normal in the medical history, physical examination, vital signs, ECG, and/or clinical laboratory determinations, may repeat at discretion of Investigator.
6. Women of childbearing potential (WOCBP; after menarche) must use either of the following methods of birth control, from Screening through the End of Study Visit:
   a. A highly effective form of birth control (confirmed by the investigator). Highly effective forms of birth control include: true sexual abstinence, a vasectomized sexual partner, Implanon, female sterilization by tubal occlusion, any effective Intrauterine device (IUD), IUD/intrauterine system (IUS), Levonorgestrel Intrauterine system, or combined (estrogen and progestogen-containing or progestogen-only) hormonal contraceptive associated with inhibition of ovulation.
   OR
   b. Two protocol acceptable methods of contraception (i.e., progestogen-only oral hormonal conception, where inhibition of ovulation is not the primary mode of action; male or female condom with or without spermicide; cap, diaphragm or sponge with spermicide) in tandem.
Women not of childbearing potential are defined as women who are either permanently sterilized (hysterectomy, bilateral oophorectomy, or bilateral salpingectomy), or who are postmenopausal. Women will be considered postmenopausal if they have been amenorrheic for 12 months prior to the planned date of the Screening Visit without an alternative medical cause. The following age specific requirements apply:
   c. women <50 years old will be considered postmenopausal if they have been amenorrheic for 12 months or more following cessation of exogenous hormonal treatment and follicle stimulating hormone (FSH) levels are in the postmenopausal range.
   d. Women ≥50 years old will be considered postmenopausal if they have been amenorrheic for 12 months or more following cessation of all exogenous hormonal treatment.
7. Has any other clinically significant medical, psychiatric and/or social reason for being ineligible as determined by the Principal Investigator.
8. Has a prior history or propensity for orthostatic hypotension, history of repeated fainting or hemodynamic instability.
9. Has a screening 12-lead ECG demonstrating QTcF >470 msec for women or >450 msec for men, and/or history or evidence of long QT syndrome.
10. Donated blood or has a significant loss of blood (≥480 mL) within 4 weeks prior to screening.
11. Donated plasma within 2 weeks prior to screening.
12. If female, has a positive serum pregnancy test at screening visit or at study check in.
13. Has a positive urine screen for drugs of abuse (including alcohol) during screening visit or at study check in.
14. Has a positive urine screen for cotinine during screening visit or at study check in.
15. Has a positive blood screen for hepatitis C antibody, hepatitis B surface antigen, hepatitis B core antibody, or HIV.
16. Has a history of significant drug allergy.
17. Has a prior exposure to dexpramipexole.
18. Has prior exposure to any investigational drug or placebo within 4 weeks or 5 half lives of the drug (whichever is longer) prior to Check-In (Day −1).
19. Has used any over the counter acid controllers/blockers within 4 weeks prior to Check-In (Day −1) or has achlorhydria.
20. Has a history of dopamine agonist use (e.g., Mirapex®, Mirapex-ER®, Requip®, bromocriptine, Sinemet®).
21. Has received any prescription (excluding acceptable birth control or hormone replacement therapy), non-prescription, nutritional supplements, or herbal medication 14 days or 5 half-lives (whichever is longer) prior to Check-In (Day −1).
22. Is unable or has difficulty swallowing or tolerating oral medication.
23. Poor venous access.
24. Pregnant women or women breastfeeding.
25. Males who are unwilling to use an acceptable method of birth control during the entire study period (i.e., condom with spermicide).

### Study Period

Five study periods of 72 hours.

### Study Duration

The entire duration of the study, including Screening, is approximately 6 weeks.
Twenty-one day screening period
Day −1—admission/check-in
Five 3-day treatment periods
Follow-up is completed 72-96 hours after last dose

### Investigational Product Administration

Participants are consuming a standard meal 2 hours before the dose. Water may be ingested ad libitum up to 1-hour predose, and after 2 hours postdose. Dexpramipexole tablets are administered orally with 240 mL of room temperature tap water. Participants may receive standardized meals and snacks after their 4-hour postdose blood draw.

### Statistical Methods—Pharmacokinetics

Plasma dexpramipexole concentrations and the PK parameters are summarized using the appropriate descriptive statistics.

To determine the relative bioavailability of the test (ER tablets) to the reference (IR tablet) formulations, an analysis of variance (ANOVA) is performed on the natural logarithm (ln)-transformed PK parameters Cₘₐₓ and AUC₀₋₂₄. The ANOVA model includes treatment, sequence, and period as fixed effects and participant nested within sequence as a random effect. Treatment differences as measured by least squares (LS) mean differences and the 90% CIs are constructed for the In-scale values of each parameter, back-transformed, and expressed as the ratio of geometric means. The ratios (and 90% CIs) for Cₘₐₓ and AUC₀₋₂₄ estimates are expressed as a percentage relative to the reference formulation. The In-transformed PK parameter AUC₀₋ₜ is compared using the same model, with the results presented as supportive information.

The comparisons of interest for relative bioavailability assessments are as follows:
ER tablet 1 versus IR tablet given g12h
ER tablet 2 versus IR tablet given g12h
ER tablet 3 versus IR tablet given g12h
ER tablet 4 versus IR tablet given g12h

### Statistical Methods—Safety

The safety summary includes the analysis of treatment-emergent adverse events (TEAE; i.e. AEs that begin after the first dose of study drug), including serious adverse events (SAEs) and the adverse events leading to study discontinuation, by treatment and overall.

Clinical laboratory results, vital signs, and ECG measurements are summarized using descriptive statistics and the changes from baseline by treatment and time point of collection. A shift table is provided for selected laboratory parameters, as appropriate.

### Analysis Population

Pharmacokinetics: The PK population includes all participants who have a calculable PK parameter for the reference treatment, and at least one test treatment in order to have a comparison of interest for the primary objective. Data from participants in this population is used for all PK summaries.

Safety: The safety population includes all participants who receive at least 1 dose of study drug. Data from participants in this population are used for demographic and safety summaries.

### Sample Size Determination

Fifteen participants are enrolled in the study, with three participants in each sequence. A sample size of 12 participants is typical for pilot relative bioavailability studies (per Food and Drug Administration [FDA] guidance) and deemed adequate for the assessment of relative bioavailability; 3 additional participants are enrolled to allow for dropouts (20% dropout rate).

### Example 9—Matrix Tablet Formulations

Formulations described below have advantageously been designed to optimize the amounts of anionic polymers and hydrophilic neutral polymers to provide suitable dissolution profiles.

A quality target product profile (QTPP) was defined for Dexpramipexole Controlled-Release Tablets and is given in Table 37.

TABLE 37
Quality target product profile (QTPP) for Dexpramipexole
Dihydrocholoride Controlled-Release Tablets.

| QTPP Element | | | Target |
|---|---|---|---|
| Dosage Form and Design | | | Matrix Tablet, Extended Release |
| Route of Administration | | | Oral |
| Dosage Strength | | | ER5: 280 mg Dexpramipexole (equivalent to 376 mg |
| | | | Dexpramipexole Dihydrocholoride) |
| | | | ER7H and ER9: 223 mg Dexpramipexole (equivalent |
| | | | to 300 mg of Dexpramipexole Dihydrocholoride) |
| Drug Product Quality | Physical | Appearance | No visual tablet defects observed. |
| Attributes | Attributes | Shape and | Oval or capsule shaped due to large tablet size |
| | | Size | |
| | | Friability | NMT 1.0% |
| | | Coating | Film coating |

| Assay | 90.0-110.0% |
|---|---|
| Content Uniformity | Conforms to USP <905> requirements (by weight |
| | variation) |
| Dissolution | ER5: release profile matching ER2 |
| | ER7H and ER9: release profile within the range of |
| | IVIVC model prediction |
| Degradation Products | Meets ICH requirements (0.2% Identification |

Threshold)

Table 38 shows the compositions evaluated and manufactured with roller compaction or direct compression processes, as indicated. Tablets “Matrix Tablet, Fast” and “Matrix Tablet, Slow” were discussed previously in Examples 5.4 and 7.

TABLE 38
Formulations used to study dexpramipexole dihydrochloride monohydrate dissolution
(tablets produced by roller compaction or direct compression, as indicated below).

| | Matrix Tablet, Fast (ER1) | | | Matrix Tablet, Slow (ER2) | | | ER5 280 mg | |
|---|---|---|---|---|---|---|---|---|
| | % w/w | % w/w | | % w/w | % w/w | | % w/w | % w/w |
| Ingredient | (core) | (total) | mg/tab | (core) | (total) | mg/tab | (core) | (total) |

Intra-Granular

| | | | | | | | | |
|---|---|---|---|---|---|---|---|---|
| Dexpramipexole | 37.53 | 36.44 | 319.01* | 35.44 | 34.41 | 319.01* | 35.54 | 34.50 |
| Dihydrochloride Monohydrate | | | | | | | | |
| Sodium | 28.00 | 27.18 | 238.00 | 33.06 | 32.09 | 297.50 | n/a | n/a |
| Carboxymethylcellulose | | | | | | | | |
| (Aqualon CMC 7HXF PH) | | | | | | | | |
| Sodium | n/a | n/a | n/a | n/a | n/a | n/a | 29.56 | 28.69 |
| Carboxymethylcellulose | | | | | | | | |
| (Blanose CMC 7HXF PH) | | | | | | | | |
| Hydroxypropyl | n/a | n/a | n/a | n/a | n/a | n/a | n/a | n/a |
| Methylcellulose (Benecel K100 | | | | | | | | |
| PH DC) | | | | | | | | |
| Carbomer (Carbopol 971P) | 12.00 | 11.65 | 102.00 | 14.17 | 13.75 | 127.50 | 12.67 | 12.30 |
| Silicified Microcrystalline | 21.22 | 20.60 | 180.36 | 16.08 | 15.61 | 144.74 | 20.99 | 20.38 |
| Cellulose (Prosolv HD90) | | | | | | | | |
| Magnesium Stearate | 0.25 | 0.24 | 2.13 | 0.25 | 0.24 | 2.25 | 0.25 | 0.24 |
| (Ligamed MF-2-V) | | | | | | | | |

Extra-Granular

| | | | | | | | | |
|---|---|---|---|---|---|---|---|---|
| Colloidal Silicon Dioxide | 0.50 | 0.49 | 4.25 | 0.50 | 0.49 | 4.50 | 0.50 | 0.49 |
| (Aerosil 200P) | | | | | | | | |
| Magnesium Stearate | 0.50 | 0.49 | 4.25 | 0.50 | 0.49 | 4.50 | 0.50 | 0.49 |
| (Ligamed MF-2-V) | | | | | | | | |
| Total (Tablet Core) | 100.00 | 97.09 | 850.00 | 100.00 | 97.09 | 900.00 | 100.00 | 97.09 |

Film Coating

| | | | | | | | | |
|---|---|---|---|---|---|---|---|---|
| Opadry II White 85F18422 | n/a | 2.91 | 25.50 | n/a | 2.9 | 27.00 | n/a | n/a |
| (Polyvinyl Alcohol, Titanium | | | | | | | | |
| Dioxide, Polyethylene Glycol, | | | | | | | | |
| Talc) | | | | | | | | |
| Opadry II 85F130089 Pink | n/a | n/a | n/a | n/a | n/a | n/a | n/a | 2.91 |
| (Polyvinyl Alcohol, Titanium | | | | | | | | |
| Dioxide, Polyethylene Glycol, | | | | | | | | |
| Talc, Ferric Oxide Red) | | | | | | | | |
| Purified water\hat{ } | n/a | n/a | 102.00 | n/a | n/a | 108.00 | n/a | n/a |
| Total (Tablet) | n/a | 100.00 | 875.50 | n/a | 100.00 | 927.00 | n/a | 100.00 |

| | | ER7H 223 mg | | | ER9 223 mg | | |
|---|---|---|---|---|---|---|---|
| | ER5 280 mg | % w/w | % w/w | | % w/w | % w/w | |
| Ingredient | mg/tab | (core) | (total) | mg/tab | (core) | (total) | mg/tab |
| Intra-Granular | | | | | | | |
| Dexpramipexole | 399.82* | 42.53 | 41.30 | 319.01* | 42.53 | 41.30 | 319.01* |
| Dihydrochloride Monohydrate | | | | | | | |
| Sodium | n/a | n/a | n/a | n/a | n/a | n/a | n/a |
| Carboxymethylcellulose | | | | | | | |
| (Aqualon CMC 7HXF PH) | | | | | | | |
| Sodium | 332.50 | n/a | n/a | n/a | 28.00 | 27.18 | 210.00 |
| Carboxymethylcellulose | | | | | | | |
| (Blanose CMC 7HXF PH) | | | | | | | |
| Hydroxypropyl | n/a | 15.00 | 14.56 | 112.50 | n/a | n/a | n/a |
| Methylcellulose (Benecel K100 | | | | | | | |
| PH DC) | | | | | | | |
| Carbomer (Carbopol 971P) | 142.50 | n/a | n/a | n/a | n/a | n/a | n/a |
| Silicified Microcrystalline | 236.11 | 41.53 | 40.32 | 311.49 | 28.53 | 27.70 | 213.99 |
| Cellulose (Prosolv HD90) | | | | | | | |
| Magnesium Stearate | 2.81 | n/a | n/a | n/a | n/a | n/a | n/a |
| (Ligamed MF-2-V) | | | | | | | |
| | Extra-Granular | | | | | | |
| Colloidal Silicon Dioxide | 5.63 | 0.47 | 0.45 | 3.50 | 0.47 | 0.45 | 3.50 |
| (Aerosil 200P) | | | | | | | |
| Magnesium Stearate | 5.63 | 0.47 | 0.45 | 3.50 | 0.47 | 0.45 | 3.50 |
| (Ligamed MF-2-V) | | | | | | | |
| Total (Tablet Core) | 1125.00 | 100.00 | 97.08 | 750.00 | 100.00 | 97.08 | 750.00 |
| | Film Coating | | | | | | |
| Opadry II White 85F18422 | n/a | n/a | n/a | n/a | n/a | n/a | n/a |
| (Polyvinyl Alcohol, Titanium | | | | | | | |
| Dioxide, Polyethylene Glycol, | | | | | | | |
| Talc) | | | | | | | |
| Opadry II 85F130089 Pink | 33.75 | n/a | 2.91 | 22.50 | n/a | 2.91 | 22.50 |
| (Polyvinyl Alcohol, Titanium | | | | | | | |
| Dioxide, Polyethylene Glycol, | | | | | | | |
| Talc, Ferric Oxide Red) | | | | | | | |
| Purified water\hat{ } | 135.00 | n/a | n/a | 90.00 | n/a | n/a | 90.00 |
| Total (Tablet) | 1158.75 | n/a | 100.00 | 772.50 | n/a | 100.00 | 772.50 |

*319.01 mg of dexpramipexole dihydrochloride monohydrate is equivalent to 300 mg dexpramipexole dihydrochloride.
*399.82 mg of dexpramipexole dihydrochloride monohydrate is equivalent to 376 mg dexpramipexole dihydrochloride.
\hat{ }Removed during processing; solids in coating solution = 20% w/w.
n/a = not applicable.

As described above, “Matrix Tablet, Fast” and “Matrix Tablet, Slow” were prepared using roller compaction. Specifically, the intra-granular ingredients shown in Table 38 were mixed into a pre-blend from which subsequently densified granules were prepared by roller compaction. The densified granules were subsequently blended with glidant (colloidal silicon dioxide, pre-lubrication) and lubricant (magnesium stearate, lubrication). The above blends were compressed into core tablets, and the core tablets were film coated using Opadry II White 85° F.18422.

Matrix Tablet “ER5 280 mg” was prepared using roller compaction. Specifically, the dexpramipexole dihydrochloride monohydrate, sodium carboxymethylcellulose, carbomer, and silicified microcrystalline cellulose, as shown in Table 38, were mixed into a pre-blend. Magnesium Stearate was subsequently added as a lubricant to the pre-blend from which subsequently densified granules were prepared by roller compaction. The densified granules were subsequently blended with glidant (colloidal silicon dioxide, pre-lubrication) and lubricant (magnesium stearate, lubrication). The above blends were compressed into core tablets, and the core tablets were film coated using Opadry II 85F130089 Pink.

Matrix Tablet “ER7H 223 mg” was prepared using direct compression. Specifically, the dexpramipexole dihydrochloride monohydrate, hydroxypropyl methylcellulose, silicified microcrystalline cellulose, and colloidal silicon dioxide, as shown in Table 38, were mixed into a blend. The above blend was subsequently blended with lubricant (magnesium stearate, lubrication). The above blend was compressed into core tablets, and the core tablets were film coated using Opadry II 85F130089 Pink.

Matrix Tablet “ER9 223 mg” was prepared using direct compression. Specifically, the dexpramipexole dihydrochloride monohydrate, sodium carboxymethylcellulose, silicified microcrystalline cellulose, and colloidal silicon dioxide, as shown in Table 38, were mixed into a blend. The above blend was subsequently blended with lubricant (magnesium stearate, lubrication). The above blend was compressed into core tablets, and the core tablets were film coated using Opadry II 85F130089 Pink.

### Example 9.1—ER5 Formulation Development

The objective of formulation ER5 was to increase the strength to 280 mg Dexpramipexole (equivalent to 376 mg Dexpramipexole diHCl) and achieve the same release profile as ER2 in 0.1N HCl media. ER2 corresponds to the two matrix tablet formulation of Table 32. The ER2 formulation was dose proportionally scaled up the from 223 mg Dexpramipexole strength to 280 mg strength, and the polymer level was adjusted since the specific surface area will reduce as the tablet gets bigger. The formulations used to study impact of the polymer level are summarized in Table 39. Aqualon's sodium carboxymethylcellulose was initially used for development, and the manufacturer discontinued Aqualon during the development of this product. Therefore, an equivalent grade, Blanose, was used.

TABLE 39
Formulation used to study the polymer level of ER5.

| | | 031466-01 | | 031466-06 | | 031466-07 | |
|---|---|---|---|---|---|---|---|
| Material | Function | % w/w | mg/unit | % w/w | mg/unit | % w/w | mg/unit |
| Dexpramipexole | Drug | 35.45 | 398.76 | 37.37 | 399.83 | 39.19 | 399.83 |
| Dihydrochloride Monohydrate | substance | | | | | | |
| Sodium | Anionic | n/a | n/a | 31.07 | 332.50 | 29.16 | 397.50 |
| Carboxymethylcellulose | Matrix | | | | | | |
| (Aqualon CMC 7HXF PH) | polymer | | | | | | |
| Sodium | | 33.06 | 371.88 | n/a | n/a | n/a | n/a |
| Carboxymethylcellulose | | | | | | | |
| (Blanose CMC 7HXF PH) | | | | | | | |
| Carbomer (Carbopol 971P) | | 14.17 | 159.38 | 13.32 | 142.50 | 12.53 | 127.80 |
| Silicified Microcrystalline | Diluent | 16.80 | 180.93 | 16.92 | 181.08 | 17.75 | 181.08 |
| Cellulose (Prosolv HD90) | | | | | | | |
| Colloidal Silicon Dioxide | Glidant | 0.50 | 5.63 | 0.53 | 5.64 | 0.55 | 5.64 |
| (Aerosil 200 Pharma) | | | | | | | |
| Magnesium Stearate | Lubricant | 0.75 | 8.44 | 0.79 | 8.46 | 0.83 | 8.46 |
| (Ligamed MF-2-V) | | | | | | | |
| Total | | 100.0 | 1125.00 | 100.00 | 1070.00 | 100.00 | 1020.30 |

The batches were made with direct compression method in a 50 g batch size. Lot 031466-01 was made by taking the final blend from ER2 engineering batch (lot AQ-23-0026) and compressing at 279 mg strength (equivalent to 398.76 mg Dexpramipexole Dihydrochloride Monohydrate). The dissolution results are provided in FIG. 28. The results show that after dose proportionally scaling up from 223 mg to 279 mg, the release was slower due to the reduced tablet specific surface area (ER2 clinical batch AQ-23-0026 vs. 031466-01). After reducing the polymer level, the dissolution was faster. Since a scale-up batch would use a roller compaction process that might slow down the dissolution, it was decided to move forward with 475 mg/unit polymer level and increased the core tablet weight to 1125 mg for a confirmation batch evaluation.

### Example 9.2—ER7H Formulation Development

The objective of ER7H and ER9 development was to use two different types of matrix polymers to achieve the target dissolution range. ER7H used Hypromellose (Methocel DC2 K100M Premium) as its matrix polymer. ER9 formulation used Sodium Carboxymethylcellulose (Blanose CMC 7HXF PH) as the matrix polymer. The batches were made with direct compression method in a 50 g batch size.

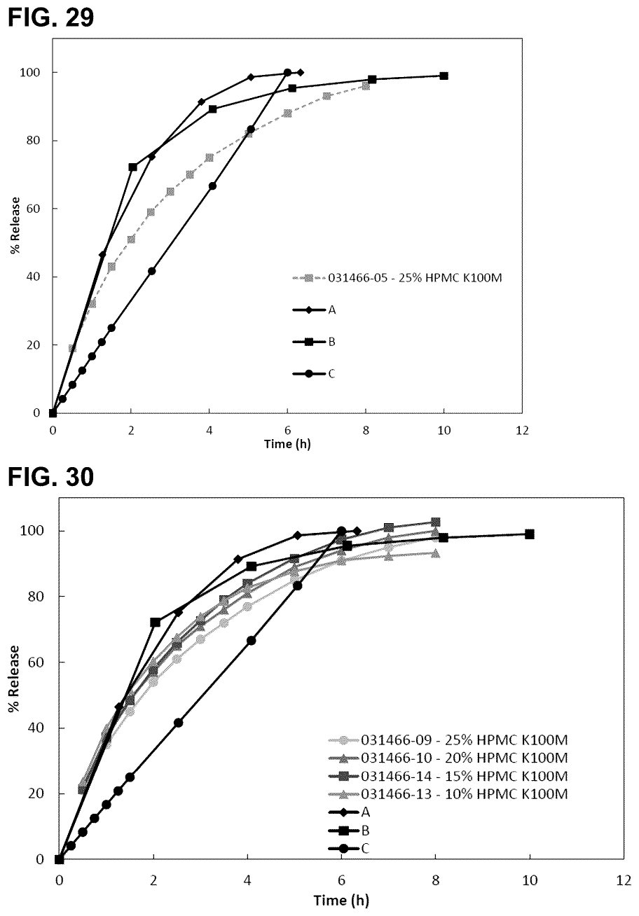

The ER7H formulation development started with 25% HPMC K100M and 850 mg tablet weight. The formulation is summarized in Table 40, and the dissolution is shown in FIG. 29. Lines A, B, and C are the target profiles. The release profile of ER7H lot 031466-05 is significantly slower than profile A and B, but still faster than profile C before 5h.

TABLE 40
Formulation of ER7H lot 031466-05 with 850 mg tablet weight.

| | 031466-05 | |
|---|---|---|
| Material | % w/w | mg/unit |
| Dexpramipexole Dihydrochloride Monohydrate | 37.53 | 319.00 |
| Hypromellose (Methocel DC2 K100M Premium) | 25.00 | 212.50 |
| Silicified Microcrystalline Cellulose (Prosolv HD90) | 36.22 | 307.88 |
| Colloidal Silicon Dioxide (Aerosil 200 Pharma) | 0.50 | 4.25 |
| Magnesium Stearate (Ligamed MF-2-V) | 0.75 | 6.38 |
| Total | 100.0 | 850.01 |

Following formulation of ER7H lot 031466-05, the objective was to increase the release rate of the formulation. Therefore, in the next round of formulation screening, the tablet weight was reduced to 700 mg to increase the specific surface area. The impact of HPMC K100M level in the formulation was also studied. The formulations studied are summarized in Table 41 and the dissolution profiles are shown in FIG. 30. The release was faster with reduction in HPMC K100M level with an exception that 10% HPMC K100M seemed to be an outlier. It was decided to move forward with 15% HPMC K100M for a confirmation batch evaluation.

TABLE 41
Formulation used to study the HPMC K100M level of ER 7H with a tablet weight of 700 mg.

| | | 031466-09 | | 031466-10 | | 031466-14 | | 031466-13 | |
|---|---|---|---|---|---|---|---|---|---|
| Material | Function | % w/w | mg/unit | % w/w | mg/unit | % w/w | mg/unit | % w/w | mg/unit |
| Dexpramipexole | Drug | 37.53 | 319.00 | 45.57 | 319.00 | 45.57 | 319.00 | 45.57 | 319.00 |
| Dihydrochloride | substance | | | | | | | | |
| Monohydrate | | | | | | | | | |
| Hypromellose | Neutral | 20.59 | 175.00 | 20.00 | 140.00 | 15.00 | 105.00 | 10.00 | 70.00 |
| (Methocel DC2 | Matrix | | | | | | | | |
| K100M Premium) | Polymer | | | | | | | | |
| Silicified | Diluent | 23.21 | 197.25 | 33.18 | 232.25 | 38.18 | 267.25 | 43.18 | 302.25 |
| Microcrystalline | | | | | | | | | |
| Cellulose (Prosolv | | | | | | | | | |
| HD90) | | | | | | | | | |
| Colloidal Silicon | Glidant | 0.41 | 3.50 | 0.50 | 3.50 | 0.50 | 3.50 | 0.50 | 3.50 |
| Dioxide (Aerosil 200 | | | | | | | | | |
| Pharma) | | | | | | | | | |
| Magnesium Stearate | Lubricant | 0.62 | 5.25 | 0.75 | 5.25 | 0.75 | 5.25 | 0.75 | 5.25 |
| (Ligamed MF-2-V) | | | | | | | | | |
| Total | | 82.53 | 700.00 | 100.00 | 700.00 | 100.00 | 700.00 | 100.00 | 700.00 |

### Example 9.3—ER9 Formulation Development

The objective of ER7H and ER9 development was to use two different types of matrix polymers to achieve the target dissolution range. ER7H used Hypromellose (Methocel DC2 K100M Premium) as its matrix polymer. ER9 formulation used Sodium Carboxymethylcellulose (Blanose CMC 7HXF PH) as the matrix polymer. The batches were made with direct compression method in a 50 g batch size.

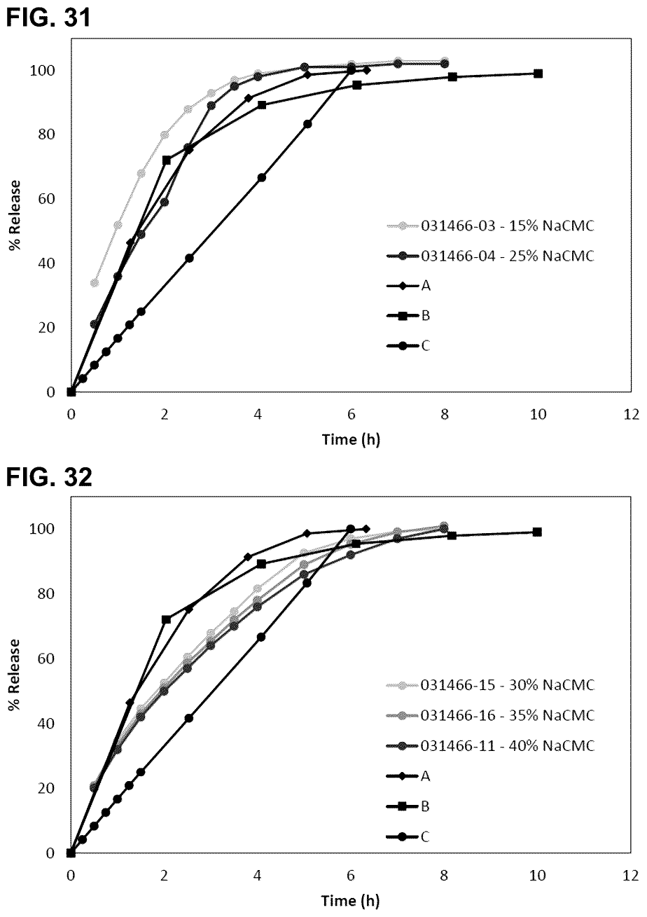

Similar to ER7H development, ER9 formulation started with 850 mg tablet weight. Two levels of polymer at 15% and 25% were studied (see Table 42). The dissolution results are shown in FIG. 31. Both formulations showed faster release than the targeted range. Therefore, higher polymer ranges were investigated.

TABLE 42
Formulation used to study the NaCMC 7HXF level
of ER9 with a tablet weight of 850 mg.

| | | 031466-03 | | 031466-04 | |
|---|---|---|---|---|---|
| Material | Function | % w/w | mg/unit | % w/w | mg/unit |
| Dexpramipexole Dihydrochloride | Drug substance | 37.53 | 319.00 | 37.53 | 319.00 |
| Monohydrate | | | | | |
| Sodium Carboxymethylcellulose | Anionic Matrix | 15.00 | 127.50 | 25.00 | 212.50 |
| (Blanose CMC 7HXF PH) | Polymer | | | | |
| Silicified Microcrystalline Cellulose | Diluent | 46.22 | 392.88 | 36.22 | 307.88 |
| (Prosolv HD90) | | | | | |
| Colloidal Silicon Dioxide (Aerosil 200 | Glidant | 0.50 | 4.25 | 0.50 | 4.25 |
| Pharma) | | | | | |
| Magnesium Stearate (Ligamed MF-2-V) | Lubricant | 0.75 | 6.38 | 0.75 | 6.38 |
| Total | | 100.00 | 850.01 | 100.00 | 850.01 |

Sodium Carboxymethylcellulose (NaCMC) 7HXF levels of 30%, 35%, and 40% with 750 mg tablet weight were evaluated (see Table 43). The dissolution results are shown in FIG. 32. All of the three dissolution profiles fell in the targeted dissolution range. A 300 NaCMC 7HXF level was chosen to allow more silicified microcrystalline cellulose in the formulation and to have a better final blend flowability and tabletability.

TABLE 43
Formulation used to study the NaCMC 7HXF level of ER9 with a tablet weight of 700 mg.

| | | 031466-15 | | 031466-16 | | 031466-11 | |
|---|---|---|---|---|---|---|---|
| Material | Function | % w/w | mg/unit | % w/w | mg/unit | % w/w | mg/unit |
| Dexpramipexole Dihydrochloride | Drug | 45.57 | 319.00 | 45.57 | 319.00 | 45.57 | 319.00 |
| Monohydrate | substance | | | | | | |
| Sodium Carboxymethylcellulose | Anionic | 30.00 | 210.00 | 35.00 | 245.00 | 40.00 | 280.00 |
| (Blanose CMC 7HXF PH) | Matrix | | | | | | |
| | Polymer | | | | | | |
| Silicified Microcrystalline | Diluent | 23.18 | 162.25 | 18.18 | 127.25 | 13.18 | 92.25 |
| Cellulose (Prosolv HD90) | | | | | | | |
| Colloidal Silicon Dioxide | Glidant | 0.50 | 3.50 | 0.50 | 3.50 | 0.50 | 3.50 |
| (Aerosil 200 Pharma) | | | | | | | |
| Magnesium Stearate (Ligamed | Lubricant | 0.75 | 5.25 | 0.75 | 5.25 | 0.75 | 5.25 |
| MF-2-V) | | | | | | | |
| Total | | 100.0 | 700.00 | 100.00 | 700.00 | 100.00 | 700.00 |

### Example 9.4—ER5 Confirmation Batches

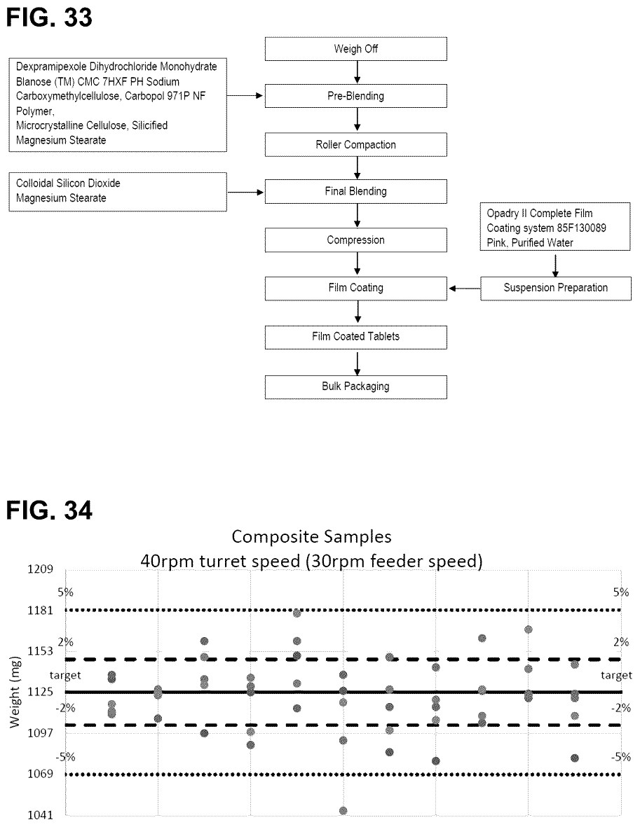

Two confirmation batches were made at batch size of 1500 tablets (1738.12 g). The formulation are summarized in Table 44. A process schematic is shown in FIG. 33. The process parameters are summarized in Table 45.

TABLE 44
Formulation of Dexpramipexole ER matrix 280 mg tablets (376
mg diHCl) confirmation batch lots 031466-17 and 031466-29.

| | | 031466-17 and -29 | | | |
|---|---|---|---|---|---|
| | | | % w/w | % w/w | qty/batch |
| Material | Function | mg/unit | core | total | (g) |

Intra-Granular

| | | | | | |
|---|---|---|---|---|---|
| Dexpramipexole Dihydrochloride | Drug Substance | 399.82 | 35.54 | 34.50 | 599.73 |
| Monohydrate * | | | | | |
| Sodium Carboxymethylcellulose | | 332.50 | 29.56 | 28.69 | 498.75 |
| (Blanose CMC 7HXF PH) | Anionic Matrix | | | | |
| Carbomer (Carbopol 971P) | Polymer | 142.50 | 12.67 | 12.30 | 213.75 |
| Silicified Microcrystalline | Diluent | 236.11 | 20.99 | 20.38 | 354.17 |
| Cellulose (Prosolv HD90) | | | | | |
| Magnesium Stearate | Lubricant | 2.81 | 0.25 | 0.24 | 4.22 |
| (Ligamed MF-2-V) | | | | | |

Extra-Granular

| | | | | | |
|---|---|---|---|---|---|
| Colloidal Silicon Dioxide | Glidant | 5.63 | 0.50 | 0.49 | 8.44 |
| (Aerosil 200P) | | | | | |
| Magnesium Stearate | Lubricant | 5.63 | 0.50 | 0.49 | 8.45 |
| (Ligamed MF-2-V) | | | | | |
| Total Core Tablet | | 1125.00 | 100.00 | 97.09 | 1687.49 |
| Opadry II 85F130089 Pink ** | Colorant | 33.75 | n/a | 2.91 | 50.63 |
| Purified water\hat{ } | Suspending Agent | 135.00 | n/a | n/a | n/a |
| Total Coated Tablet | | 1158.75 | 99.75 | 100.00 | 1738.12 |

* 399.83 mg of dexpramipexole dihydrochloride monohydrate is equivalent to 376 mg dexpramipexole dihydrochloride.
** Opadry II 85F130089 PINK was added at a theoretical weight gain of 3.00% w/w of the core tablet. Opadry coating dispersion made at a 20% solids content.
\hat{ }Removed during processing.

TABLE 45
Process parameters of Dexpramipexole ER matrix 280 mg tablets
(376 mg diHCl) confirmation batch lots 031466-17 and 031466-29.
Process Parameters

| Blending | Equipment | Diffusion blender |
|---|---|---|
| | Pre-Mix (revolutions) | 250 |
| | Lubrication (revolutions) | 75 |
| Roller | Equipment | Gerteis Mini-Polygran |
| Compaction | Roller Type | Two Knurled |
| | Granulator Rotor | Star |
| | Granulator Screen | 1.0 mm regular wire mesh |
| | Roller Speed (rpm) | 5 |
| | Roller Pressure (kN/cm) | 3 |
| | Gap Size (mm) | 1.5 |
| | Gap Control | ON, 2 medium |
| | Agitator Speed (rpm) | 10 |
| | Granulator Speed | 45 rpm/45 rpm |
| | CW/CCW | |
| | Granulator Angle | 250°/350° |
| | CW/CCW | |
| Blending | Equipment | Diffusion blender |
| | Pre-Mix (revolutions) | 250 |
| | Lubrication (revolutions) | 75 |
| Compression | Equipment | Korsch XL100 |
| | Tooling | 0.3937″ × 0.7874″ Modified |
| | | Oval |
| | Target Tablet Weight | 1125 |
| | (mg) | |
| | Target Hardness (kp) | 25 |
| | Turret Speed (rpm) | 20 |
| | Feedframe Speed (rpm) | 25 |
| Film | Equipment | LDCS 5 |
| Coating | Pan Size (inch) | 12 |
| | Nozzle Size (mm) | 0.5 |
| | Atomization Pressure | 18 |
| | (psi) | |
| | Drum Speed (rpm) | 12 |
| | Target Product | 41 |
| | Temperature (° C.) | |
| | Spray Rate (g/min) | 7.0 |

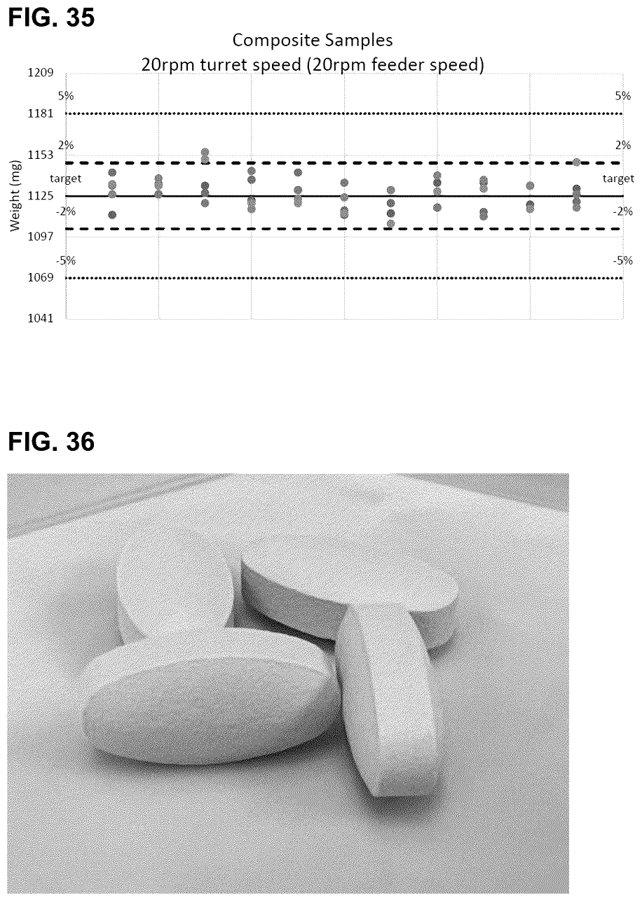

Blend bulk density was measured for lot 031466-17: 0.516 g/ml for milled granules and 0.533 g/ml for the final blend. The compression of both batches was performed on a Korsch XL 100 tablet press. Both batches showed similar final blend flowability. The impact of compression speed on tablet weight variation was studied on the first batch 031466-17. Turret speeds of 40 rpm and 20 rpm were studied. The feed frame impeller speed was adjusted accordingly. 55 tablets from each compression speed were evaluated for tablet weight (see FIGS. 34 and 35). Tablet weight variation was ±5% at 40 rpm turret speed compared to +2% at 20 rpm turret speed. Therefore, the 20 rpm turret speed was chosen for the second confirmation batch 031466-29. The in-process tablet variations are summarized in Tables 46 and 47. The appearance of coated tablets from lot 031466-29 is shown in FIG. 36.

TABLE 46
In-process tablet weight, thickness, harness
and tensile strength of Lot 031466-17.
Lot 031466-17

| | | | |
|---|---|---|---|
| Tablet weight | Thickness | Hardness | Tensile strength |
| (mg) | (mm) | (kp) | (MPa) |

Beginning of Run

| | | | |
|---|---|---|---|
| 1129 | 6.97 | 27.6 | 1.9 |
| 1116 | 6.97 | 25.8 | 1.7 |
| 1132 | 6.99 | 27.5 | 1.8 |
| 1125 | 6.97 | 27.0 | 1.8 |
| 1125 | 6.98 | 26.5 | 1.8 |
| 1129 | 6.98 | 27.8 | 1.9 |
| 1147 | 7.04 | 29.3 | 1.9 |
| 1131 | 6.98 | 26.9 | 1.8 |
| 1135 | 6.99 | 27.9 | 1.9 |
| 1148 | 7.02 | 31.9 | 2.1 |

End of Run

| | | | |
|---|---|---|---|
| 1106 | 6.90 | 23.9 | 1.6 |
| 1119 | 6.94 | 26.2 | 1.8 |
| 1125 | 6.97 | 28.2 | 1.9 |
| 1116 | 6.95 | 26.2 | 1.8 |
| 1141 | 6.99 | 29.3 | 2.0 |
| 1107 | 6.92 | 24.6 | 1.7 |
| 1140 | 7.04 | 30.1 | 2.0 |
| 1117 | 6.95 | 25.7 | 1.7 |
| 1128 | 7.01 | 27.5 | 1.8 |
| 1108 | 6.95 | 23.6 | 1.6 |

Average

| | | | |
|---|---|---|---|
| 1126 | 6.98 | 27.2 | 1.8 |

TABLE 47
In-process tablet weight, thickness, harness
and tensile strength of Lot 031466-29.
Lot 031466-17

| | | | |
|---|---|---|---|
| Tablet weight | | | Tensile strength |
| (mg) | Thickness (mm) | Hardness (kp) | (MPa) |

Beginning of Run

| | | | |
|---|---|---|---|
| 1109 | 6.93 | 22.2 | 1.5 |
| 1104 | 6.91 | 20.6 | 1.4 |
| 1122 | 6.94 | 23.0 | 1.6 |
| 1143 | 7.00 | 27.3 | 1.8 |
| 1114 | 6.94 | 22.3 | 1.5 |
| 1117 | 6.96 | 23.3 | 1.6 |
| 1120 | 6.94 | 23.0 | 1.6 |
| 1128 | 6.96 | 23.7 | 1.6 |
| 1128 | 6.96 | 24.0 | 1.6 |
| 1120 | 6.95 | 24.2 | 1.6 |

Middle of Run

| | | | |
|---|---|---|---|
| 1131 | 6.95 | 24.4 | 1.6 |
| 1135 | 7.00 | 25.5 | 1.7 |
| 1118 | 6.93 | 22.9 | 1.6 |
| 1122 | 6.95 | 23.4 | 1.6 |
| 1141 | 6.99 | 27.4 | 1.8 |
| 1128 | 6.96 | 23.4 | 1.6 |
| 1122 | 6.95 | 23.6 | 1.6 |
| 1128 | 6.98 | 23.6 | 1.6 |
| 1122 | 6.96 | 23.1 | 1.6 |
| 1122 | 6.97 | 24.4 | 1.6 |

End of Run

| | | | |
|---|---|---|---|
| 1120 | 6.93 | 23.2 | 1.6 |
| 1139 | 6.98 | 20.8 | 1.4 |
| 1113 | 6.94 | 25.3 | 1.7 |
| 1135 | 6.97 | 24.0 | 1.6 |
| 1142 | 6.99 | 23.8 | 1.6 |
| 1132 | 6.97 | — | — |
| 1101 | 6.90 | 18.9 | 1.3 |
| 1132 | 6.98 | 24.1 | 1.6 |
| 1104 | 6.91 | 18.9 | 1.3 |
| 1147 | 7.01 | 26.4 | 1.8 |

Average

| | | | |
|---|---|---|---|
| 1125 | 6.96 | 23.5 | 1.6 |

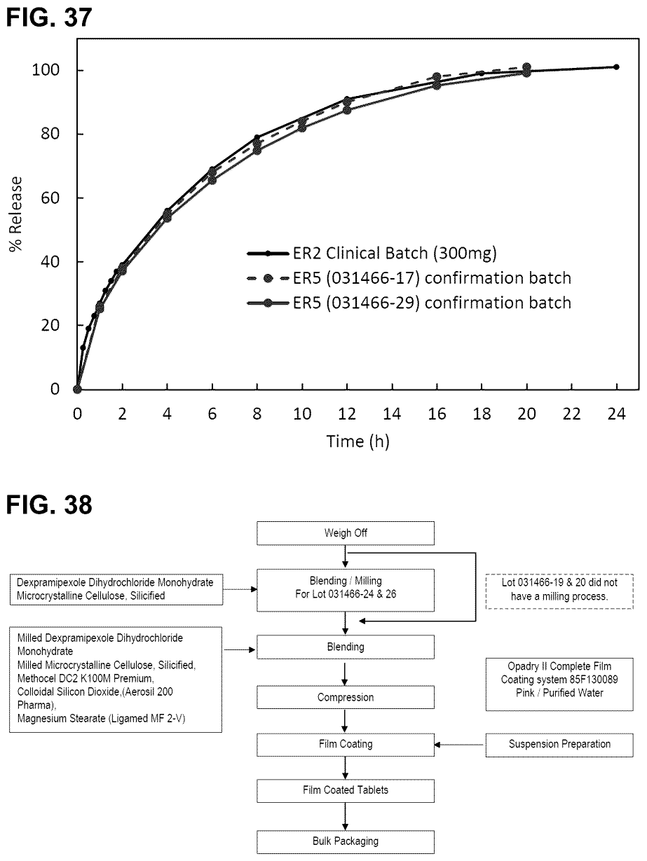

The dissolution of the coated tablets are shown in FIG. 37. Both confirmation batches showed similar dissolution profiles, and both profiles are similar to the dissolution profile of the clinical batch ER2 of Table 32.

### Example 9.5—ER7H Confirmation Batches

The ER7H confirmation batch was first manufactured using a formulation with 700 mg core tablet weight (lot: 031466-19). The core tablet weight was increased to 750 mg in a second confirmation batch (lot: 031466-24) to improve the tablet hardness and to match the size with the ER9 formulation. The formulations are summarized in Table 48. The process schematic is shown in FIG. 38. Both lots used the direction compression process. However, for lot 031466-24, the API and the silicified microcrystalline cellulose were first co-milled to reduce the API size and improve the tablet tabletability.

TABLE 48
Formulation of Dexpramipexole ER matrix 223 mg tablets (300 mg
diHCl) confirmation batch lots 031466-19 and lot 031466-24.

| | | 031466-19 | | | 031466-24 | | | |
|---|---|---|---|---|---|---|---|---|
| | | | % w/w | qty/batch | | % w/w | % w/w | qty/batch |
| Material | Function | mg/unit | core | (g) | mg/unit | core | total | g |
| Dexpramipexole | Drug | 319.01 | 45.57 | 638.02 | 319.01 | 42.53 | 41.30 | 638.02 |
| Dihydrochloride | Substance | | | | | | | |
| Monohydrate * | | | | | | | | |
| Hypromellose | Neutral | 122.50 | 17.50 | 245.00 | 112.50 | 15.00 | 14.56 | 225.00 |
| (Methocel DC2 | Matrix | | | | | | | |
| K100M | Polymer | | | | | | | |
| Premium) | | | | | | | | |
| Silicified | Diluent | 251.49 | 35.93 | 502.98 | 311.49 | 41.53 | 40.32 | 622.98 |
| Microcrystalline | | | | | | | | |
| Cellulose | | | | | | | | |
| (Prosolv HD90) | | | | | | | | |
| Colloidal Silicon | Glidant | 3.50 | 0.50 | 7.00 | 3.50 | 0.47 | 0.45 | 7.00 |
| Dioxide (Aerosil | | | | | | | | |
| 200 Pharma) | | | | | | | | |
| Magnesium | Lubricant | 3.50 | 0.50 | 7.00 | 3.50 | 0.47 | 0.45 | 7.00 |
| Stearate | | | | | | | | |
| (Ligamed MF-2-V) | | | | | | | | |
| Total Core | | 700.00 | 100.00 | 1400.00 | 750.00 | 100.00 | 97.09 | 1500.00 |
| Tablet | | | | | | | | |
| Opadry II | Colorant | Not performed | | | 22.50 | n/a | 2.91 | 45.00 |
| 85F130089 Pink ** | | | | | | | | |
| Purified water\hat{ } | Suspending | | | | 90.00 | n/a | n/a | n/a |
| | Agent | | | | | | | |
| Total Coated | | | | | 772.50 | n/a | 100.00 | 1545.00 |
| Tablet | | | | | | | | |

* 319.01 mg of dexpramipexole dihydrochloride monohydrate is equivalent to 300.00 mg dexpramipexole dihydrochloride.
** Opadry II 85F130089 PINK was added at a theoretical weight gain of 3.00% w/w of the core tablet. Opadry coating dispersion made at a 20% solids content.
\hat{ }Removed during processing.

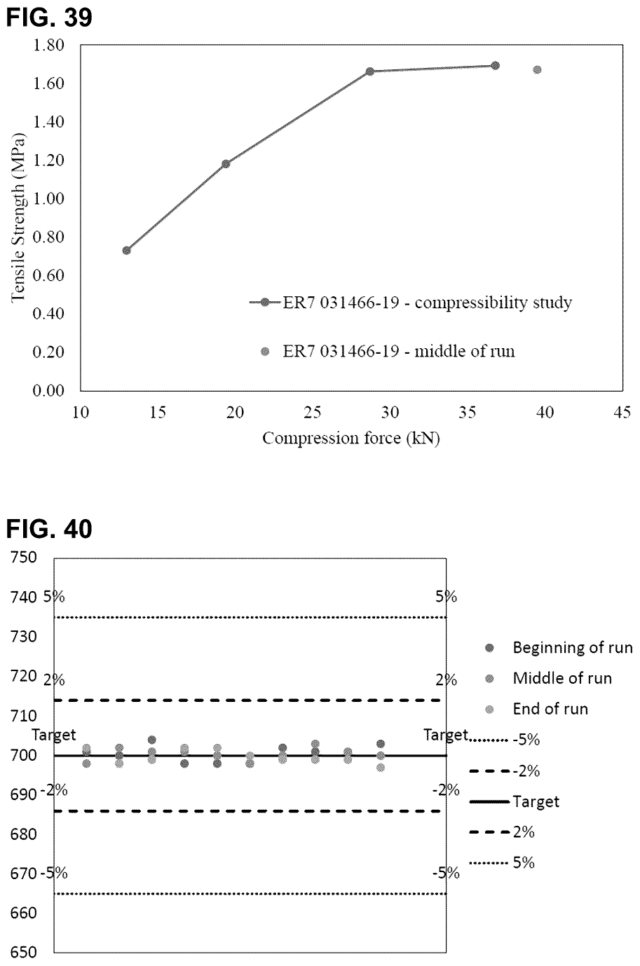

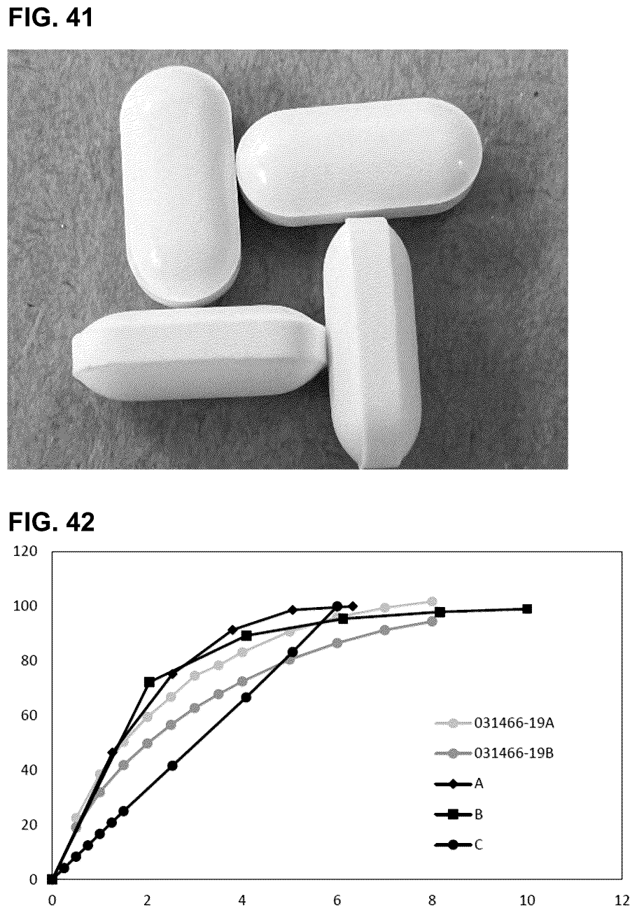

The process parameters of the first confirmation batch is summarized in Table 49. The bulk density of the final blend is 0.56 g/ml. A tabletability study was carried out on a Korsch XL 100 press before the actual compression process, and the results are shown in FIG. 39. The tablet tensile strength plateaued at around 1.7 MPa when the compression force was increased to 28.7 kN. The inprocess tablet weight, thickness, and hardness are summarized in Table 50, and the tablet weight variation is plotted in FIG. 40. The tablets showed consistent weight (weight variation less than ±2%). There was no capping or chipping discovered for the tablets (see FIG. 41).

TABLE 49
Process parameters Dexpramipexole ER matrix 223 mg
tablets (300 mg diHCl) confirmation batch lot 031466-19.
Process Parameters

| Blending | Equipment | Diffusion blender |
|---|---|---|
| | Pre-Mix (revolutions) | 300 |
| | Lubrication (revolutions) | 80 |
| Compression | Equipment | Korsch XL100 |
| | Tooling | 0.3050″ × 0.6700″ Capsule |
| | Target Tablet Weight | 700 |
| | (mg) | |
| | Turret Speed (rpm) | 20 |
| | Feedframe Speed (rpm) | 25 |

TABLE 50
In-process tablet weight, thickness, harness
and tensile strength of Lot 031466-19.
Lot 031466-19

| | | | |
|---|---|---|---|
| Tablet weight | | | Tensile strength |
| (mg) | Thickness (mm) | Hardness (kp) | (MPa) |

Beginning of Run

| | | | |
|---|---|---|---|
| 701 | 6.03 | 16.2 | 1.9 |
| 700 | 6.09 | 16.4 | 1.8 |
| 704 | 6.08 | 17.3 | 2.0 |
| 698 | 6.10 | 16.0 | 1.8 |
| 698 | 6.10 | 17.6 | 2.0 |
| 698 | 6.08 | 16.5 | 1.9 |
| 702 | 6.08 | 17.1 | 1.9 |
| 701 | 6.06 | 17.0 | 1.9 |
| 701 | 6.06 | 17.6 | 2.0 |
| 703 | 6.07 | 17.0 | 1.9 |

Middle of Run

| | | | |
|---|---|---|---|
| 698 | 6.06 | 16.3 | 1.9 |
| 702 | 6.10 | 15.4 | 1.7 |
| 701 | 6.07 | 15.2 | 1.7 |
| 701 | 6.06 | 15.9 | 1.8 |
| 700 | 6.02 | 15.8 | 1.8 |
| 698 | 6.06 | 15.4 | 1.8 |
| 700 | 6.07 | 16.0 | 1.8 |
| 703 | 6.08 | 15.7 | 1.8 |
| 701 | 6.09 | 15.9 | 1.8 |
| 700 | 6.09 | 15.0 | 1.7 |

End of Run

| | | | |
|---|---|---|---|
| 702 | 6.09 | 15.2 | 1.7 |
| 698 | 6.06 | 16.4 | 1.9 |
| 699 | 6.06 | 16.6 | 1.9 |
| 702 | 6.09 | 16.8 | 1.9 |
| 702 | 6.09 | 15.5 | 1.7 |
| 700 | 6.10 | 15.5 | 1.7 |
| 699 | 6.05 | 16.0 | 1.8 |
| 699 | 6.07 | 14.9 | 1.7 |
| 699 | 6.08 | 16.5 | 1.9 |
| 697 | 6.09 | 15.3 | 1.7 |

Average

| | | | |
|---|---|---|---|
| 700 | 6.07 | 16.1 | 1.8 |

During compression, the press needs to be maintained at around 40 kN to get ˜1.7 MPa. Generally, a tensile strength greater than 1.7 MPa is recommended to ensure a tablet is mechanically strong enough to withstand downstream manufacturing steps and distribution (Pitt, K. G. and Heasley M. G. Determination of the tensile strength of elongated tablets. Powder Technology 238 (2013) 169-175). It was decided to not further coat the tablets and adjust the formulation to target a tensile strength higher than 1.7 MPa.

With the goal of increasing the tensile strength of the tablets, two 50 g small batches (denoted as 031466-19A and 031466-19B) were made by adding: A) 50 mg/tab worth of silicified microcrystalline cellulose; or B) 50 mg/tab worth of Copovidone (Kollidon VA 64 Fine) to the 031466-19 final blend and bringing the tablet weight to 750 mg. The formulations of 031466-19A and 031466-19B are summarized in Table 51.

TABLE 51
Formulation of 031466-19A and 031466-19B.

| | | 031466-19A | | 031466-19B | |
|---|---|---|---|---|---|
| Material | Function | mg/unit | % w/w | mg/unit | % w/w |
| Dexpramipexole Dihydrochloride | Drug Substance | 319.01 | 42.53 | 319.01 | 42.53 |
| Monohydrate | | | | | |
| Hypromellose (Methocel DC2 | Neutral Matrix | 122.50 | 16.33 | 122.50 | 16.33 |
| K100M Premium) | Polymer | | | | |
| Silicified Microcrystalline Cellulose | Diluent | 251.49 | 33.53 | 251.49 | 33.53 |
| (Prosolv HD90) | | | | | |
| Colloidal Silicon Dioxide | Glidant | 3.50 | 0.47 | 3.50 | 0.47 |
| (Aerosil 200P) | | | | | |
| Magnesium Stearate | Lubricant | 3.50 | 0.47 | 3.50 | 0.47 |
| (Ligamed MF-2-V) | | | | | |
| Final Blend from 031466-19 | | 700.00 | 93.33 | 700.00 | 93.33 |
| Silicified Microcrystalline Cellulose | Diluent | 50.00 | 6.67 | — | — |
| (Prosolv HD90) | | | | | |
| Copovidone (Kollidon VA64 Fine) | Binder | — | — | 50.00 | 6.67 |
| Total Coated Tablet | | 750.00 | 100.00 | 750.00 | 100.00 |

The resulting tablet hardness from the three formulations, 031466-19, 031466-19A, and 031466-19B, were compared by compressing in a manual press at 5000 psi (results shown in Table 52). The dissolutions are shown in FIG. 42. Both the hardness and tensile strength increased after the adjustment. However, the dissolution of 031466-19B slowed down significantly after adding the new binder Copovidone (Kollidon VA64 Fine). Therefore, the second confirmation batch was manufactured by increasing the silicified microcrystalline cellulose in the formulation.

TABLE 52
Tablet hardness of 031466-19 (700 mg tablet weight)
vs. 031466-19A and 031466-19B (750 mg tablet weight).

| | | | | | Tensile |
|---|---|---|---|---|---|
| | Compression | | Thickness | Hardness | Strength |
| Formulation ER7 | force/pressure | | (mm) | (kp) | (MPa) |
| 700 mg tab | 39.5 | kN | 6.07 | 16.1 | 1.8 |
| (Korsch XL100 press) | | | | | |
| 700 mg tab | 5000 | psi | 6.04 | 17.1 | 2.0 |
| (manual press) | | | | | |
| 750 mg tab | 5000 | psi | 6.40 | 20.1 | 2.1 |
| (additional 50 mg/ | | | | | |
| tab SMCC) | | | | | |
| 750 mg tab | 5000 | psi | 6.47 | 25.2 | 2.6 |
| (additional 50 | | | | | |
| mg/tab Kollidon | | | | | |

VA64 Fine)

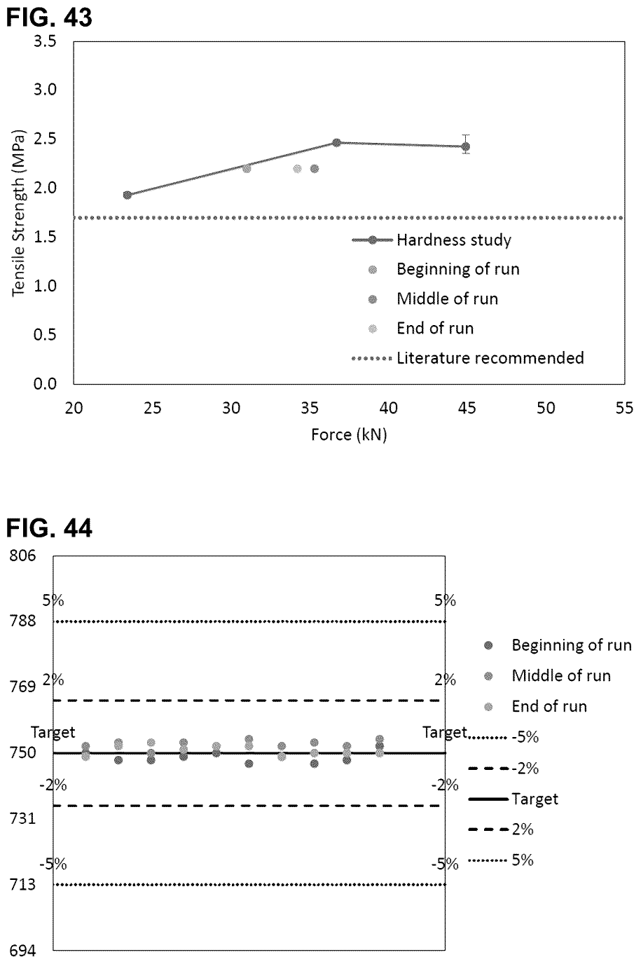

The process parameters of the second confirmation batch are summarized in Table 53. The final blend bulk density of the second confirmation batch 031466-24 was 0.57 g/ml. Similarly to the first batch, a tabletability study was carried out on a Korsch XL100 press before the actual compression process, and the results are shown in FIG. 43.

TABLE 53
Process parameters Dexpramipexole ER matrix 223 mg
tablets (300 mg diHCl) confirmation batch lot 031466-24.
Process Parameters

| Blending | Equipment | Diffusion blender |
|---|---|---|
| | Pre-Mix (revolutions) | 100 |
| Milling | Equipment | Quadro Comil 197S |
| | Impeller Diameter (mm) | 108.485 |
| | Screen | 2A024R01823 |
| | | (0.610 mm opening) |
| | Speed (rpm) | 1720 |
| Blending | Equipment | Diffusion blender |
| | Pre-Mix (revolutions) | 300 |
| | Lubrication (revolutions) | 80 |
| Compression | Equipment | Korsch XL100 |
| | Tooling | 0.3050″ × 0.6700″ |
| | | Capsule Shape |
| | Target Tablet | 750 |
| | Weight (mg) | |
| | Turret Speed (rpm) | 20 |
| | Feedframe Speed (rpm) | 25 |
| Film | Equipment | LDCS 5 |
| Coating | Pan Size (inch) | 12 |
| | Nozzle Size (mm) | 0.5 |
| | Atomization Pressure (psi) | 18 |
| | Drum Speed (rpm) | 10 |
| | Target Product | 42 |
| | Temperature | |
| | (° C.) | |
| | Spray Rate (g/min) | 7 |

After the formulation adjustment, tablet hardness plateaued at around 2.5 MPa. The actual compression was performed at 2.2 MPa. The in-process tablet weight, thickness and hardness are summarized in Table 54, and the tablet weight variation is plotted in FIG. 44. The tablets showed consistent weight (weight variation less than ±2%).

TABLE 54
In-process tablet weight, thickness, harness
and tensile strength of Lot 031466-19.
Lot 031466-19

| | | | |
|---|---|---|---|
| Tablet weight | Thickness | Hardness | Tensile strength |
| (mg) | (mm) | (kp) | (MPa) |

Beginning of Run

| | | | |
|---|---|---|---|
| 750 | 6.34 | 21.3 | 2.3 |
| 748 | 6.32 | 20.8 | 2.2 |
| 748 | 6.32 | 20.7 | 2.2 |
| 749 | 6.32 | 21.0 | 2.2 |
| 750 | 6.33 | 21.0 | 2.2 |
| 747 | 6.32 | 21.2 | 2.3 |
| 749 | 6.33 | 21.4 | 2.3 |
| 747 | 6.32 | 21.1 | 2.2 |
| 748 | 6.33 | 21.9 | 2.3 |
| 752 | 6.33 | 21.7 | 2.3 |

Middle of Run

| | | | |
|---|---|---|---|
| 752 | 6.32 | 20.2 | 2.1 |
| 753 | 6.35 | 20.0 | 2.1 |
| 750 | 6.31 | 20.8 | 2.2 |
| 753 | 6.33 | 20.0 | 2.1 |
| 752 | 6.33 | 20.3 | 2.2 |
| 754 | 6.35 | 20.6 | 2.2 |
| 752 | 6.33 | 20.9 | 2.2 |
| 753 | 6.33 | 20.9 | 2.2 |
| 752 | 6.33 | 21.1 | 2.2 |
| 754 | 6.33 | 21.4 | 2.3 |

End of Run

| | | | |
|---|---|---|---|
| 749 | 6.31 | 19.9 | 2.1 |
| 752 | 6.33 | 20.1 | 2.1 |
| 753 | 6.34 | 20.1 | 2.1 |
| 751 | 6.33 | 20.9 | 2.2 |
| 752 | 6.34 | 21.2 | 2.2 |
| 752 | 6.32 | 21.0 | 2.2 |
| 749 | 6.31 | 20.9 | 2.2 |
| 750 | 6.32 | 20.7 | 2.2 |
| 750 | 6.31 | 20.5 | 2.2 |
| 750 | 6.31 | 21.1 | 2.2 |

Average

| | | | |
|---|---|---|---|
| 751 | 6.33 | 20.8 | 2.2 |

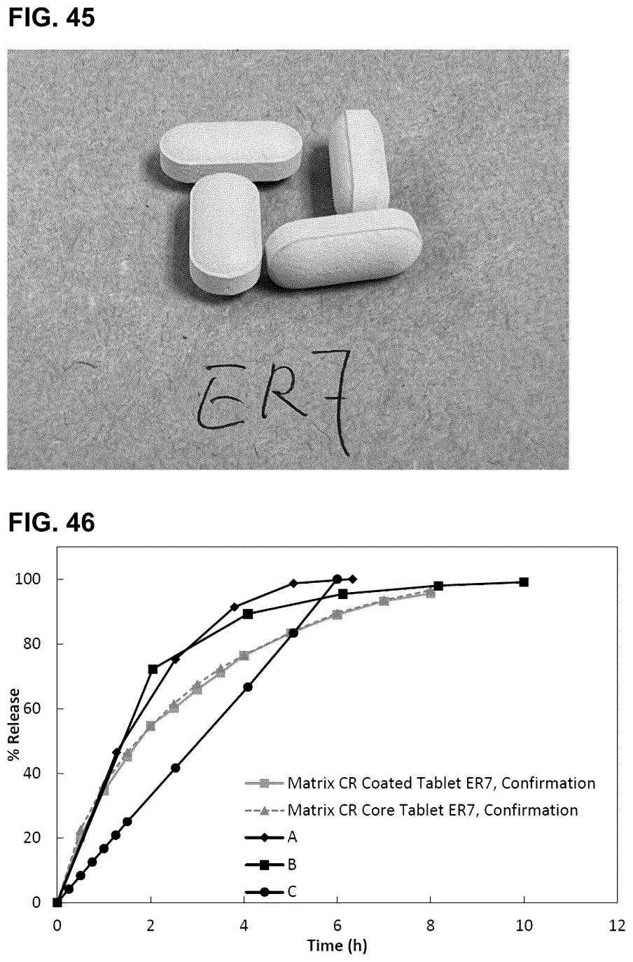

There was no capping or chipping discovered for the tablets. The film coating was also smooth and without any tablet defects (see FIG. 45). The dissolution profiles of both core and film coated tablets are shown in FIG. 46. The dissolution is within the targeted profile range.

### Example 9.6—HPMC K100M Material Grade Comparison

Due to a material supply concern, there was a need for switching the HPMC K100M from Colorcon Methocel grade to Ashland Benecel grade. The impact of material grade on the process performance and dissolution was evaluated. The formulations are summarized in Table 55. Lots 031466-31 and 32 are manufactured at around 100 g batch size.

TABLE 55
Formulations used to study the impact of HPMC K100M grade.

| | | 031466-24 | | 031466-31 | | 031466-32 | |
|---|---|---|---|---|---|---|---|
| Material | Function | mg/unit | % w/w | mg/unit | % w/w | mg/unit | % w/w |
| Dexpramipexole Dihydrochloride | Drug | 319.01 | 42.53 | 319.01 | 42.53 | 319.01 | 42.53 |
| Monohydrate | Substance | | | | | | |
| Hypromellose (Methocel DC2 | Neutral | 112.50 | 15.00 | — | — | — | — |
| K100M Premium) | Matrix | | | | | | |
| Hypromellose (Methocel K100M | Polymer | — | — | 112.50 | 15.00 | — | — |
| Premium CR) | | | | | | | |
| Hypromellose (Benecel K100M PH | | — | — | — | — | 112.50 | 15.00 |
| DC) | | | | | | | |
| Silicified Microcrystalline | Diluent | 311.49 | 41.53 | 311.49 | 41.53 | 311.49 | 41.53 |
| Cellulose (Prosolv HD90) | | | | | | | |
| Colloidal Silicon Dioxide | Glidant | 3.50 | 0.47 | 3.50 | 0.47 | 3.50 | 0.47 |
| (Aerosil 200P) | | | | | | | |
| Magnesium Stearate | Lubricant | 3.50 | 0.47 | 3.50 | 0.47 | 3.50 | 0.47 |
| (Ligamed MF-2-V) | | | | | | | |
| Total | | 750.00 | 100.00 | 750.00 | 100.00 | 750.00 | 100.00 |

The final blend of ER7H confirmation batch and the two batches made with the new HTPMC grade were characterized for bulk density and angle of repose (results see Table 56). All three blends showed similar bulk density and flowability.

TABLE 56
Impact of HPMC grade on the final blend flowability.

| Lot number | 031466-24 | 031466-31 | 031466-32 |
|---|---|---|---|
| HPMC Grade | Methocel | Methocel K100M | Benecel K100M |
| | K100M DC2 | Premium CR | PH DC |
| Bulk Density | 0.57 | 0.54 | 0.53 |
| (g/ml) | | | |
| Angle of Repose | 32 | 31 | 32 |
| (degree) | | | |

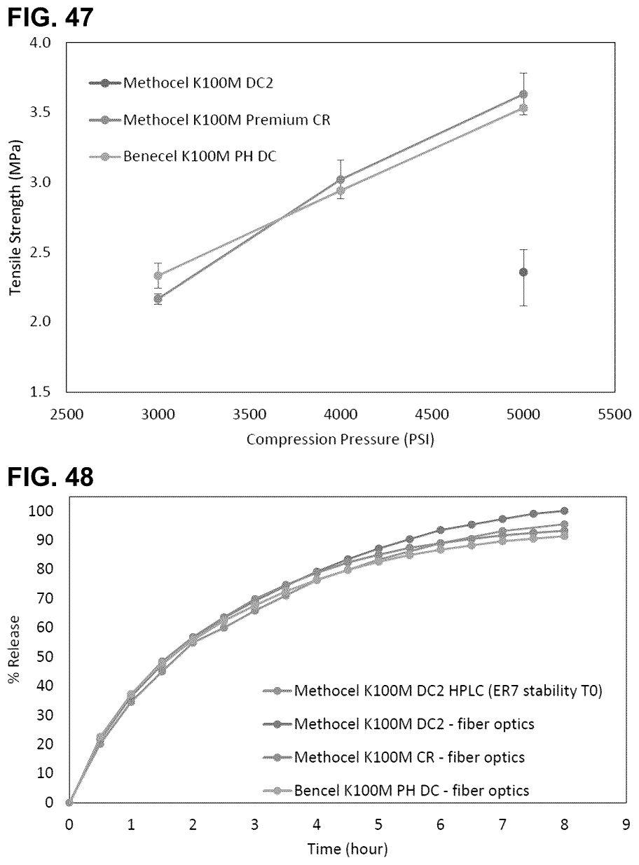

The impact of HPMC grade on the tabletability of the blend was evaluated with a manual press (see FIG. 47). The Methocel K100M DC2 that was used in the confirmation batch 031466-24 needed 5000 psi compression pressure to reach 2.0-2.5 MPa tensile strength, while batches using Methocel K100M Premium CR and Benecel K100M PH DC only needed 3000 psi to reach similar tensile strength. Furthermore, the tablet tensile strength was able to reach above 3.5 MPa for Methocel K100M Premium CR and Benecel K100M PH DC when compressed at 5000 psi.

The impact of HPMC grade on dissolution is shown in FIG. 48. The dissolution test was carried out with a fiber optics system. The fiber optics method was confirmed by comparing the results against the results obtained by the HPLC method for Methocel K100M DC2 batch (031466-24). Dissolution from all three HPMC grades were similar.

To conclude, changing the HTPMC to Benecel K100M PH DC grade should not affect the product quality and will be used for the clinical batches.

### Example 9.7—ER9 Confirmation Batches

Similar to ER7, an ER9 confirmation batch was first manufactured using a formulation with 700 mg core tablet weight (lot: 031466-20). The core tablet weigh was increased to 750 mg in a second confirmation batch (lot: 031466-26) to improve the tablet hardness. The formulations are summarized in Table 57. The manufacturing process was the same as ER7H and the schematic is shown in FIG. 38. Both lots used direction compression process. However, for lot 031466-26, the API and the silicified microcrystalline cellulose were first co-milled to reduce the API size and improve the tablet tabletability.

TABLE 57
Formulation of Dexpramipexole ER matrix 223 mg tablets (300 mg
diHCl) confirmation batch lots 031466-20 and lot 031466-26.

| | | 031466-20 | | | 031466-26 | | | |
|---|---|---|---|---|---|---|---|---|
| | | | % w/w | qty/batch | | % w/w | % w/w | qty/batch |
| Material | Function | mg/unit | core | (g) | mg/unit | core | total | (g) |
| Dexpramipexole | Drug | 319.01 | 45.57 | 638.02 | 319.01 | 42.53 | 41.30 | 638.02 |
| Dihydrochloride | Substance | | | | | | | |
| Monohydrate * | | | | | | | | |
| Sodium | Anionic | 210.00 | 30.00 | 420.00 | 210.00 | 28.00 | 27.18 | 420.00 |
| Carboxymethylcellulose | Matrix | | | | | | | |
| (Blanose CMC | polymer | | | | | | | |
| 7HXF PH) | | | | | | | | |
| Silicified | Diluent | 163.99 | 23.43 | 327.98 | 213.99 | 28.53 | 27.70 | 427.98 |
| Microcrystalline | | | | | | | | |
| Cellulose | | | | | | | | |
| (Prosolv HD90) | | | | | | | | |
| Colloidal Silicon | Glidant | 3.50 | 0.50 | 7.00 | 3.50 | 0.47 | 0.45 | 7.00 |
| Dioxide (Aerosil | | | | | | | | |
| 200 Pharma) | | | | | | | | |
| Magnesium | Lubricant | 3.50 | 0.50 | 7.00 | 3.50 | 0.47 | 0.45 | 7.00 |
| Stearate | | | | | | | | |
| (Ligamed MF-2-V) | | | | | | | | |
| Total Core | | 700.00 | 100.00 | 1400.00 | 750.00 | 100.00 | 97.09 | 1500.00 |
| Tablet | | | | | | | | |
| Opadry II | Colorant | Not performed | | | 22.50 | n/a | 2.91 | 45.00 |
| 85F130089 Pink ** | | | | | | | | |
| Purified water\hat{ } | Suspending | | | | 90.00 | n/a | n/a | n/a |
| | Agent | | | | | | | |
| Total Coated | | | | | 772.50 | n/a | 100.00 | 1545.00 |
| Tablet | | | | | | | | |

* 319.01 mg of dexpramipexole dihydrochloride monohydrate is equivalent to 300.00 mg dexpramipexole dihydrochloride.
** Opadry II 85F130089 PINK was added at a theoretical weight gain of 3.00% w/w of the core tablet. Opadry coating dispersion made at a 20% solids content.
\hat{ }Removed during processing.

The process parameters of the first confirmation batch are summarized in Table 58.

TABLE 58
Process parameters Dexpramipexole ER matrix 223 mg
tablets (300 mg diHCl) confirmation batch lot 031466-20.
Process Parameters

| Blending | Equipment | Diffusion blender |
|---|---|---|
| | Pre-Mix (revolutions) | 300 |
| | Lubrication (revolutions) | 80 |
| Compression | Equipment | Korsch XL100 |
| | Tooling | 0.3050″ × 0.6700″ |
| | | Capsule Shape |
| | Target Tablet | 750 |
| | Weight (mg) | |
| | Turret Speed (rpm) | 20 |
| | Feedframe Speed (rpm) | 25 |

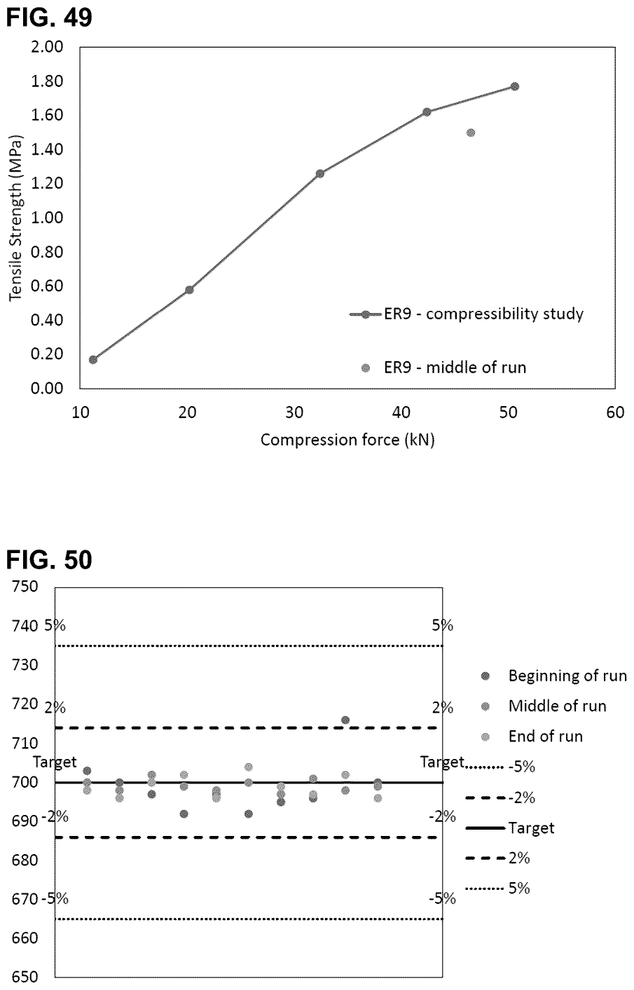

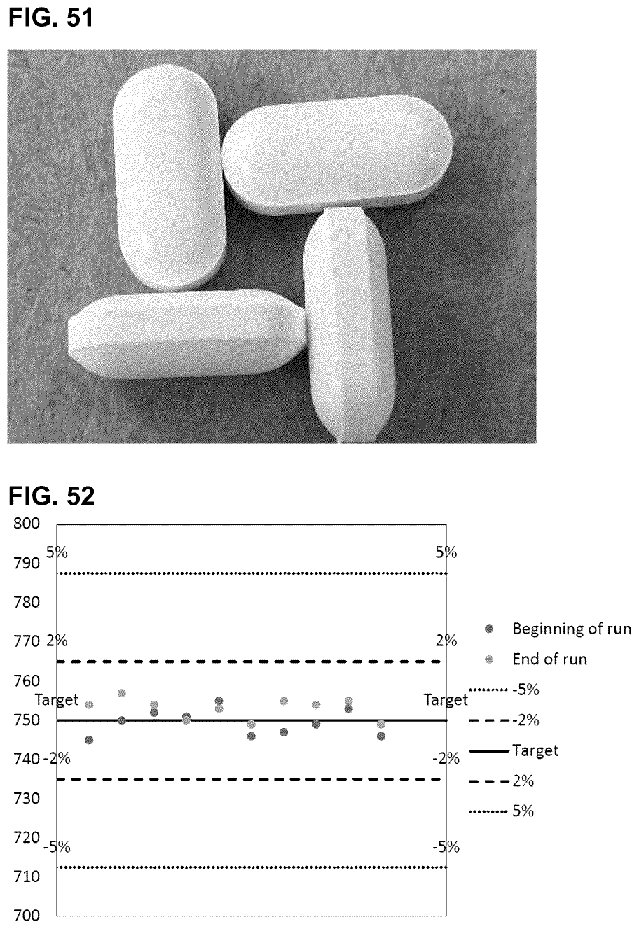

The bulk density of the final blend was 0.56 g/ml. A tabletability study was carried out on a Korsch XL100 press before the actual compression process, and the results are shown in FIG. 49. The tablet hardness increased to around 1.8 MPa. The compression force reached tooling maximum of 50 kN. The in-process tablet weight, thickness, and hardness are summarized in Table 59, and the tablet weight variation is plotted in FIG. 50. The average tablet hardness is 1.6 MPa (lower than the recommended 1.7 MPa). The tablets showed consistent weight (weight variation less than ±2% except for one tablet). There was no capping or chipping discovered for the tablets (see FIG. 51).

TABLE 59
In-process tablet weight, thickness, harness
and tensile strength of Lot 031466-20.
Lot 031466-20

| | | | |
|---|---|---|---|
| Tablet weight | Thickness | Hardness | Tensile strength |
| (mg) | (mm) | (kp) | (MPa) |

Beginning of Run

| | | | |
|---|---|---|---|
| 703 | 6.02 | 14.4 | 1.7 |
| 700 | 6.04 | 14.3 | 1.6 |
| 697 | 5.97 | 14.1 | 1.6 |
| 692 | 5.96 | 14.3 | 1.7 |
| 697 | 6.02 | 14.1 | 1.6 |
| 692 | 5.96 | 14.0 | 1.6 |
| 695 | 5.83 | 21.4 | 2.6 |
| 696 | 5.98 | 14.6 | 1.7 |
| 716 | 6.09 | 13.5 | 1.5 |
| 700 | 6.01 | 14.1 | 1.6 |

Middle of Run

| | | | |
|---|---|---|---|
| 700 | 6.02 | 12.9 | 1.5 |
| 698 | 5.99 | 13.6 | 1.6 |
| 702 | 6.00 | 13.6 | 1.6 |
| 699 | 5.98 | 14.0 | 1.6 |
| 698 | 6.0 | 13.3 | 1.5 |
| 700 | 6.03 | 14.2 | 1.6 |
| 697 | 6.02 | 13.4 | 1.5 |
| 701 | 6.04 | 13.5 | 1.5 |
| 698 | 6.01 | 13.1 | 1.5 |
| 699 | 6.01 | 13.5 | 1.6 |

End of Run

| | | | |
|---|---|---|---|
| 698 | 5.99 | 13.4 | 1.6 |
| 696 | 6.00 | 13.5 | 1.6 |
| 700 | 6.01 | 14.3 | 1.6 |
| 702 | 5.99 | 14.2 | 1.6 |
| 696 | 5.97 | 13.9 | 1.6 |
| 704 | 6.01 | 14.3 | 1.6 |
| 699 | 6.01 | 13.3 | 1.5 |
| 697 | 6.01 | 13.7 | 1.6 |
| 702 | 6.04 | 13.7 | 1.6 |
| 696 | 5.96 | — | — |

Average

| | | | |
|---|---|---|---|
| 699 | 6.00 | 14.1 | 1.6 |

To further investigate the impact of tablet hardness on the tablet appearance integrity, a friability study was conducted for up to 500 revolutions at 25 rpm (see Table 60). USP <1216> Tablet Friability recommends friability to be less than 1.000 after 100 revolutions. The 031466-20 core tablets had a 0.2% weight loss after 500 revolutions and didn't show any tablet defects. The results indicated a lower risk of tablet hardness. Nevertheless, a formulation adjustment was carried out to improve the tablet hardness.

TABLE 60
Friability study on core tablets of lot 031466-20.

| | Tablet Weight | Friability | Any defects in the |
|---|---|---|---|
| Revolutions | (n = 10), mg | (%) | appearance? |
| 0 | 7000 | n/a | No |
| 100 | 7002 | 0 | No |
| 200 | 7006 | 0 | No |
| 500 | 6986 | 0.2 | no |

Two 50 g small batches (denoted as 031466-20A and 031466-20B) were made by adding A) 50 mg/tab worth of silicified microcrystalline cellulose; or B) 50 mg/tab worth of Copovidone (Kollidon VA 64 Fine) to the 031466-20 final blend and bringing the tablet weight to 750 mg. The formulations of 031466-20A and 031466-20B are summarized in Table 61. The resulting tablet hardness from the three formulations, 031466-20, 031466-20A and 031466-20B, were compared by compressing in a manual press at 5000 psi (see Table 62).

TABLE 61
Formulation of 031466-20A and 031466-20B.

| | | 031466-20A | | 031466-20B | |
|---|---|---|---|---|---|
| Material | Function | mg/unit | % w/w | mg/unit | % w/w |
| Dexpramipexole Dihydrochloride | Drug Substance | 319.01 | 42.53 | 319.01 | 42.53 |
| Monohydrate | | | | | |
| Sodium Carboxymethylcellulose | Anioinc Matrix | 210.00 | 28.00 | 210.00 | 28.00 |
| (Blanose CMC 7HXF PH) | Polymer | | | | |
| Silicified Microcrystalline Cellulose | Diluent | 163.99 | 21.87 | 163.99 | 21.87 |
| (Prosolv HD90) | | | | | |
| Colloidal Silicon Dioxide | Glidant | 3.50 | 0.47 | 3.50 | 0.47 |
| (Aerosil 200P) | | | | | |
| Magnesium Stearate | Lubricant | 3.50 | 0.47 | 3.50 | 0.47 |
| (Ligamed MF-2-V) | | | | | |
| Final Blend from 031466-20 | | 700.00 | 93.33 | 700.00 | 93.33 |
| Silicified Microcrystalline Cellulose | Diluent | 50.00 | 6.67 | — | — |
| (Prosolv HD90) | | | | | |
| Copovidone (Kollidon VA64 Fine) | Binder | — | — | 50.00 | 6.67 |
| Total Coated Tablet | | 750.00 | 100.00 | 750.00 | 100.00 |

TABLE 62
Tablet hardness of 031466-20 (700 mg tablet weight)
vs. 031466-20A and 031466-20B (750 mg tablet weight).

| | Compression | | | | Tensile |
|---|---|---|---|---|---|
| | force/ | | Thickness | Hardness | Strength |
| Formulation ER7 | pressure | | (mm) | (kp) | (MPa) |
| 700 mg tab | 46.5 | kN | 6.00 | 14.1 | 1.6 |
| (Korsch XL100 press) | | | | | |
| 700 mg tab | 5000 | psi | 6.17 | 15.1 | 1.7 |
| (manual press) | | | | | |
| 750 mg tab | 5000 | psi | 6.42 | 15.6 | 1.6 |
| (additional | | | | | |
| 50 mg/tab SMCC) | | | | | |
| 750 mg tab | 5000 | psi | 6.39 | 25.4 | 2.7 |
| (additional | | | | | |
| 50 mg/tab Kollidon | | | | | |

VA64 Fine)

Tablet hardness remained similar with additional 50 mg/tab SMCC. However, the tablet hardness increased significantly from 15 kp to 25 kp with additional 50 mg/tab Kollidon VA64 Fine. Therefore, the formulation with additional 50 mg/tab Kollidon VA64 Fine was compressed on a larger scale on Korsch XL 100 press (denoted as lot 031466-23). The in-process tablet weight, thickness and hardness are summarized in Table 63, and the tablet weight variation is plotted in FIG. 52. The tablets showed good hardness and weight consistency.

TABLE 63
In-process tablet weight, thickness, harness
and tensile strength of Lot 031466-23.
Lot 031466-23

| | | | |
|---|---|---|---|
| Tablet weight | Thickness | Hardness | Tensile strength |
| (mg) | (mm) | (kp) | (MPa) |

Beginning of Run

| | | | |
|---|---|---|---|
| 745 | 6.25 | 25.1 | 2.7 |
| 750 | 6.30 | 17.6 | 1.9 |
| 752 | 6.29 | 26.5 | 2.8 |
| 751 | 6.33 | 25.9 | 2.7 |
| 755 | 6.35 | 24.7 | 2.6 |
| 746 | 6.27 | 25.9 | 2.8 |
| 747 | 6.34 | 24.9 | 2.6 |
| 749 | 6.29 | 25.8 | 2.8 |
| 753 | 6.36 | 25.9 | 2.7 |
| 746 | 6.34 | 25.9 | 2.7 |

End of Run

| | | | |
|---|---|---|---|
| 754 | 6.26 | 24.6 | 2.7 |
| 757 | 6.31 | 26.0 | 2.8 |
| 754 | 6.36 | 25.7 | 2.7 |
| 750 | 6.27 | 26.1 | 2.8 |
| 753 | 6.30 | 26.8 | 2.9 |
| 749 | 6.27 | 25.4 | 2.7 |
| 755 | 6.31 | 25.9 | 2.8 |
| 754 | 6.30 | 26.7 | 2.9 |
| 755 | 6.30 | 26.8 | 2.9 |
| 749 | 6.27 | 25.7 | 2.9 |

Average

| | | | |
|---|---|---|---|
| 751.2 | 6.30 | 25.4 | 2.7 |

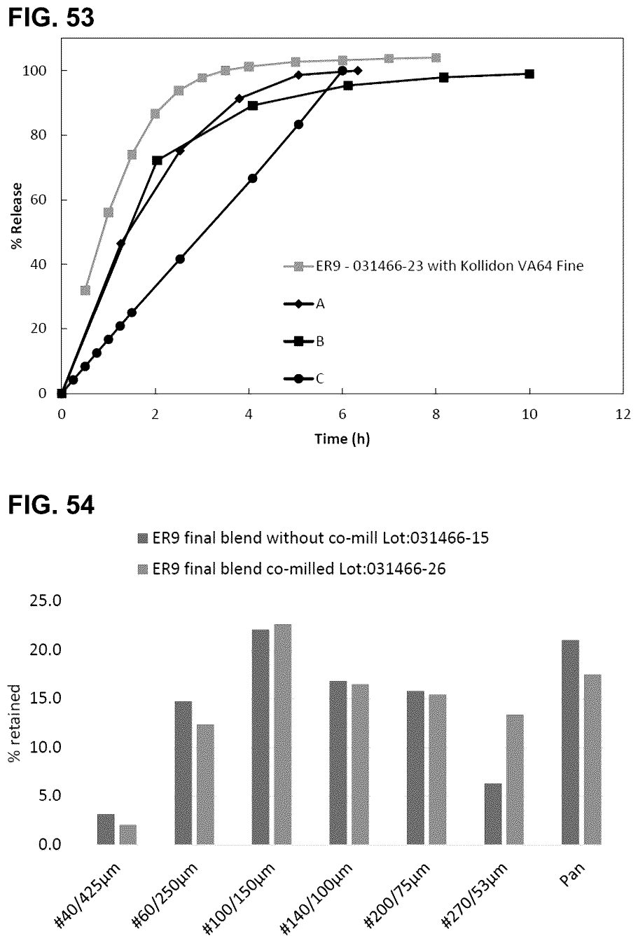

The dissolution results of lot 031466-23 is shown in FIG. 53. The release was sped up significantly with the presence of Kollidon VA64 Fine in the formulation. Since the formulation was faster than the targeted range, the second confirmation batch did not use Kollidon VA64 Fine, and instead adopted the extra SMCC method as shown in Table 57 above.

The process parameters of the second confirmation batch are summarized in Table 64. The final blend bulk density of the second confirmation batch 031466-26 was 0.61 g/ml.

TABLE 64
Process parameters Dexpramipexole ER matrix 223 mg tablets
(300 mg diHCl) confirmation batch lot 031466-26.
Process Parameters

| Blending | Equipment | Diffusion blender |
|---|---|---|
| | Pre-Mix (revolutions) | 100 |
| Milling | Equipment | Quadro Comil 197S |
| | Impeller Diameter (mm) | 108.485 |
| | Screen | 2A024R01823 (0.610 mm opening) |
| | Speed (rpm) | 1720 |
| Blending | Equipment | Diffusion blender |
| | Pre-Mix (revolutions) | 300 |
| | Lubrication (revolutions) | 80 |
| Compression | Equipment | Korsch XL100 |
| | Tooling | 0.3050″ × 0.6700″ Capsule Shape |
| | Target Tablet Weight (mg) | 750 |
| | Turret Speed (rpm) | 20 |
| | Feedframe Speed (rpm) | 25 |
| Film | Equipment | LDCS 5 |
| Coating | Pan Size (inch) | 12 |
| | Nozzle Size (mm) | 0.5 |
| | Atomization Pressure (psi) | 18 |
| | Drum Speed (rpm) | 10 |
| | Target Product Temperature (° C.) | 42 |
| | Spray Rate (g/min) | 7 |

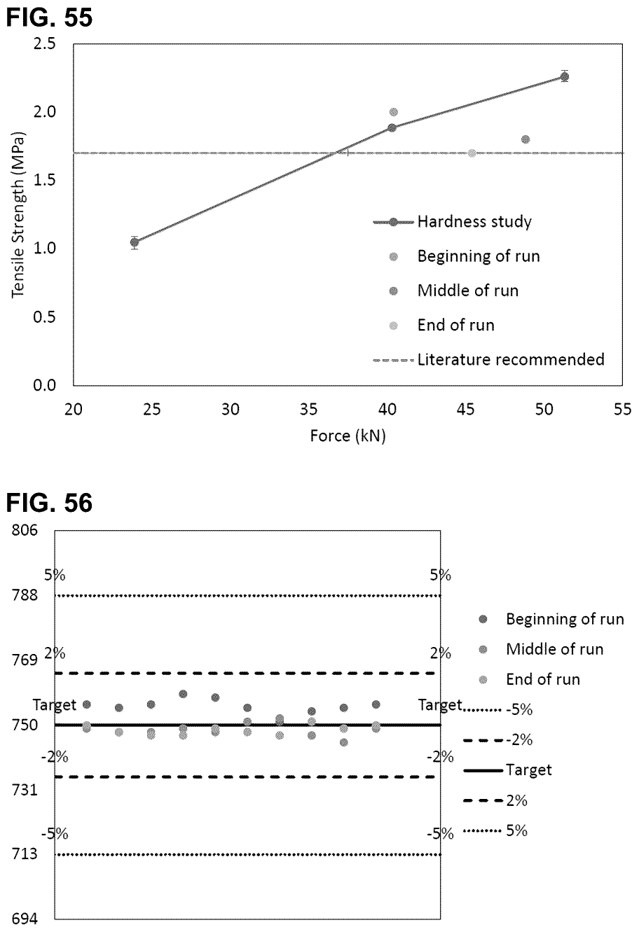

Since the second confirmation batch incorporated an API co-mill process, the particles size distribution (PSD) of the final blend was compared to the PSD of a development batch that without an API co-mill process (FIG. 54). After co-milling, the final blend had more fine particles as the number of fine particles retained on the #270/53 um screen increased. Increasing the fine portion of particles can fill the voids between large particles and improve the interparticle bonding. Thus, the increment in the fine particle portion should help improve the tablet tabletability. A tabletability study was carried out on a Korsch XL100 press, and the results are shown in FIG. 55. After the formulation adjustment and incorporating the API co-mill process, the tablet tensile strength was able to reach around 2.2 MPa at 50 kN compared to 1.8 MPa at 50 kN for the first confirmation batch. The in-process tablet weight, thickness and hardness are summarized in Table 65, and the tablet weight variation is plotted in FIG. 56.

TABLE 65
In-process tablet weight, thickness, harness and
tensile strength of Lot 031466-26.
Lot 031466-26

| | | | |
|---|---|---|---|
| Tablet weight | | | Tensile strength |
| (mg) | Thickness (mm) | Hardness (kp) | (MPa) |

Beginning of Run

| | | | |
|---|---|---|---|
| 756 | 6.31 | 18.3 | 1.9 |
| 755 | 6.31 | 18.3 | 1.9 |
| 756 | 6.31 | 19.2 | 2.0 |
| 759 | 6.32 | 19.3 | 2.0 |
| 758 | 6.32 | 18.8 | 2.0 |
| 755 | 6.32 | 19.3 | 2.0 |
| 751 | 6.28 | 18.7 | 2.0 |
| 754 | 6.33 | 18.7 | 2.0 |
| 755 | 6.30 | 18.1 | 1.9 |
| 756 | 6.30 | 18.9 | 2.0 |

Middle of Run

| | | | |
|---|---|---|---|
| 749 | 6.26 | 16.5 | 1.8 |
| 748 | 6.28 | 16.8 | 1.8 |
| 748 | 6.28 | 16.9 | 1.8 |
| 749 | 6.26 | 16.4 | 1.8 |
| 748 | 6.29 | 17.1 | 1.8 |
| 751 | 6.27 | 17.1 | 1.8 |
| 752 | 6.30 | 16.8 | 1.8 |
| 747 | 6.25 | 16.9 | 1.8 |
| 745 | 6.23 | 15.9 | 1.7 |
| 749 | 6.27 | 17.0 | 1.8 |

End of Run

| | | | |
|---|---|---|---|
| 750 | 6.32 | 16.5 | 1.8 |
| 748 | 6.30 | 15.4 | 1.6 |
| 747 | 6.27 | 16.0 | 1.7 |
| 747 | 6.29 | 16.3 | 1.7 |
| 749 | 6.30 | 16.0 | 1.7 |
| 748 | 6.29 | 16.4 | 1.8 |
| 747 | 6.27 | 16.3 | 1.8 |
| 751 | 6.32 | 16.5 | 1.8 |
| 749 | 6.31 | 16.4 | 1.7 |
| 750 | 6.28 | 16.6 | 1.8 |

Average

| | | | |
|---|---|---|---|
| 751 | 6.29 | 17.2 | 1.8 |

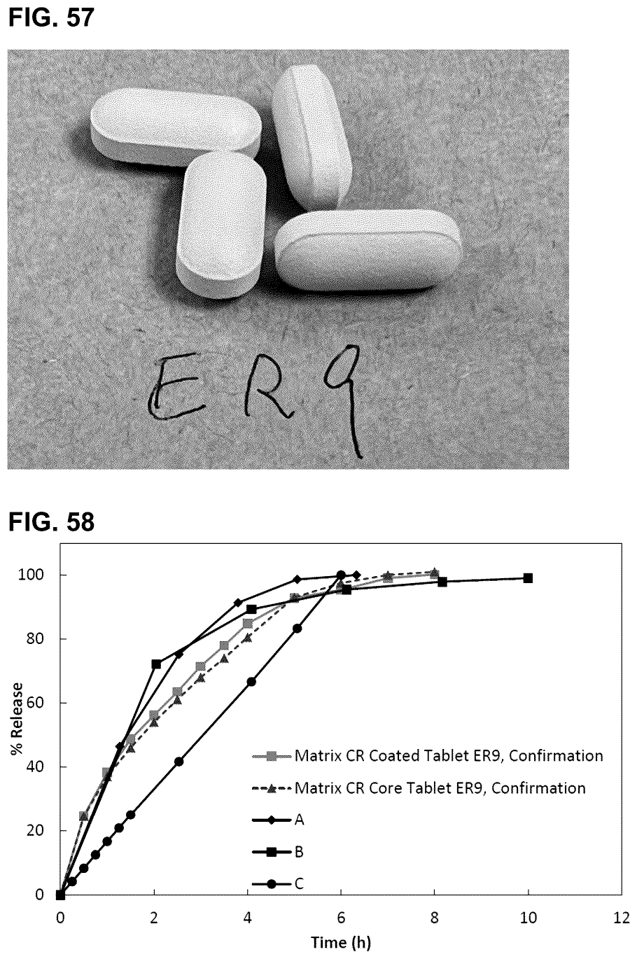

The tablet weight was consistent (variation less than 2 T). There was no capping or chipping discovered for the tablets. The film coating was also smooth and without any tablet defects (see FIG. 57). The dissolution profiles of both core and film coated tablets are shown in FIG. 58. The profile between the core and film coated tablets were similar. The dissolution was within the targeted profile range.

### Example 9.8—ER5 Clinical Trial Material Batch

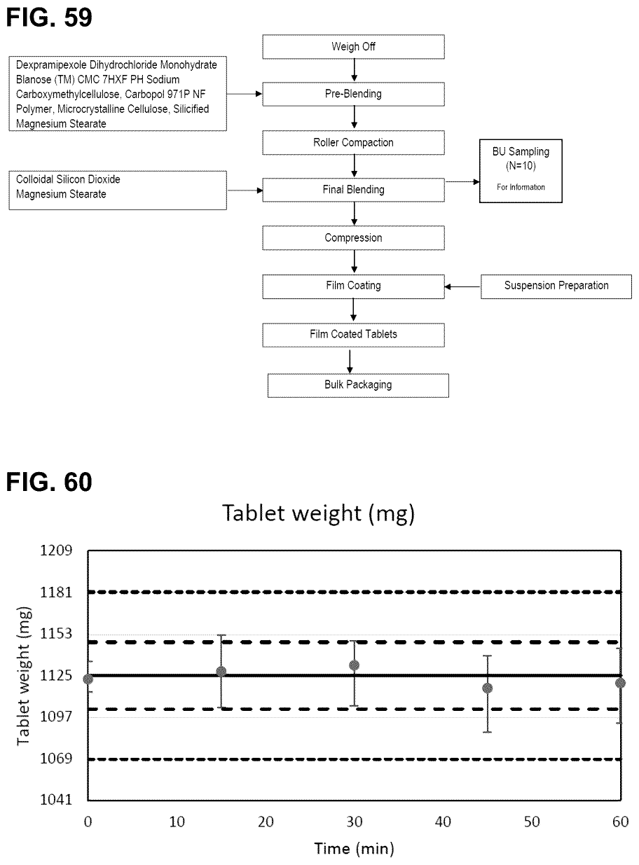

ER5 Clinical Trial Material (CTM) batch was manufactured at 8.691 kg scale (7,500 tablets). The formulation details are provided in Table 66. The manufacturing process is provided in FIG. 59, and the process parameters are presented in Table 67.

TABLE 66
Formulation composition of ER5 CTM batch (batch 24JM-085)

| | | | | |
|---|---|---|---|---|
| | | | | Theoretical |
| | | Qty/tablet | | Batch |
| Material | Function | (mg) | % w/w | Weight (g) |

Intra-Granular (IG) - Roller Compaction

| | | | | |
|---|---|---|---|---|
| Dexpramipexole Dihydrochloride | Drug Substance | 399.82 | 34.50 | 2998.65 |
| Monohydrate * | | | | |
| Sodium Carboxymethylcellulose | Anionic Matrix | 332.50 | 28.69 | 2493.75 |
| (Blanose CMC 7HXF PH) | Polymer | | | |
| Carbomer (Carbopol 971P) | | 142.50 | 12.30 | 1068.75 |
| Silicified Microcrystalline Cellulose | Diluent | 236.11 | 20.38 | 1770.83 |
| (Prosolv HD90) | | | | |
| Magnesium Stearate | Lubricant | 2.81 | 0.24 | 21.08 |
| (Ligamed MF-2-V) | | | | |

Extra-Granular (EG) Final Blend

| | | | | |
|---|---|---|---|---|
| Colloidal Silicon Dioxide | Glidant | 5.63 | 0.49 | 42.23 |
| (Aerosil 200P) | | | | (52.00\hat{ }\hat{ }) |
| Magnesium Stearate | Lubricant | 5.63 | 0.49 | 42.23 |
| (Ligamed MF-2-V) | | | | (52.00\hat{ }\hat{ }) |
| Total Core Tablet | | 1125.00 | 97.09 | 8437.52 |

Film Coating

| | | | | |
|---|---|---|---|---|
| Opadry II 85F130089 Pink ** | Colorant | 33.75 | 2.91 | 253.13 |
| | | | | (379.70) |
| Purified water\hat{ } | Suspending | 135.00 | n/a | 1012.50 |
| | Agent | | | (1518.75) |
| Total Coated Tablet | | 1158.75 | 100 | 8691 |

* 399.82 mg of dexpramipexole dihydrochloride monohydrate is equivalent to 376.00 mg dexpramipexole dihydrochloride.
** Opadry II 85F130089 PINK was added at a theoretical weight gain of 3.00% w/w of the core tablet. Opadry made at a 20% solids content with approximately 50% suspension overage to accommodate spray efficiency.
\hat{ }Removed during processing.
\hat{ }\hat{ }Primary weights adjusted to accommodate process loss during transfer and weight adjustment based on roller compaction yield. Final formulated weights per w/w described in table above.

TABLE 67
Process parameters of Dexpramipexole ER matrix 280
mg tablets (376 mg diHCl) CTM batch.
Process Parameters

| Blending | Equipment | Diffusion blender |
|---|---|---|
| | Pre-Mix (revolutions) | 240 |
| | Lubrication (revolutions) | 60 |
| Roller | Equipment | Gerteis Mini-Polygran |
| Compaction | Roller Type | Two Knurled |
| | Granulator Rotor | Star |
| | Granulator Screen | 1.0 mm regular wire mesh |
| | Roller Speed (rpm) | 3 |
| | Roller Pressure (kN/cm) | 5 |
| | Gap Size (mm) | 2.5 |
| | Gap Control | ON |
| | Agitator Speed (rpm) | 10 |
| | Granulator Speed CW/CCW | 70 rpm/70 rpm |
| | Granulator Angle CW/CCW | 250°/350° |
| Blending | Equipment | Diffusion blender |
| | Pre-Mix (revolutions) | 240 |
| | Lubrication (revolutions) | 60 |
| Compression | Equipment | Korsch XL100 |
| | Tooling | 0.3937″ × 0.7874″ Modified Oval |
| | Target Tablet Weight (mg) | 1125 |
| | Target Hardness (kp) | 25 |
| | Turret Speed (rpm) | 25 |
| | Feedframe Speed (rpm) | 30 |
| Film | Equipment | O'Hara Coating Pan |
| Coating | Pan Size (inch) | 19 |
| | Nozzle Size (mm) | 1.2 |
| | Atomization Pressure (psi) | 20 |
| | Drum Speed (rpm) | 12 |
| | Inlet Air Volume (CFM) | 180 |
| | Target Exhaust Air Temperature (° C.) | 48.5 |
| | Spray Rate (g/min) | 22 |

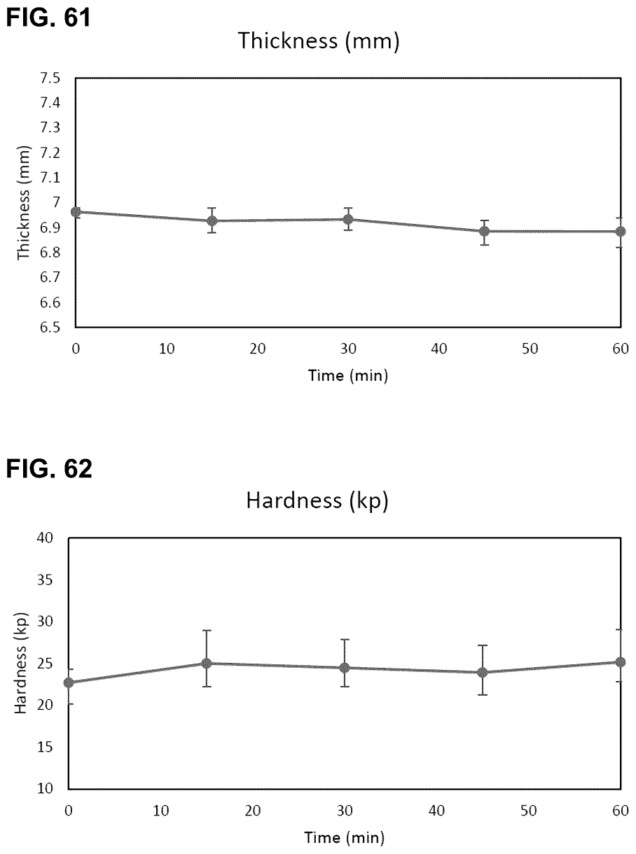

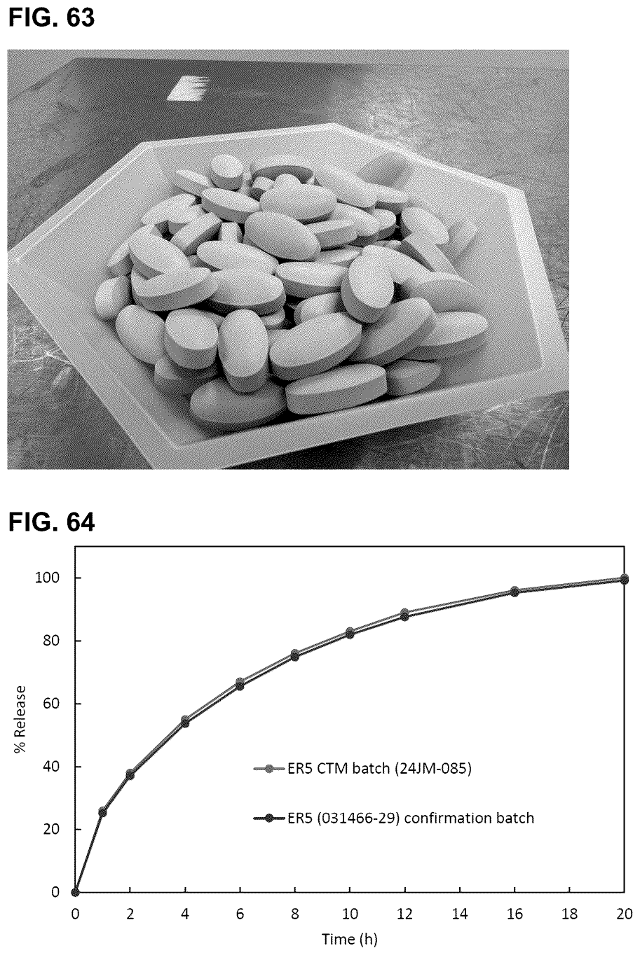

Start-up testing of the compression process was successful (see Table 68). The compression process was smooth (see FIGS. 60-62) during the second attempt as the tablet weight, thickness and hardness are within the targeted range. No defect was observed on the tablet appearance. After film coating, tablets reached targeted weight gain and showed good appearance (see FIG. 63). The dissolution profile matched with the non-GMP confirmation batch discussed in Example 9.4 ER5 Confirmation Batches (see FIG. 64).

TABLE 68
Start-up testing of batch 24JM-085.
Batch 24JM-085

| | | | |
|---|---|---|---|
| Tablet weight | Thickness | Hardness | Tensile strength |
| (mg) | (mm) | (kp) | (MPa) |

Start-Up Testing Set 1

| | | | |
|---|---|---|---|
| 1096.1 | 6.88 | 21.6 | 1.5 |
| 1118.1 | 6.92 | 20.0 | 1.4 |
| 1111.3 | 6.90 | 19.1 | 1.3 |
| 1100.2 | 6.89 | 19.9 | 1.4 |
| 1096.9 | 6.90 | 23.4 | 1.6 |
| 1109.6 | 6.92 | 30.2 | 2.1 |
| 1114.3 | 6.95 | 22.2 | 1.5 |
| 1091.1 | 6.88 | 17.8 | 1.2 |
| 1107.2 | 6.92 | 19.5 | 1.3 |
| 1097.9 | 6.90 | 19.0 | 1.3 |

Start-Up Testing Set 2

| | | | |
|---|---|---|---|
| 1113.7 | 6.94 | 18.1 | 1.2 |
| 1103.0 | 6.93 | 20.5 | 1.4 |
| 1114.8 | 6.95 | 19.4 | 1.3 |
| 1097.3 | 6.92 | 20.2 | 1.4 |
| 1125.7 | 6.93 | 21.0 | 1.4 |
| 1101.1 | 6.91 | 19.6 | 1.3 |
| 1095.4 | 6.91 | 22.2 | 1.5 |
| 1108.5 | 6.94 | 19.7 | 1.3 |
| 1103.7 | 6.92 | 19.1 | 1.3 |
| 1104.2 | 6.91 | 19.1 | 1.3 |

Start-Up Testing Set 3

| | | | |
|---|---|---|---|
| 1138.0 | 6.91 | 23.6 | 1.6 |
| 1118.7 | 6.86 | 28.3 | 1.9 |
| 1117.4 | 6.89 | 26.4 | 1.8 |
| 1138.1 | 6.91 | 25.2 | 1.7 |
| 1107.4 | 6.83 | 26.5 | 1.8 |
| 1124.7 | 6.86 | 22.9 | 1.6 |
| 1135.2 | 6.88 | 25.4 | 1.7 |
| 1108.1 | 6.83 | 28.8 | 2.0 |
| 1129.7 | 6.88 | 27.5 | 1.9 |
| 1125.1 | 6.86 | 25.7 | 1.8 |

Average

| | | | |
|---|---|---|---|
| 1112 | 6.90 | 22.4 | 1.5 |

Target

| | | | |
|---|---|---|---|
| 1125.00 (1040.63- | n/a | 25 (15-30) | n/a |
| 1209.38) | | | |

### Example 9.9—ER7H Clinical Trial Material Batch

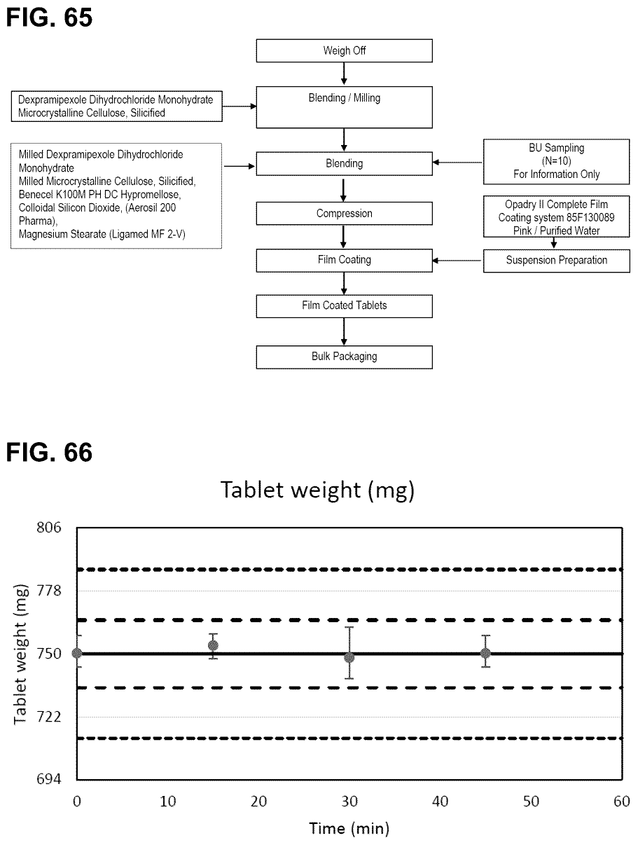

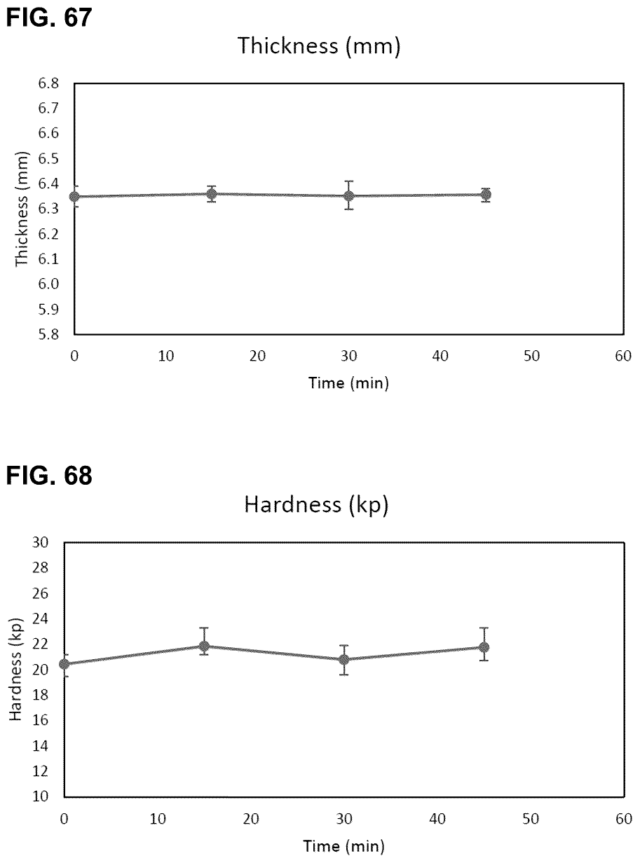

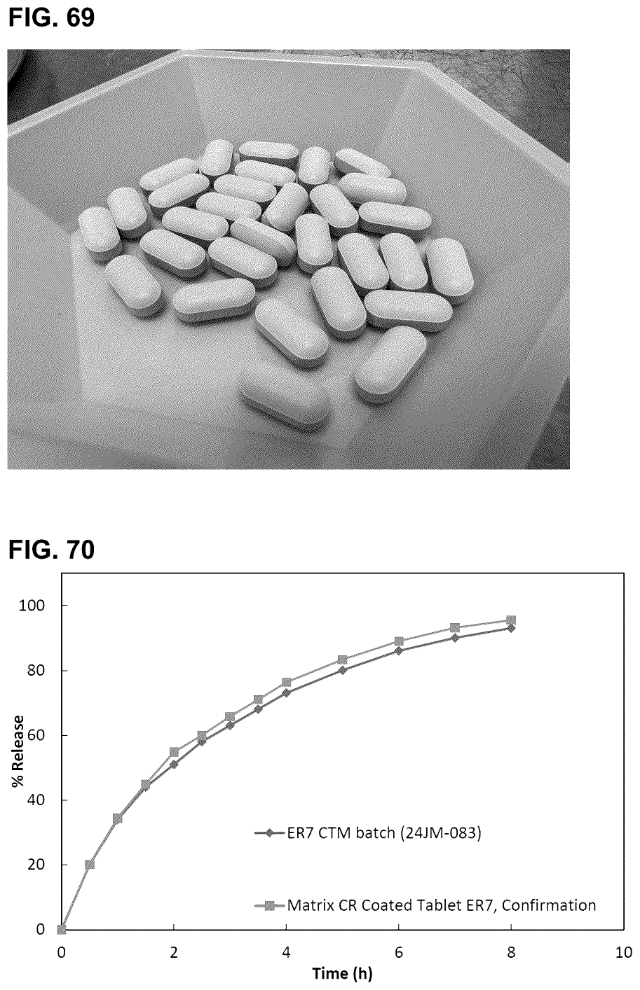

ER7H CTM batch was manufactured at 7.725 kg scale (10,000 tablets). The matrix polymer hypromellose was switched to Ashland Benecel K100M PH DC grade as discussed in Example 9.6—HTPMC K100M Material Grade Comparison. The formulation table is provided in Table 69. The manufacturing process is provided in FIG. 65, and the process parameters are presented in Table 70. All three sets of start-up samples of the compression were within the targeted range (see Table 71). The in-process tablet weight, thickness, and hardness had minor variation throughout the process (see FIGS. 66-68). Tablets showed good appearance and were absent of defects. Tablets had a smooth and even layer of coating after the film coating process (see FIG. 69). The dissolution of the drug product was similar to the confirmation batch discussed in Example 9.5—ER7H Confirmation Batches (see FIG. 70).

TABLE 69
Formulation composition of ER7H CTM batch (batch 24JM-083)

| | | | | Theoretical |
|---|---|---|---|---|
| | | Qty/tablet | | Batch |
| Material | Function | (mg) | % w/w | Weight (g) |
| Dexpramipexole Dihydrochloride | Drug Substance | 319.01 | 41.30 | 3190.10 |
| Monohydrate * | | | | |
| Benecel K100M PH DC Hypromellose | Neutral Matrix | 112.50 | 14.56 | 1125.00 |
| | Polymer | | | |
| Silicified Microcrystalline Cellulose | Diluent | 311.49 | 40.32 | 3114.90 |
| (Prosolv HD90) | | | | |
| Magnesium Stearate | Glidant | 3.50 | 0.45 | 35.00 |
| (Ligamed MF-2-V) | | | | |
| Colloidal Silicon Dioxide | Lubricant | 3.50 | 0.45 | 35.00 |
| (Aerosil 200P) | | | | |
| Core Tablet Total | | 750.00 | 97.08 | 7500.00 |

Film Coating

| | | | | |
|---|---|---|---|---|
| Opadry II 85F130089 Pink ** | Colorant | 22.50 | 2.91 | 225.00 |
| | | | | (337.50) |
| Purified water\hat{ } | Suspending | 90.00 | n/a | 900.00 |
| | Agent | | | (1350.00) |
| Total Coated Tablet | | 772.50 | 100.0 | 7725.00 |

* 319.01 mg of dexpramipexole dihydrochloride monohydrate is equivalent to 300.00 mg dexpramipexole dihydrochloride.
** Opadry II 85F130089 PINK was added at a theoretical weight gain of 3.00% w/w of the core tablet. Opadry made at a 20% solids content with approximately 50% suspension overage to accommodate spray efficiency.
\hat{ }Removed during processing.

TABLE 70
Process parameters of Dexpramipexole ER matrix 223
mg tablets (300 mg diHCl) CTM batch.
Process Parameters

| Blending | Equipment | Diffusion blender |
|---|---|---|
| | Pre-Mix (revolutions) | 100 |
| Milling | Equipment | Quadro Comil 197S |
| | Impeller Diameter (mm) | 108.485 |
| | Screen | 2A024R01823 (0.610 mm opening) |
| | Speed (rpm) | 1400 |
| Blending | Equipment | Diffusion blender |
| | Pre-Mix (revolutions) | 300 |
| | Lubrication (revolutions) | 60 |
| Compression | Equipment | Korsch XL100 |
| | Tooling | 0.3050″ × 0.6700″ Capsule Shape |
| | Target Tablet Weight (mg) | 750 |
| | Tablet Hardness (kp) | 20 |
| | Turret Speed (rpm) | 20 |
| | Feedframe Speed (rpm) | 30 |
| Film | Equipment | O'Hara Coating Pan |
| Coating | Pan Size (inch) | 19 |
| | Nozzle Size (mm) | 1.2 |
| | Atomization Pressure (psi) | 20 |
| | Drum Speed (rpm) | 12 |
| | Inlet Air Volume (CFM) | 180 |
| | Target Product Temperature (° C.) | 48.5 |
| | Spray Rate (g/min) | 22 |

TABLE 71
Start-up testing of batch 24JM-083.
Batch 24JM-083

| | | | |
|---|---|---|---|
| Tablet weight | Thickness | Hardness | Tensile strength |
| (mg) | (mm) | (kp) | (MPa) |

Start-Up Testing Set 1

| | | | |
|---|---|---|---|
| 754 | 6.26 | 21.9 | 1.9 |
| 752 | 6.30 | 21.8 | 1.8 |
| 751 | 6.29 | 21.5 | 1.8 |
| 753 | 6.29 | 21.8 | 1.9 |
| 749 | 6.28 | 21.6 | 1.8 |
| 749 | 6.28 | 22.2 | 1.9 |
| 750 | 6.27 | 20.8 | 1.8 |
| 746 | 6.26 | 20.3 | 1.7 |
| 745 | 6.26 | 21.2 | 1.8 |
| 750 | 6.28 | 20.4 | 1.7 |

Start-Up Testing Set 2

| | | | |
|---|---|---|---|
| 743 | 6.25 | 22.5 | 1.9* |
| 744.0 | 6.26 | 21.6 | 1.8 |
| 744 | 6.26 | 21.5 | 1.8 |
| 743 | 6.25 | 22.2 | 1.9 |
| 745 | 6.26 | 21.7 | 1.9 |
| 740 | 6.28 | 22.2 | 1.9 |
| 746 | 6.26 | 21.4 | 1.8 |
| 746 | 6.27 | 22.2 | 1.9 |
| 750 | 6.26 | 21.9 | 1.9 |
| 743 | 6.28 | 21.7 | 1.8 |

Start-Up Testing Set 3

| | | | |
|---|---|---|---|
| 754 | 6.38 | 21.7 | 1.8 |
| 742 | 6.33 | 21.9 | 1.8 |
| 748 | 6.34 | 21.9 | 1.8 |
| 746 | 6.33 | 22.9 | 1.9 |
| 748 | 6.36 | 23.1 | 1.9 |
| 749 | 6.35 | 23.4 | 2.0 |
| 756 | 6.36 | 21.9 | 1.8 |
| 751 | 6.35 | 21.5 | 1.8 |
| 754 | 6.37 | 21.6 | 1.8 |
| 747 | 6.33 | 22.8 | 1.9 |

Average

| | | | |
|---|---|---|---|
| 748 | 6.30 | 21.8 | 1.9 |

Target

| | | | |
|---|---|---|---|
| 750.00 (693.75- | n/a | 20 (15-30) | n/a |
| 806.25) | | | |

### Example 9.9—ER7H Clinical Trial Material Batch

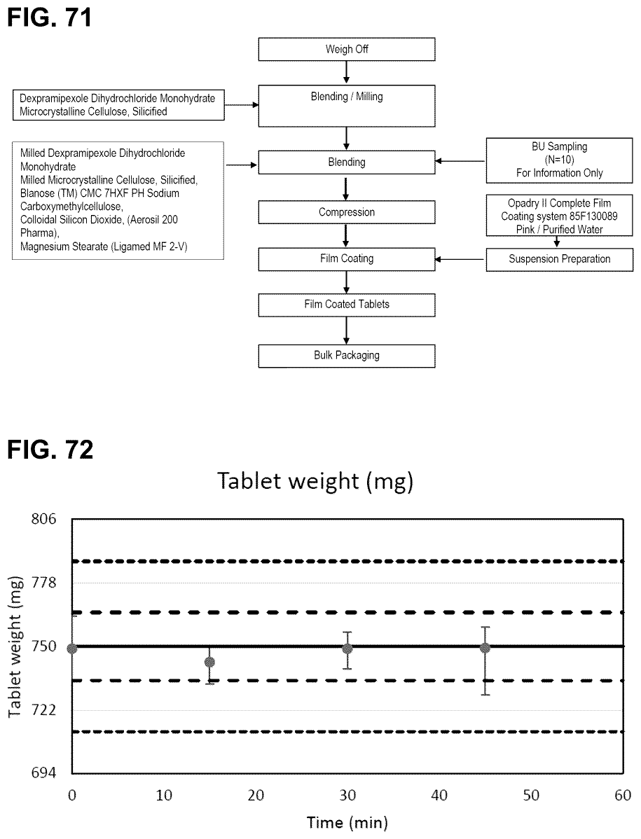

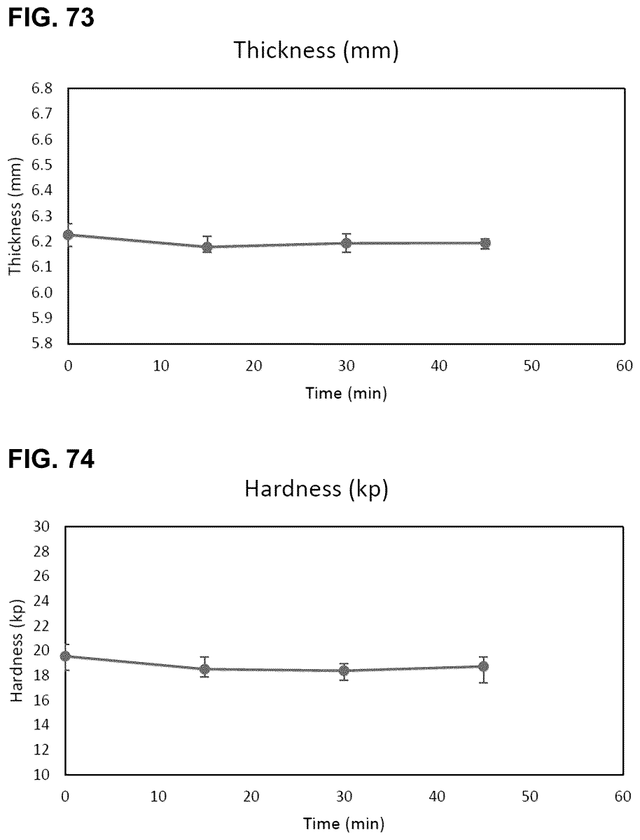

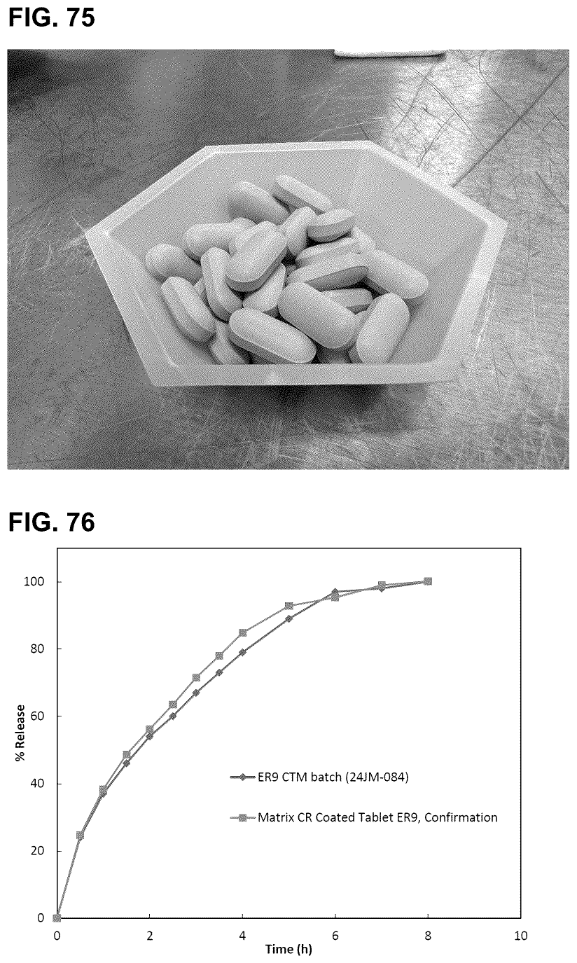

ER9 CTM batch was manufactured at 7.725 kg scale (10,000 tablets). The matrix polymer hypromellose was Blanose (TM) CMC 7HXF PH. The formulation table is provided in Table 72. The manufacturing process is provided in FIG. 71, and the process parameters are presented in Table 73. All three sets of start-up samples of the compression were within the targeted range (see Table 74). The in-process tablet weight, thickness, and hardness had minor variation throughout the process (see FIGS. 72-74). Tablets showed good appearance and were absent of defects. Tablets had a smooth and even layer of coating after the film coating process (see FIG. 75). The dissolution of the drug product was similar to the confirmation batch discussed in Example 9.7—ER9 Confirmation Batches (see FIG. 76).

TABLE 72
Formulation composition of ER7H CTM batch (batch 24JM-084)

| | | | | Theoretical |
|---|---|---|---|---|
| | | Qty/tablet | | Batch |
| Material | Function | (mg) | % w/w | Weight (g) |
| Dexpramipexole Dihydrochloride | Drug Substance | 319.01 | 41.30 | 3190.10 |
| Monohydrate * | | | | |
| Sodium Carboxymethylcellulose (Blanose | Anionic Matrix | 210.00 | 27.18 | 2100.00 |
| (TM) CMC 7HXF PH) | Polymer | | | |
| Silicified Microcrystalline Cellulose | Diluent | 213.99 | 27.70 | 2139.90 |
| (Prosolv HD90) | | | | |
| Magnesium Stearate | Lubricant | 3.50 | 0.45 | 35.00 |
| (Ligamed MF-2-V) | | | | |
| Colloidal Silicon Dioxide | Glidant | 3.50 | 0.45 | 35.00 |
| (Aerosil 200P) | | | | |
| Core Tablet Total | | 750.00 | 97.08 | 7500.00 |

Film Coating

| | | | | |
|---|---|---|---|---|
| Opadry II 85F130089 Pink ** | Colorant | 22.50 | 2.91 | 225.00 |
| | | | | (338.00) |
| Purified water\hat{ } | Suspending | 90.00 | n/a | 900.00 |
| | Agent | | | (1350.00) |
| Total Coated Tablet | | 772.50 | 100 | 7725.00 |

* 319.01 mg of dexpramipexole dihydrochloride monohydrate is equivalent to 300.00 mg dexpramipexole dihydrochloride.
** Opadry II 85F130089 PINK was added at a theoretical weight gain of 3.00% w/w of the core tablet. Opadry made at a 20% solids content with approximately 50% suspension overage to accommodate spray efficiency.
\hat{ }Removed during processing.

TABLE 73
Process parameters of Dexpramipexole ER matrix 223
mg tablets (300 mg diHCl) CTM batch.
Process Parameters

| Blending | Equipment | Diffusion blender |
|---|---|---|
| | Pre-Mix (revolutions) | 100 |
| Milling | Equipment | Quadro Comil 197S |
| | Impeller Diameter (mm) | 108.485 |
| | Screen | 2A024R01823 (0.610 mm opening) |
| | Speed (rpm) | 1400 |
| Blending | Equipment | Diffusion blender |
| | Pre-Mix (revolutions) | 300 |
| | Lubrication (revolutions) | 60 |
| Compression | Equipment | Korsch XL100 |
| | Tooling | 0.3050″ × 0.6700″ Capsule Shape |
| | Target Tablet Weight (mg) | 750 |
| | Tablet Hardness (kp) | 20 |
| | Turret Speed (rpm) | 20 |
| | Feedframe Speed (rpm) | 30 |
| Film | Equipment | O'Hara Coating Pan |
| Coating | Pan Size (inch) | 19 |
| | Nozzle Size (mm) | 1.2 |
| | Atomization Pressure (psi) | 20 |
| | Drum Speed (rpm) | 12 |
| | Inlet Air Volume (CFM) | 180 |
| | Target Product Temperature (° C.) | 48.5 |
| | Spray Rate (g/min) | 22 |

TABLE 74
Start-up testing of batch 24JM-084.
Batch 24JM-084

| | | | |
|---|---|---|---|
| Tablet weight | Thickness | Hardness | Tensile strength |
| (mg) | (mm) | (kp) | (MPa) |

Start-Up Testing Set 1

| | | | |
|---|---|---|---|
| 747.3 | 6.25 | 17.7 | 1.5 |
| 742.1 | 6.26 | 17.4 | 1.5 |
| 747.4 | 6.20 | 18.9 | 1.6 |
| 747.5 | 6.20 | 18.5 | 1.6 |
| 750.2 | 6.25 | 18.3 | 1.6 |
| 747.7 | 6.25 | 18.0 | 1.5 |
| 742.0 | 6.20 | 18.1 | 1.6 |
| 749.5 | 6.24 | 18.2 | 1.6 |
| 738.5 | 6.21 | 18.4 | 1.6 |
| 738.7 | 6.21 | 18.4 | 1.6 |

Start-Up Testing Set 2

| | | | |
|---|---|---|---|
| 747.3 | 6.25 | 18.2 | 1.6 |
| 750.0 | 6.24 | 178 | 1.5 |
| 746.8 | 6.21 | 18.3 | 1.6 |
| 746.6 | 6.20 | 18.4 | 1.6 |
| 746.1 | 6.25 | 18.9 | 1.6 |
| 753.6 | 6.23 | 20.5 | 1.8 |
| 750.0 | 6.20 | 19.8 | 1.7 |
| 760.0 | 6.19 | 19.3 | 1.7 |
| 753.6 | 6.29 | 20.2 | 1.7 |
| 745.3 | 6.25 | 18.3 | 1.6 |

Start-Up Testing Set 3

| | | | |
|---|---|---|---|
| 751.4 | 6.23 | 17.8 | 1.5 |
| 756.5 | 6.22 | 15.2 | 1.3 |
| 746.8 | 6.24 | 19.1 | 1.6 |
| 752.6 | 6.22 | 18.4 | 1.6 |
| 751.0 | 6.20 | 18.4 | 1.6 |
| 754.2 | 6.24 | 18.3 | 1.6 |
| 751.4 | 6.25 | 17.8 | 1.5 |
| 751.1 | 6.20 | 16.7 | 1.5 |
| 743.0 | 6.19 | 18.7 | 1.6 |
| 750.2 | 6.26 | 17.2 | 1.5 |

Average

| | | | |
|---|---|---|---|
| 749 | 6.23 | 18.3 | 1.6 |

Target

| | | | |
|---|---|---|---|
| 750.00 (693.75- | n/a | 20 (10-25) | n/a |
| 806.25) | | | |

### Example 9.10—Supplier Pre-Milled API Study

During the development of ER7H and ER9 formulation, it was discovered in the first confirmation batches of ER7H (batch 031466-19) and ER9 (batch 031466-20) that tablet tensile strength was close or below the literature recommendation of 1.7 MPa. Therefore, a step of co-milling API and silicified microcrystalline cellulose at the very beginning of the manufacturing process was included to reduce the API size and thus increase the tabletability and tensile strength. In the second confirmation batch of ER7H (batch 031466-24) and ER9 (batch 031466-26), it was confirmed that the tablet tensile strength increased to above 1.7 MPa after incorporating a co-milling step at the beginning of manufacturing. With this information, the ER7H and ER9 CTM batches also had a co-milling step (see FIGS. 65 and 71, there is a “blending/milling” step before blending with other excipients).

In this study, a milled API lot was used to investigate the feasibility of removing the co-milling step and simplify the manufacturing process.

Table 75 shows the particle size distribution comparison. Lot R00096710 is the reference API lot that has been used for the CTM batches. AI8368-F is the milled API lot. The milled API lot has a smaller d10 and d90 size compared to the reference. Their d50 are similar.

TABLE 75
Particle size distribution of milled API vs. regular API.

| Lot | AI8368-F (milled API) (μm) | R00096710 (reference) (μm) |
|---|---|---|
| d10 | 12.0 | 67.6 |
| d50 | 149.6 | 154.5 |
| d90 | 335.2 | 378.3 |

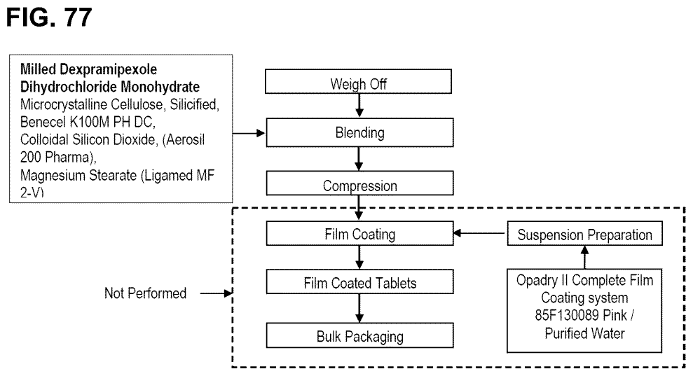

A lab-scale batch using the ER7H formulation with the milled API lot was investigated. The formulation is presented in Table 76. Since the co-milling step was removed, the process was carried out as shown in FIG. 77. Compared to the ER7H CTM batch manufacturing process (FIG. 65), there was no milling step after the weigh off step. The milled API was blended with other excipients directly. Film coating and packaging steps were not performed for this lab-scale batch. The process parameters are summarized in Table 77.

TABLE 76
Formulation used to study the milled API.

| | | 031466-33 | |
|---|---|---|---|
| Material | Function | mg/unit | % w/w |
| Dexpramipexole Dihydrochloride | Drug | 319.01 | 42.53 |
| Monohydrate (milled API) | Substance | | |
| Hypromellose (Benecel K100M PH DC) | Neutral | 112.50 | 15.00 |
| | Matrix | | |
| | Polymer | | |
| Silicified Microcrystalline Cellulose | Diluent | 311.49 | 41.53 |
| (Prosolv HD90) | | | |
| Colloidal Silicon Dioxide | Glidant | 3.50 | 0.47 |
| (Aerosil 200P) | | | |
| Magnesium Stearate | Lubricant | 3.50 | 0.47 |
| (Ligamed MF-2-V) | | | |
| Uncoated Tablet Total | | 750.00 | 100.00 |

TABLE 77
Process parameters of lab scale batch using milled
Dexpramipexole Dihydrochloride Monohydrate.
Process Parameters

| Blending | Equipment | Diffusion blender |
|---|---|---|
| | Pre-Mix (revolutions) | 300 |
| | Lubrication (revolutions) | 80 |
| Compression | Equipment | Korsch XL100 |
| | Tooling | 0.3050″ × 0.6700″ |
| | | Capsule Shape |
| | Target Tablet Weight (mg) | 750 |
| | Turret Speed (rpm) | 20-40 |
| | Feedframe Speed (rpm) | 20-30 |

Two sets of tooling were setup on the Korsch XL100 to keep the balance across the compression turret. Tablet samples compressed at different compression forces and press speeds were collected. During the compression, tablets started to pop from the lower punches/dies during ejection and tablet picking occurred after 5 min of run at 20 rpm turret speed. Tooling was taken out for polishing to rule out tooling defect. After the polishing, tablets still popped, but picking was not observed. Tablets were hard enough to resist the picking.

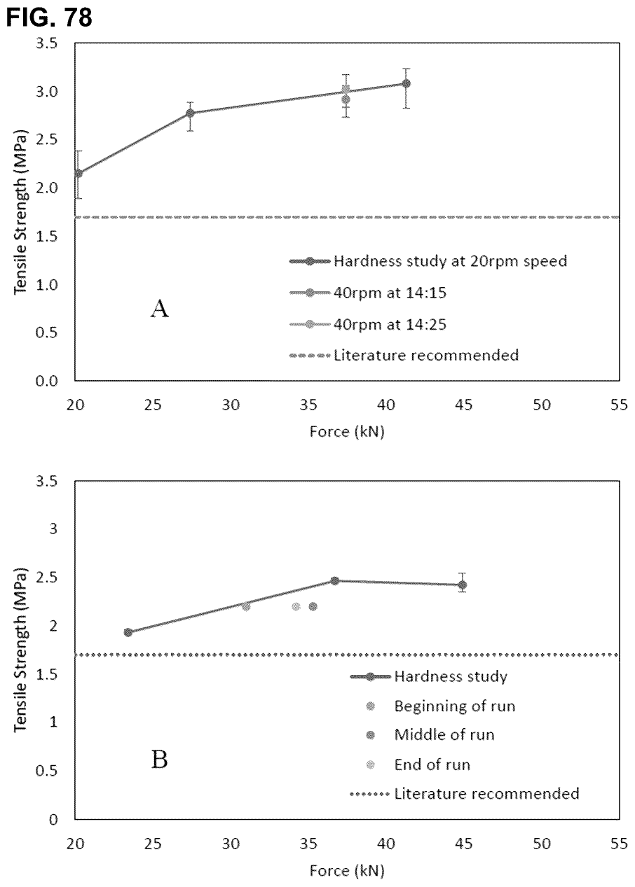

The tabletibility results are shown in FIG. 78. The tablet tensile strength was compared between the batch with milled-API (FIG. 78A) and the batch with regular API incorporated with a co-milling step (FIG. 78B). At the same turret speed of 20 rpm, the batch made with milled API plateaued at around 3.0 MPa after 28 kN of compression force, while the batch with regular API incorporated with a co-milling step plateaued at a lower tensile strength around 2.5 MPa at higher compression force of above 35 kN. Therefore, milled API improved the tabletibility even without a co-milling step. Furthermore, the milled API batch was able to reach 3.0 MPa tensile strength even at 40 rpm turret speed. Tablets usually show lower tensile strength at a higher turret speed due to less dwell time of materials in the die. This also supports the conclusion that milled API is preferred for this process.

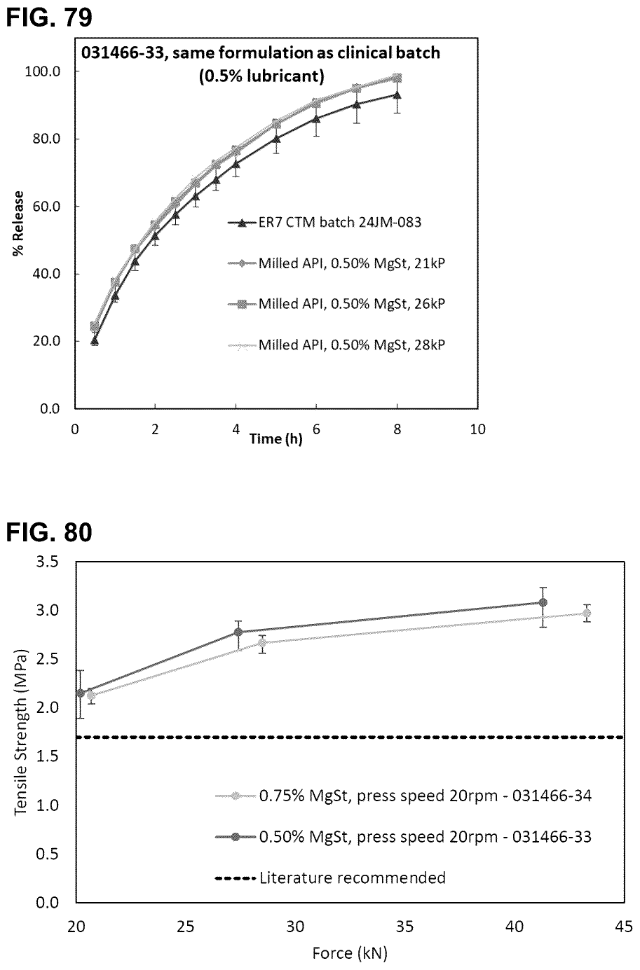

Tablets from different hardnesses were collected and tested for dissolution confirmation. The results were compared with ER7H CTM batch 24JM-083 in FIG. 79. Tablets with hardness of 21 kp, 26 kp and 28 kp (corresponding to 2.2 MPa, 2.8 MPa and 3.1 MPa in FIG. 78A, respectively) showed the same dissolution release. This is because the tablet hardness mainly affects the erosion during dissolution, and matrix tablet release is dominated by diffusion release mechanism. Since the dissolution profile is independent of tablet hardness within the studied range, the compression process can be operated at a wide range. The dissolution profile is also similar to the ER7H CTM batch. The difference is within 5%.

To address the tablets popping during ejection and prevent tablet picking, batch 031466-34 was manufactured, and the lubricant level was increased to 0.75% in the core tablet. The formulation is shown in Table 78. The process parameters are the same as batch 031466-33 as shown in FIG. 77 and Table 77.

TABLE 78
Formulation used to study the milled
API with increased lubricant level.

| | | 031466-34 | |
|---|---|---|---|
| Material | Function | mg/unit | % w/w |
| Dexpramipexole Dihydrochloride | Drug | 319.01 | 42.53 |
| Monohydrate (milled API) | Substance | | |
| Hypromellose (Benecel K100M PH DC) | Neutral | 112.50 | 15.00 |
| | Matrix | | |
| | Polymer | | |
| Silicified Microcrystalline Cellulose | Diluent | 309.39 | 41.25 |
| (Prosolv HD90) | | | |
| Colloidal Silicon Dioxide | Glidant | 3.50 | 0.47 |
| (Aerosil 200P) | | | |
| Magnesium Stearate | Lubricant | 5.60 | 0.75 |
| (Ligamed MF-2-V) | | | |
| Uncoated Tablet Total | | 750.00 | 100.00 |

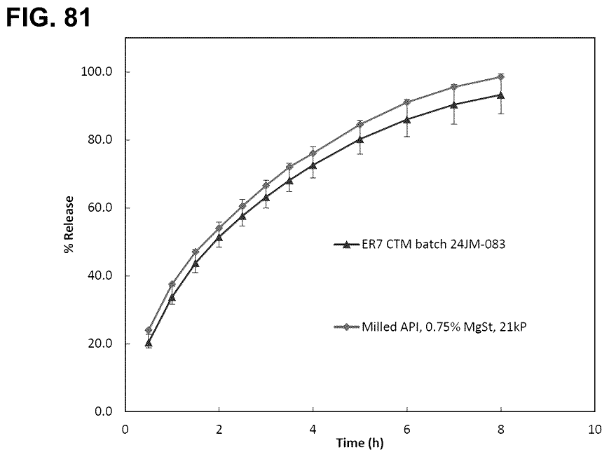

The tabletability results are shown in FIG. 80. Tablet tensile strength decreased slightly as a result of the extra lubricant in the formulation. The tensile strength at 20 kN compression force was still well above the literature recommended level of 1.7 MPa. Tablet popping or picking was not observed during the compression process. The dissolution profile is shown in FIG. 81. The dissolution profile was similar to the ER7H CTM batch. The difference is within 5%.

To conclude, with milled API, the co-milling step of API and SMCC can be removed from the manufacturing process. As a result, the lubricant level in the core tablet was increased to 0.75% in the formulation to prevent tablet popping or picking during the compression process.

### Example 9 Conclusions

In this development, three matrix formulations were developed for Dexpramipexole controlled release tablets. The highlights of the formulations are:
Formulation ER5 comprises a combination of two anionic polymers (Carbopol and sodium carboxymethylcellulose) with 280 mg Dexpramipexole (equivalent to 376 mg Dexpramipexole diHCl).
Formulation ER7H comprises a neutral polymer (hypromellose) with 223 mg Dexpramipexole (equivalent to 300 mg of Dexpramipexole diHCl).
Formulation ER9 comprises an anionic polymer (sodium carboxymethylcellulose) with 223 mg Dexpramipexole (equivalent to 300 mg of Dexpramipexole diHCl).

All formulations met the requirements of targeted dissolution profile.
ER5 is comparable to ER2 clinical dissolution (see FIG. 37).
ER7H and ER8 are within the dissolution target range predicted by IVIVC studies (see FIGS. 46 and 58).

During the development, it was observed that the formulations with sodium carboxymethylcellulose (ER5 and ER9) had a tendency to have an increased release after storage in accelerated stability condition, i.e. 40 C, 75% RH. Nevertheless, ER5 and ER9 were stable under the controlled room temperature and meet the dissolution criteria for clinical dosing.

Milled API (lot AI8368-F) was studied with ER7H formulation. It was concluded that the co-milling step of API and SMCC can be removed from the process. However, the lubricant level in the core tablet needed to be increased to 0.7500 in the formulation.

Additionally, the matrix tablet formulations shown in Table 38 were given to healthy human volunteers once a day (QD; quaque die) to assess bioavailability of dexpramipexole, as described in Example 8, above. The relative bioavailabilities of ER5, ER7, and ER9 formulations are shown in Table 79.

TABLE 79
Pharmacokinetics Relative Bioavailability Assessment (IR vs. ER)

| | | | Test | | Reference | | Geometric | | Intra- |
|---|---|---|---|---|---|---|---|---|---|
| Parameter | | | Geometric | | Geometric | | Mean | 90% | Subject |
| (unit) | Ref. | Test | LSMs (n) | | LSMs (n) | | Ratio (%) | CI | CV (%) |
| AUC₀₋₂₄ | IR | ER5 | 5041 | (18) | 6047 | (18) | 83.36 | 78.11- | 11.8 |
| (h*ng/mL) | | | | | | | | 88.97 | |
| | | ER7 | 6202 | (17) | 6047 | (18) | 102.58 | 95.99- | |
| | | | | | | | | 109.61 | |
| | | ER9 | 6036 | (18) | 6047 | (18) | 99.82 | 93.53- | |
| | | | | | | | | 106.54 | |
| AUC₀₋₄₈ | IR | ER5 | 6935 | (18) | 7302 | (18) | 94.97 | 87.65- | 14.5 |
| (h*ng/mL) | | | | | | | | 102.90 | |
| | | ER7 | 7096 | (17) | 7302 | (18) | 97.17 | 89.55- | |
| | | | | | | | | 105.45 | |
| | | ER9 | 6995 | (18) | 7302 | (18) | 95.79 | 88.41- | |
| | | | | | | | | 103.79 | |
| AUC₀₋₉₆ | IR | ER5 | 7429 | (18) | 7549 | (18) | 98.41 | 90.48- | 15.2 |
| (h*ng/mL) | | | | | | | | 107.04 | |
| | | ER7 | 7319 | (17) | 7549 | (18) | 96.96 | 89.00- | |
| | | | | | | | | 105.63 | |
| | | ER9 | 7210 | (18) | 7549 | (18) | 95.52 | 87.82- | |
| | | | | | | | | 103.89 | |
| Cₘₐₓ | IR | ER5 | 329.4 | (18) | 495.4 | (18) | 66.50 | 61.93- | 12.9 |
| (ng/mL) | | | | | | | | 71.41 | |
| | | ER7 | 456.9 | (17) | 495.4 | (18) | 92.23 | 85.78- | |
| | | | | | | | | 99.17 | |
| | | ER9 | 433.5 | (18) | 495.4 | (18) | 87.52 | 81.50- | |
| | | | | | | | | 93.97 | |

Acceptance Criteria: all AUC 90% CIs within 80-125%. AUC 0-48 & 0-96 most important for multiple dosing.
The ANOVA model includes treatment, sequence, and period as fixed effects and participant nested within sequence as a random effect.. Intra-Subject CV % = 100 × (square root (exp[MSE] − 1)), where MSE = residual variance from ANOVA; CI = Confidence Interval; CV = Coefficient of Variation; ER = Extended Release; IR = Immediate Release; LSM = Least-Squares Mean; n = Number of Subjects. Subject 2012 excluded from ER7H formulations due to early termination in Period 5.

### Conclusion on Examples

As outlined above, sustained release formulations in the form of an orally deliverable tablet cannot exceed a certain maximum weight (typically about 1500 mg), as the tablet must remain swallowable for a human. The comparably high amount of dexpramipexole that is typically used per day (generally, about 50 mg or more dexpramipexole dihydrochloride equivalent, such as about 75 mg, about 150 mg, or about 300 mg dexpramipexole dihydrochloride equivalent, per day) thus restricts the possibilities to formulate a sustained release composition that is suitable for once daily oral administration to a human. This was considered as problematic as a higher amount of drug usually also requires for a higher amount of additional ingredients such as diluents or binders in the sustained release formulation to provide for a composition that can be administered once daily. Further, the high solubility of dexpramipexole dihydrochloride monohydrate across the physiological pH range further complicated the development of a formulation that is suitable for once daily oral administration and from which the release of the active ingredient is at least partly depending on diffusion (such as matrix tablets). Thus, it could not have been reasonably expected that formulating the drug as a sustained release pharmaceutical composition, and let alone in the form of a matrix tablet, that is suitable for once daily oral administration to a human is possible. However, herein, matrix tablet formulations that are suitable for once daily oral administration to a human were developed.

Herein, different matrix tablets containing a comparably high amount of a pharmaceutically acceptable salt of dexpramipexole were evaluated. The matrices tested included hydrophilic neutral polymers (hypromellose, hydroxypropyl cellulose, and poly(ethylene oxide)), lipophilic materials (glycerol dibehenate, carnuba wax), combinations of hydrophilic neutral polymers with lipophilic materials, and hydrophilic anionic polymers (carbomer and sodium carboxymethylcellulose). Out of some of the tested compositions, only those comprising amounts of anionic polymer within the ranges specified herein (i.e., within about 30% to about 60% of one or more anionic polymer by weight of the tablet core) revealed a beneficial in vitro dissolution profile (that makes the tablets suitable for once daily oral administration to a human) and suitable tablet properties (such as tablet hardness).

For some of the tested compositions, although hydrophilic neutral polymers (in particular hypromellose) were included in sustained release, once daily formulations comprising pramipexole within the art, sustained release formulations based on these types of polymers, but lacking the specified amounts of anionic polymer, did not result in an in vitro dissolution profile of dexpramipexole that would make the tablets suitable for once daily oral administration to a human (see in particular Example 1, wherein the tablets were not formulated according to the present disclosure).

Further, although an in principle promising in vitro dissolution profile was achieved for some tablets with lipophilic materials (wherein these tablets did not comprise anionic polymers at the amounts specified herein), mechanic properties such as tensile strengths of those tablets were too low, rendering them unsuitable for (in particular routine and larger-scale) manufacturing (see in particular Example 2, wherein the tablets were not formulated according to the present disclosure).

In strong contrast, matrix tablets comprising one or more anionic polymers at the amounts specified herein (i.e., at about 30% to about 60% by weight of the tablet core) provided in vitro dissolution profiles that render those tablets suitable for once daily oral administration to a human. Also, strong tablets suitable for manufacture also at a large scale were obtained when using anionic polymers at the amounts specified herein. Such beneficial properties (e.g., suitable in vitro dissolution profile and tensile strength) were observed not only by including one type of anionic polymer, but also by combining two different types of anionic polymers (in particular, crosslinked acrylic acid polymer and sodium carboxymethylcellulose) in the tablet core. Such beneficial properties are also observed with a neutral polymer alone at the amounts specified herein (i.e., at about 5% to about 25% by weight of the tablet core). If more than one type of anionic polymer was included, beneficial properties were maintained over a wide range of weight ratios between those anionic polymers (see in particular Example 3).

It was also shown that the in vitro dissolution profiles of matrix tablets comprising one or more anionic polymers at the amounts specified herein were generally independent of the specific manufacturing process and the specific manufacturing scale. Further, the in vitro dissolution profiles of matrix tablets comprising one or more anionic polymers and/or one or more neutral polymers at the amounts specified herein are generally independent of the specific manufacturing process and the specific manufacturing scale. However, comparison of different manufacturing processes revealed that the roller compaction process was particularly favorable for production of the sustained release dexpramipexole formulations of the present disclosure. The roller compaction process was shown to be, for example, very robust as it reduced variability between dissolution profiles of different formulations (see in particular Example 4). Example 5 provides a detailed description and evaluation of such a beneficial manufacturing process according to the present disclosure.

The tablets formulated according to the present disclosure were further demonstrated to be stable over several weeks under CRT conditions (see in particular Example 6).

Two exemplary tablet formulations (“slow” and “fast”) comprising anionic polymers in the amounts as described herein were prepared for evaluation in clinical trials (see in particular Examples 7 and 8).

Following the studies described above, further matrix tablet formulations were prepared. Advantageously, these formulations comprise one or more anionic polymers (carbomer and sodium carboxymethylcellulose), one or more neutral polymers (hypromellose, hydroxypropyl cellulose, and poly(ethylene oxide)), or a combination of one or more anionic polymers with one or more neutral polymers at the amounts specified herein. Although some studies (see Example 1) indicated that when hydrophilic neutral polymers (in particular hypromellose) were included in sustained release, once daily formulation did not result in an in vitro dissolution profile of dexpramipexole that would make the tablets suitable for once daily oral administration to a human, further studies evaluated the amount of neutral polymer in the tablet core. Example 9 provides a detailed description and evaluation of such advantageous formulations. Specifically, a matrix tablet formulation comprising one or more neutral polymers constituting about 5% to about 25% by weight of the tablet core results in an in vitro dissolution profile of dexpramipexole that would make the tablets suitable for once daily oral administration to a human.

The five matrix tablet formulations described in Example 9 comprising one or more anionic polymers and/or one or more neutral polymers in the amounts as described herein were also prepared and evaluated in clinical trials.

1. A sustained release pharmaceutical composition in the form of an orally deliverable tablet comprising a tablet core and dexpramipexole, or a pharmaceutically acceptable salt thereof, in an amount of about 50 mg to about 500 mg of dexpramipexole dihydrochloride equivalent, wherein the tablet core comprises a homogeneous mixture of the amount of dexpramipexole, or a pharmaceutically acceptable salt thereof, and one or more polymers, wherein the one or more polymers constitute about 5% to about 60% by weight of the tablet core, and wherein the weight of the tablet is about 1500 mg or less.

2-4. (canceled)

5. The composition of claim 1, wherein the amount of dexpramipexole, or a pharmaceutically acceptable salt thereof, is about 300 mg to about 400 mg of dexpramipexole dihydrochloride equivalent.

6. (canceled)

7. The composition of claim 65, wherein the amount of dexpramipexole, or a pharmaceutically acceptable salt thereof, is about 376 mg of dexpramipexole dihydrochloride equivalent.

8. The composition of claim 1, wherein the one or more polymers are anionic polymers or neutral polymers.

9. (canceled)

10. (canceled)

11. The composition of claim 8, wherein the weight ratio of dexpramipexole dihydrochloride equivalent to the one or more polymers is about 0.7:1 to about 3.0:1.

12. (canceled)

13. The composition of claim 11, wherein the weight ratio of dexpramipexole dihydrochloride equivalent to the one or more polymers is about 0.9:1.

14. (canceled)

15. (canceled)

16. The composition of claim 1, wherein the pharmaceutically acceptable salt of dexpramipexole is dexpramipexole dihydrochloride monohydrate.

17. The composition of claim 8, wherein the one or more anionic polymers are selected from carboxymethylcellulose, an acrylic acid polymer, a methyl methacrylic acid polymer, a methyl methacrylic acid copolymer, a methacrylic acid polymer, a methacrylic acid copolymer, alignate, carrageenan, xanthan gum, or arabic gum.

18. The composition of claim 17, wherein the one or more anionic polymers are selected from carboxymethylcellulose and an acrylic acid polymer.

19-42. (canceled)

43. The composition of claim 8, wherein the homogeneous mixture comprises a first anionic polymer and a second anionic polymer and wherein the first anionic polymer is carboxymethylcellulose and the second anionic polymer is an acrylic acid polymer.

44-46. (canceled)

47. The composition of claim 43, wherein the amount of carboxymethylcellulose is about 27% to about 34% of sodium carboxymethylcellulose equivalent by weight of the tablet core and the amount of the acrylic acid polymer is about 11% to about 15% by weight of the tablet core.

48-59. (canceled)

60. The composition of claim 8, wherein the one or more neutral polymers is hydroxypropyl methylcellulose or silicified hydroxypropyl methylcellulose.

61. (canceled)

62. (canceled)

63. The composition of claim 1, wherein the homogeneous mixture further comprises a microcrystalline cellulose, magnesium stearate, colloidal silicon dioxide, or any combination thereof.

64. (canceled)

65. The composition of claim 63, wherein the microcrystalline cellulose is silicified microcrystalline cellulose.

66. The composition of claim 1, wherein the homogeneous mixture consists essentially of:
(a) about 315 mg to about 325 mg dexpramipexole dihydrochloride monohydrate;
(b) about 235 mg to about 245 mg sodium carboxymethylcellulose;
(c) about 95 mg to about 105 mg crosslinked acrylic acid polymer;
(d) about 175 mg to about 185 mg silicified microcrystalline cellulose;
(e) about 5 mg to about 7 mg magnesium stearate; and
(f) about 3 mg to about 5 mg colloidal silicon dioxide.

67. The composition of claim 66, wherein the homogeneous mixture consists essentially of:
(a) about 319 mg dexpramipexole dihydrochloride monohydrate;
(b) about 238 mg sodium carboxymethylcellulose;
(c) about 102 mg crosslinked acrylic acid polymer;
(d) about 180 mg silicified microcrystalline cellulose;
(e) about 6 mg magnesium stearate; and
(f) about 4 mg colloidal silicon dioxide.

68. The composition of claim 1, wherein the homogeneous mixture consists essentially of:
(a) about 315 mg to about 325 mg dexpramipexole dihydrochloride monohydrate;
(b) about 295 mg to about 305 mg sodium carboxymethylcellulose;
(c) about 125 mg to about 135 mg crosslinked acrylic acid polymer;
(d) about 140 mg to about 150 mg silicified microcrystalline cellulose;
(e) about 6 mg to about 8 mg magnesium stearate; and
(f) about 4 mg to about 6 mg colloidal silicon dioxide.

69. The composition of claim 68, wherein the homogeneous mixture consists essentially of:
(a) about 319 mg dexpramipexole dihydrochloride monohydrate;
(b) about 298 mg sodium carboxymethylcellulose;
(c) about 128 mg crosslinked acrylic acid polymer;
(d) about 145 mg silicified microcrystalline cellulose;
(e) about 7 mg magnesium stearate; and
(f) about 5 mg colloidal silicon dioxide.

70. The composition of claim 1, wherein the homogeneous mixture consists essentially of:
a. about 390 mg to about 410 mg dexpramipexole dihydrochloride monohydrate;
b. about 325 mg to about 341 mg sodium carboxymethylcellulose;
c. about 135 mg to about 145 mg crosslinked acrylic acid polymer;
d. about 230 mg to about 240 mg silicified microcrystalline cellulose;
e. about 5 mg to about 10 mg magnesium stearate; and
f. about 4 mg to about 7 mg colloidal silicon dioxide.

71. The composition of claim 70, wherein the homogeneous mixture consists essentially of:
a. about 400 mg dexpramipexole dihydrochloride monohydrate;
b. about 333 mg sodium carboxymethylcellulose;
c. about 143 mg crosslinked acrylic acid polymer;
d. about 236 mg silicified microcrystalline cellulose;
e. about 8 mg magnesium stearate; and
f. about 6 mg colloidal silicon dioxide.

72. The composition of claim 1, wherein the homogeneous mixture consists essentially of:
a. about 315 mg to about 325 mg dexpramipexole dihydrochloride monohydrate;
b. about 110 mg to about 120 mg hydroxypropyl methylcellulose or silicified hydroxypropyl methylcellulose;
c. about 310 mg to about 320 mg silicified microcrystalline cellulose;
d. about 3 mg to about 5 mg magnesium stearate; and
e. about 3 mg to about 5 mg colloidal silicon dioxide.

73. The composition of claim 72, wherein the homogeneous mixture consists essentially of:
a. about 319 mg dexpramipexole dihydrochloride monohydrate;
b. about 113 mg hydroxypropyl methylcellulose or silicified hydroxypropyl methylcellulose;
c. about 313 mg silicified microcrystalline cellulose;
d. about 4 mg magnesium stearate; and
e. about 4 mg colloidal silicon dioxide.

74. The composition of claim 1, wherein the homogeneous mixture consists essentially of:
a. about 315 mg to about 325 mg dexpramipexole dihydrochloride monohydrate;
b. about 200 mg to about 220 mg sodium carboxymethylcellulose;
c. about 210 mg to about 220 mg silicified microcrystalline cellulose;
d. about 3 mg to about 5 mg magnesium stearate; and
e. about 3 mg to about 5 mg colloidal silicon dioxide.

75. The composition of claim 74, wherein the homogeneous mixture consists essentially of:
a. about 319 mg dexpramipexole dihydrochloride monohydrate;
b. about 210 mg sodium carboxymethylcellulose;
c. about 214 mg silicified microcrystalline cellulose;
d. about 4 mg magnesium stearate; and
e. about 4 mg colloidal silicon dioxide.

76-78. (canceled)

79. The composition of claim 1, wherein the weight of the tablet is about 1300 mg or less.

80-88. (canceled)

89. The composition of claim 1, wherein about 40% to about 80% of dexpramipexole, or the pharmaceutically acceptable salt thereof, is released at about 12 hours of incubation of the tablet in about 900 mL of 50 mM monobasic potassium phosphate buffer, pH 6.8, at a temperature of 37±0.5° C. as measured using an USP type I apparatus operated at a spindle rotation speed of about 100 rpm.

90. (canceled)

91. (canceled)

92. The composition of claim 1, comprising 0.015% or less pramipexole, or a pharmaceutically acceptable salt thereof, by weight of the tablet.

93. A method of treating or preventing asthma in a human subject in need thereof, the method comprising orally administering to the human subject the composition of claim 1.

94. (canceled)

95. A method of treating or preventing chronic obstructive pulmonary disease in a human subject in need thereof, the method comprising orally administering to the human subject the composition of claim 1.

96. A method of treating or preventing an eosinophilic disorder in a human subject in need thereof, the method comprising orally administering to the human subject the composition of claim 1.

97-101. (canceled)

102. A method of manufacturing densified granules for preparing a sustained release pharmaceutical composition comprising dexpramipexole, or a pharmaceutically acceptable salt thereof, the method comprising:
preparing a pre-blend by mixing dexpramipexole, or a pharmaceutically acceptable salt thereof, and one or more polymers; and
preparing densified granules from the pre-blend;
wherein dexpramipexole, or the pharmaceutically acceptable salt thereof, constitutes about 30% to about 50% of dexpramipexole dihydrochloride monohydrate equivalent by weight of the densified granules and the one or more polymers constitute about 5% to about 60% by weight of the densified granules.

103. (canceled)

104. The method of claim 102, wherein the one or more polymers are anionic polymers.

105-112. (canceled)

113. The method of claim 104, wherein the one or more anionic polymers are selected from carboxymethylcellulose, an acrylic acid polymer, a methacrylic acid polymer, a methacrylic acid copolymer, alignate, carrageenan, xanthan gum, or arabic gum.

114-127. (canceled)

128. The method of claim 102, wherein the one or more polymers are neutral polymers.

129-131. (canceled)

132. A method of manufacturing a blend for preparing a sustained release pharmaceutical composition comprising dexpramipexole, or a pharmaceutically acceptable salt thereof, the method comprising:
preparing a pre-blend by mixing dexpramipexole, or a pharmaceutically acceptable salt thereof, and one or more polymers;
preparing densified granules from the pre-blend; and
preparing a blend by mixing the densified granules with a glidant and a lubricant;
wherein dexpramipexole, or the pharmaceutically acceptable salt thereof, constitutes about 30% to about 50% of dexpramipexole dihydrochloride monohydrate equivalent by weight of the blend and the one or more polymers constitute about 5% to about 60% by weight of the blend.

133. The method of claim 132, wherein the one or more polymers are anionic polymers.

134. (canceled)

135. The method of claim 132, wherein the one or more polymers are neutral polymers.

136. (canceled)

137. A method of manufacturing a sustained release pharmaceutical composition in the form of an orally deliverable tablet comprising a tablet core with dexpramipexole, or a pharmaceutically acceptable salt thereof, the method comprising:
preparing a pre-blend by mixing dexpramipexole, or a pharmaceutically acceptable salt thereof, and one or more polymers;
preparing densified granules from the pre-blend;
preparing a blend by mixing the densified granules with a glidant and a lubricant; and
compressing the blend into a tablet core;
wherein dexpramipexole, or the pharmaceutically acceptable salt thereof, constitutes about 30% to about 50% of dexpramipexole dihydrochloride monohydrate equivalent by weight of the blend and the one or more polymers constitute about 5% to about 50% by weight of the blend.

138. The method of claim 137, wherein the one or more polymers are anionic polymers.

139-165. (canceled)

166. The method of claim 137, wherein the one or more polymers are neutral polymers.

167-184. (canceled)

185. The method of claim 137, wherein preparing the blend comprises:
mixing the densified granules with the glidant thereby providing a mixture of the densified granules and the glidant; and
mixing the mixture of the densified granules and the glidant with the lubricant thereby providing the blend.

186. The method of claim 102, wherein preparing the pre-blend comprises:
mixing dexpramipexole, or a pharmaceutically acceptable salt thereof, the one or more polymers and a microcrystalline cellulose thereby providing a mixture of dexpramipexole, or a pharmaceutically acceptable salt thereof, the one or more polymers and the microcrystalline cellulose; and
mixing the mixture of dexpramipexole, or a pharmaceutically acceptable salt thereof, the one or more polymers and the microcrystalline cellulose with magnesium stearate thereby providing the pre-blend.

187-190. (canceled)

191. The method of claim 102, wherein the densified granules are prepared by dry granulation.

192. The method of claim 102, wherein the densified granules are prepared by roller compaction.

193-249. (canceled)
